SEQUENCE LISTING (103 sequences)

SEQ ID NO: 1 - DNA, 20 bp, synthetic construct
  source 1..20: mol_type = other DNA
tgggtggattacctgaggtc

SEQ ID NO: 2 - DNA, 17 bp, synthetic construct
  source 1..17: mol_type = other DNA
ccaggctggactcgaac

SEQ ID NO: 3 - DNA, 20 bp, synthetic construct
  source 1..20: mol_type = other DNA
gaatcacttgaacccgggag

SEQ ID NO: 4 - DNA, 22 bp, synthetic construct
  source 1..22: mol_type = other DNA
ccacctcctctaacttcacctc

SEQ ID NO: 5 - DNA, 21 bp, synthetic construct
  source 1..21: mol_type = other DNA
gttctgattcttgggggttcc

SEQ ID NO: 6 - DNA, 21 bp, synthetic construct
  source 1..21: mol_type = other DNA
ggaagctgagttggcaggatc

SEQ ID NO: 7 - DNA, 18 bp, synthetic construct
  source 1..18: mol_type = other DNA
cgacagacatggtccctg

SEQ ID NO: 8 - DNA, 25 bp, synthetic construct
  source 1..25: mol_type = other DNA
gttgctgagtagatgcagtctactg

SEQ ID NO: 9 - DNA, 24 bp, synthetic construct
  source 1..24: mol_type = other DNA
tttagagggagacacaagttgata

SEQ ID NO: 10 - DNA, 130 bp, synthetic construct
  source 1..130: mol_type = other DNA
gaccgcttcacttagagtgaaggtgggctgcttgcatcagcctaatgtcgagaagtgctttcttcggaaagtaaccctcgaaacaaagaaaggaatgcaacgagggagacacaagttgatattttatttt

SEQ ID NO: 11 - DNA, 128 bp, synthetic construct
  source 1..128: mol_type = other DNA
gaccgcttcacttagagtgaaggtgggctgcttgcatcagcctaatgtcgagaagtgctttcttcggaaagtaaccctcgaaacaaagaaaggaatgcaacgagggagacacaagttgattttatttt

SEQ ID NO: 12 - DNA, 126 bp, synthetic construct
  source 1..126: mol_type = other DNA
gaccgcttcacttagagtgaaggtgggctgcttgcatcagcctaatgtcgagaagtgctttcttcggaaagtaaccctcgaaacaaagaaaggaatgcaacgagggagacacaagttttttatttt

SEQ ID NO: 13 - DNA, 124 bp, synthetic construct
  source 1..124: mol_type = other DNA
gaccgcttcacttagagtgaaggtgggctgcttgcatcagcctaatgtcgagaagtgctttcttcggaaagtaaccctcgaaacaaagaaaggaatgcaacgagggagacacaagttttatttt

SEQ ID NO: 14 - DNA, 122 bp, synthetic construct
  source 1..122: mol_type = other DNA
gaccgcttcacttagagtgaaggtgggctgcttgcatcagcctaatgtcgagaagtgctttcttcggaaagtaaccctcgaaacaaagaaaggaatgcaacgagggagacacattttatttt

SEQ ID NO: 15 - DNA, 120 bp, synthetic construct
  source 1..120: mol_type = other DNA
gaccgcttcacttagagtgaaggtgggctgcttgcatcagcctaatgtcgagaagtgctttcttcggaaagtaaccctcgaaacaaagaaaggaatgcaacgagggagacattttatttt

SEQ ID NO: 16 - DNA, 118 bp, synthetic construct
  source 1..118: mol_type = other DNA
gaccgcttcacttagagtgaaggtgggctgcttgcatcagcctaatgtcgagaagtgctttcttcggaaagtaaccctcgaaacaaagaaaggaatgcaacgagggagattttatttt

SEQ ID NO: 17 - DNA, 24 bp, synthetic construct
  source 1..24: mol_type = other DNA
ttatgggctgttgtcagacccaac

SEQ ID NO: 18 - RNA, 213 nt, synthetic construct
  source 1..213: mol_type = other RNA
ggtggagcggttcacgaccgcgacctcaacgccgcccggaacatcaagcgggaagggctttcgcaaatcgtcgtcgcggggcacgcggagacgttaaacgctcggggagagggtgtcagacctgcgatagcgggcagccctcgatgaagcgagaatccaacggctttagccgttggagtgtcaagggctgttgtcagacccaacttttatttt

SEQ ID NO: 19 - RNA, 211 nt, synthetic construct
  source 1..211: mol_type = other RNA
ggtggagcggttcacgaccgcgacctcaacgccgcccggaacatcaagcgggaagggctttcgcaaatcgtcgtcgcggggcacgcggagacgttaaacgctcggggagagggtgtcagacctgcgatagcgggcagccctcgatgaagcgagaatccaacggctttagccgttggagtgtcaagggctgttgtcagacccattttatttt

SEQ ID NO: 20 - RNA, 209 nt, synthetic construct
  source 1..209: mol_type = other RNA
ggtggagcggttcacgaccgcgacctcaacgccgcccggaacatcaagcgggaagggctttcgcaaatcgtcgtcgcggggcacgcggagacgttaaacgctcggggagagggtgtcagacctgcgatagcgggcagccctcgatgaagcgagaatccaacggctttagccgttggagtgtcaagggctgttgtcagaccttttatttt

SEQ ID NO: 21 - RNA, 207 nt, synthetic construct
  source 1..207: mol_type = other RNA
ggtggagcggttcacgaccgcgacctcaacgccgcccggaacatcaagcgggaagggctttcgcaaatcgtcgtcgcggggcacgcggagacgttaaacgctcggggagagggtgtcagacctgcgatagcgggcagccctcgatgaagcgagaatccaacggctttagccgttggagtgtcaagggctgttgtcagattttatttt

SEQ ID NO: 22 - RNA, 205 nt, synthetic construct
  source 1..205: mol_type = other RNA
ggtggagcggttcacgaccgcgacctcaacgccgcccggaacatcaagcgggaagggctttcgcaaatcgtcgtcgcggggcacgcggagacgttaaacgctcggggagagggtgtcagacctgcgatagcgggcagccctcgatgaagcgagaatccaacggctttagccgttggagtgtcaagggctgttgtcattttatttt

SEQ ID NO: 23 - RNA, 203 nt, synthetic construct
  source 1..203: mol_type = other RNA
ggtggagcggttcacgaccgcgacctcaacgccgcccggaacatcaagcgggaagggctttcgcaaatcgtcgtcgcggggcacgcggagacgttaaacgctcggggagagggtgtcagacctgcgatagcgggcagccctcgatgaagcgagaatccaacggctttagccgttggagtgtcaagggctgttgtttttatttt

SEQ ID NO: 24 - RNA, 201 nt, synthetic construct
  source 1..201: mol_type = other RNA
ggtggagcggttcacgaccgcgacctcaacgccgcccggaacatcaagcgggaagggctttcgcaaatcgtcgtcgcggggcacgcggagacgttaaacgctcggggagagggtgtcagacctgcgatagcgggcagccctcgatgaagcgagaatccaacggctttagccgttggagtgtcaagggctgttttttatttt

SEQ ID NO: 25 - DNA, 21 bp, synthetic construct
  source 1..21: mol_type = other DNA
ccaaagaagaagcggaaggtc

SEQ ID NO: 26 - protein, 7 aa, synthetic construct
PKKKRKV

SEQ ID NO: 27 - DNA, 1173 bp, synthetic construct
  source 1..1173: mol_type = other DNA
atgaccacccaccgcaaggtgtacagataccgcatcgagcccacccccgtgcaggagagcaagctgtacatgctggccggcagccggagattcgtgttcaactgggccctggcccggagaagagagcactacgccgaaaccggcaagaccctgggatacaacgcccaggcaggagagctgaccgccctgaaaaaccaagaagaaaccagctggctgaaagagtccgactcacaactgctgcagcaggcactgaaagacgtggaaagagccttcgtgaacttcttcgaaaaacgggccagattccccagattcaaaagcaagaaaaccgacacccccagattcagaatcccccaaagagtgagaatagagggcagcagagtgtacgtgcccaaggtggggtgggtgaagctgagaaagagtcaggagatagaaggaaaaaccaaaagcgccaccttcaagagggaggcagatggccattggtacgtgctgctggtgagtgagttcgagatgcctgatgtgcctctgcccccagtgcccgagagcgaggtggtgggaatcgacctgggactgaaggacttctacgtcctgtctgacgggggcaggaaggaggcccccagattcgctagaaagggccagcgaaaactgagaagagctgcaagaagacatagcaagtgcacccggggaagcaacagaaaggccaaggcaaagaggaagctggcaagagtgcacagacagatcgcaaaccagagaaaagacttcgtgcacaaggccaccagcggcctcgtgcagcagtaccaaggcttctgcatcgaaaacctgagcatcaagggcatggccaaaaccaaactgtcaaaaagcgtgctggacgcagccctgggggaatttagaagacagctggcctataaagcccagtggcatagaaagtggctggcagtgatagacagatggttccccagcagcaagctgtgcggagagtgcggcagcattaacgccgatctgaccctgagcgacagagaatggacctgcgagtgtggcgcagtgcacgacagagatctgaacgccgctcgcaacatcaagagagagggcctgagtcagattgtggtggccggacatgccgaaaccctgaacgccagaggggagggcgtgagacccgccattgccggaagccctaga

SEQ ID NO: 28 - protein, 391 aa, synthetic construct
MTTHRKVYRYRIEPTPVQESKLYMLAGSRRFVFNWALARRREHYAETGKTLGYNAQAGELTALKNQEETSWLKESDSQLLQQALKDVERAFVNFFEKRARFPRFKSKKTDTPRFRIPQRVRIEGSRVYVPKVGWVKLRKSQEIEGKTKSATFKREADGHWYVLLVSEFEMPDVPLPPVPESEVVGIDLGLKDFYVLSDGGRKEAPRFARKGQRKLRRAARRHSKCTRGSNRKAKAKRKLARVHRQIANQRKDFVHKATSGLVQQYQGFCIENLSIKGMAKTKLSKSVLDAALGEFRRQLAYKAQWHRKWLAVIDRWFPSSKLCGECGSINADLTLSDREWTCECGAVHDRDLNAARNIKREGLSQIVVAGHAETLNARGEGVRPAIAGSPR

SEQ ID NO: 29 - DNA, 1173 bp, synthetic construct
  source 1..1173: mol_type = other DNA
atgaccacccaccgcaaggtgtacagataccgcatcgagcccacccccgtgcaggagagcaagctgtacatgctggccggcagccggagattcgtgttcaactgggccctggcccggagaagagagcactacgccgaaaccggcaagaccctgggatacaacgcccaggcaggagagctgaccgccctgaaaaaccaagaagaaaccagctggctgaaagagtccgactcacaactgctgcagcaggcactgaaagacgtggaaagagccttcgtgaacttcttcgaaaaacgggccagattccccagattcaaaagcaagaaaaccgacacccccagattcagaatcccccaaagagtgagaatagagggcagcagagtgtacgtgcccaaggtggggtgggtgaagctgagaaagagtcaggagatagaaggaaaaaccaaaagcgccaccttcaagagggaggcagatggccattggtacgtgctgctggtgagtgagttcgagatgcctgatgtgcctctgcccccagtgcccgagagcgaggtggtgggaatcgccctgggactgaaggacttctacgtcctgtctgacgggggcaggaaggaggcccccagattcgctagaaagggccagcgaaaactgagaagagctgcaagaagacatagcaagtgcacccggggaagcaacagaaaggccaaggcaaagaggaagctggcaagagtgcacagacagatcgcaaaccagagaaaagacttcgtgcacaaggccaccagcggcctcgtgcagcagtaccaaggcttctgcatcgaaaacctgagcatcaagggcatggccaaaaccaaactgtcaaaaagcgtgctggacgcagccctgggggaatttagaagacagctggcctataaagcccagtggcatagaaagtggctggcagtgatagacagatggttccccagcagcaagctgtgcggagagtgcggcagcattaacgccgatctgaccctgagcgacagagaatggacctgcgagtgtggcgcagtgcacgacagagatctgaacgccgctcgcaacatcaagagagagggcctgagtcagattgtggtggccggacatgccgaaaccctgaacgccagaggggagggcgtgagacccgccattgccggaagccctaga

SEQ ID NO: 30 - protein, 391 aa, synthetic construct
MTTHRKVYRYRIEPTPVQESKLYMLAGSRRFVFNWALARRREHYAETGKTLGYNAQAGELTALKNQEETSWLKESDSQLLQQALKDVERAFVNFFEKRARFPRFKSKKTDTPRFRIPQRVRIEGSRVYVPKVGWVKLRKSQEIEGKTKSATFKREADGHWYVLLVSEFEMPDVPLPPVPESEVVGIALGLKDFYVLSDGGRKEAPRFARKGQRKLRRAARRHSKCTRGSNRKAKAKRKLARVHRQIANQRKDFVHKATSGLVQQYQGFCIENLSIKGMAKTKLSKSVLDAALGEFRRQLAYKAQWHRKWLAVIDRWFPSSKLCGECGSINADLTLSDREWTCECGAVHDRDLNAARNIKREGLSQIVVAGHAETLNARGEGVRPAIAGSPR

SEQ ID NO: 31 - DNA, 99 bp, synthetic construct
  source 1..99: mol_type = other DNA
agcagggctgaccccaagaagaagaggaaggtgtcgccagggatccgtcgacttgacgcgttgatatcaacaagtttgtacaaaaaagcaggctacaaa

SEQ ID NO: 32 - protein, 33 aa, synthetic construct
SRADPKKKRKVSPGIRRLDALISTSLYKKAGYK

SEQ ID NO: 33 - DNA, 1593 bp, synthetic construct
  source 1..1593: mol_type = other DNA
gaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttt

SEQ ID NO: 34 - protein, 531 aa, synthetic construct
EASGSGRADALDDFDLDMLGSDALDDFDLDMLGSDALDDFDLDMLGSDALDDFDLDMLINSRSSGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 35 - DNA, 48 bp, synthetic construct
  source 1..48: mol_type = other DNA
aaaaggccggcggccacgaaaaaggccggccaggcaaaaaagaaaaag

SEQ ID NO: 36 - protein, 16 aa, synthetic construct
KRPAATKKAGQAKKKK

SEQ ID NO: 37 - DNA, 642 bp, synthetic construct
  source 1..642: mol_type = other DNA
atggcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacacaggctatggaactcagtcaactccccagggatatggctcgactggcggctatggcagtagccagagctcccaatcgtcttacgggcagcagtcctcctaccctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtgga

SEQ ID NO: 38 - protein, 214 aa, synthetic construct
MASNDYTQQATQSYGAYPTQPGQGYSQQSSQPYGQQSYSGYSQSTDTSGYGQSSYSSYGQSQNTGYGTQSTPQGYGSTGGYGSSQSSQSSYGQQSSYPGYGQQPAPSSTSGSYGSSSQSSSYGQPQSGSYSQQPSYGGQQQSYGQQQSYNPPQGYGQQNQYNSSSGGGGGGGGGGNYGQDQSSMSSGGGSGGGYGNQDQSGGGGSGGYGQQDRG

SEQ ID NO: 39 - DNA, 183 bp, synthetic construct
  source 1..183: mol_type = other DNA
gtggagcggttcacgaccgcgacctcaacgccgcccggaacatcaagcgggaagggctttcgcaaatcgtcgtcgcggggcacgcggagacgttaaacgctcggggagagggtgtcagacctgcgatagcgggcagccctcgatgaagcgagaatccaacggctttagccgttggagtgtcaa

SEQ ID NO: 40 - DNA, 54 bp, synthetic construct
  source 1..54: mol_type = other DNA
gagggcagaggaagtctgctaacatgcggtgacgtcgaggagaatcctggccca

SEQ ID NO: 41 - DNA, 714 bp, synthetic construct
  source 1..714: mol_type = other DNA
gtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 42 - DNA, 241 bp, synthetic construct
  source 1..241: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggac

SEQ ID NO: 43 - DNA, 2865 bp, synthetic construct
  source 1..2865: mol_type = other DNA
atgaccacccaccgcaaggtgtacagataccgcatcgagcccacccccgtgcaggagagcaagctgtacatgctggccggcagccggagattcgtgttcaactgggccctggcccggagaagagagcactacgccgaaaccggcaagaccctgggatacaacgcccaggcaggagagctgaccgccctgaaaaaccaagaagaaaccagctggctgaaagagtccgactcacaactgctgcagcaggcactgaaagacgtggaaagagccttcgtgaacttcttcgaaaaacgggccagattccccagattcaaaagcaagaaaaccgacacccccagattcagaatcccccaaagagtgagaatagagggcagcagagtgtacgtgcccaaggtggggtgggtgaagctgagaaagagtcaggagatagaaggaaaaaccaaaagcgccaccttcaagagggaggcagatggccattggtacgtgctgctggtgagtgagttcgagatgcctgatgtgcctctgcccccagtgcccgagagcgaggtggtgggaatcgacctgggactgaaggacttctacgtcctgtctgacgggggcaggaaggaggcccccagattcgctagaaagggccagcgaaaactgagaagagctgcaagaagacatagcaagtgcacccggggaagcaacagaaaggccaaggcaaagaggaagctggcaagagtgcacagacagatcgcaaaccagagaaaagacttcgtgcacaaggccaccagcggcctcgtgcagcagtaccaaggcttctgcatcgaaaacctgagcatcaagggcatggccaaaaccaaactgtcaaaaagcgtgctggacgcagccctgggggaatttagaagacagctggcctataaagcccagtggcatagaaagtggctggcagtgatagacagatggttccccagcagcaagctgtgcggagagtgcggcagcattaacgccgatctgaccctgagcgacagagaatggacctgcgagtgtggcgcagtgcacgacagagatctgaacgccgctcgcaacatcaagagagagggcctgagtcagattgtggtggccggacatgccgaaaccctgaacgccagaggggagggcgtgagacccgccattgccggaagccctagaagcagggctgaccccaagaagaagaggaaggtgtcgccagggatccgtcgacttgacgcgttgatatcaacaagtttgtacaaaaaagcaggctacaaagaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttt

SEQ ID NO: 44 - protein, 955 aa, synthetic construct
MTTHRKVYRYRIEPTPVQESKLYMLAGSRRFVFNWALARRREHYAETGKTLGYNAQAGELTALKNQEETSWLKESDSQLLQQALKDVERAFVNFFEKRARFPRFKSKKTDTPRFRIPQRVRIEGSRVYVPKVGWVKLRKSQEIEGKTKSATFKREADGHWYVLLVSEFEMPDVPLPPVPESEVVGIDLGLKDFYVLSDGGRKEAPRFARKGQRKLRRAARRHSKCTRGSNRKAKAKRKLARVHRQIANQRKDFVHKATSGLVQQYQGFCIENLSIKGMAKTKLSKSVLDAALGEFRRQLAYKAQWHRKWLAVIDRWFPSSKLCGECGSINADLTLSDREWTCECGAVHDRDLNAARNIKREGLSQIVVAGHAETLNARGEGVRPAIAGSPRSRADPKKKRKVSPGIRRLDALISTSLYKKAGYKEASGSGRADALDDFDLDMLGSDALDDFDLDMLGSDALDDFDLDMLGSDALDDFDLDMLINSRSSGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 45 - DNA, 3555 bp, synthetic construct
  source 1..3555: mol_type = other DNA
atgaccacccaccgcaaggtgtacagataccgcatcgagcccacccccgtgcaggagagcaagctgtacatgctggccggcagccggagattcgtgttcaactgggccctggcccggagaagagagcactacgccgaaaccggcaagaccctgggatacaacgcccaggcaggagagctgaccgccctgaaaaaccaagaagaaaccagctggctgaaagagtccgactcacaactgctgcagcaggcactgaaagacgtggaaagagccttcgtgaacttcttcgaaaaacgggccagattccccagattcaaaagcaagaaaaccgacacccccagattcagaatcccccaaagagtgagaatagagggcagcagagtgtacgtgcccaaggtggggtgggtgaagctgagaaagagtcaggagatagaaggaaaaaccaaaagcgccaccttcaagagggaggcagatggccattggtacgtgctgctggtgagtgagttcgagatgcctgatgtgcctctgcccccagtgcccgagagcgaggtggtgggaatcgccctgggactgaaggacttctacgtcctgtctgacgggggcaggaaggaggcccccagattcgctagaaagggccagcgaaaactgagaagagctgcaagaagacatagcaagtgcacccggggaagcaacagaaaggccaaggcaaagaggaagctggcaagagtgcacagacagatcgcaaaccagagaaaagacttcgtgcacaaggccaccagcggcctcgtgcagcagtaccaaggcttctgcatcgaaaacctgagcatcaagggcatggccaaaaccaaactgtcaaaaagcgtgctggacgcagccctgggggaatttagaagacagctggcctataaagcccagtggcatagaaagtggctggcagtgatagacagatggttccccagcagcaagctgtgcggagagtgcggcagcattaacgccgatctgaccctgagcgacagagaatggacctgcgagtgtggcgcagtgcacgacagagatctgaacgccgctcgcaacatcaagagagagggcctgagtcagattgtggtggccggacatgccgaaaccctgaacgccagaggggagggcgtgagacccgccattgccggaagccctagaagcagggctgaccccaagaagaagaggaaggtgtcgccagggatccgtcgacttgacgcgttgatatcaacaagtttgtacaaaaaagcaggctacaaagaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgtttaaaaggccggcggccacgaaaaaggccggccaggcaaaaaagaaaaagatggcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacacaggctatggaactcagtcaactccccagggatatggctcgactggcggctatggcagtagccagagctcccaatcgtcttacgggcagcagtcctcctaccctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtgga

SEQ ID NO: 46 - protein, 1185 aa, synthetic construct
MTTHRKVYRYRIEPTPVQESKLYMLAGSRRFVFNWALARRREHYAETGKTLGYNAQAGELTALKNQEETSWLKESDSQLLQQALKDVERAFVNFFEKRARFPRFKSKKTDTPRFRIPQRVRIEGSRVYVPKVGWVKLRKSQEIEGKTKSATFKREADGHWYVLLVSEFEMPDVPLPPVPESEVVGIALGLKDFYVLSDGGRKEAPRFARKGQRKLRRAARRHSKCTRGSNRKAKAKRKLARVHRQIANQRKDFVHKATSGLVQQYQGFCIENLSIKGMAKTKLSKSVLDAALGEFRRQLAYKAQWHRKWLAVIDRWFPSSKLCGECGSINADLTLSDREWTCECGAVHDRDLNAARNIKREGLSQIVVAGHAETLNARGEGVRPAIAGSPRSRADPKKKRKVSPGIRRLDALISTSLYKKAGYKEASGSGRADALDDFDLDMLGSDALDDFDLDMLGSDALDDFDLDMLGSDALDDFDLDMLINSRSSGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLFKRPAATKKAGQAKKKKMASNDYTQQATQSYGAYPTQPGQGYSQQSSQPYGQQSYSGYSQSTDTSGYGQSSYSSYGQSQNTGYGTQSTPQGYGSTGGYGSSQSSQSSYGQQSSYPGYGQQPAPSSTSGSYGSSSQSSSYGQPQSGSYSQQPSYGGQQQSYGQQQSYNPPQGYGQQNQYNSSSGGGGGGGGGGNYGQDQSSMSSGGGSGGGYGNQDQSGGGGSGGYGQQDRG

SEQ ID NO: 47 - DNA, 3555 bp, synthetic construct
  source 1..3555: mol_type = other DNA
atgaccacccaccgcaaggtgtacagataccgcatcgagcccacccccgtgcaggagagcaagctgtacatgctggccggcagccggagattcgtgttcaactgggccctggcccggagaagagagcactacgccgaaaccggcaagaccctgggatacaacgcccaggcaggagagctgaccgccctgaaaaaccaagaagaaaccagctggctgaaagagtccgactcacaactgctgcagcaggcactgaaagacgtggaaagagccttcgtgaacttcttcgaaaaacgggccagattccccagattcaaaagcaagaaaaccgacacccccagattcagaatcccccaaagagtgagaatagagggcagcagagtgtacgtgcccaaggtggggtgggtgaagctgagaaagagtcaggagatagaaggaaaaaccaaaagcgccaccttcaagagggaggcagatggccattggtacgtgctgctggtgagtgagttcgagatgcctgatgtgcctctgcccccagtgcccgagagcgaggtggtgggaatcgacctgggactgaaggacttctacgtcctgtctgacgggggcaggaaggaggcccccagattcgctagaaagggccagcgaaaactgagaagagctgcaagaagacatagcaagtgcacccggggaagcaacagaaaggccaaggcaaagaggaagctggcaagagtgcacagacagatcgcaaaccagagaaaagacttcgtgcacaaggccaccagcggcctcgtgcagcagtaccaaggcttctgcatcgaaaacctgagcatcaagggcatggccaaaaccaaactgtcaaaaagcgtgctggacgcagccctgggggaatttagaagacagctggcctataaagcccagtggcatagaaagtggctggcagtgatagacagatggttccccagcagcaagctgtgcggagagtgcggcagcattaacgccgatctgaccctgagcgacagagaatggacctgcgagtgtggcgcagtgcacgacagagatctgaacgccgctcgcaacatcaagagagagggcctgagtcagattgtggtggccggacatgccgaaaccctgaacgccagaggggagggcgtgagacccgccattgccggaagccctagaagcagggctgaccccaagaagaagaggaaggtgtcgccagggatccgtcgacttgacgcgttgatatcaacaagtttgtacaaaaaagcaggctacaaagaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgtttaaaaggccggcggccacgaaaaaggccggccaggcaaaaaagaaaaagatggcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacacaggctatggaactcagtcaactccccagggatatggctcgactggcggctatggcagtagccagagctcccaatcgtcttacgggcagcagtcctcctaccctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtgga

SEQ ID NO: 48 - protein, 1185 aa, synthetic construct
MTTHRKVYRYRIEPTPVQESKLYMLAGSRRFVFNWALARRREHYAETGKTLGYNAQAGELTALKNQEETSWLKESDSQLLQQALKDVERAFVNFFEKRARFPRFKSKKTDTPRFRIPQRVRIEGSRVYVPKVGWVKLRKSQEIEGKTKSATFKREADGHWYVLLVSEFEMPDVPLPPVPESEVVGIDLGLKDFYVLSDGGRKEAPRFARKGQRKLRRAARRHSKCTRGSNRKAKAKRKLARVHRQIANQRKDFVHKATSGLVQQYQGFCIENLSIKGMAKTKLSKSVLDAALGEFRRQLAYKAQWHRKWLAVIDRWFPSSKLCGECGSINADLTLSDREWTCECGAVHDRDLNAARNIKREGLSQIVVAGHAETLNARGEGVRPAIAGSPRSRADPKKKRKVSPGIRRLDALISTSLYKKAGYKEASGSGRADALDDFDLDMLGSDALDDFDLDMLGSDALDDFDLDMLGSDALDDFDLDMLINSRSSGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLFKRPAATKKAGQAKKKKMASNDYTQQATQSYGAYPTQPGQGYSQQSSQPYGQQSYSGYSQSTDTSGYGQSSYSSYGQSQNTGYGTQSTPQGYGSTGGYGSSQSSQSSYGQQSSYPGYGQQPAPSSTSGSYGSSSQSSSYGQPQSGSYSQQPSYGGQQQSYGQQQSYNPPQGYGQQNQYNSSSGGGGGGGGGGNYGQDQSSMSSGGGSGGGYGNQDQSGGGGSGGYGQQDRG

SEQ ID NO: 49 - DNA, 3555 bp, synthetic construct; identical to SEQ ID NO: 45
  source 1..3555: mol_type = other DNA

SEQ ID NO: 50 - protein, 1185 aa, synthetic construct; identical to SEQ ID NO: 46

SEQ ID NO: 51 - DNA, 3600 bp, synthetic construct
  source 1..3600: mol_type = other DNA
ccaaagaagaagcggaaggtcggtatccacggagtcccagcagccatgaccacccaccgcaaggtgtacagataccgcatcgagcccacccccgtgcaggagagcaagctgtacatgctggccggcagccggagattcgtgttcaactgggccctggcccggagaagagagcactacgccgaaaccggcaagaccctgggatacaacgcccaggcaggagagctgaccgccctgaaaaaccaagaagaaaccagctggctgaaagagtccgactcacaactgctgcagcaggcactgaaagacgtggaaagagccttcgtgaacttcttcgaaaaacgggccagattccccagattcaaaagcaagaaaaccgacacccccagattcagaatcccccaaagagtgagaatagagggcagcagagtgtacgtgcccaaggtggggtgggtgaagctgagaaagagtcaggagatagaaggaaaaaccaaaagcgccaccttcaagagggaggcagatggccattggtacgtgctgctggtgagtgagttcgagatgcctgatgtgcctctgcccccagtgcccgagagcgaggtggtgggaatcgacctgggactgaaggacttctacgtcctgtctgacgggggcaggaaggaggcccccagattcgctagaaagggccagcgaaaactgagaagagctgcaagaagacatagcaagtgcacccggggaagcaacagaaaggccaaggcaaagaggaagctggcaagagtgcacagacagatcgcaaaccagagaaaagacttcgtgcacaaggccaccagcggcctcgtgcagcagtaccaaggcttctgcatcgaaaacctgagcatcaagggcatggccaaaaccaaactgtcaaaaagcgtgctggacgcagccctgggggaatttagaagacagctggcctataaagcccagtggcatagaaagtggctggcagtgatagacagatggttccccagcagcaagctgtgcggagagtgcggcagcattaacgccgatctgaccctgagcgacagagaatggacctgcgagtgtggcgcagtgcacgacagagatctgaacgccgctcgcaacatcaagagagagggcctgagtcagattgtggtggccggacatgccgaaaccctgaacgccagaggggagggcgtgagacccgccattgccggaagccctagaagcagggctgaccccaagaagaagaggaaggtgtcgccagggatccgtcgacttgacgcgttgatatcaacaagtttgtacaaaaaagcaggctacaaagaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgtttaaaaggccggcggccacgaaaaaggccggccaggcaaaaaagaaaaagatggcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacacaggctatggaactcagtcaactccccagggatatggctcgactggcggctatggcagtagccagagctcccaatcgtcttacgggcagcagtcctcctaccctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtgga

SEQ ID NO: 52 - DNA, 8297 bp, synthetic construct
  source 1..8297: mol_type = other DNA
tttagcgcgtgcgccaattctgcagacaaatggctctagaggtaccgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgcgccaccatggccccaaagaagaagcggaaggtcggtatccacggagtcccagcagccatgaccacccaccgcaaggtgtacagataccgcatcgagcccacccccgtgcaggagagcaagctgtacatgctggccggcagccggagattcgtgttcaactgggccctggcccggagaagagagcactacgccgaaaccggcaagaccctgggatacaacgcccaggcaggagagctgaccgccctgaaaaaccaagaagaaaccagctggctgaaagagtccgactcacaactgctgcagcaggcactgaaagacgtggaaagagccttcgtgaacttcttcgaaaaacgggccagattccccagattcaaaagcaagaaaaccgacacccccagattcagaatcccccaaagagtgagaatagagggcagcagagtgtacgtgcccaaggtggggtgggtgaagctgagaaagagtcaggagatagaaggaaaaaccaaaagcgccaccttcaagagggaggcagatggccattggtacgtgctgctggtgagtgagttcgagatgcctgatgtgcctctgcccccagtgcccgagagcgaggtggtgggaatcgacctgggactgaaggacttctacgtcctgtctgacgggggcaggaaggaggcccccagattcgctagaaagggccagcgaaaactgagaagagctgcaagaagacatagcaagtgcacccggggaagcaacagaaaggccaaggcaaagaggaagctggcaagagtgcacagacagatcgcaaaccagagaaaagacttcgtgcacaaggccaccagcggcctcgtgcagcagtaccaaggcttctgcatcgaaaacctgagcatcaagggcatggccaaaaccaaactgtcaaaaagcgtgctggacgcagccctgggggaatttagaagacagctggcctataaagcccagtggcatagaaagtggctggcagtgatagacagatggttccccagcagcaagctgtgcggagagtgcggcagcattaacgccgatctgaccctgagcgacagagaatggacctgcgagtgtggcgcagtgcacgacagagatctgaacgccgctcgcaacatcaagagagagggcctgagtcagattgtggtggccggacatgccgaaaccctgaacgccagaggggagggcgtgagacccgccattgccggaagccctagaagcagggctgaccccaagaagaagaggaaggtgtcgccagggatccgtcgacttgacgcgttgatatcaacaagtttgtacaaaaaagcaggctacaaagaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgtttaaaaggccggcggccacgaaaaaggccggccaggcaaaaaagaaaaagatggcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacacaggctatggaactcagtcaactccccagggatatggctcgactggcggctatggcagtagccagagctcccaatcgtcttacgggcagcagtcctcctaccctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtggagaattcggcagtggagagggcagaggaagtctgctaacatgcggtgacgtcgaggagaatcctggcccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtggagcggttcacgaccgcgacctcaacgccgcccggaacatcaagcgggaagggctttcgcaaatcgtcgtcgcggggcacgcggagacgttaaacgctcggggagagggtgtcagacctgcgatagcgggcagccctcgatgaagcgagaatccaacggctttagccgttggagtgtcaagggctgttgtcagacccaacttttattttttgttttagagctagaaatagtaagttaaaataaggctagtccgtt

SEQ ID NO: 53 - DNA, 8295 bp, synthetic construct
  source 1..8295: mol_type = other DNA
tttagcgcgtgcgccaattctgcagacaaatggctctagaggtaccgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgcgccaccatggccccaaagaagaagcggaaggtcggtatccacggagtcccagcagccatgaccacccaccgcaaggtgtacagataccgcatcgagcccacccccgtgcaggagagcaagctgtacatgctggccggcagccggagattcgtgttcaactgggccctggcccggagaagagagcactacgccgaaaccggcaagaccctgggatacaacgcccaggcaggagagctgaccgccctgaaaaaccaagaagaaaccagctggctgaaagagtccgactcacaactgctgcagcaggcactgaaagacgtggaaagagccttcgtgaacttcttcgaaaaacgggccagattccccagattcaaaagcaagaaaaccgacacccccagattcagaatcccccaaagagtgagaatagagggcagcagagtgtacgtgcccaaggtggggtgggtgaagctgagaaagagtcaggagatagaaggaaaaaccaaaagcgccaccttcaagagggaggcagatggccattggtacgtgctgctggtgagtgagttcgagatgcctgatgtgcctctgcccccagtgcccgagagcgaggtggtgggaatcgacctgggactgaaggacttctacgtcctgtctgacgggggcaggaaggaggcccccagattcgctagaaagggccagcgaaaactgagaagagctgcaagaagacatagcaagtgcacccggggaagcaacagaaaggccaaggcaaagaggaagctggcaagagtgcacagacagatcgcaaaccagagaaaagacttcgtgcacaaggccaccagcggcctcgtgcagcagtaccaaggcttctgcatcgaaaacctgagcatcaagggcatggccaaaaccaaactgtcaaaaagcgtgctggacgcagccctgggggaatttagaagacagctggcctataaagcccagtggcatagaaagtggctggcagtgatagacagatggttccccagcagcaagctgtgcggagagtgcggcagcattaacgccgatctgaccctgagcgacagagaatggacctgcgagtgtggcgcagtgcacgacagagatctgaacgccgctcgcaacatcaagagagagggcctgagtcagattgtggtggccggacatgccgaaaccctgaacgccagaggggagggcgtgagacccgccattgccggaagccctagaagcagggctgaccccaagaagaagaggaaggtgtcgccagggatccgtcgacttgacgcgttgatatcaacaagtttgtacaaaaaagcaggctacaaagaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgtttaaaaggccggcggccacgaaaaaggccggccaggcaaaaaagaaaaagatggcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacacaggctatggaactcagtcaactccccagggatatggctcgactggcggctatggcagtagccagagctcccaatcgtcttacgggcagcagtcctcctaccctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtggagaattcggcagtggagagggcagaggaagtctgctaacatgcggtgacgtcgaggagaatcctggcccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtggagcggttcacgaccgcgacctcaacgccgcccggaacatcaagcgggaagggctttcgcaaatcgtcgtcgcggggcacgcggagacgttaaacgctcggggagagggtgtcagacctgcgatagcgggcagccctcgatgaagcgagaatccaacggctttagccgttggagtgtcaagggctgttgtcagacccattttattttttgttttagagctagaaatagtaagttaaaataaggctagtccgtt

SEQ ID NO: 54 - DNA, 8293 bp, synthetic construct
  source 1..8293: mol_type = other DNA
tttagcgcgtgcgccaattctgcagacaaatggctctagaggtaccgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgcgccaccatggccccaaagaagaagcggaaggtcggtatccacggagtcccagcagccatgaccacccaccgcaaggtgtacagataccgcatcgagcccacccccgtgcaggagagcaagctgtacatgctggccggcagccggagattcgtgttcaactgggccctggcccggagaagagagcactacgccgaaaccggcaagaccctgggatacaacgcccaggcaggagagctgaccgccctgaaaaaccaagaagaaaccagctggctgaaagagtccgactcacaactgctgcagcaggcactgaaagacgtggaaagagccttcgtgaacttcttcgaaaaacgggccagattccccagattcaaaagcaagaaaaccgacacccccagattcagaatcccccaaagagtgagaatagagggcagcagagtgtacgtgcccaaggtggggtgggtgaagctgagaaagagtcaggagatagaaggaaaaaccaaaagcgccaccttcaagagggaggcagatggccattggtacgtgctgctggtgagtgagttcgagatgcctgatgtgcctctgcccccagtgcccgagagcgaggtggtgggaatcgacctgggactgaaggacttctacgtcctgtctgacgggggcaggaaggaggcccccagattcgctagaaagggccagcgaaaactgagaagagctgcaagaagacatagcaagtgcacccggggaagcaacagaaaggccaaggcaaagaggaagctggcaagagtgcacagacagatcgcaaaccagagaaaagacttcgtgcacaaggccaccagcggcctcgtgcagcagtaccaaggcttctgcatcgaaaacctgagcatcaagggcatggccaaaaccaaactgtcaaaaagcgtgctggacgcagccctgggggaatttagaagacagctggcctataaagcccagtggcatagaaagtggctggcagtgatagacagatggttccccagcagcaagctgtgcggagagtgcggcagcattaacgccgatctgaccctgagcgacagagaatggacctgcgagtgtggcgcagtgcacgacagagatctgaacgccgctcgcaacatcaagagagagggcctgagtcagattgtggtggccggacatgccgaaaccctgaacgccagaggggagggcgtgagacccgccattgccggaagccctagaagcagggctgaccccaagaagaagaggaaggtgtcgccagggatccgtcgacttgacgcgttgatatcaacaagtttgtacaaaaaagcaggctacaaagaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgtttaaaaggccggcggccacgaaaaaggccggccaggcaaaaaagaaaaagatggcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacacaggctatggaactcagtcaactccccagggatatggctcgactggcggctatggcagtagccagagctcccaatcgtcttacgggcagcagtcctcctaccctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtggagaattcggcagtggagagggcagaggaagtctgctaacatgcggtgacgtcgaggagaatcctggcccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtggagcggttcacgaccgcgacctcaacgccgcccggaacatcaagcgggaagggctttcgcaaatcgtcgtcgcggggcacgcggagacgttaaacgctcggggagagggtgtcagacctgcgatagcgggcagccctcgatgaagcgagaatccaacggctttagccgttggagtgtcaagggctgttgtcagaccttttattttttgttttagagctagaaatagtaagttaaaataaggctagtccgtt

SEQ ID NO: 55 - DNA, 8291 bp, synthetic construct
  source 1..8291: mol_type = other DNA
tttagcgcgtgcgccaattctgcagacaaatggctctagaggtaccgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgcgccaccatggccccaaagaagaagcggaaggtcggtatccacggagtcccagcagccatgaccacccaccgcaaggtgtacagataccgcatcgagcccacccccgtgcaggagagcaagctgtacatgctggccggcagccggagattcgtgttcaactgggccctggcccggagaagagagcactacgccgaaaccggcaagaccctgggatacaacgcccaggcaggagagctgaccgccctgaaaaaccaagaagaaaccagctggctgaaagagtccgactcacaactgctgcagcaggcactgaaagacgtggaaagagccttcgtgaacttcttcgaaaaacgggccagattccccagattcaaaagcaagaaaaccgacacccccagattcagaatcccccaaagagtgagaatagagggcagcagagtgtacgtgcccaaggtggggtgggtgaagctgagaaagagtcaggagatagaaggaaaaaccaaaagcgccaccttcaagagggaggcagatggccattggtacgtgctgctggtgagtgagttcgagatgcctgatgtgcctctgcccccagtgcccgagagcgaggtggtgggaatcgacctgggactgaaggacttctacgtcctgtctgacgggggcaggaaggaggcccccagattcgctagaaagggccagcgaaaactgagaagagctgcaagaagacatagcaagtgcacccggggaagcaacagaaaggccaaggcaaagaggaagctggcaagagtgcacagacagatcgcaaaccagagaaaagacttcgtgcacaaggccaccagcggcctcgtgcagcagtaccaaggcttctgcatcgaaaacctgagcatcaagggcatggccaaaaccaaactgtcaaaaagcgtgctggacgcagccctgggggaatttagaagacagctggcctataaagcccagtggcatagaaagtggctggcagtgatagacagatggttccccagcagcaagctgtgcggagagtgcggcagcattaacgccgatctgaccctgagcgacagagaatggacctgcgagtgtggcgcagtgcacgacagagatctgaacgccgctcgcaacatcaagagagagggcctgagtcagattgtggtggccggacatgccgaaaccctgaacgccagaggggagggcgtgagacccgccattgccggaagccctagaagcagggctgaccccaagaagaagaggaaggtgtcgccagggatccgtcgacttgacgcgttgatatcaacaagtttgtacaaaaaagcaggctacaaagaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgtttaaaaggccggcggccacgaaaaaggccggccaggcaaaaaagaaaaagatggcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacacaggctatggaactcagtcaactccccagggatatggctcgactggcggctatggcagtagccagagctcccaatcgtcttacgggcagcagtcctcctaccctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtggagaattcggcagtggagagggcagaggaagtctgctaacatgcggtgacgtcgaggagaatcctggcccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtggagcggttcacgaccgcgacctcaacgccgcccggaacatcaagcgggaagggctttcgcaaatcgtcgtcgcggggcacgcggagacgttaaacgctcggggagagggtgtcagacctgcgatagcgggcagccctcgatgaagcgagaatccaacggctttagccgttggagtgtcaagggctgttgtcagattttattttttgttttagagctagaaatagtaagttaaaataaggctagtccgtt

SEQ ID NO: 56 - DNA, 8289 bp, synthetic construct
  source 1..8289: mol_type = other DNA
tttagcgcgtgcgccaattctgcagacaaatggctctagaggtaccgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgcgccaccatggccccaaagaagaagcggaaggtcggtatccacggagtcccagcagccatgaccacccaccgcaaggtgtacagataccgcatcgagcccacccccgtgcaggagagcaagctgtacatgctggccggcagccggagattcgtgttcaactgggccctggcccggagaagagagcactacgccgaaaccggcaagaccctgggatacaacgcccaggcaggagagctgaccgccctgaaaaaccaagaagaaaccagctggctgaaagagtccgactcacaactgctgcagcaggcactgaaagacgtggaaagagccttcgtgaacttcttcgaaaaacgggccagattccccagattcaaaagcaagaaaaccgacacccccagattcagaatcccccaaagagtgagaatagagggcagcagagtgtacgtgcccaaggtggggtgggtgaagctgagaaagagtcaggagatagaaggaaaaaccaaaagcgccaccttcaagagggaggcagatggccattggtacgtgctgctggtgagtgagttcgagatgcctgatgtgcctctgcccccagtgcccgagagcgaggtggtgggaatcgacctgggactgaaggacttctacgtcctgtctgacgggggcaggaaggaggcccccagattcgctagaaagggccagcgaaaactgagaagagctgcaagaagacatagcaagtgcacccggggaagcaacagaaaggccaaggcaaagaggaagctggcaagagtgcacagacagatcgcaaaccagagaaaagacttcgtgcacaaggccaccagcggcctcgtgcagcagtaccaaggcttctgcatcgaaaacctgagcatcaagggcatggccaaaaccaaactgtcaaaaagcgtgctggacgcagccctgggggaatttagaagacagctggcctataaagcccagtggcatagaaagtggctggcagtgatagacagatggttccccagcagcaagctgtgcggagagtgcggcagcattaacgccgatctgaccctgagcgacagagaatggacctgcgagtgtggcgcagtgcacgacagagatctgaacgccgctcgcaacatcaagagagagggcctgagtcagattgtggtggccggacatgccgaaaccctgaacgccagaggggagggcgtgagacccgccattgccggaagccctagaagcagggctgaccccaagaagaagaggaaggtgtcgccagggatccgtcgacttgacgcgttgatatcaacaagtttgtacaaaaaagcaggctacaaagaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgtttaaaaggccggcggccacgaaaaaggccggccaggcaaaaaagaaaaagatggcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacacaggctatggaactcagtcaactccccagggatatggctcgactggcggctatggcagtagccagagctcccaatcgtcttacgggcagcagtcctcctaccctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtggagaattcggcagtggagagggcagaggaagtctgctaacatgcggtgacgtcgaggagaatcctggcccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtggagcggttcacgaccgcgacctcaacgccgcccggaacatcaagcgggaagggctttcgcaaatcgtcgtcgcggggcacgcggagacgttaaacgctcggggagagggtgtcagacctgcgatagcgggcagccctcgatgaagcgagaatccaacggctttagccgttggagtgtcaagggctgttgtcattttattttttgttttagagctagaaatagtaagttaaaataaggctagtccgtt

SEQ ID NO: 57 - DNA, 8287 bp, synthetic construct
  source 1..8287: mol_type = other DNA
tttagcgcgtgcgccaattctgcagacaaatggctctagaggtaccgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgcgccaccatggccccaaagaagaagcggaaggtcggtatccacggagtcccagcagccatgaccacccaccgcaaggtgtacagataccgcatcgagcccacccccgtgcaggagagcaagctgtacatgctggccggcagccggagattcgtgttcaactgggccctggcccggagaagagagcactacgccgaaaccggcaagaccctgggatacaacgcccaggcaggagagctgaccgccctgaaaaaccaagaagaaaccagctggctgaaagagtccgactcacaactgctgcagcaggcactgaaagacgtggaaagagccttcgtgaacttcttcgaaaaacgggccagattccccagattcaaaagcaagaaaaccgacacccccagattcagaatcccccaaagagtgagaatagagggcagcagagtgtacgtgcccaaggtggggtgggtgaagctgagaaagagtcaggagatagaaggaaaaaccaaaagcgccaccttcaagagggaggcagatggccattggtacgtgctgctggtgagtgagttcgagatgcctgatgtgcctctgcccccagtgcccgagagcgaggtggtgggaatcgacctgggactgaaggacttctacgtcctgtctgacgggggcaggaaggaggcccccagattcgctagaaagggccagcgaaaactgagaagagctgcaagaagacatagcaagtgcacccggggaagcaacagaaaggccaaggcaaagaggaagctggcaagagtgcacagacagatcgcaaaccagagaaaagacttcgtgcacaaggccaccagcggcctcgtgcagcagtaccaaggcttctgcatcgaaaacctgagcatcaagggcatggccaaaaccaaactgtcaaaaagcgtgctggacgcagccctgggggaatttagaagacagctggcctataaagcccagtggcatagaaagtggctggcagtgatagacagatggttccccagcagcaagctgtgcggagagtgcggcagcattaacgccgatctgaccctgagcgacagagaatggacctgcgagtgtggcgcagtgcacgacagagatctgaacgccgctcgcaacatcaagagagagggcctgagtcagattgtggtggccggacatgccgaaaccctgaacgccagaggggagggcgtgagacccgccattgccggaagccctagaagcagggctgaccccaagaagaagaggaaggtgtcgccagggatccgtcgacttgacgcgttgatatcaacaagtttgtacaaaaaagcaggctacaaagaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgtttaaaaggccggcggccacgaaaaaggccggccaggcaaaaaagaaaaagatggcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacacaggctatggaactcagtcaactccccagggatatggctcgactggcggctatggcagtagccagagctcccaatcgtcttacgggcagcagtcctcctaccctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtggagaattcggcagtggagagggcagaggaagtctgctaacatgcggtgacgtcgaggagaatcctggcccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtggagcggttcacgaccgcgacctcaacgccgcccggaacatcaagcgggaagggctttcgcaaatcgtcgtcgcggggcacgcggagacgttaaacgctcggggagagggtgtcagacctgcgatagcgggcagccctcgatgaagcgagaatccaacggctttagccgttggagtgtcaagggctgttgtttttattttttgttttagagctagaaatagtaagttaaaataaggctagtccgtt

SEQ ID NO: 58 - DNA, 8285 bp, synthetic construct
  source 1..8285: mol_type = other DNA
tttagcgcgtgcgccaattctgcagacaaatggctctagaggtaccgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgcgccaccatggccccaaagaagaagcggaaggtcggtatccacggagtcccagcagccatgaccacccaccgcaaggtgtacagataccgcatcgagcccacccccgtgcaggagagcaagctgtacatgctggccggcagccggagattcgtgttcaactgggccctggcccggagaagagagcactacgccgaaaccggcaagaccctgggatacaacgcccaggcaggagagctgaccgccctgaaaaaccaagaagaaaccagctggctgaaagagtccgactcacaactgctgcagcaggcactgaaagacgtggaaagagccttcgtgaacttcttcgaaaaacgggccagattccccagattcaaaagcaagaaaaccgacacccccagattcagaatcccccaaagagtgagaatagagggcagcagagtgtacgtgcccaaggtggggtgggtgaagctgagaaagagtcaggagatagaaggaaaaaccaaaagcgccaccttcaagagggaggcagatggccattggtacgtgctgctggtgagtgagttcgagatgcctgatgtgcctctgcccccagtgcccgagagcgaggtggtgggaatcgacctgggactgaaggacttctacgtcctgtctgacgggggcaggaaggaggcccccagattcgctagaaagggccagcgaaaactgagaagagctgcaagaagacatagcaagtgcacccggggaagcaacagaaaggccaaggcaaagaggaagctggcaagagtgcacagacagatcgcaaaccagagaaaagacttcgtgcacaaggccaccagcggcctcgtgcagcagtaccaaggcttctgcatcgaaaacctgagcatcaagggcatggccaaaaccaaactgtcaaaaagcgtgctggacgcagccctgggggaatttagaagacagctggcctataaagcccagtggcatagaaagtggctggcagtgatagacagatggttccccagcagcaagctgtgcggagagtgcggcagcattaacgccgatctgaccctgagcgacagagaatggacctgcgagtgtggcgcagtgcacgacagagatctgaacgccgctcgcaacatcaagagagagggcctgagtcagattgtggtggccggacatgccgaaaccctgaacgccagaggggagggcgtgagacccgccattgccggaagccctagaagcagggctgaccccaagaagaagaggaaggtgtcgccagggatccgtcgacttgacgcgttgatatcaacaagtttgtacaaaaaagcaggctacaaagaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgtttaaaaggccggcggccacgaaaaaggccggccaggcaaaaaagaaaaagatggcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacacaggctatggaactcagtcaactccccagggatatggctcgactggcggctatggcagtagccagagctcccaatcgtcttacgggcagcagtcctcctaccctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtggagaattcggcagtggagagggcagaggaagtctgctaacatgcggtgacgtcgaggagaatcctggcccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtggagcggttcacgaccgcgacctcaacgccgcccggaacatcaagcgggaagggctttcgcaaatcgtcgtcgcggggcacgcggagacgttaaacgctcggggagagggtgtcagacctgcgatagcgggcagccctcgatgaagcgagaatccaacggctttagccgttggagtgtcaagggctgttttttattttttgttttagagctagaaatagtaagttaaaataaggctagtccgtt

SEQ ID NO: 59 - DNA, 8297 bp, synthetic construct
  source 1..8297: mol_type = other DNA
tttagcgcgtgcgccaattctgcagacaaatggctctagaggtaccgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgcgccaccatggccccaaagaagaagcggaaggtcggtatccacggagtcccagcagccatgaccacccaccgcaaggtgtacagataccgcatcgagcccacccccgtgcaggagagcaagctgtacatgctggccggcagccggagattcgtgttcaactgggccctggcccggagaagagagcactacgccgaaaccggcaagaccctgggatacaacgcccaggcaggagagctgaccgccctgaaaaaccaagaagaaaccagctggctgaaagagtccgactcacaactgctgcagcaggcactgaaagacgtggaaagagccttcgtgaacttcttcgaaaaacgggccagattccccagattcaaaagcaagaaaaccgacacccccagattcagaatcccccaaagagtgagaatagagggcagcagagtgtacgtgcccaaggtggggtgggtgaagctgagaaagagtcaggagatagaaggaaaaaccaaaagcgccaccttcaagagggaggcagatggccattggtacgtgctgctggtgagtgagttcgagatgcctgatgtgcctctgcccccagtgcccgagagcgaggtggtgggaatcgccctgggactgaaggacttctacgtcctgtctgacgggggcaggaaggaggcccccagattcgctagaaagggccagcgaaaactgagaagagctgcaagaagacatagcaagtgcacccggggaagcaacagaaaggccaaggcaaagaggaagctggcaagagtgcacagacagatcgcaaaccagagaaaagacttcgtgcacaaggccaccagcggcctcgtgcagcagtaccaaggcttctgcatcgaaaacctgagcatcaagggcatggccaaaaccaaactgtcaaaaagcgtgctggacgcagccctgggggaatttagaagacagctggcctataaagcccagtggcatagaaagtggctggcagtgatagacagatggttccccagcagcaagctgtgcggagagtgcggcagcattaacgccgatctgaccctgagcgacagagaatggacctgcgagtgtggcgcagtgcacgacagagatctgaacgccgctcgcaacatcaagagagagggcctgagtcagattgtggtggccggacatgccgaaaccctgaacgccagaggggagggcgtgagacccgccattgccggaagccctagaagcagggctgaccccaagaagaagaggaaggtgtcgccagggatccgtcgacttgacgcgttgatatcaacaagtttgtacaaaaaagcaggctacaaagaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgtttaaaaggccggcggccacgaaaaaggccggccaggcaaaaaagaaaaagatggcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacacaggctatggaactcagtcaactccccagggatatggctcgactggcggctatggcagtagccagagctcccaatcgtcttacgggcagcagtcctcctaccctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtggagaattcggcagtggagagggcagaggaagtctgctaacatgcggtgacgtcgaggagaatcctggcccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtggagcggttcacgaccgcgacctcaacgccgcccggaacatcaagcgggaagggctttcgcaaatcgtcgtcgcggggcacgcggagacgttaaacgctcggggagagggtgtcagacctgcgatagcgggcagccctcgatgaagcgagaatccaacggctttagccgttggagtgtcaagggctgttgtcagacccaacttttattttttgttttagagctagaaatagtaagttaaaataaggctagtccgtt

SEQ ID NO: 60 - RNA, 188 nt, synthetic construct
  source 1..188: mol_type = other RNA
ggtggagacctcaacgccgcccggaacatcaagcgggaagggctttcgcaaatcgtcgtcgcggggcacgcggagacgttaaacgctcggggagagggtgtcagacctgcgatagcgggcagccctcgatgaagcgagaatccaacggctttagccgttggagtgtcaagggctgttgtttttatttt

SEQ ID NO: 61 - RNA, 194 nt, synthetic construct
  source 1..194: mol_type = other RNA
ggtggagcggttcacgaccgcgccgcccggaacatcaagcgggaagggctttcgcaaatcgtcgtcgcggggcacgcggagacgttaaacgctcggggagagggtgtcagacctgcgatagcgggcagccctcgatgaagcgagaatccaacggctttagccgttggagtgtcaagggctgttgtttttatttt

SEQ ID NO: 62 - RNA, 175 nt, synthetic construct
  source 1..175: mol_type = other RNA
ggtggagcggttcacgaccgcgacctcaactttcgcaaatcgtcgtcgcggggcacgcggagacgttaaacgctcggggagagggtgtcagacctgcgatagcgggcagccctcgatgaagcgagaatccaacggctttagccgttggagtgtcaagggctgttgtttttatttt

SEQ ID NO: 63 - RNA, 178 nt, synthetic construct
  source 1..178: mol_type = other RNA
ggtggagcggttcacgaccgcgacctcaacgccgcccggaacatcaagcgggaagggctttcgcaaatttaaacgctcggggagagggtgtcagacctgcgatagcgggcagccctcgatgaagcgagaatccaacggctttagccgttggagtgtcaagggctgttgtttttatttt

SEQ ID NO: 64 - RNA, 188 nt, synthetic construct
  source 1..188: mol_type = other RNA
ggtggagcggttcacgaccgcgacctcaacgccgcccggaacatcaagcgggaagggctttcgcaaatcgtcgtcgcggggcacgcggagacggagggtgtcagacctgcgatagcgggcagccctcgatgaagcgagaatccaacggctttagccgttggagtgtcaagggctgttgtttttatttt

SEQ ID NO: 65 - RNA, 169 nt, synthetic construct
  source 1..169: mol_type = other RNA
ggtggagcggttcacgaccgcgacctcaacgccgcccggaacatcaagcgggaagggctttcgcaaatcgtcgtcgcggggcacgcggagacgttaaacgctcggggagatgaagcgagaatccaacggctttagccgttggagtgtcaagggctgttgtttttatttt

SEQ ID NO: 66 - RNA, 181 nt, synthetic construct
  source 1..181: mol_type = other RNA
ggtggagcggttcacgaccgcgacctcaacgccgcccggaacatcaagcgggaagggctttcgcaaatcgtcgtcgcggggcacgcggagacgttaaacgctcggggagagggtgtcagacctgcgatagcgggcagccctcgatgaagcgagaagtgtcaagggctgttgtttttatttt

SEQ ID NO: 67 - RNA, 193 nt, synthetic construct
  source 1..193: mol_type = other RNA
ggtggagcggttcacgaccgcgacctcaacgccgcccggaacatcaagcgggaagggccgtcgtcgcggggcacgcggagacgttaaacgctcggggagagggtgtcagacctgcgatagcgggcagccctcgatgaagcgagaatccaacggctttagccgttggagtgtcaagggctgttgtttttatttt

SEQ ID NO: 68 - RNA, 179 nt, synthetic construct
  source 1..179: mol_type = other RNA
ggtggagccgcccggaacatcaagcgggaagggctttcgcaaatcgtcgtcgcggggcacgcggagacgttaaacgctcggggagagggtgtcagacctgcgatagcgggcagccctcgatgaagcgagaatccaacggctttagccgttggagtgtcaagggctgttgtttttatttt

SEQ ID NO: 69 - RNA, 151 nt, synthetic construct
  source 1..151: mol_type = other RNA
ggtggatttcgcaaatcgtcgtcgcggggcacgcggagacgttaaacgctcggggagagggtgtcagacctgcgatagcgggcagccctcgatgaagcgagaatccaacggctttagccgttggagtgtcaagggctgttgtttttatttt

SEQ ID NO: 70 - RNA, 141 nt, synthetic construct
  source 1..141: mol_type = other RNA
ggtggacgtcgtcgcggggcacgcggagacgttaaacgctcggggagagggtgtcagacctgcgatagcgggcagccctcgatgaagcgagaatccaacggctttagccgttggagtgtcaagggctgttgtttttatttt

SEQ ID NO: 71 - RNA, 198 nt, synthetic construct
  source 1..198: mol_type = other RNA
ggtggagacctcaacgccgcccggaacatcaagcgggaagggctttcgcaaatcgtcgtcgcggggcacgcggagacgttaaacgctcggggagagggtgtcagacctgcgatagcgggcagccctcgatgaagcgagaatccaacggctttagccgttggagtgtcaagggctgttgtcagacccaacttttatttt

SEQ ID NO: 72 - RNA, 204 nt, synthetic construct
  source 1..204: mol_type = other RNA
ggtggagcggttcacgaccgcgccgcccggaacatcaagcgggaagggctttcgcaaatcgtcgtcgcggggcacgcggagacgttaaacgctcggggagagggtgtcagacctgcgatagcgggcagccctcgatgaagcgagaatccaacggctttagccgttggagtgtcaagggctgttgtcagacccaacttttatttt

SEQ ID NO: 73 - RNA, 185 nt, synthetic construct
  source 1..185: mol_type = other RNA
ggtggagcggttcacgaccgcgacctcaactttcgcaaatcgtcgtcgcggggcacgcggagacgttaaacgctcggggagagggtgtcagacctgcgatagcgggcagccctcgatgaagcgagaatccaacggctttagccgttggagtgtcaagggctgttgtcagacccaacttttatttt

SEQ ID NO: 74 - RNA, 188 nt, synthetic construct
  source 1..188: mol_type = other RNA
ggtggagcggttcacgaccgcgacctcaacgccgcccggaacatcaagcgggaagggctttcgcaaatttaaacgctcggggagagggtgtcagacctgcgatagcgggcagccctcgatgaagcgagaatccaacggctttagccgttggagtgtcaagggctgttgtcagacccaacttttatttt

SEQ ID NO: 75 - RNA, 198 nt, synthetic construct
  source 1..198: mol_type = other RNA
ggtggagcggttcacgaccgcgacctcaacgccgcccggaacatcaagcgggaagggctttcgcaaatcgtcgtcgcggggcacgcggagacggagggtgtcagacctgcgatagcgggcagccctcgatgaagcgagaatccaacggctttagccgttggagtgtcaagggctgttgtcagacccaacttttatttt

SEQ ID NO: 76 - RNA, 179 nt, synthetic construct
  source 1..179: mol_type = other RNA
ggtggagcggttcacgaccgcgacctcaacgccgcccggaacatcaagcgggaagggctttcgcaaatcgtcgtcgcggggcacgcggagacgttaaacgctcggggagatgaagcgagaatccaacggctttagccgttggagtgtcaagggctgttgtcagacccaacttttatttt

SEQ ID NO: 77 - RNA, 191 nt, synthetic construct
  source 1..191: mol_type = other RNA
ggtggagcggttcacgaccgcgacctcaacgccgcccggaacatcaagcgggaagggctttcgcaaatcgtcgtcgcggggcacgcggagacgttaaacgctcggggagagggtgtcagacctgcgatagcgggcagccctcgatgaagcgagaagtgtcaagggctgttgtcagacccaacttttatttt

SEQ ID NO: 78 - RNA, 203 nt, synthetic construct
  source 1..203: mol_type = other RNA
ggtggagcggttcacgaccgcgacctcaacgccgcccggaacatcaagcgggaagggccgtcgtcgcggggcacgcggagacgttaaacgctcggggagagggtgtcagacctgcgatagcgggcagccctcgatgaagcgagaatccaacggctttagccgttggagtgtcaagggctgttgtcagacccaacttttatttt

SEQ ID NO: 79 - RNA, 189 nt, synthetic construct
  source 1..189: mol_type = other RNA
ggtggagccgcccggaacatcaagcgggaagggctttcgcaaatcgtcgtcgcggggcacgcggagacgttaaacgctcggggagagggtgtcagacctgcgatagcgggcagccctcgatgaagcgagaatccaacggctttagccgttggagtgtcaagggctgttgtcagacccaacttttatttt

SEQ ID NO: 80 - RNA, 161 nt, synthetic construct
  source 1..161: mol_type = other RNA
ggtggatttcgcaaatcgtcgtcgcggggcacgcggagacgttaaacgctcggggagagggtgtcagacctgcgatagcgggcagccctcgatgaagcgagaatccaacggctttagccgttggagtgtcaagggctgttgtcagacccaacttttatttt

SEQ ID NO: 81 - RNA, 151 nt, synthetic construct
  source 1..151: mol_type = other RNA
ggtggacgtcgtcgcggggcacgcggagacgttaaacgctcggggagagggtgtcagacctgcgatagcgggcagccctcgatgaagcgagaatccaacggctttagccgttggagtgtcaagggctgttgtcagacccaacttttatttt

SEQ ID NO: 82 - DNA, 159 bp, synthetic construct
  source 1..159: mol_type = other DNA
ggcagccacatgttctatgccgtgaggaggggccgcaagaccggggtctttctgacctggaatgagtgcagagcacaggtggaccggtttcctgctgccagatttaagaagtttgccacagaggatgaggcctgggcctttgtcaggaaatctgcaagc

SEQ ID NO: 83 - protein, 53 aa, synthetic construct
GSHMFYAVRRGRKTGVFLTWNECRAQVDRFPAARFKKFATEDEAWAFVRKSAS

SEQ ID NO: 84 - DNA, 192 bp, synthetic construct
  source 1..192: mol_type = other DNA
atggccaccgtgaagtttaaatataagggcgaggagaaggaggtggatattagcaagatcaaaaaggtgtggagggtgggcaagatgattagcttcacctacgacgagggcggcggcaagacaggccggggcgcagtgagcgagaaggatgcccccaaggagctgctgcagatgctggagaagcagaagaag

SEQ ID NO: 85 - protein, 64 aa, synthetic construct
MATVKFKYKGEEKEVDISKIKKVWRVGKMISFTYDEGGGKTGRGAVSEKDAPKELLQMLEKQKK

SEQ ID NO: 86 - DNA, 3600 bp, synthetic construct; identical to SEQ ID NO: 51
  source 1..3600: mol_type = other DNA

SEQ ID NO: 87 - protein, 1200 aa, synthetic construct
PKKKRKVGIHGVPAAMTTHRKVYRYRIEPTPVQESKLYMLAGSRRFVFNWALARRREHYAETGKTLGYNAQAGELTALKNQEETSWLKESDSQLLQQALKDVERAFVNFFEKRARFPRFKSKKTDTPRFRIPQRVRIEGSRVYVPKVGWVKLRKSQEIEGKTKSATFKREADGHWYVLLVSEFEMPDVPLPPVPESEVVGIDLGLKDFYVLSDGGRKEAPRFARKGQRKLRRAARRHSKCTRGSNRKAKAKRKLARVHRQIANQRKDFVHKATSGLVQQYQGFCIENLSIKGMAKTKLSKSVLDAALGEFRRQLAYKAQWHRKWLAVIDRWFPSSKLCGECGSINADLTLSDREWTCECGAVHDRDLNAARNIKREGLSQIVVAGHAETLNARGEGVRPAIAGSPRSRADPKKKRKVSPGIRRLDALISTSLYKKAGYKEASGSGRADALDDFDLDMLGSDALDDFDLDMLGSDALDDFDLDMLGSDALDDFDLDMLINSRSSGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLFKRPAATKKAGQAKKKKMASNDYTQQATQSYGAYPTQPGQGYSQQSSQPYGQQSYSGYSQSTDTSGYGQSSYSSYGQSQNTGYGTQSTPQGYGSTGGYGSSQSSQSSYGQQSSYPGYGQQPAPSSTSGSYGSSSQSSSYGQPQSGSYSQQPSYGGQQQSYGQQQSYNPPQGYGQQNQYNSSSGGGGGGGGGGNYGQDQSSMSSGGGSGGGYGNQDQSGGGGSGGYGQQDRG

SEQ ID NO: 88 - DNA, 3807 bp, synthetic construct
  source 1..3807: mol_type = other DNA
ccaaagaagaagcggaaggtcggtatccacggagtcccagcagccatgaccacccaccgcaaggtgtacagataccgcatcgagcccacccccgtgcaggagagcaagctgtacatgctggccggcagccggagattcgtgttcaactgggccctggcccggagaagagagcactacgccgaaaccggcaagaccctgggatacaacgcccaggcaggagagctgaccgccctgaaaaaccaagaagaaaccagctggctgaaagagtccgactcacaactgctgcagcaggcactgaaagacgtggaaagagccttcgtgaacttcttcgaaaaacgggccagattccccagattcaaaagcaagaaaaccgacacccccagattcagaatcccccaaagagtgagaatagagggcagcagagtgtacgtgcccaaggtggggtgggtgaagctgagaaagagtcaggagatagaaggaaaaaccaaaagcgccaccttcaagagggaggcagatggccattggtacgtgctgctggtgagtgagttcgagatgcctgatgtgcctctgcccccagtgcccgagagcgaggtggtgggaatcgacctgggactgaaggacttctacgtcctgtctgacgggggcaggaaggaggcccccagattcgctagaaagggccagcgaaaactgagaagagctgcaagaagacatagcaagtgcacccggggaagcaacagaaaggccaaggcaaagaggaagctggcaagagtgcacagacagatcgcaaaccagagaaaagacttcgtgcacaaggccaccagcggcctcgtgcagcagtaccaaggcttctgcatcgaaaacctgagcatcaagggcatggccaaaaccaaactgtcaaaaagcgtgctggacgcagccctgggggaatttagaagacagctggcctataaagcccagtggcatagaaagtggctggcagtgatagacagatggttccccagcagcaagctgtgcggagagtgcggcagcattaacgccgatctgaccctgagcgacagagaatggacctgcgagtgtggcgcagtgcacgacagagatctgaacgccgctcgcaacatcaagagagagggcctgagtcagattgtggtggccggacatgccgaaaccctgaacgccagaggggagggcgtgagacccgccattgccggaagccctagatctggtggttctcccaagaagaagaggaaagtcggtagttccggatctggcagccacatgttctatgccgtgaggaggggccgcaagaccggggtctttctgacctggaatgagtgcagagcacaggtggaccggtttcctgctgccagatttaagaagtttgccacagaggatgaggcctgggcctttgtcaggaaatctgcaagctctggaggatctagcggaggatcctctggcagcgagacaccaggaacaagcgagtcagcaacaccagagagcagtggcggcagcagcggcggcagcagcgaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgtttaaaaggccggcggccacgaaaaaggccggccaggcaaaaaagaaaaagatggcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacacaggctatggaactcagtcaactccccagggatatggctcgactggcggctatggcagtagccagagctcccaatcgtcttacgggcagcagtcctcctaccctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtgga

SEQ ID NO: 89 - protein, 1269 aa, synthetic construct
PKKKRKVGIHGVPAAMTTHRKVYRYRIEPTPVQESKLYMLAGSRRFVFNWALARRREHYAETGKTLGYNAQAGELTALKNQEETSWLKESDSQLLQQALKDVERAFVNFFEKRARFPRFKSKKTDTPRFRIPQRVRIEGSRVYVPKVGWVKLRKSQEIEGKTKSATFKREADGHWYVLLVSEFEMPDVPLPPVPESEVVGIDLGLKDFYVLSDGGRKEAPRFARKGQRKLRRAARRHSKCTRGSNRKAKAKRKLARVHRQIANQRKDFVHKATSGLVQQYQGFCIENLSIKGMAKTKLSKSVLDAALGEFRRQLAYKAQWHRKWLAVIDRWFPSSKLCGECGSINADLTLSDREWTCECGAVHDRDLNAARNIKREGLSQIVVAGHAETLNARGEGVRPAIAGSPRSGGSPKKKRKVGSSGSGSHMFYAVRRGRKTGVFLTWNECRAQVDRFPAARFKKFATEDEAWAFVRKSASSGGSSGGSSGSETPGTSESATPESSGGSSGGSSEASGSGRADALDDFDLDMLGSDALDDFDLDMLGSDALDDFDLDMLGSDALDDFDLDMLINSRSSGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLFKRPAATKKAGQAKKKKMASNDYTQQATQSYGAYPTQPGQGYSQQSSQPYGQQSYSGYSQSTDTSGYGQSSYSSYGQSQNTGYGTQSTPQGYGSTGGYGSSQSSQSSYGQQSSYPGYGQQPAPSSTSGSYGSSSQSSSYGQPQSGSYSQQPSYGGQQQSYGQQQSYNPPQGYGQQNQYNSSSGGGGGGGGGGNYGQDQSSMSSGGGSGGGYGNQDQSGGGGSGGYGQQDRG

SEQ ID NO: 90 - DNA, 3906 bp, synthetic construct
  source 1..3906: mol_type = other DNA
ccaaagaagaagcggaaggtcggtatccacggagtcccagcagccatgaccacccaccgcaaggtgtacagataccgcatcgagcccacccccgtgcaggagagcaagctgtacatgctggccggcagccggagattcgtgttcaactgggccctggcccggagaagagagcactacgccgaaaccggcaagaccctgggatacaacgcccaggcaggagagctgaccgccctgaaaaaccaagaagaaaccagctggctgaaagagtccgactcacaactgctgcagcaggcactgaaagacgtggaaagagccttcgtgaacttcttcgaaaaacgggccagattccccagattcaaaagcaagaaaaccgacacccccagattcagaatcccccaaagagtgagaatagagggcagcagagtgtacgtgcccaaggtggggtgggtgaagctgagaaagagtcaggagatagaaggaaaaaccaaaagcgccaccttcaagagggaggcagatggccattggtacgtgctgctggtgagtgagttcgagatgcctgatgtgcctctgcccccagtgcccgagagcgaggtggtgggaatcgacctgggactgaaggacttctacgtcctgtctgacgggggcaggaaggaggcccccagattcgctagaaagggccagcgaaaactgagaagagctgcaagaagacatagcaagtgcacccggggaagcaacagaaaggccaaggcaaagaggaagctggcaagagtgcacagacagatcgcaaaccagagaaaagacttcgtgcacaaggccaccagcggcctcgtgcagcagtaccaaggcttctgcatcgaaaacctgagcatcaagggcatggccaaaaccaaactgtcaaaaagcgtgctggacgcagccctgggggaatttagaagacagctggcctataaagcccagtggcatagaaagtggctggcagtgatagacagatggttccccagcagcaagctgtgcggagagtgcggcagcattaacgccgatctgaccctgagcgacagagaatggacctgcgagtgtggcgcagtgcacgacagagatctgaacgccgctcgcaacatcaagagagagggcctgagtcagattgtggtggccggacatgccgaaaccctgaacgccagaggggagggcgtgagacccgccattgccggaagccctagaagcagggctgaccccaagaagaagaggaaggtgtcgccagggatccgtcgacttgacgcgttgatatcaacaagtttgtacaaaaaagcaggctacaaagaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgtttaaaaggccggcggccacgaaaaaggccggccaggcaaaaaagaaaaagtctggtggttctcccaagaagaagaggaaagtcggtagttccggatctggcagccacatgttctatgccgtgaggaggggccgcaagaccggggtctttctgacctggaatgagtgcagagcacaggtggaccggtttcctgctgccagatttaagaagtttgccacagaggatgaggcctgggcctttgtcaggaaatctgcaagctctggaggatctagcggaggatcctctggcagcgagacaccaggaacaagcgagtcagcaacaccagagagcagtggcggcagcagcggcggcagcagcatggcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacacaggctatggaactcagtcaactccccagggatatggctcgactggcggctatggcagtagccagagctcccaatcgtcttacgggcagcagtcctcctaccctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtgga

SEQ ID NO: 91 - protein, 1302 aa, synthetic construct
PKKKRKVGIHGVPAAMTTHRKVYRYRIEPTPVQESKLYMLAGSRRFVFNWALARRREHYAETGKTLGYNAQAGELTALKNQEETSWLKESDSQLLQQALKDVERAFVNFFEKRARFPRFKSKKTDTPRFRIPQRVRIEGSRVYVPKVGWVKLRKSQEIEGKTKSATFKREADGHWYVLLVSEFEMPDVPLPPVPESEVVGIDLGLKDFYVLSDGGRKEAPRFARKGQRKLRRAARRHSKCTRGSNRKAKAKRKLARVHRQIANQRKDFVHKATSGLVQQYQGFCIENLSIKGMAKTKLSKSVLDAALGEFRRQLAYKAQWHRKWLAVIDRWFPSSKLCGECGSINADLTLSDREWTCECGAVHDRDLNAARNIKREGLSQIVVAGHAETLNARGEGVRPAIAGSPRSRADPKKKRKVSPGIRRLDALISTSLYKKAGYKEASGSGRADALDDFDLDMLGSDALDDFDLDMLGSDALDDFDLDMLGSDALDDFDLDMLINSRSSGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLFKRPAATKKAGQAKKKKSGGSPKKKRKVGSSGSGSHMFYAVRRGRKTGVFLTWNECRAQVDRFPAARFKKFATEDEAWAFVRKSASSGGSSGGSSGSETPGTSESATPESSGGSSGGSSMASNDYTQQATQSYGAYPTQPGQGYSQQSSQPYGQQSYSGYSQSTDTSGYGQSSYSSYGQSQNTGYGTQSTPQGYGSTGGYGSSQSSQSSYGQQSSYPGYGQQPAPSSTSGSYGSSSQSSSYGQPQSGSYSQQPSYGGQQQSYGQQQSYNPPQGYGQQNQYNSSSGGGGGGGGGGNYGQDQSSMSSGGGSGGGYGNQDQSGGGGSGGYGQQDRG

SEQ ID NO: 92 - DNA, 3807 bp, synthetic construct
  source 1..3807: mol_type = other DNA
ccaaagaagaagcggaaggtcggtatccacggagtcccagcagccatgaccacccaccgcaaggtgtacagataccgcatcgagcccacccccgtgcaggagagcaagctgtacatgctggccggcagccggagattcgtgttcaactgggccctggcccggagaagagagcactacgccgaaaccggcaagaccctgggatacaacgcccaggcaggagagctgaccgccctgaaaaaccaagaagaaaccagctggctgaaagagtccgactcacaactgctgcagcaggcactgaaagacgtggaaagagccttcgtgaacttcttcgaaaaacgggccagattccccagattcaaaagcaagaaaaccgacacccccagattcagaatcccccaaagagtgagaatagagggcagcagagtgtacgtgcccaaggtggggtgggtgaagctgagaaagagtcaggagatagaaggaaaaaccaaaagcgccaccttcaagagggaggcagatggccattggtacgtgctgctggtgagtgagttcgagatgcctgatgtgcctctgcccccagtgcccgagagcgaggtggtgggaatcgacctgggactgaaggacttctacgtcctgtctgacgggggcaggaaggaggcccccagattcgctagaaagggccagcgaaaactgagaagagctgcaagaagacatagcaagtgcacccggggaagcaacagaaaggccaaggcaaagaggaagctggcaagagtgcacagacagatcgcaaaccagagaaaagacttcgtgcacaaggccaccagcggcctcgtgcagcagtaccaaggcttctgcatcgaaaacctgagcatcaagggcatggccaaaaccaaactgtcaaaaagcgtgctggacgcagccctgggggaatttagaagacagctggcctataaagcccagtggcatagaaagtggctggcagtgatagacagatggttccccagcagcaagctgtgcggagagtgcggcagcattaacgccgatctgaccctgagcgacagagaatggacctgcgagtgtggcgcagtgcacgacagagatctgaacgccgctcgcaacatcaagagagagggcctgagtcagattgtggtggccggacatgccgaaaccctgaacgccagaggggagggcgtgagacccgccattgccggaagccctagaagcagggctgaccccaagaagaagaggaaggtgtcgccagggatccgtcgacttgacgcgttgatatcaacaagtttgtacaaaaaagcaggctacaaagaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgtttaaaaggccggcggccacgaaaaaggccggccaggcaaaaaagaaaaagatggcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacacaggctatggaactcagtcaactccccagggatatggctcgactggcggctatggcagtagccagagctcccaatcgtcttacgggcagcagtcctcctaccctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtggatctggtggttctcccaagaagaagaggaaagtcggtagttccggatctggcagccacatgttctatgccgtgaggaggggccgcaagaccggggtctttctgacctggaatgagtgcagagcacaggtggaccggtttcctgctgccagatttaagaagtttgccacagaggatgaggcctgggcctttgtcaggaaatctgcaagc

SEQ ID NO: 93 - protein, 1269 aa, synthetic construct
PKKKRKVGIHGVPAAMTTHRKVYRYRIEPTPVQESKLYMLAGSRRFVFNWALARRREHYAETGKTLGYNAQAGELTALKNQEETSWLKESDSQLLQQALKDVERAFVNFFEKRARFPRFKSKKTDTPRFRIPQRVRIEGSRVYVPKVGWVKLRKSQEIEGKTKSATFKREADGHWYVLLVSEFEMPDVPLPPVPESEVVGIDLGLKDFYVLSDGGRKEAPRFARKGQRKLRRAARRHSKCTRGSNRKAKAKRKLARVHRQIANQRKDFVHKATSGLVQQYQGFCIENLSIKGMAKTKLSKSVLDAALGEFRRQLAYKAQWHRKWLAVIDRWFPSSKLCGECGSINADLTLSDREWTCECGAVHDRDLNAARNIKREGLSQIVVAGHAETLNARGEGVRPAIAGSPRSRADPKKKRKVSPGIRRLDALISTSLYKKAGYKEASGSGRADALDDFDLDMLGSDALDDFDLDMLGSDALDDFDLDMLGSDALDDFDLDMLINSRSSGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLFKRPAATKKAGQAKKKKMASNDYTQQATQSYGAYPTQPGQGYSQQSSQPYGQQSYSGYSQSTDTSGYGQSSYSSYGQSQNTGYGTQSTPQGYGSTGGYGSSQSSQSSYGQQSSYPGYGQQPAPSSTSGSYGSSSQSSSYGQPQSGSYSQQPSYGGQQQSYGQQQSYNPPQGYGQQNQYNSSSGGGGGGGGGGNYGQDQSSMSSGGGSGGGYGNQDQSGGGGSGGYGQQDRGSGGSPKKKRKVGSSGSGSHMFYAVRRGRKTGVFLTWNECRAQVDRFPAARFKKFATEDEAWAFVRKSAS

SEQ ID NO: 94 - DNA, 3822 bp, synthetic construct
  source 1..3822: mol_type = other DNA
ccaaagaagaagcggaaggtcggtatccacggagtcccagcagccatggccaccgtgaagtttaaatataagggcgaggagaaggaggtggatattagcaagatcaaaaaggtgtggagggtgggcaagatgattagcttcacctacgacgagggcggcggcaagacaggccggggcgcagtgagcgagaaggatgcccccaaggagctgctgcagatgctggagaagcagaagaagggcactagtggcggaggctccggcggtggcatgaccacccaccgcaaggtgtacagataccgcatcgagcccacccccgtgcaggagagcaagctgtacatgctggccggcagccggagattcgtgttcaactgggccctggcccggagaagagagcactacgccgaaaccggcaagaccctgggatacaacgcccaggcaggagagctgaccgccctgaaaaaccaagaagaaaccagctggctgaaagagtccgactcacaactgctgcagcaggcactgaaagacgtggaaagagccttcgtgaacttcttcgaaaaacgggccagattccccagattcaaaagcaagaaaaccgacacccccagattcagaatcccccaaagagtgagaatagagggcagcagagtgtacgtgcccaaggtggggtgggtgaagctgagaaagagtcaggagatagaaggaaaaaccaaaagcgccaccttcaagagggaggcagatggccattggtacgtgctgctggtgagtgagttcgagatgcctgatgtgcctctgcccccagtgcccgagagcgaggtggtgggaatcgacctgggactgaaggacttctacgtcctgtctgacgggggcaggaaggaggcccccagattcgctagaaagggccagcgaaaactgagaagagctgcaagaagacatagcaagtgcacccggggaagcaacagaaaggccaaggcaaagaggaagctggcaagagtgcacagacagatcgcaaaccagagaaaagacttcgtgcacaaggccaccagcggcctcgtgcagcagtaccaaggcttctgcatcgaaaacctgagcatcaagggcatggccaaaaccaaactgtcaaaaagcgtgctggacgcagccctgggggaatttagaagacagctggcctataaagcccagtggcatagaaagtggctggcagtgatagacagatggttccccagcagcaagctgtgcggagagtgcggcagcattaacgccgatctgaccctgagcgacagagaatggacctgcgagtgtggcgcagtgcacgacagagatctgaacgccgctcgcaacatcaagagagagggcctgagtcagattgtggtggccggacatgccgaaaccctgaacgccagaggggagggcgtgagacccgccattgccggaagccctagaagcagggctgaccccaagaagaagaggaaggtgtcgccagggatccgtcgacttgacgcgttgatatcaacaagtttgtacaaaaaagcaggctacaaagaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgtttaaaaggccggcggccacgaaaaaggccggccaggcaaaaaagaaaaagatggcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacacaggctatggaactcagtcaactccccagggatatggctcgactggcggctatggcagtagccagagctcccaatcgtcttacgggcagcagtcctcctaccctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtgga

SEQ ID NO: 95 - protein, 1274 aa, synthetic construct
PKKKRKVGIHGVPAAMATVKFKYKGEEKEVDISKIKKVWRVGKMISFTYDEGGGKTGRGAVSEKDAPKELLQMLEKQKKGTSGGGSGGGMTTHRKVYRYRIEPTPVQESKLYMLAGSRRFVFNWALARRREHYAETGKTLGYNAQAGELTALKNQEETSWLKESDSQLLQQALKDVERAFVNFFEKRARFPRFKSKKTDTPRFRIPQRVRIEGSRVYVPKVGWVKLRKSQEIEGKTKSATFKREADGHWYVLLVSEFEMPDVPLPPVPESEVVGIDLGLKDFYVLSDGGRKEAPRFARKGQRKLRRAARRHSKCTRGSNRKAKAKRKLARVHRQIANQRKDFVHKATSGLVQQYQGFCIENLSIKGMAKTKLSKSVLDAALGEFRRQLAYKAQWHRKWLAVIDRWFPSSKLCGECGSINADLTLSDREWTCECGAVHDRDLNAARNIKREGLSQIVVAGHAETLNARGEGVRPAIAGSPRSRADPKKKRKVSPGIRRLDALISTSLYKKAGYKEASGSGRADALDDFDLDMLGSDALDDFDLDMLGSDALDDFDLDMLGSDALDDFDLDMLINSRSSGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLFKRPAATKKAGQAKKKKMASNDYTQQATQSYGAYPTQPGQGYSQQSSQPYGQQSYSGYSQSTDTSGYGQSSYSSYGQSQNTGYGTQSTPQGYGSTGGYGSSQSSQSSYGQQSSYPGYGQQPAPSSTSGSYGSSSQSSSYGQPQSGSYSQQPSYGGQQQSYGQQQSYNPPQGYGQQNQYNSSSGGGGGGGGGGNYGQDQSSMSSGGGSGGGYGNQDQSGGGGSGGYGQQDRG

SEQ ID NO: 96 - DNA, 3822 bp, synthetic construct
  source 1..3822: mol_type = other DNA
ccaaagaagaagcggaaggtcggtatccacggagtcccagcagccatgaccacccaccgcaaggtgtacagataccgcatcgagcccacccccgtgcaggagagcaagctgtacatgctggccggcagccggagattcgtgttcaactgggccctggcccggagaagagagcactacgccgaaaccggcaagaccctgggatacaacgcccaggcaggagagctgaccgccctgaaaaaccaagaagaaaccagctggctgaaagagtccgactcacaactgctgcagcaggcactgaaagacgtggaaagagccttcgtgaacttcttcgaaaaacgggccagattccccagattcaaaagcaagaaaaccgacacccccagattcagaatcccccaaagagtgagaatagagggcagcagagtgtacgtgcccaaggtggggtgggtgaagctgagaaagagtcaggagatagaaggaaaaaccaaaagcgccaccttcaagagggaggcagatggccattggtacgtgctgctggtgagtgagttcgagatgcctgatgtgcctctgcccccagtgcccgagagcgaggtggtgggaatcgacctgggactgaaggacttctacgtcctgtctgacgggggcaggaaggaggcccccagattcgctagaaagggccagcgaaaactgagaagagctgcaagaagacatagcaagtgcacccggggaagcaacagaaaggccaaggcaaagaggaagctggcaagagtgcacagacagatcgcaaaccagagaaaagacttcgtgcacaaggccaccagcggcctcgtgcagcagtaccaaggcttctgcatcgaaaacctgagcatcaagggcatggccaaaaccaaactgtcaaaaagcgtgctggacgcagccctgggggaatttagaagacagctggcctataaagcccagtggcatagaaagtggctggcagtgatagacagatggttccccagcagcaagctgtgcggagagtgcggcagcattaacgccgatctgaccctgagcgacagagaatggacctgcgagtgtggcgcagtgcacgacagagatctgaacgccgctcgcaacatcaagagagagggcctgagtcagattgtggtggccggacatgccgaaaccctgaacgccagaggggagggcgtgagacccgccattgccggaagccctagaagcagggctgaccccaagaagaagaggaaggtgtcgccagggatccgtcgacttgacgcgttgatatcaacaagtttgtacaaaaaagcaggctacaaagaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgtttaaaaggccggcggccacgaaaaaggccggccaggcaaaaaagaaaaagatggcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacacaggctatggaactcagtcaactccccagggatatggctcgactggcggctatggcagtagccagagctcccaatcgtcttacgggcagcagtcctcctaccctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtggaggcactagtggcggaggctccggcggtggcatggccaccgtgaagtttaaatataagggcgaggagaaggaggtggatattagcaagatcaaaaaggtgtggagggtgggcaagatgattagcttcacctacgacgagggcggcggcaagacaggccggggcgcagtgagcgagaaggatgcccccaaggagctgctgcagatgctggagaagcagaagaag

SEQ ID NO: 97 - protein, 1274 aa, synthetic construct
PKKKRKVGIHGVPAAMTTHRKVYRYRIEPTPVQESKLYMLAGSRRFVFNWALARRREHYAETGKTLGYNAQAGELTALKNQEETSWLKESDSQLLQQALKDVERAFVNFFEKRARFPRFKSKKTDTPRFRIPQRVRIEGSRVYVPKVGWVKLRKSQEIEGKTKSATFKREADGHWYVLLVSEFEMPDVPLPPVPESEVVGIDLGLKDFYVLSDGGRKEAPRFARKGQRKLRRAARRHSKCTRGSNRKAKAKRKLARVHRQIANQRKDFVHKATSGLVQQYQGFCIENLSIKGMAKTKLSKSVLDAALGEFRRQLAYKAQWHRKWLAVIDRWFPSSKLCGECGSINADLTLSDREWTCECGAVHDRDLNAARNIKREGLSQIVVAGHAETLNARGEGVRPAIAGSPRSRADPKKKRKVSPGIRRLDALISTSLYKKAGYKEASGSGRADALDDFDLDMLGSDALDDFDLDMLGSDALDDFDLDMLGSDALDDFDLDMLINSRSSGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLFKRPAATKKAGQAKKKKMASNDYTQQATQSYGAYPTQPGQGYSQQSSQPYGQQSYSGYSQSTDTSGYGQSSYSSYGQSQNTGYGTQSTPQGYGSTGGYGSSQSSQSSYGQQSSYPGYGQQPAPSSTSGSYGSSSQSSSYGQPQSGSYSQQPSYGGQQQSYGQQQSYNPPQGYGQQNQYNSSSGGGGGGGGGGNYGQDQSSMSSGGGSGGGYGNQDQSGGGGSGGYGQQDRGGTSGGGSGGGMATVKFKYKGEEKEVDISKIKKVWRVGKMISFTYDEGGGKTGRGAVSEKDAPKELLQMLEKQKK

SEQ ID NO: 98 - DNA, 8225 bp, synthetic construct
  source 1..8225: mol_type = other DNA
tttagcgcgtgcgccaattctgcagacaaatggctctagaggtaccgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgcgccaccatggccccaaagaagaagcggaaggtcggtatccacggagtcccagcagccatgaccacccaccgcaaggtgtacagataccgcatcgagcccacccccgtgcaggagagcaagctgtacatgctggccggcagccggagattcgtgttcaactgggccctggcccggagaagagagcactacgccgaaaccggcaagaccctgggatacaacgcccaggcaggagagctgaccgccctgaaaaaccaagaagaaaccagctggctgaaagagtccgactcacaactgctgcagcaggcactgaaagacgtggaaagagccttcgtgaacttcttcgaaaaacgggccagattccccagattcaaaagcaagaaaaccgacacccccagattcagaatcccccaaagagtgagaatagagggcagcagagtgtacgtgcccaaggtggggtgggtgaagctgagaaagagtcaggagatagaaggaaaaaccaaaagcgccaccttcaagagggaggcagatggccattggtacgtgctgctggtgagtgagttcgagatgcctgatgtgcctctgcccccagtgcccgagagcgaggtggtgggaatcgacctgggactgaaggacttctacgtcctgtctgacgggggcaggaaggaggcccccagattcgctagaaagggccagcgaaaactgagaagagctgcaagaagacatagcaagtgcacccggggaagcaacagaaaggccaaggcaaagaggaagctggcaagagtgcacagacagatcgcaaaccagagaaaagacttcgtgcacaaggccaccagcggcctcgtgcagcagtaccaaggcttctgcatcgaaaacctgagcatcaagggcatggccaaaaccaaactgtcaaaaagcgtgctggacgcagccctgggggaatttagaagacagctggcctataaagcccagtggcatagaaagtggctggcagtgatagacagatggttccccagcagcaagctgtgcggagagtgcggcagcattaacgccgatctgaccctgagcgacagagaatggacctgcgagtgtggcgcagtgcacgacagagatctgaacgccgctcgcaacatcaagagagagggcctgagtcagattgtggtggccggacatgccgaaaccctgaacgccagaggggagggcgtgagacccgccattgccggaagccctagaagcagggctgaccccaagaagaagaggaaggtgtcgccagggatccgtcgacttgacgcgttgatatcaacaagtttgtacaaaaaagcaggctacaaagaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgtttaaaaggccggcggccacgaaaaaggccggccaggcaaaaaagaaaaagatggcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacacaggctatggaactcagtcaactccccagggatatggctcgactggcggctatggcagtagccagagctcccaatcgtcttacgggcagcagtcctcctaccctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtggagaattcggcagtggagagggcagaggaagtctgctaacatgcggtgacgtcgaggagaatcctggcccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtggacgtcgtcgcggggcacgcggagacgttaaacgctcggggagagggtgtcagacctgcgatagcgggcagccctcgatgaagcgagaatccaacggctttagccgttggagtgtcaatcttcatcccttttattttttgttttagagctagaaatagtaagttaaaataaggctagtccgtt

SEQ ID NO: 99 - DNA, 8432 bp, synthetic construct
  source 1..8432: mol_type = other DNA
tttagcgcgtgcgccaattctgcagacaaatggctctagaggtaccgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgcgccaccatggccccaaagaagaagcggaaggtcggtatccacggagtcccagcagccatgaccacccaccgcaaggtgtacagataccgcatcgagcccacccccgtgcaggagagcaagctgtacatgctggccggcagccggagattcgtgttcaactgggccctggcccggagaagagagcactacgccgaaaccggcaagaccctgggatacaacgcccaggcaggagagctgaccgccctgaaaaaccaagaagaaaccagctggctgaaagagtccgactcacaactgctgcagcaggcactgaaagacgtggaaagagccttcgtgaacttcttcgaaaaacgggccagattccccagattcaaaagcaagaaaaccgacacccccagattcagaatcccccaaagagtgagaatagagggcagcagagtgtacgtgcccaaggtggggtgggtgaagctgagaaagagtcaggagatagaaggaaaaaccaaaagcgccaccttcaagagggaggcagatggccattggtacgtgctgctggtgagtgagttcgagatgcctgatgtgcctctgcccccagtgcccgagagcgaggtggtgggaatcgacctgggactgaaggacttctacgtcctgtctgacgggggcaggaaggaggcccccagattcgctagaaagggccagcgaaaactgagaagagctgcaagaagacatagcaagtgcacccggggaagcaacagaaaggccaaggcaaagaggaagctggcaagagtgcacagacagatcgcaaaccagagaaaagacttcgtgcacaaggccaccagcggcctcgtgcagcagtaccaaggcttctgcatcgaaaacctgagcatcaagggcatggccaaaaccaaactgtcaaaaagcgtgctggacgcagccctgggggaatttagaagacagctggcctataaagcccagtggcatagaaagtggctggcagtgatagacagatggttccccagcagcaagctgtgcggagagtgcggcagcattaacgccgatctgaccctgagcgacagagaatggacctgcgagtgtggcgcagtgcacgacagagatctgaacgccgctcgcaacatcaagagagagggcctgagtcagattgtggtggccggacatgccgaaaccctgaacgccagaggggagggcgtgagacccgccattgccggaagccctagatctggtggttctcccaagaagaagaggaaagtcggtagttccggatctggcagccacatgttctatgccgtgaggaggggccgcaagaccggggtctttctgacctggaatgagtgcagagcacaggtggaccggtttcctgctgccagatttaagaagtttgccacagaggatgaggcctgggcctttgtcaggaaatctgcaagctctggaggatctagcggaggatcctctggcagcgagacaccaggaacaagcgagtcagcaacaccagagagcagtggcggcagcagcggcggcagcagcgaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgtttaaaaggccggcggccacgaaaaaggccggccaggcaaaaaagaaaaagatggcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacacaggctatggaactcagtcaactccccagggatatggctcgactggcggctatggcagtagccagagctcccaatcgtcttacgggcagcagtcctcctaccctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtggagaattcggcagtggagagggcagaggaagtctgctaacatgcggtgacgtcgaggagaatcctggcccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtggacgtcgtcgcggggcacgcggagacgttaaacgctcggggagagggtgtcagacctgcgatagcgggcagccctcgatgaagcgagaatccaacggctttagccgttggagtgtcaatcttcatcccttttattttttgttttagagctagaaatagtaagttaaaataaggctagtccgtt

SEQ ID NO: 100 - DNA, 8531 bp, synthetic construct
  source 1..8531: mol_type = other DNA
tttagcgcgtgcgccaattctgcagacaaatggctctagaggtaccgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgcgccaccatggccccaaagaagaagcggaaggtcggtatccacggagtcccagcagccatgaccacccaccgcaaggtgtacagataccgcatcgagcccacccccgtgcaggagagcaagctgtacatgctggccggcagccggagattcgtgttcaactgggccctggcccggagaagagagcactacgccgaaaccggcaagaccctgggatacaacgcccaggcaggagagctgaccgccctgaaaaaccaagaagaaaccagctggctgaaagagtccgactcacaactgctgcagcaggcactgaaagacgtggaaagagccttcgtgaacttcttcgaaaaacgggccagattccccagattcaaaagcaagaaaaccgacacccccagattcagaatcccccaaagagtgagaatagagggcagcagagtgtacgtgcccaaggtggggtgggtgaagctgagaaagagtcaggagatagaaggaaaaaccaaaagcgccaccttcaagagggaggcagatggccattggtacgtgctgctggtgagtgagttcgagatgcctgatgtgcctctgcccccagtgcccgagagcgaggtggtgggaatcgacctgggactgaaggacttctacgtcctgtctgacgggggcaggaaggaggcccccagattcgctagaaagggccagcgaaaactgagaagagctgcaagaagacatagcaagtgcacccggggaagcaacagaaaggccaaggcaaagaggaagctggcaagagtgcacagacagatcgcaaaccagagaaaagacttcgtgcacaaggccaccagcggcctcgtgcagcagtaccaaggcttctgcatcgaaaacctgagcatcaagggcatggccaaaaccaaactgtcaaaaagcgtgctggacgcagccctgggggaatttagaagacagctggcctataaagcccagtggcatagaaagtggctggcagtgatagacagatggttccccagcagcaagctgtgcggagagtgcggcagcattaacgccgatctgaccctgagcgacagagaatggacctgcgagtgtggcgcagtgcacgacagagatctgaacgccgctcgcaacatcaagagagagggcctgagtcagattgtggtggccggacatgccgaaaccctgaacgccagaggggagggcgtgagacccgccattgccggaagccctagaagcagggctgaccccaagaagaagaggaaggtgtcgccagggatccgtcgacttgacgcgttgatatcaacaagtttgtacaaaaaagcaggctacaaagaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgtttaaaaggccggcggccacgaaaaaggccggccaggcaaaaaagaaaaagtctggtggttctcccaagaagaagaggaaagtcggtagttccggatctggcagccacatgttctatgccgtgaggaggggccgcaagaccggggtctttctgacctggaatgagtgcagagcacaggtggaccggtttcctgctgccagatttaagaagtttgccacagaggatgaggcctgggcctttgtcaggaaatctgcaagctctggaggatctagcggaggatcctctggcagcgagacaccaggaacaagcgagtcagcaacaccagagagcagtggcggcagcagcggcggcagcagcatggcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacacaggctatggaactcagtcaactccccagggatatggctcgactggcggctatggcagtagccagagctcccaatcgtcttacgggcagcagtcctcctaccctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtggagaattcggcagtggagagggcagaggaagtctgctaacatgcggtgacgtcgaggagaatcctggcccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtggacgtcgtcgcggggcacgcggagacgttaaacgctcggggagagggtgtcagacctgcgatagcgggcagccctcgatgaagcgagaatccaacggctttagccgttggagtgtcaatcttcatcccttttattttttgttttagagctagaaatagtaagttaaaataaggctagtccgtt

SEQ ID NO: 101 - DNA, 8432 bp, synthetic construct
  source 1..8432: mol_type = other DNA
tttagcgcgtgcgccaattctgcagacaaatggctctagaggtaccgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgcgccaccatggccccaaagaagaagcggaaggtcggtatccacggagtcccagcagccatgaccacccaccgcaaggtgtacagataccgcatcgagcccacccccgtgcaggagagcaagctgtacatgctggccggcagccggagattcgtgttcaactgggccctggcccggagaagagagcactacgccgaaaccggcaagaccctgggatacaacgcccaggcaggagagctgaccgccctgaaaaaccaagaagaaaccagctggctgaaagagtccgactcacaactgctgcagcaggcactgaaagacgtggaaagagccttcgtgaacttcttcgaaaaacgggccagattccccagattcaaaagcaagaaaaccgacacccccagattcagaatcccccaaagagtgagaatagagggcagcagagtgtacgtgcccaaggtggggtgggtgaagctgagaaagagtcaggagatagaaggaaaaaccaaaagcgccaccttcaagagggaggcagatggccattggtacgtgctgctggtgagtgagttcgagatgcctgatgtgcctctgcccccagtgcccgagagcgaggtggtgggaatcgacctgggactgaaggacttctacgtcctgtctgacgggggcaggaaggaggcccccagattcgctagaaagggccagcgaaaactgagaagagctgcaagaagacatagcaagtgcacccggggaagcaacagaaaggccaaggcaaagaggaagctggcaagagtgcacagacagatcgcaaaccagagaaaagacttcgtgcacaaggccaccagcggcctcgtgcagcagtaccaaggcttctgcatcgaaaacctgagcatcaagggcatggccaaaaccaaactgtcaaaaagcgtgctggacgcagccctgggggaatttagaagacagctggcctataaagcccagtggcatagaaagtggctggcagtgatagacagatggttccccagcagcaagctgtgcggagagtgcggcagcattaacgccgatctgaccctgagcgacagagaatggacctgcgagtgtggcgcagtgcacgacagagatctgaacgccgctcgcaacatcaagagagagggcctgagtcagattgtggtggccggacatgccgaaaccctgaacgccagaggggagggcgtgagacccgccattgccggaagccctagaagcagggctgaccccaagaagaagaggaaggtgtcgccagggatccgtcgacttgacgcgttgatatcaacaagtttgtacaaaaaagcaggctacaaagaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgtttaaaaggccggcggccacgaaaaaggccggccaggcaaaaaagaaaaagatggcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacacaggctatggaactcagtcaactccccagggatatggctcgactggcggctatggcagtagccagagctcccaatcgtcttacgggcagcagtcctcctaccctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtggatctggtggttctcccaagaagaagaggaaagtcggtagttccggatctggcagccacatgttctatgccgtgaggaggggccgcaagaccggggtctttctgacctggaatgagtgcagagcacaggtggaccggtttcctgctgccagatttaagaagtttgccacagaggatgaggcctgggcctttgtcaggaaatctgcaagcgaattcggcagtggagagggcagaggaagtctgctaacatgcggtgacgtcgaggagaatcctggcccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtggacgtcgtcgcggggcacgcggagacgttaaacgctcggggagagggtgtcagacctgcgatagcgggcagccctcgatgaagcgagaatccaacggctttagccgttggagtgtcaatcttcatcccttttattttttgttttagagctagaaatagtaagttaaaataaggctagtccgtt

SEQ ID NO: 102 - DNA, 8447 bp, synthetic construct
  source 1..8447: mol_type = other DNA
tttagcgcgtgcgccaattctgcagacaaatggctctagaggtaccgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgcgccaccatggccccaaagaagaagcggaaggtcggtatccacggagtcccagcagccatggccaccgtgaagtttaaatataagggcgaggagaaggaggtggatattagcaagatcaaaaaggtgtggagggtgggcaagatgattagcttcacctacgacgagggcggcggcaagacaggccggggcgcagtgagcgagaaggatgcccccaaggagctgctgcagatgctggagaagcagaagaagggcactagtggcggaggctccggcggtggcatgaccacccaccgcaaggtgtacagataccgcatcgagcccacccccgtgcaggagagcaagctgtacatgctggccggcagccggagattcgtgttcaactgggccctggcccggagaagagagcactacgccgaaaccggcaagaccctgggatacaacgcccaggcaggagagctgaccgccctgaaaaaccaagaagaaaccagctggctgaaagagtccgactcacaactgctgcagcaggcactgaaagacgtggaaagagccttcgtgaacttcttcgaaaaacgggccagattccccagattcaaaagcaagaaaaccgacacccccagattcagaatcccccaaagagtgagaatagagggcagcagagtgtacgtgcccaaggtggggtgggtgaagctgagaaagagtcaggagatagaaggaaaaaccaaaagcgccaccttcaagagggaggcagatggccattggtacgtgctgctggtgagtgagttcgagatgcctgatgtgcctctgcccccagtgcccgagagcgaggtggtgggaatcgacctgggactgaaggacttctacgtcctgtctgacgggggcaggaaggaggcccccagattcgctagaaagggccagcgaaaactgagaagagctgcaagaagacatagcaagtgcacccggggaagcaacagaaaggccaaggcaaagaggaagctggcaagagtgcacagacagatcgcaaaccagagaaaagacttcgtgcacaaggccaccagcggcctcgtgcagcagtaccaaggcttctgcatcgaaaacctgagcatcaagggcatggccaaaaccaaactgtcaaaaagcgtgctggacgcagccctgggggaatttagaagacagctggcctataaagcccagtggcatagaaagtggctggcagtgatagacagatggttccccagcagcaagctgtgcggagagtgcggcagcattaacgccgatctgaccctgagcgacagagaatggacctgcgagtgtggcgcagtgcacgacagagatctgaacgccgctcgcaacatcaagagagagggcctgagtcagattgtggtggccggacatgccgaaaccctgaacgccagaggggagggcgtgagacccgccattgccggaagccctagaagcagggctgaccccaagaagaagaggaaggtgtcgccagggatccgtcgacttgacgcgttgatatcaacaagtttgtacaaaaaagcaggctacaaagaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgtttaaaaggccggcggccacgaaaaaggccggccaggcaaaaaagaaaaagatggcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacacaggctatggaactcagtcaactccccagggatatggctcgactggcggctatggcagtagccagagctcccaatcgtcttacgggcagcagtcctcctaccctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtggagaattcggcagtggagagggcagaggaagtctgctaacatgcggtgacgtcgaggagaatcctggcccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtggacgtcgtcgcggggcacgcggagacgttaaacgctcggggagagggtgtcagacctgcgatagcgggcagccctcgatgaagcgagaatccaacggctttagccgttggagtgtcaatcttcatcccttttattttttgttttagagctagaaatagtaagttaaaataaggctagtccgtt

SEQ ID NO: 103 - DNA, 8447 bp, synthetic construct
  source 1..8447: mol_type = other DNA
tttagcgcgtgcgccaattctgcagacaaatggctctagaggtaccgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgcgccaccatggccccaaagaagaagcggaaggtcggtatccacggagtcccagcagccatgaccacccaccgcaaggtgtacagataccgcatcgagcccacccccgtgcaggagagcaagctgtacatgctggccggcagccggagattcgtgttcaactgggccctggcccggagaagagagcactacgccgaaaccggcaagaccctgggatacaacgcccaggcaggagagctgaccgccctgaaaaaccaagaagaaaccagctggctgaaagagtccgactcacaactgctgcagcaggcactgaaagacgtggaaagagccttcgtgaacttcttcgaaaaacgggccagattccccagattcaaaagcaagaaaaccgacacccccagattcagaatcccccaaagagtgagaatagagggcagcagagtgtacgtgcccaaggtggggtgggtgaagctgagaaagagtcaggagatagaaggaaaaaccaaaagcgccaccttcaagagggaggcagatggccattggtacgtgctgctggtgagtgagttcgagatgcctgatgtgcctctgcccccagtgcccgagagcgaggtggtgggaatcgacctgggactgaaggacttctacgtcctgtctgacgggggcaggaaggaggcccccagattcgctagaaagggccagcgaaaactgagaagagctgcaagaagacatagcaagtgcacccggggaagcaacagaaaggccaaggcaaagaggaagctggcaagagtgcacagacagatcgcaaaccagagaaaagacttcgtgcacaaggccaccagcggcctcgtgcagcagtaccaaggcttctgcatcgaaaacctgagcatcaagggcatggccaaaaccaaactgtcaaaaagcgtgctggacgcagccctgggggaatttagaagacagctggcctataaagcccagtggcatagaaagtggctggcagtgatagacagatggttccccagcagcaagctgtgcggagagtgcggcagcattaacgccgatctgaccctgagcgacagagaatggacctgcgagtgtggcgcagtgcacgacagagatctgaacgccgctcgcaacatcaagagagagggcctgagtcagattgtggtggccggacatgccgaaaccctgaacgccagaggggagggcgtgagacccgccattgccggaagccctagaagcagggctgaccccaagaagaagaggaaggtgtcgccagggatccgtcgacttgacgcgttgatatcaacaagtttgtacaaaaaagcaggctacaaagaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgtttaaaaggccggcggccacgaaaaaggccggccaggcaaaaaagaaaaagatggcctcaaacgattatacccaacaagcaacccaaagctatggggcctaccccacccagcccgggcagggctattcccagcagagcagtcagccctacggacagcagagttacagtggttatagccagtccacggacacttcaggctatggccagagcagctattcttcttatggccagagccagaacacaggctatggaactcagtcaactccccagggatatggctcgactggcggctatggcagtagccagagctcccaatcgtcttacgggcagcagtcctcctaccctggctatggccagcagccagctcccagcagcacctcgggaagttacggtagcagttctcagagcagcagctatgggcagccccagagtgggagctacagccagcagcctagctatggtggacagcagcaaagctatggacagcagcaaagctataatccccctcagggctatggacagcagaaccagtacaacagcagcagtggtggtggaggtggaggtggaggtggaggtaactatggccaagatcaatcctccatgagtagtggtggtggcagtggtggcggttatggcaatcaagaccagagtggtggaggtggcagcggtggctatggacagcaggaccgtggaggcactagtggcggaggctccggcggtggcatggccaccgtgaagtttaaatataagggcgaggagaaggaggtggatattagcaagatcaaaaaggtgtggagggtgggcaagatgattagcttcacctacgacgagggcggcggcaagacaggccggggcgcagtgagcgagaaggatgcccccaaggagctgctgcagatgctggagaagcagaagaaggaattcggcagtggagagggcagaggaagtctgctaacatgcggtgacgtcgaggagaatcctggcccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtggacgtcgtcgcggggcacgcggagacgttaaacgctcggggagagggtgtcagacctgcgatagcgggcagccctcgatgaagcgagaatccaacggctttagccgttggagtgtcaatcttcatcccttttattttttgttttagagctagaaatagtaagttaaaataaggctagtccgtt

(19) United States
(12) Patent Application Publication
(10) Pub. No.: US 2026/0055417 A1
(43) Pub. Date: Feb. 26, 2026

(54) OPTIMIZED TARGET-SPECIFIC GENE EXPRESSION REGULATION TECHNOLOGY BASED ON THE ULTRA-MINIATURIZED OMEGA SYSTEM

(71) Applicant: CHUNG ANG UNIVERSITY INDUSTRY ACADEMIC COOPERATION FOUNDATION, Seoul (KR)

(72) Inventors: Seung Hwan LEE, Sejong-si (KR); Yeounsun OH, Seoul (KR)

(21) Appl. No.: 19/262,714

(22) Filed: Jul. 8, 2025

(30) Foreign Application Priority Data
Jul. 9, 2024 (KR) 10-2024-0090691

(51) Int. Cl. C12N 15/67; C07K 14/47

(52) U.S. Cl. CPC C12N 15/67; C07K 14/47

(57) ABSTRACT

The present invention relates to an optimized technology for regulating target-specific gene expression based on an ultra-miniaturized OMEGA system, and a gene expression regulatory system that can enhance gene regulatory efficiency using TnpB is constructed. In the present invention, mutated TnpB with an inactivated DNA cleavage function, or engineered reRNA was developed, establishing the optimal conditions that can yield gene expression efficiency using original TnpB without any introduced mutations, and the vector for regulating gene expression of the present invention is small enough to be loaded in AAVs. Thus, it can be effectively used in a gene expression regulation technology.

### CROSS-REFERENCE TO RELATED APPLICATION

This application claims priority to and the benefit of Korean Patent Application No. 10-2024-0090691, filed on Jul. 9, 2024, the disclosure of which is incorporated herein by reference in its entirety.

### REFERENCE TO A SEQUENCE LISTING SUBMITTED AS AN XML FILE

The Sequence Listing written in the XML file titled “206132-0193-00US_SequenceListing.xml” in XML format, with a creation date of Jul. 8, 2025, and 272,823 bytes in size, is hereby incorporated by reference in its entirety.

### BACKGROUND

### 1. Field of the Invention

The present invention relates to an optimized target-specific gene expression regulation technology based on the ultra-miniaturized OMEGA system.

### 2. Discussion of Related Art

Since the CRISPR system can direct Cas proteins to specific gene sequences with high specificity, various modules have been connected to Cas proteins to develop imaging technologies or various editing technologies. To apply such technologies, it is important to deliver Cas proteins in a safe and efficient manner. In addition, adeno-associated viruses (AAVs) are widely used as delivery vehicles for therapeutic purposes. AAVs are widely used as very safe vehicles due to their low immunogenicity and lack of integration into the genome.

However, AAV can only deliver genetic materials up to approximately 4.7 Kb, and it cannot deliver larger-sized systems. In addition, conventional CRISPR systems face efficiency limitations due to their large size and require additional efforts to split proteins for delivery and then reassemble them for operation. Therefore, there is a growing demand for more compact genome editing systems that can operate efficiently.

Meanwhile, TnpB is a protein belonging to the obligate mobile element guided activity (OMEGA) RNA-guided nuclease family, which is the ancestor of the Cas12-series proteins and present within transposons and serves to maintain the transposons in the genome. TnpB is significantly smaller than conventional Cas proteins, making it suitable for application to various delivery vehicles. It is also attracting attention as a useful protein that can be integrated into various technologies.

Nevertheless, there are few reports on gene regulatory systems that utilize TnpB to increase the efficiency of gene regulation.

### PRIOR ART DOCUMENTS

### [Patent Document]

Korean Laid-Open Patent Publication No. 10-2024-0036632.

### SUMMARY OF THE INVENTION

An object of the present invention is to provide a composition for regulating target gene expression, including a first component comprising a transposon-associated ribonucleoprotein (TnpB) and a second component comprising a right-end transposon element-derived RNA (reRNA) as active ingredients, wherein the composition comprises first and second components each selected from the group consisting of the following, in which the first component is any one selected from the group consisting of:
i) TnpB comprising an amino acid sequence represented by SEQ ID NOL 28; and
ii) TnpB engineered to inactivate its DNA cleavage activity; and
the second component is any one selected from the group consisting of:
i) reRNA comprising a nucleotide sequence represented by SEQ ID NO: 18;
ii) engineered reRNA comprising a scaffold sequence and a spacer sequence, in which the reRNA further comprises a spacer sequence based on SEQ ID NO: 39 and is engineered to have a spacer sequence length of 2 to 30 nt; and
iii) reRNA comprising an additionally engineered scaffold sequence and a spacer sequence, in which the reRNA comprises a scaffold sequence that is the same or added based on engineered reRNA comprising SEQ ID NO: 70 and is further engineered to have a scaffold sequence length of 121 to 183 nt.

Another object of the present invention is to provide a kit including the first component and the second component.

Yet another object of the present invention is to provide an ultra-miniaturized vector for regulating target gene expression, including a nucleotide sequence encoding the first component and the second component.

Yet another object of the present invention is to provide an ultrasmall composition for regulating target gene expression, the composition including above composition or the vector as an active ingredient.

Yet another object of the present invention is to provide an ultra-miniaturized kit for regulating target gene expression, including the composition or the vector; and instructions.

Yet another object of the present invention is to provide a method for regulating target gene expression, the method including a step of transfecting a host cell with the composition or the vector.

Yet another object of the present invention is to provide an engineered transposon-associated ribonucleoprotein (TnpB), including:

an amino acid sequence with the mutated amino acid 187 from the N-terminus of the amino acid sequence represented by SEQ ID NO:28.

Yet another object of the present invention is to provide an engineered right-end transposon element-derived RNA (reRNA), comprising a scaffold sequence and a spacer sequence,
wherein the reRNA further comprises a spacer sequence based on SEQ ID NO: 39, and is engineered to have a spacer sequence length of 2 to 30 nt.

However, technical problems to be solved in the present invention are not limited to the above-described problems, and other problems which are not described herein will be fully understood by those of ordinary skill in the art from the following descriptions.

### Technical Solution

The present invention is to provide a composition for regulating target gene expression, including a first component comprising a transposon-associated ribonucleoprotein (TnpB) and a second component comprising a right-end transposon element-derived RNA (reRNA) as active ingredients, wherein the composition comprises first and second components each selected from the group consisting of the following, in which the first component is any one selected from the group consisting of:

i) TnpB comprising an amino acid sequence represented by SEQ ID NOL 28; and
ii) TnpB engineered to inactivate its DNA cleavage activity; and
the second component is any one selected from the group consisting of:
i) reRNA comprising a nucleotide sequence represented by SEQ ID NO: 18;
ii) engineered reRNA comprising a scaffold sequence and a spacer sequence, in which the reRNA further comprises a spacer sequence based on SEQ ID NO: 39 and is engineered to have a spacer sequence length of 2 to 30 nt; and
iii) reRNA comprising an additionally engineered scaffold sequence and a spacer sequence, in which the reRNA comprises a scaffold sequence that is the same or added based on engineered reRNA comprising SEQ ID NO: 70 and is further engineered to have a scaffold sequence length of 121 to 183 nt.

In one embodiment of the present invention, the engineered TnpB may have a mutation at the 187th amino acid from the N-terminus of the amino acid sequence represented by SEQ ID NO: 28, but is not limited thereto.

In one embodiment of the present invention, the mutation may be one or more selected from the group consisting of insertion, substitution, and deletion, but is not limited thereto.

In one embodiment of the present invention, the engineered TnpB may include an amino acid sequence with the D187A mutation from the N-terminus of the amino acid sequence represented by SEQ ID NO:28, but is not limited thereto.

In one embodiment of the present invention, the engineered TnpB may include the amino acid sequence represented by SEQ ID NO: 30, but is not limited thereto.

In one embodiment of the present invention, the engineered TnpB may be encoded by a nucleotide sequence of SEQ ID NO: 29, but is not limited thereto.

In one embodiment of the present invention, the ii) engineered reRNA may include any one nucleotide sequence selected from the group consisting of SEQ ID NOs: 19 to 24, but is not limited thereto.

In one embodiment of the present invention, the iii) reRNA with an additionally engineered scaffold sequence may be further engineered so that one or more nucleotide from nucleotides 7 to 68 from the 5′ end of a nucleotide sequence represented by SEQ ID NO: 23 are removed, but is not limited thereto.

In one embodiment of the present invention, the iii) reRNA with an additionally engineered scaffold sequence may include any one nucleotide sequence selected from the group consisting of SEQ ID NOs: 60 to 70, but is not limited thereto.

In one embodiment of the present invention, the ii) engineered reRNA or the iii) reRNA with an additionally engineered scaffold sequence may induces inactivation of the DNA cleavage activity of TnpB, but is not limited thereto.

In one embodiment of the present invention, the first component and the second component may be each independently selected from the group consisting of the following, but is not limited thereto:
i) engineered TnpB comprising an amino acid sequence represented by SEQ ID NO: 30, and reRNA comprising the nucleotide sequence represented by SEQ ID NO: 18;
ii) TnpB comprising the amino acid sequence represented by SEQ ID NO: 28, and reRNA or engineered reRNA comprising any one nucleotide sequence selected from the group consisting of SEQ ID NOs: 18 to 24; and
iii) TnpB comprising the amino acid sequence represented by SEQ ID NO: 28, and reRNA with an additionally engineered scaffold sequence comprising any one nucleotide sequence selected from the group consisting of SEQ ID NOs: 60 to 70.

In one embodiment of the present invention, the first component may be further include VP64-p65-Rta (VPR), but is not limited thereto.

In one embodiment of the present invention, the first component may include an amino acid sequence represented by SEQ ID NO: 44 or 46, but is not limited thereto.

In one embodiment of the present invention, the first component may be further include a Fus1 intrinsically disordered region (FUS IDR), but is not limited thereto.

In one embodiment of the present invention, the first and second components may be included as separate compositions or the same composition, but is not limited thereto.

In one embodiment of the present invention, the first and second components may be simultaneously, separately, or sequentially administered, but is not limited thereto.

The present invention provides a kit including the first component and the second component.

The present invention provides an ultra-miniaturized vector for regulating target gene expression, including a nucleotide sequence encoding the first component and the second component.

In one embodiment of the present invention, the vector may include any one selected from the group consisting of the following, but is not limited thereto:
i) a nucleotide sequence encoding any one selected from the group consisting of TnpB comprising an amino acid sequence represented by SEQ ID NO: 28, and the engineered TnpB; and
ii) any one nucleotide sequence selected from the group consisting of reRNA comprising a nucleotide sequence represented by SEQ ID NO: 18, the engineered reRNA of claim 1, and the reRNA with an additionally engineered scaffold sequence.

In one embodiment of the present invention, the vector may include any one nucleotide sequence selected from the group consisting of the following, but is not limited thereto:
i) a nucleotide sequence encoding engineered TnpB comprising an amino acid sequence represented by SEQ ID NO: 30, and reRNA comprising the nucleotide sequence represented by SEQ ID NO: 18;
ii) a nucleotide sequence encoding TnpB comprising the amino acid sequence represented by SEQ ID NO: 28, and reRNA or engineered reRNA comprising any one nucleotide sequence selected from the group consisting of SEQ ID NOs: 18 to 24; and
iii) a nucleotide sequence encoding TnpB comprising the amino acid sequence represented by SEQ ID NO: 28, and reRNA with an additionally engineered scaffold sequence comprising any one nucleotide sequence selected from the group consisting of SEQ ID NOs: 60 to 70.

In one embodiment of the present invention, the vector may additionally include a nucleotide sequence operably encoding one or more selected from the group consisting of SV40 nuclear localization sequence (NLS), a linker, VP64-p65-Rta (VPR), a nucleoplasmin NLS, and a Fus1 intrinsically disordered region (FUS IDR), but is not limited thereto.

In one embodiment of the present invention, the vector may additionally include a nucleotide sequence operably encoding one or more selected from the group consisting of T2A, EGFP, and U6, but is not limited thereto.

In one embodiment of the present invention, the vector may sequentially include a cytomegalovirus promoter (CMV promoter), a SV40 nuclear localization sequence (NLS), the TnpB of claim 1, a linker, VP64-p65-Rta (VPR), a nucleoplasmin NLS, a Fus1 intrinsically disordered region (FUS IDR), T2A, a nucleotide sequence encoding a U6 promoter, and the right-end transposon element-derived RNA (reRNA), but is not limited thereto.

In one embodiment of the present invention, the vector may include any one selected from the group consisting of the nucleotide sequences represented by SEQ ID NOs: 52 to 59, but is not limited thereto.

In one embodiment of the present invention, the vector may be further include hybridization domain (HBD) or Sso7d DNA-binding domain, but is not limited thereto.

In one embodiment of the present invention, the HBD may be located at any one position selected from the group consisting of the following, but is not limited thereto:
i) between TnpB and the linker;
ii) between the nucleoplasmin NLS and the FUS IDR; and
iii) between the FUS IDR and T2A.

In one embodiment of the present invention, the Sso7d DNA-binding domain may be located at any one position selected from the group consisting of the following, but is not limited thereto:
i) between the SV40 NLS and TnpB; and
ii) between the FUS IDR and T2A.

In one embodiment of the present invention, the vector may include a nucleotide sequence represented by any one selected from the group consisting of SEQ ID NOs: 99 to 103, but is not limited thereto.

In one embodiment of the present invention, the vector may any one selected from the group consisting of an adeno-associated virus vector, a lentivirus vector, a retrovirus vector, an adenovirus vector, and a herpes simplex virus vector, but is not limited thereto.

The present invention provides an ultrasmall composition for regulating target gene expression, including the composition or the vector as an active ingredient.

The present invention provides an ultra-miniaturized kit for regulating target gene expression, including the composition or the vector; and instructions.

The present invention provides a method for regulating target gene expression, the method including a step of transfecting a host cell with the composition or the vector.

The present invention provides an engineered transposon-associated ribonucleoprotein (TnpB), including:

an amino acid sequence with the mutated amino acid 187 from the N-terminus of the amino acid sequence represented by SEQ ID NO:28.

In one embodiment of the present invention, the TnpB include an amino acid sequence with the D187A mutation from the N-terminus of the amino acid sequence represented by SEQ ID NO: 28, but is not limited thereto.

In one embodiment of the present invention, the engineered TnpB may include an amino acid sequence comprising SEQ ID NO: 30, but is not limited thereto.

In one embodiment of the present invention, the engineered TnpB may be encoded by a nucleotide sequence comprising SEQ ID NO: 29, but is not limited thereto.

In one embodiment of the present invention, the engineered TnpB may exhibits inactivated DNA cleavage activity, but is not limited thereto.

In one embodiment of the present invention, the engineered TnpB may forms a complex with right-end transposon element-derived RNA (reRNA), but is not limited thereto.

In one embodiment of the present invention, the reRNA may include any one nucleotide sequence selected from the group consisting of SEQ ID NOs: 18 to 24 and SEQ ID NOs: 60 to 81, but is not limited thereto.

In one embodiment of the present invention, the engineered TnpB may be further include one or more selected from the group consisting of VP64-p65-Rta (VPR) and a Fus1 intrinsically disordered region (FUS IDR), but is not limited thereto.

In one embodiment of the present invention, the engineered TnpB may be further include one or more of linker or nucleoplasmin NLS, but is not limited thereto.

In one embodiment of the present invention, the engineered TnpB may include an amino acid sequence represented by SEQ ID NO: 50, but is not limited thereto.

The present invention provides engineered right-end transposon element-derived RNA (reRNA), including a scaffold sequence and a spacer sequence,
wherein the reRNA further include a spacer sequence based on SEQ ID NO: 39, and is engineered to have a spacer sequence length of 2 to 30 nt.

In one embodiment of the present invention, the engineered reRNA may include a nucleotide sequence selected from the group consisting of SEQ ID NOs: 18 to 24, but is not limited thereto.

In one embodiment of the present invention, the engineered reRNA may include a scaffold sequence that is the same or added based on engineered reRNA comprising SEQ ID NO: 70, and may be further engineered to have a scaffold sequence length of 121 to 183 nt, but is not limited thereto.

In one embodiment of the present invention, the scaffold sequence may be engineered so that one or more nucleotide of nucleotides 7 to 68 from the 5′ end of the nucleotide sequence represented by SEQ ID NO: 23 are removed, but is not limited thereto.

In one embodiment of the present invention, the engineered reRNA may include any one nucleotide sequence selected from the group consisting of SEQ ID NOs: 60 to 70, but is not limited thereto.

In one embodiment of the present invention, the engineered reRNA may induces inactivation of the DNA cleavage activity of TnpB, but is not limited thereto.

In one embodiment of the present invention, the engineered reRNA may forms a complex with TnpB, but is not limited thereto.

In one embodiment of the present invention, the TnpB may include any one of the amino acid sequences represented by SEQ ID NOs: 28 and 30, but is not limited thereto.

Additionally, the present invention provides use of the vector or the composition for regulating target gene expression.

Additionally, the present invention provides use of the vector or the composition for preparing an agent for regulating target gene expression.

### BRIEF DESCRIPTION OF THE DRAWINGS

FIGS. 1A to 1E relate to construction of an ISDge10TnpB operating system for introducing an ISDge10TnpB-based ultraminiaturized gene activation system.

FIG. 1A is a schematic diagram of an expression vector for verifying the activity of ISDge10TnpB in human-derived cell lines.

FIG. 1B shows the activity of ultraminiaturized ISDge10TnpB in human-derived cell lines.

FIG. 1C is a schematic diagram of an expression vector for an ISDge10TnpB-based ultraminiaturized gene activation system.

FIG. 1D is a schematic diagram of an expression vector for an ISDge10TnpB-based ultraminiaturized gene activation system, in which a mutation was introduced to eliminate DNA cleavage activity.

FIG. 1E shows a comparison of intracellular gene editing efficiency between CWCas12f and ISDge10TnpB to develop a gene activation system conjugated with a transcriptional activation domain, wherein a system in which the transcriptional activation domain was conjugated to CWCas12f was used as a control. In this experiment, a mutation was introduced to confirm whether DNA cleavage efficiency is abolished.

FIG. 2A shows a schematic diagram of the gene activation mechanism and design through reRNA engineering for ISDge10 optimization of a ultraminiaturized gene activation system. The engineered reRNA allows control of the expression level of a target gene in a ultraminiaturized gene activation system (ISDge10TnpB-VPR), and the activity of ISDge10TnpB can be regulated depending on the engineering of reRNA.

FIGS. 2B to 2F show experimental results for constructing an optimized gene activation system based on ultraminiaturized ISDge10TnpB.

FIG. 2B shows a gene expression validation system using a fluorescent reporter.

FIG. 2C shows gene expression activation results of CWCas12f-VPR regulated by sgRNA.

FIG. 2D shows gene expression activation results of ISDge10TnpB-VPR regulated by reRNA, demonstrating that regulation by gRNA or reRNA leads to higher gene expression activation than a mutant version in which DNA cleavage activity was eliminated.

FIGS. 2E and 2F show DNA cleavage efficiency results for engineered versions of gRNA or reRNA in CWCas12f-VPR and ISDge10TnpB, respectively. The results demonstrate that shorter versions did not induce DNA cleavage but did induce target gene expression activation.

FIG. 2G shows a schematic diagram of the gene cargo size of systems compatible with an AAV vector for efficient and safe delivery, demonstrating that a system based on miniaturized proteins can be loaded together with various modules.

FIG. 3 shows gene cleavage efficiency when using reRNA engineered in the scaffold sequence and spacer sequence.

FIG. 4 provides a comparative analysis of gene expression activation efficiency when using reRNA engineered in the scaffold sequence and spacer sequence.

FIG. 5A shows a schematic design of vectors in which various domains are additionally conjugated to ISDge10TnpB, and FIG. 5B provides a comparative analysis of gene expression activation efficiency using the vectors.

### DETAILED DESCRIPTION OF EXEMPLARY EMBODIMENTS

TnpB, a protein belonging to the Obligate Mobile Element Guided Activity (OMEGA) RNA-guided nuclease family, is known as an ancestor of Cas12 family proteins, and is known to cleave double-stranded and single-stranded DNA substrates in a target-specific manner by forming a DNA-RNA hybrid duplex at a target site using the endonuclease TnpB and reRNA. Compared to conventional Cas proteins, TnpB is much smaller in size, which may be advantageous for application to various delivery vehicles. The present inventors have completed the present invention by engineering TnpB and reRNA to identify the most active combination that enables highly accurate regulation of gene expression without inducing DNA cleavage, while taking advantage of the small size of TnpB, so that it can be used as a genome editing tool in vivo and may be usefully applied in future gene therapy and biological research.

The newly developed aspects of the present invention are as follows:

Target-specific gene expression activation can be achieved using the ISDge10 TnpB-VPR system developed by conjugating an optimized transcriptional activation domain, linker, and NLS sequences to the miniaturized protein ISDge10 TnpB.

The ISDge10 TnpB-VPR system was further optimized through reRNA engineering that adjusts the length of the DNA-RNA hybrid formed by the reRNA, which confers target specificity. Using this approach, a transcriptional regulator can be conjugated to a CRISPR-Cas effector that does not induce DNA double-strand breaks, thereby enabling efficient and safe activation of specific gene expression.

Unlike conventional CRISPR genome editing technologies, the present invention allows precise regulation of target gene expression without cutting or nicking DNA, making it relatively safer and optimally suited for application in gene therapy, with a potentially broad impact.

Because gene expression can be regulated efficiently and with high accuracy based on the miniaturized ISDge10 TnpB, not only activation but also repression of expression is possible.

The gene regulation system provided in the present invention is extremely small in size and enables efficient transcriptional regulation, and thus may serve as a foundation for the development of useful technologies not only for studies on gene function but also for future gene therapy applications.

The gene size for expressing the miniaturized ISDge10 TnpB system falls within the maximum packaging capacity of an AAV system, allowing safe and efficient delivery, and therefore, when applied to the development of gene therapeutics for human use, it has a significant potential impact on the medical market.

CRISPR genome editing technology consists of a CRISPR-Cas protein that recognizes a specific DNA sequence and a guide RNA that provides target sequence specificity. This enables gene-specific regulation by designing an appropriate guide RNA for each target, making it a highly impactful technology. For the application and utilization of genome editing technologies, not only high efficiency but also safe and efficient delivery is critical. The adeno-associated virus (AAV) vector, optimized for delivery to human cells, has a limited genome packaging capacity, making it difficult to deliver large genetic cargos and resulting in low delivery efficiency. Accordingly, gene editing and regulation technologies using CRISPR systems that are significantly smaller than conventional systems are gaining attention for effective delivery using AAV vectors. Among human-derived diseases, not only those caused by mutations in specific genes but also those resulting from abnormal regulation of gene expression have been widely reported. Rather than direct gene editing, which may cause unintended genetic mutations, approaches that regulate the expression levels of genes for therapeutic purposes are receiving increasing attention. The present invention enables gene expression regulation by linking a transcriptional activation module to ISDge10 TnpB, a miniaturized protein that is significantly smaller than those used in conventional technologies. This was achieved by engineering the reRNA of ISDge10 TnpB to control the length of the RNA-DNA hybrid duplex formed with the target DNA, thereby enabling efficient target-specific gene expression regulation without inducing DNA double-strand breaks. Unlike conventional genome editing technologies, the present invention enables regulation of target gene expression without damaging DNA, making it relatively safe. Moreover, due to its extremely small size, it can be delivered safely and efficiently, making it an optimized technology for gene therapy applications.

That is, the inventors of the present invention confirmed that the effect was remarkably superior when target gene expression was regulated by using a combination of a TnpB mutant with inactivated DNA cleavage activity or reRNA, or a combination of TnpB retaining DNA cleavage activity with reRNA engineered to adjust the spacer sequence length.

Accordingly, the present invention provides an engineered transposon-associated ribonucleoprotein (TnpB), including:
an amino acid sequence with the mutated amino acid 187 from the N-terminus of the amino acid sequence represented by SEQ ID NO: 28.

In one embodiment of the present invention, the TnpB may comprise an amino acid sequence comprising a D187A mutation from the N-terminus of the amino acid sequence represented by SEQ ID NO: 28, but is not limited thereto. Accordingly, the present invention provides an engineered transposon-associated ribonucleoprotein (TnpB), comprising an amino acid sequence comprising a D187A mutation from the N-terminus of the amino acid sequence represented by SEQ ID NO: 28, or comprising the amino acid sequence represented by SEQ ID NO: 30. In one embodiment of the present invention, the engineered TnpB may be encoded by a nucleotide sequence comprising SEQ ID NO: 29, but is not limited thereto.

In the present invention, the term “engineered TnpB” may be used interchangeably with “TnpB variant” or “TnpB with abolished cleavage activity.” That is, the engineered TnpB may refer to a TnpB protein in which the DNA cleavage activity is inactivated by having one or more mutations in the amino acid sequence. In this case, the amino acid sequence may correspond to SEQ ID NO: 30 of the present invention. In one embodiment of the present invention, it was confirmed that DNA cleavage activity of TnpB was abolished even when using the amino acid sequence of SEQ ID NO: 30, which comprises only a D187A mutation from the N-terminus of the amino acid sequence represented by SEQ ID NO: 28.

In one embodiment of the present invention, the engineered TnpB may have inactivated DNA cleavage activity, but is not limited thereto. In one embodiment of the present invention, it was confirmed that the TnpB with inactivated DNA cleavage activity according to the present invention exhibited excellent gene expression regulation efficiency and more precise regulatory activity, even though the DNA cleavage activity was inactivated.

In one embodiment of the present invention, the engineered TnpB may form a complex with reRNA (right-end transposon element-derived RNA), but is not limited thereto.

The TnpB of the present invention is a protein having gene cleavage activity and is known as a gene editing protein. A protein having gene cleavage activity performs the gene editing process in conjunction with RNA that leads to the target gene. According to common knowledge in the art, TnpB forms a complex with reRNA during the gene editing process, and therefore the term “complex” may refer to the TnpB-reRNA complex. Although the TnpB-reRNA complex exhibits gene editing activity, it is not necessarily required that the complex be formed at the time of TnpB administration for gene editing, and the same may apply after the gene editing process.

In one embodiment of the present invention, the reRNA may comprise a nucleotide sequence selected from the group consisting of SEQ ID NOs: 18 to 24 and SEQ ID NOs: 60 to 81, but is not limited thereto.

In one embodiment of the present invention, the engineered TnpB may be additionally conjugated with one or more selected from the group consisting of VPR (VP64-p65-Rta) and FUS IDR (Fus1 intrinsically disordered region), but is not limited thereto.

In one embodiment of the present invention, the engineered TnpB may additionally comprise one or more selected from linker or nucleoplasmin NLS (nucleoplasmin NLS), but is not limited thereto.

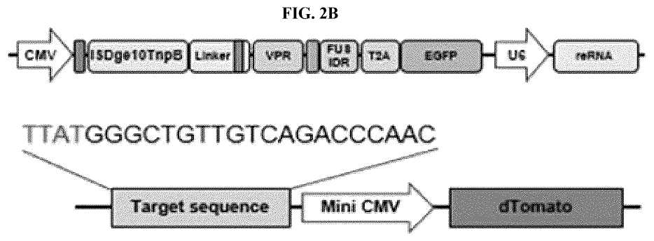

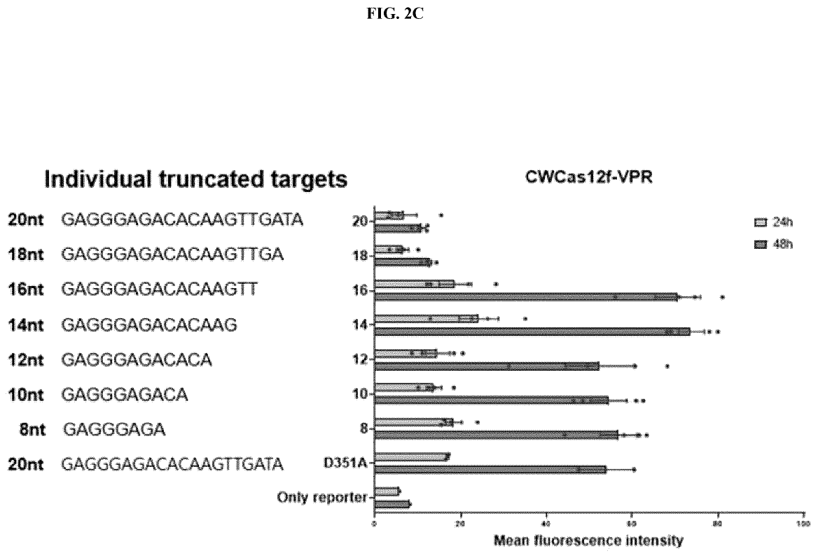

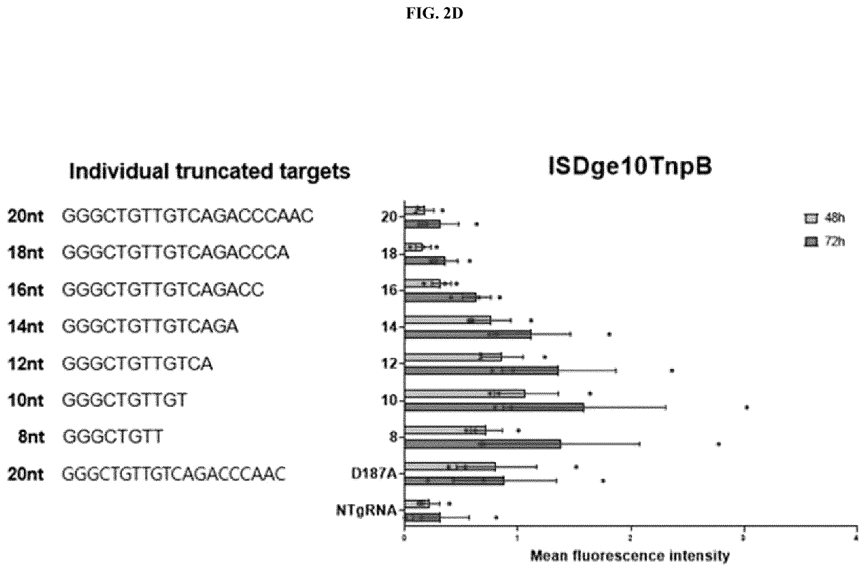

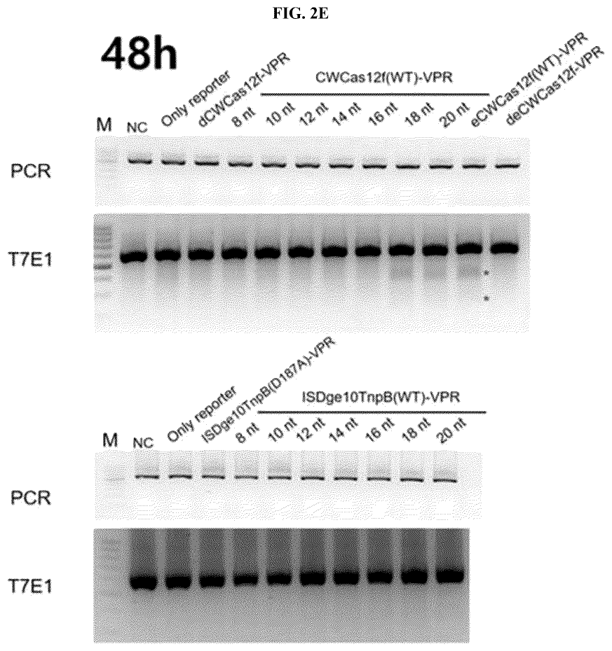

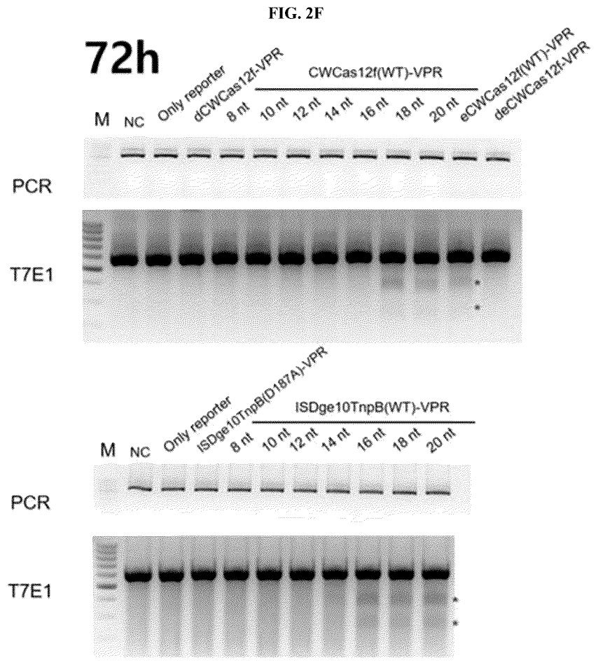

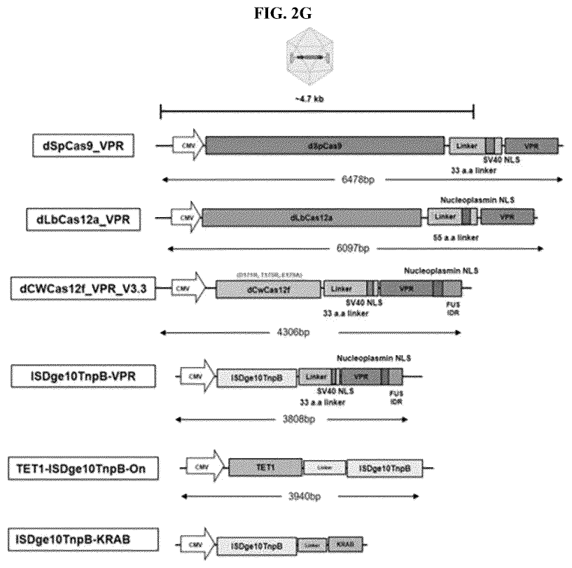

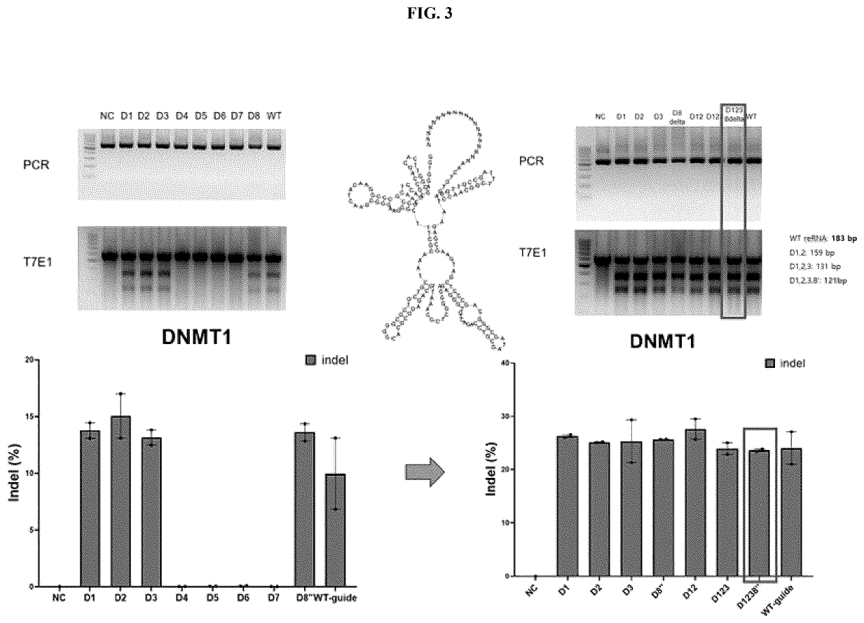

In the present invention, TnpB may form a complex by being sequentially conjugated with VPR and FUS IDR (FIG. 2A). The TnpB of the present invention is conjugated to VPR via a linker of the present invention. In addition, in the present invention, the FUS IDR may be conjugated to the VPR conjugated to TnpB, and the method of conjugation may be any conventionally applicable method known in the art. In the present invention, the nucleoplasmin NLS may be positioned between the VPR conjugated to TnpB and the FUS IDR. Such a conjugation method and order can be clearly identified through the amino acid sequence represented by SEQ ID NO: 50. However, since TnpB, VPR, and FUS IDR can be conjugated by methods commonly used in gene editing, they are not limited by the methods of the present invention.

In one embodiment of the present invention, the engineered TnpB may comprise the amino acid sequence represented by SEQ ID NO: 50, but is not limited thereto.

Additionally, the present invention provides an engineered reRNA (right-end transposon element-derived RNA) comprising a scaffold sequence and a spacer sequence,
wherein the reRNA is engineered by adding a spacer sequence to SEQ ID NO: 39 such that the spacer sequence has a length of 2 to 30 nt. For example, 2 to 28 nt, 2 to 24, 2 to 22, 2 to 20, 4 to 30 nt, 4 to 28 nt, 4 to 24, 4 to 22, 4 to 20, 6 to 30 nt, 6 to 28 nt, 6 to 24, 6 to 22, 6 to 20, 8 to 30 nt, 8 to 28 nt, 8 to 24, 8 to 22, 8 to 20, but is not limited thereto.

In the present invention, “reRNA” refers to an RNA that imparts target specificity to TnpB and may be understood as a type of guide RNA. Guide RNA (gRNA) or single guide RNA (sgRNA) is known as a short RNA sequence that serves as a guide for Cas9 endonuclease or other Cas proteins capable of cleaving double-stranded DNA for use in gene editing. The reRNA may form a complex (RNP) with TnpB and function as a gene editing system through DNA cleavage, but is not limited thereto.

In the present invention, the term “engineered reRNA” may refer to a reRNA that is engineered to induce inactivation of the DNA cleavage activity of TnpB. In the present invention, one or more of the spacer sequence or the scaffold sequence of the reRNA was engineered to inactivate the DNA cleavage activity of TnpB. The reRNA may comprise a scaffold sequence and a spacer sequence. Accordingly, the term “engineered reRNA” may broadly include a reRNA in which the spacer sequence is engineered, a reRNA in which the scaffold sequence is engineered, and a reRNA in which both the spacer sequence and scaffold sequence are engineered. In the present invention, both a reRNA in which the spacer sequence is engineered and a reRNA in which both the spacer sequence and the scaffold sequence are engineered were prepared and used in the examples. Therefore, to clearly distinguish the two, the reRNA with an engineered spacer sequence is referred to as “engineered reRNA,” and the reRNA with both the spacer sequence and scaffold sequence engineered is referred to as “scaffold sequence-further engineered reRNA,” but the invention is not limited thereto and each term may be clearly understood according to the technical context.

Additionally, in the present invention, spacer sequences having lengths of 2 to 30 nt, and more specifically 8 to 20 nt, were generated from a nucleotide sequence in which the entire spacer sequence of SEQ ID NO: 39 was removed, and these were represented by SEQ ID NOs: 18 to 24. At that time, the reRNA having a spacer sequence with a length of 20 nt was the longest and thus used as the reference sequence. Since this sequence was used in combination with the engineered TnpB to evaluate gene expression regulation efficiency, the nucleotide sequence represented by SEQ ID NO: 18, which includes the 20 nt spacer sequence, was designated as the reference and labeled as reRNA for distinction. However, since SEQ ID NO: 18 was also prepared by engineering according to the method of the present invention, SEQ ID NOs: 18 to 24 are all included as engineered reRNAs in the present invention.

In the present invention, the term “spacer sequence” may refer to the portion of a guide RNA sequence that provides target specificity for a gene-editing protein and forms a hybrid duplex with the target DNA for gene editing. In the present invention, it may refer to the sequence that forms an RNA-DNA hybrid duplex between reRNA, a type of guide RNA for the gene-editing protein TnpB, and the target DNA. In the present invention, the length of the sequence forming the RNA-DNA hybrid duplex between the reRNA and the target DNA was adjusted through engineering of the spacer sequence of the reRNA.

In the present invention, the term “scaffold sequence” may refer to the portion of a guide RNA sequence that is recognized by a gene-editing protein and confers target specificity. In the present invention, it may refer to a portion of the reRNA sequence, which is a type of guide RNA for the gene-editing protein TnpB, that is recognized by TnpB. In the present invention, the length of the sequence recognized by TnpB was adjusted through engineering of the scaffold sequence of the reRNA.

First, in the present invention, it was confirmed that when the length of the spacer sequence of the reRNA was adjusted, the DNA cleavage activity of TnpB was inactivated while still exhibiting excellent gene expression regulatory activity, and the optimal spacer sequence length range for achieving the highest gene expression regulatory activity was identified to be 8 to 20 nt, more specifically 10 nt.

Additionally, in the present invention, it was confirmed that further adjustment of the length of the scaffold sequence of the reRNA also resulted in effects equal to or greater than those observed when adjusting the length of the spacer sequence. The engineered reRNA in the present invention was produced by demarcating the region corresponding to structural sequences from position 7 to 68 from the 5′ end of the reRNA represented by Sequence No. 23, and then removing one or more sections within that region. In the present invention, based on the scaffold sequence of the engineered reRNA comprising SEQ ID NO: 70, eight regions (Nos. 1 to 8) were designated, and reRNAs with each of the respective regions deleted were prepared. These reRNAs were analyzed for gene expression regulation activity in combination with wild-type TnpB retaining DNA cleavage activity. As a result, it was confirmed that reRNAs in which region 1, 2, 3, or 8 was deleted in the scaffold sequence exhibited excellent gene expression regulation activity while inactivating the DNA cleavage activity of TnpB. In particular, when all of regions 1 to 3 and 8 were deleted, resulting in a scaffold sequence length of 121 nt, it was confirmed that the gene expression regulation activity remained excellent while the length was minimized, thereby establishing an ultra-miniaturized gene expression regulation system comprising this configuration. In the present invention, in order to minimize the length of the reRNA, the scaffold sequence length was further engineered while maintaining the spacer sequence length at 10 nt. Accordingly, it can be understood from the embodiments of the present invention that when the same mutations are introduced into the scaffold sequences of reRNAs comprising spacer sequences with lengths of 8 to 20 nt, similar effects would be obtained. Therefore, the total length of reRNA can be at least 129 nt (8nt, D1238) and at most 203 nt (20nt, X).

Additionally, although not explicitly described, it can be readily inferred from the disclosure and experimental data of the present invention that a scaffold sequence-further engineered reRNA, presumed to be disclosed in the present invention, when used in combination with TnpB, would exhibit superior gene expression regulation effects compared to wild-type reRNA or exhibit effects equal to or greater than those of an engineered reRNA in which only the spacer sequence is engineered.

In the present invention, SEQ ID NO: 39 may refer to a reRNA sequence used in the present invention in which the spacer sequence has been entirely removed. Since the reRNA of the present invention follows the rule of having a “g” at the very beginning and [ttttatttt] after the spacer sequence, it can be inferred that a sequence comprising “g” at the 5′ end and [ttttatttt] at the 3′ end of SEQ ID NO: 39, although not explicitly described, is disclosed in the present invention. Furthermore, it is evident that the addition of a spacer sequence based on SEQ ID NO: 39 refers to the addition of nucleotides corresponding to the spacer sequence upstream of [ttttatttt], as indicated in Table 2 of the present invention.

In one embodiment of the present invention, the engineered reRNA may comprise a nucleotide sequence selected from the group consisting of SEQ ID NOs: 18 to 24, but is not limited thereto.

In one embodiment of the present invention, the engineered reRNA may be further engineered based on an engineered reRNA comprising SEQ ID NO: 70 such that the scaffold sequence is the same or extended, and the scaffold sequence length becomes 121 to 183 nt, but is not limited thereto. In this case, a scaffold sequence length of 121 nt may refer to a scaffold sequence in which all of regions 1 to 3 and 8 as described above are deleted. In addition, a scaffold sequence length of 183 nt may refer to a scaffold sequence from which the shortest length has been deleted. Therefore, in the present invention, the minimum length of the scaffold sequence of the reRNA may be 121 nt, and the maximum length may be 183 nt.

In the present invention, it was confirmed that adjusting the length of the scaffold sequence maintained or induced inactivation of the DNA cleavage activity of TnpB while still exhibiting excellent gene expression regulation activity, and in particular, the most favorable effects were observed when regions 1, 2, 3, and 8 (D1, D2, D3, and D8); regions 1 and 2 (D12); regions 1 to 3 (D123); or regions 1 to 3 and 8 (D1238 delta) were deleted, respectively. Such effects may be attributable not only to the length of the scaffold sequence but also to the specificity of the deleted regions. Since deletion of regions 4 to 7 resulted in markedly low gene expression regulation activity, the excellent gene expression regulation effect achieved by the combination of the scaffold sequence length and the deleted regions in the present invention has been demonstrated.

In one embodiment of the present invention, the scaffold sequence may be further engineered such that one or more nucleotides among the 7th to 68th nucleotides from the 5′ end of the nucleotide sequence represented by SEQ ID NO: 23 are deleted, but is not limited thereto.

In this case, the method of engineering based on SEQ ID NO: 23 is described according to the method for preparing the scaffold sequence of the present invention. Meanwhile, as described above, SEQ ID NO: 70 in the present invention may refer to the nucleotide sequence having the shortest scaffold sequence length, and thus the engineered reRNA comprising the scaffold sequence of the present invention may be expressed as being added based on the scaffold sequence of SEQ ID NO: 70. The reRNA comprising the scaffold sequence that is further engineered in the present invention may be described based on the shortest scaffold sequence or based on the preparation method, and this is merely a difference in expression, while the final reRNA product prepared is the same.

In one embodiment of the present invention, the engineered reRNA may comprise a nucleotide sequence selected from the group consisting of SEQ ID NOs: 60 to 70, but is not limited thereto. In this case, the engineered reRNA refers to a scaffold sequence-further engineered reRNA.

In the present invention, the scaffold sequence-further engineered reRNA was constructed based on a spacer sequence length of 10 nt, and the specific sequences of the scaffold sequence-further engineered reRNAs were described for cases where the spacer sequence length was 10 nt (SEQ ID NOs: 60 to 70) or 20 nt (SEQ ID NOs: 71 to 81). Meanwhile, the present invention provides reRNAs having spacer sequence lengths of 8 to 20 nt and discloses in detail the preparation method of the scaffold sequence-further engineered reRNAs, and thus, even though the nucleotide sequences of the scaffold sequence-further engineered reRNAs based on spacer sequence lengths other than 10 nt and 20 nt are not explicitly described, they are understood to be provided by the present invention.

In one embodiment of the present invention, the engineered reRNA may be designed to induce inactivation of the DNA cleavage activity of TnpB, but is not limited thereto. In this case, the engineered reRNA that induces inactivation of the DNA cleavage activity of TnpB may refer to a reRNA having a spacer sequence length of 8 to 20 nt, such as a reRNA represented by one or more nucleotide sequences selected from the group consisting of SEQ ID NOs: 18 to 24.

In one embodiment of the present invention, the engineered reRNA may form a complex with TnpB, but is not limited thereto.

In one embodiment of the present invention, the TnpB may comprise any one of the amino acid sequences represented by SEQ ID NO: 28 or SEQ ID NO: 30, but is not limited thereto.

Therefore, the present invention provides a composition for regulating target gene expression, including a first component comprising a transposon-associated ribonucleoprotein (TnpB) and a second component comprising a right-end transposon element-derived RNA (reRNA) as active ingredients,
wherein the composition comprises first and second components each selected from the group consisting of the following, in which the first component is any one selected from the group consisting of:
i) TnpB comprising an amino acid sequence represented by SEQ ID NOL 28; and
ii) TnpB engineered to inactivate its DNA cleavage activity; and
the second component is any one selected from the group consisting of:
i) reRNA comprising a nucleotide sequence represented by SEQ ID NO: 18;
ii) engineered reRNA comprising a scaffold sequence and a spacer sequence, in which the reRNA further comprises a spacer sequence based on SEQ ID NO: 39 and is engineered to have a spacer sequence length of 2 to 30 nt; and
iii) reRNA comprising an additionally engineered scaffold sequence and a spacer sequence, in which the reRNA comprises a scaffold sequence that is the same or added based on engineered reRNA comprising SEQ ID NO: 70 and is further engineered to have a scaffold sequence length of 121 to 183 nt.

In one embodiment of the present invention, the engineered TnpB may have a mutation at the 187th amino acid from the N-terminus of the amino acid sequence represented by SEQ ID NO: 28, but is not limited thereto.

In one embodiment of the present invention, the mutation may be one or more selected from the group consisting of insertion, substitution, and deletion, but is not limited thereto. In the present invention, it was confirmed that the DNA cleavage activity of TnpB was inactivated when a mutation caused by amino acid substitution was introduced, but is not limited thereto. In particular, aspartic acid (Asp or D) does not necessarily have to be substituted with alanine (Ala or A), and it may be substituted with other amino acids.

In one embodiment of the present invention, the engineered TnpB may include an amino acid sequence with the D187A mutation from the N-terminus of the amino acid sequence represented by SEQ ID NO:28, but is not limited thereto. Therefore, in one embodiment of the present invention, the engineered TnpB may include the amino acid sequence represented by SEQ ID NO: 30, but is not limited thereto.

In one embodiment of the present invention, the engineered TnpB may be encoded by a nucleotide sequence of SEQ ID NO: 29, but is not limited thereto.

In one embodiment of the present invention, the ii) engineered reRNA may include any one nucleotide sequence selected from the group consisting of SEQ ID NOs: 19 to 24, but is not limited thereto.

In one embodiment of the present invention, the iii) reRNA with an additionally engineered scaffold sequence may be further engineered so that one or more nucleotide from nucleotides 7 to 68 from the 5′ end of a nucleotide sequence represented by SEQ ID NO: 23 are removed, but is not limited thereto.

As described above, the scaffold sequence of the present invention was divided into regions 1 to 8 based on the 5′ end of the nucleotide sequence represented by SEQ ID NO: 23, as follows:

Region 1 (deletion of nucleotides from the 7th to the 21st), Region 2 (deletion of nucleotides from the 22nd to the 30th), Region 3 (deletion of nucleotides from the 31st to the 58th), Region 4 (deletion of nucleotides from the 69th to the 93rd), Region 5 (deletion of nucleotides from the 94th to the 108th), Region 6 (deletion of nucleotides from the 109th to the 142nd), Region 7 (deletion of nucleotides from the 156th to the 177th), Region 8 (deletion of nucleotides from the 59th to the 68th), Region 1-2 (deletion of all nucleotides from the 7th to the 30th), Region 1-3 (deletion of all nucleotides from the 7th to the 58th), and Region 1-3 and Region 8 (deletion of all nucleotides from the 7th to the 68th).

In one embodiment of the present invention, the iii) reRNA with an additionally engineered scaffold sequence may include any one nucleotide sequence selected from the group consisting of SEQ ID NOs: 60 to 70, but is not limited thereto.

In one embodiment of the present invention, the ii) engineered reRNA or the iii) reRNA with an additionally engineered scaffold sequence may induces inactivation of the DNA cleavage activity of TnpB, but is not limited thereto.

In one embodiment of the present invention, the first component and the second component may be each independently selected from the group consisting of the following, but is not limited thereto:
i) engineered TnpB comprising an amino acid sequence represented by SEQ ID NO: 30, and reRNA comprising the nucleotide sequence represented by SEQ ID NO: 18;
ii) TnpB comprising the amino acid sequence represented by SEQ ID NO: 28, and reRNA or engineered reRNA comprising any one nucleotide sequence selected from the group consisting of SEQ ID NOs: 18 to 24; and
iii) TnpB comprising the amino acid sequence represented by SEQ ID NO: 28, and reRNA with an additionally engineered scaffold sequence comprising any one nucleotide sequence selected from the group consisting of SEQ ID NOs: 60 to 70.

As described above, using any one of the nucleotide sequences represented by SEQ ID NOs: 71 to 81 may result in the same or greater gene expression regulation activity, and the same result may also be obtained when using an engineered reRNA (in a broad sense) that, although not explicitly described, can be inferred to be disclosed in the present invention.

In this case, the first component and the second component may refer to the combination of the first component and the second component. Since the first component is a TnpB protein and the second component is a reRNA that imparts gene target specificity to the TnpB protein, it is clearly inferred that the combination of each component constitutes a gene expression regulation system.

In one embodiment of the present invention, the first component may be further include VP64-p65-Rta (VPR), but is not limited thereto.

In one embodiment of the present invention, the first component may include an amino acid sequence represented by SEQ ID NO: 44 or 46, but is not limited thereto.

In one embodiment of the present invention, the first component may be further include a Fus1 intrinsically disordered region (FUS IDR), but is not limited thereto.

In one embodiment of the present invention, the first and second components may be included as separate compositions or the same composition, but is not limited thereto.

As described above, since the first component and the second component are a TnpB protein and a reRNA that imparts gene target specificity to the TnpB protein, respectively, the first component and the second component, when included and treated within a single composition, may exhibit gene expression regulation effects as a component of a gene expression regulation system. In addition, it is clearly understood by those skilled in the art that even when each component is included in separate compositions, gene expression regulation activity may be exhibited if they are appropriately treated to form a complex for gene expression regulation. Accordingly, the first component and the second component of the present invention may be included independently in respective compositions, and may also be included together in a single composition.

If each component is included in a separate composition, then in one embodiment of the present invention, the first component and the second component may be administered simultaneously, separately, or sequentially, but are not limited thereto.

The present invention provides a kit including the first component and the second component.

In the present invention, the term “kit” refers to the tool itself comprising the first component and the second component of the present invention. The kit of the present invention may further include other components, compositions, solutions, or devices conventionally required for storage and handling of the above materials. Specifically, each component may be applied one or more times without limitation in the number of applications, there is no limitation on the order in which each material is applied, and each material may be applied simultaneously or sequentially.

In the present invention, the kit may include a container, instructions, and the like. The container may serve to package the above substance, and may also serve to store and secure it. The material of the container may take the form of, for example, a bottle, tub, sachet, envelope, tube, or ampoule, and may be formed partially or entirely from plastic, glass, paper, foil, wax, or the like. The container may be equipped with a cap that is initially a part of the container or is completely or partially detachable and attachable to the container by mechanical, adhesive, or other means, and may also be equipped with a stopper that allows access to the contents with a syringe needle. The kit may include an external package, and the external package may include instructions for use of the components.

Additionally, the present invention provides an ultra-miniaturized vector for regulating target gene expression, including a nucleotide sequence encoding the first component and the second component.

In one embodiment of the present invention, the vector may include any one selected from the group consisting of the following, but is not limited thereto:
i) a nucleotide sequence encoding any one selected from the group consisting of TnpB comprising an amino acid sequence represented by SEQ ID NO: 28, and the engineered TnpB; and
ii) any one nucleotide sequence selected from the group consisting of reRNA comprising a nucleotide sequence represented by SEQ ID NO: 18, the engineered reRNA of claim 1, and the reRNA with an additionally engineered scaffold sequence.

In one embodiment of the present invention, the vector may include any one nucleotide sequence selected from the group consisting of the following, but is not limited thereto:
i) a nucleotide sequence encoding engineered TnpB comprising an amino acid sequence represented by SEQ ID NO: 30, and reRNA comprising the nucleotide sequence represented by SEQ ID NO: 18;
ii) a nucleotide sequence encoding TnpB comprising the amino acid sequence represented by SEQ ID NO: 28, and reRNA or engineered reRNA comprising any one nucleotide sequence selected from the group consisting of SEQ ID NOs: 18 to 24; and
iii) a nucleotide sequence encoding TnpB comprising the amino acid sequence represented by SEQ ID NO: 28, and reRNA with an additionally engineered scaffold sequence comprising any one nucleotide sequence selected from the group consisting of SEQ ID NOs: 60 to 70.

In one embodiment of the present invention, the vector may additionally include a nucleotide sequence operably encoding one or more selected from the group consisting of SV40 nuclear localization sequence (NLS), a linker, VP64-p65-Rta (VPR), a nucleoplasmin NLS, and a Fus1 intrinsically disordered region (FUS IDR), but is not limited thereto.

In one embodiment of the present invention, the vector may additionally include a nucleotide sequence operably encoding one or more selected from the group consisting of T2A, EGFP, and U6, but is not limited thereto.

In the present invention, “NLS (Nuclear Localization Sequence, or Signal)” refers to a peptide of a certain length, or the sequence thereof, that serves as a kind of “tag” attached to a transport-targeted protein for transporting a substance from outside the nucleus into the nucleus by nuclear transport. Specifically, the NLS may be an SV40 NLS or a nucleoplasmin NLS, but is not limited thereto. For example, the SV40 NLS may be represented by the amino acid sequence of SEQ ID NO: 26, or may be encoded by a nucleotide sequence comprising a nucleotide sequence (e.g., SEQ ID NO: 25) that encodes the amino acid sequence, but is not limited thereto. In addition, the nucleoplasmin NLS may be represented by the amino acid sequence of SEQ ID NO: 36, or may be encoded by a nucleotide sequence comprising a nucleotide sequence (e.g., SEQ ID NO: 35) that encodes the amino acid sequence, but is not limited thereto. The term “NLS” as used in the present specification includes all meanings recognizable by those skilled in the art and may be interpreted appropriately depending on the context.

In the present invention, one or more components of the vector of the present invention may be linked by a “linker” of the present invention. The linker of the present invention may comprise an SV40 NLS therein, but is not limited thereto.

In one embodiment of the present invention, the vector may comprise a nucleotide sequence encoding CMV promoter (cytomegalovirus promoter), SV40 NLS (nuclear localization sequence), the TnpB, linker, VPR (VP64-p65-Rta), nucleoplasmin NLS (nucleoplasmin nuclear localization sequence), FUS IDR (Fus1 intrinsically disordered region), T2A, and U6 promoter, and a nucleotide sequence operably linked to the reRNA (right-end transposon element-derived RNA), but is not limited thereto.

In addition, In one embodiment of the present invention, the vector may sequentially include a cytomegalovirus promoter (CMV promoter), a SV40 nuclear localization sequence (NLS), the TnpB of claim 1, a linker, VP64-p65-Rta (VPR), a nucleoplasmin NLS, a Fus1 intrinsically disordered region (FUS IDR), T2A, a nucleotide sequence encoding a U6 promoter, and the right-end transposon element-derived RNA (reRNA), but is not limited thereto.

In this case, the TnpB or the reRNA may include the engineered TnpB, the engineered reRNA, and the scaffold sequence-engineered reRNA of the present invention. Whether each is applicable can be clearly inferred and understood according to the combinations disclosed in the present invention. For example, when the TnpB is engineered, it may be combined with a wild-type (WT) reRNA in which neither the spacer sequence nor the scaffold sequence is engineered, a reRNA in which the spacer sequence is engineered, or a scaffold sequence-engineered reRNA.

In one embodiment of the present invention, the vector may include any one selected from the group consisting of the nucleotide sequences represented by SEQ ID NOs: 52 to 59, but is not limited thereto.

In one embodiment of the present invention, the vector may be further include

hybridization domain (HBD) or Sso7d DNA-binding domain, but is not limited thereto. In one embodiment of the present invention, the HBD may located at any one position selected from the group consisting of the following, but is not limited thereto:
i) between TnpB and the linker;
ii) between the nucleoplasmin NLS and the FUS IDR; and
iii) between the FUS IDR and T2A.

In one embodiment of the present invention, the Sso7d DNA-binding domain may located at any one position selected from the group consisting of the following, but is not limited thereto:
i) between the SV40 NLS and TnpB; and
ii) between the FUS IDR and T2A.

Therefore, in one embodiment of the present invention, the HBD may be located at any one position selected from the group consisting of the following:
i) between TnpB and the linker;
ii) between the nucleoplasmin NLS and the FUS IDR; and
iii) between the FUS IDR and T2A, and
the Sso7d DNA-binding domain may be located at any one position selected from the group consisting of the following, but is not limited thereto:
i) between the SV40 NLS and TnpB; and
ii) between the FUS IDR and T2A.

In one embodiment of the present invention, the vector may include a nucleotide sequence represented by any one selected from the group consisting of SEQ ID NOs: 99 to 103, but is not limited thereto.

In one embodiment of the present invention, the vector may any one selected from the group consisting of an adeno-associated virus vector, a lentivirus vector, a retrovirus vector, an adenovirus vector, and a herpes simplex virus vector, but is not limited thereto.

In the present invention, “regulation of gene expression” includes both increasing and decreasing the expression of a target gene. In this case, the level of regulation is not limited by quantitative values. Accordingly, the target gene expression level may be arbitrarily regulated by the gene expression regulation vector of the present invention or a gene expression regulation system comprising the same, using methods generally practiced in the art.

In the present invention, a system for regulating the expression of a target gene using the vector may be defined as a “miniaturized target gene expression regulation system,” and the present invention provides a method for regulating the expression of a target gene using the system.

In the present invention, the term “miniaturized” (or ultra-miniaturized, ultrasmall) may refer to the size of a vector that includes sequences for expressing the core components of the present invention, TnpB protein and reRNA, being miniaturized. The present invention demonstrated, with specific experimental data, that even when using an AAV vector-which had limitations in general use for constructing gene expression regulation systems in the art due to its small size-it is possible to include all essential components and achieve excellent gene expression regulation activity. Accordingly, in the present invention, “miniaturized” may refer to a size that enables operation of a gene expression regulation system using an AAV vector, but is not limited thereto.

The present invention provides an ultrasmall composition for regulating target gene expression, including the composition or the vector as an active ingredient.

The present invention provides an infected host cell using a composition comprising, as an active ingredient, the composition, the vector, or one or more of the composition or the vector.

The present invention provides a method for regulating target gene expression, the method including a step of transfecting a host cell with the composition or the vector. The method for regulating target gene expression of the present invention may include not only a step of infecting a host cell with the composition or the vector, but also additional steps that are generally included before or after the step. For example, it may include a step of pretreating the host cell before infection with the above-described substance, or general processes additionally performed to regulate gene expression from the infected host cell. In addition, the step may be performed one or more times in the present invention, and may be appropriately performed according to the desired level of gene expression regulation without limitation in the number of repetitions.

Hereinafter, the components of a vector for expressing a gene expression regulation system in a cell are described.

### Nucleic Acids Encoding Components of the Gene Expression Regulation System:

Since the purpose of the vector is to express each component of the gene expression regulation system in a cell, the sequence of the vector must essentially comprise one or more nucleic acid sequence encoding each component of the gene expression regulation system. Specifically, the sequence of the vector includes a nucleic acid sequence encoding a guide RNA and/or TnpB protein (including variants) contained in the gene expression regulation system to be expressed. In this case, the sequence of the vector may include not only a nucleic acid sequence encoding wild-type guide RNA and wild-type TnpB protein, but also a nucleic acid sequence encoding guide RNA and codon-optimized TnpB protein or engineered TnpB protein, depending on the intended purpose.

### Regulatory/Control Elements:

In order to express the vector in a cell, it must include one or more regulatory/control elements. Specifically, the regulatory/control elements may include a promoter, enhancer, intron, polyadenylation signal, Kozak consensus sequence, internal ribosome entry site (IRES), splice acceptor, 2A sequence, and/or replication origin, but are not limited thereto. The replication origin may be an fl origin, SV40 origin, pMB1 origin, adenoviral origin, AAV origin, and/or BBV origin, but is not limited thereto.

### Promoter:

To express the expression target of the vector in a cell, the promoter sequence must be operably linked to the sequence encoding each component so that RNA transcription factors can be activated within the cell. The promoter sequence may be designed differently depending on the corresponding RNA transcription factor or expression environment, and is not limited as long as it can appropriately express components of the gene expression regulation system in the cell. The promoter sequence may be one that promotes transcription by RNA polymerase (e.g., RNA Pol I, Pol II, or Pol III). For example, the promoter may be one selected from the group consisting of SV40 early promoter, mouse mammary tumor virus long terminal repeat (LTR) promoter, adenovirus major late promoter (AdMLP), herpes simplex virus (HSV) promoter, cytomegalovirus (CMV) promoter such as CMV immediate early promoter region (CMVIE), rous sarcoma virus (RSV) promoter, human U6 small nuclear promoter (U6) (Miyagishi et al., Nature Biotechnology 20, 497-500 (2002)), enhanced U6 promoter (e.g., Xia et al., Nucleic Acids Res. 2003 Sep. 1; 31 (17)), and human H1 promoter (H1), but is not limited thereto.

### Termination Signal:

When the vector sequence includes the promoter sequence, transcription of the sequence operably linked to the promoter is induced by RNA transcription factors, and a sequence that induces transcription termination by such RNA transcription factors is referred to as a termination signal. The termination signal may vary depending on the type of promoter sequence. For example, when the promoter is a U6 or H1 promoter, it recognizes a consecutive thymidine sequence (e.g., a TTTTTT (T6) sequence) as a termination signal.

### Additional Expression Elements:

The vector may include a nucleic acid sequence encoding additional expression elements that a person skilled in the art desires to express as needed. For example, the additional expression element may be one of various tags, but is not limited thereto. For example, the additional expression element may be a herbicide resistance gene such as glyphosate, glufosinate ammonium, or phosphinothricin, or an antibiotic resistance gene such as ampicillin, kanamycin, G418, bleomycin, hygromycin, or chloramphenicol, but is not limited thereto.

### Form of the Expression Vector:

The expression vector may be designed in a linear or circular vector form.

The present invention provides an ultra-miniaturized kit for regulating target gene expression, including the composition or the vector; and instructions.

In the present invention, the term “kit” refers to a tool comprising the composition or vector of the present invention to regulate the expression of a target gene. The kit of the present invention may further include other components, compositions, solutions, or devices conventionally required for storage and handling of the above materials. Specifically, each component may be applied one or more times without limitation in the number of applications, there is no limitation on the order in which each material is applied, and each material may be applied simultaneously or sequentially.

In the present invention, the kit may include a container, instructions, and the like. The container may serve to package the above substance, and may also serve to store and secure it. The material of the container may take the form of, for example, a bottle, tub, sachet, envelope, tube, or ampoule, and may be formed partially or entirely from plastic, glass, paper, foil, wax, or the like. The container may be equipped with a cap that is initially a part of the container or is completely or partially detachable and attachable to the container by mechanical, adhesive, or other means, and may also be equipped with a stopper that allows access to the contents with a syringe needle. The kit may include an external package, and the external package may include instructions for use of the components.

In the present invention, the term “comprising” does not exclude the presence of other components unless otherwise specified, but rather means that other components may be additionally included. Throughout the present invention, the terms such as “a step of ˜” or “the step of ˜” do not mean “a step for ˜.”

The nucleotide sequences of each component of the vector of the present invention, the amino acids encoded therefrom, the expression cassette comprising the same, and the recombinant vector comprising the same may be represented by, include, or encode each of the sequence identification numbers designated in the present invention, but are not limited thereto, and variants of the nucleotide sequences are also encompassed within the scope of the present invention.

The nucleic acid molecules of the nucleotide sequences (used interchangeably with polynucleotide sequences) of the present invention encompass functional equivalents thereof, such as variants in which part of the base sequence is altered by deletion, substitution, or insertion but still performs the same functional role as the original nucleic acid molecule. That is, nucleotide sequences having at least 70%, more preferably at least 80%, even more preferably at least 90%, and most preferably at least 95% sequence homology with the nucleotide sequence of the present invention may be included. For example, it includes polynucleotides having sequence homology of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%.

The “% sequence homology” for nucleotides is determined by comparing two optimally aligned sequences and their comparison regions, wherein part of the nucleotide sequence in the comparison region may include additions or deletions (i.e., gaps) relative to the reference sequence for the optimal alignment of the two sequences (which does not include insertions or deletions).

In addition, the same concept as the above-described nucleotide sequence may be applied to the amino acid sequences of the present invention. That is, the amino acid sequences encompass variants that can perform functionally equivalent actions, and may include sequences having at least 70%, more preferably at least 80%, even more preferably at least 90%, and most preferably at least 95% sequence homology with the amino acid sequences represented by the sequence identification numbers described herein. For example, it includes amino acid sequences having sequence homology of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%.

Hereinafter, preferred examples are presented to help understand the present invention. However, the following examples are provided only to help understand the present invention more easily, and the contents of the present invention are not limited by the following examples.

### EXAMPLES

### Summary of the Present Invention: Optimization and Engineering of ISDge10TnpB-Based Ultra-Miniaturized Gene Activation System (ISDge10 TnpB-VPR)

An expression vector based on a cytomegalovirus (CMV) promoter for a CRISPRa system expression vector was used to promote the activation of target gene expression in human cell lines. For optimization, an ISDge10 TnpB-based ultra-miniaturized gene activation system was designed as an all-in-one system that connects a transcription activation factor to ISDge10 TnpB using a linker and enables expression and transcription together with reRNA imparting target specificity. To construct the ISDge10 TnpB-based ultra-miniaturized gene activation system (ISDge10 TnpB-VPR) with improved activity through reRNA engineering, an activation test was performed on human-derived cell lines using a fluorescent reporter.

### Methods of Evaluating Operating Efficiency for Gene Expression Activation and Target-Specific Gene Editing Efficiency for Target Nucleotide Sequence of Gene in Human-Derived Cell Line

A human-derived cell line (HEK293FT) was purchased from Invitrogen (R70007) and cultured in Dulbecco's modified Eagle's medium (DMEM) containing 10% FBS (Gibco) at 37° C. in the presence of 5% CO₂. The cell line was maintained at 70% confluency by sub-culturing in 5% CO₂ at 37° C. every 48 hours, and 2×10⁵ cells were seeded in a 24-well plate for targeted gene regulation. 24 hours after inoculation, 240 pmol of an ultra-miniaturized gene activation system (ISDge10 TnpB-VPR) expression plasmid and 40.7 fmol of a reporter plasmid (when needed) were delivered to the cells together with 1.5 μL of Lipofectamine 3000 reagent and 1 uL of p3000 reagent (Thermo Fisher Scientific) when the cells reached 60% confluency. In this study, an ATCC-certified cell line was used.

gDNA extraction or fluorescence analysis was performed 24, 48, and 72 hours after transfection. To confirm the change in target gene expression in cells, when CWCas12f-VPR was delivered using a DNeasy blood & tissue kit (Qiagen), total gDNA was extracted from the cultured cells 24 and 48 hours after transfection, and when ISDge10TnpB was delivered using a DNeasy blood & tissue kit (Qiagen), total gDNA was extracted from the cultured cells 48 and 72 hours after transfection. Subsequently, to confirm the exact nucleotide sequence of an edited site of the target gene, nested PCR (denaturation: 98° C.-30 sec, primer annealing: 58° C.-30 sec, elongation: 72° C.-30 sec, 35 cycles) were repeatedly performed on a PCR amplicon (DNMT1) obtained using a DNA primer (Table 1) corresponding to a target gene locus. Subsequently, using the obtained PCR amplicon, annealing for hybridization was performed (denaturation: 95° C., 5 min, annealing: 95-85° C.-2° C./see, 85-25° C.-0.1° C./sec). Afterward, incubation was performed at 37° C. for 25 minutes by adding T7 endonuclease I, and DNA cleavage was confirmed by 2% agarose gel electrophoresis. DNA cleavage efficiency was confirmed with cleavage images.

TABLE 1

| | Target gene | |
|---|---|---|
| | (Primer | |
| No. | direction) | DNA (5′ to 3′) |
| seq. 1 | DNMT1_F1 | TGGGTGGATTACCTGAGGTC |
| seq. 2 | DNMT1_R1 | CCAGGCTGGACTCGAAC |
| seq. 3 | DNMT1_F2 | GAATCACTTGAACCCGGGAG |
| seq. 4 | DNMT1_R2 | CCACCTCCTCTAACTTCACCTC |
| seq. 5 | NLRC4_F1 | GTTCTGATTCTTGGGGGTTCC |
| seq. 6 | NLRC4_R1 | GGAAGCTGAGTTGGCAGGATC |
| seq. 7 | NLRC4_F2 | CGACAGACATGGTCCCTG |
| seq. 8 | NLRC4_R2 | GTTGCTGAGTAGATGCAGTCTACTG |

### Method of Confirming the Activity of Target Fluorescent Protein Expression in Human-Derived Cell Line to Evaluate Expression Activation

Reporter analysis was performed by targeting various diverse versions of the ISDge10 TnpB-based ultra-miniaturized gene activation systems (ISDge10 TnpB-VPR) to a mini-CMV-based dTomato expression reporter system in order to induce the expression of a fluorescent reporter. First, the reporter system was constructed to optimize the most effective version of the ISDge10 TnpB-based target-specific expression activator. dTomato was expressed using an appropriate target and a TAM sequence, which are located upstream of the dTomato sequence recognized by ISDge10 TnpB. The transfected cells were observed using a fluorescence microscope 24 to 48 or 48 to 72 hours after transfection. The mean fluorescence intensity was quantified using the Image J program, and calculated based on the definition of the region of interest (ROI). The information of a binding sequence for the ISDge10 TnpB-based ultra-miniaturized gene activation system (ISDge10 TnpB-VPR) and the operated sgRNA or reRNA sequence is shown in Table 2 below. Here, Table 2 shows the information of target genes and the sgRNA or reRNA sequences, which were identified to verify the operation of the CRISPR activation system, and the TAM (TTAT) of ISDge10TnpB and the PAM sequence (TTTR) of CWCas12f in the target gene used in the present invention are represented by italics. In addition, a spacer sequence in the sgRNA or reRNA sequence for each gene is underlined.

TABLE 2

| | | Target | |
|---|---|---|---|
| | | (protospacer) | |
| | | sequence | |
| | | with PAM | sgRNA sequence |
| No. | Gene | (5′ to 3′) | (5′ to 3′) |
| seq. 9/10 | 20 | TTTAGAGGGAG | gACCGCTTCACTTAGAGTGAAGGTGGGCTGCTTGCATCAGCC |
| | NLRC4 | ACACAAGTTG | TAATGTCGAGAAGTGCTTTCTTCGGAAAGTAACCCTCGAAA |
| | (CWCas12f) | ATA | CAAAGAAAGGAATGCAACGAGGGAGACACAAGTTGATAtttta |
| | | | tttt |
| seq. 9/11 | NLRC4 | | gACCGCTTCACTTAGAGTGAAGGTGGGCTGCTTGCATCAGCC |
| | 18 | | TAATGTCGAGAAGTGCTTTCTTCGGAAAGTAACCCTCGAAA |
| | (CWCas12f) | | CAAAGAAAGGAATGCAACGAGGGAGACACAAGTTGAttttatttt |
| seq. 9/12 | NLRC4 | | gACCGCTTCACTTAGAGTGAAGGTGGGCTGCTTGCATCAGCC |
| | 16 | | TAATGTCGAGAAGTGCTTTCTTCGGAAAGTAACCCTCGAAA |
| | (CWCas12f) | | CAAAGAAAGGAATGCAACGAGGGAGACACAAGTTttttatttt |
| seq. 9/13 | NLRC4 | | gACCGCTTCACTTAGAGTGAAGGTGGGCTGCTTGCATCAGCC |
| | 14 | | TAATGTCGAGAAGTGCTTTCTTCGGAAAGTAACCCTCGAAA |
| | (CWCas12f) | | CAAAGAAAGGAATGCAACGAGGGAGACACAAGttttatttt |
| seq. 9/14 | NLRC4 | | gACCGCTTCACTTAGAGTGAAGGTGGGCTGCTTGCATCAGCC |
| | 12 | | TAATGTCGAGAAGTGCTTTCTTCGGAAAGTAACCCTCGAAA |
| | (CWCas12f) | | CAAAGAAAGGAATGCAACGAGGGAGACACAttttatttt |
| seq. 9/15 | NLRC4 | | gACCGCTTCACTTAGAGTGAAGGTGGGCTGCTTGCATCAGCC |
| | 10 | | TAATGTCGAGAAGTGCTTTCTTCGGAAAGTAACCCTCGAAA |
| | (CWCas12f) | | CAAAGAAAGGAATGCAACGAGGGAGACAttttatttt |
| seq. 9/16 | NLRC4 | | gACCGCTTCACTTAGAGTGAAGGTGGGCTGCTTGCATCAGCC |
| | 8 | | TAATGTCGAGAAGTGCTTTCTTCGGAAAGTAACCCTCGAAA |
| | (CWCas12f) | | CAAAGAAAGGAATGCAACGAGGGAGAttttatttt |
| seq. 17/18 | DNMT1 | TTATGGGCTGT | gGTGGAGCGGTTCACGACCGCGACCTCAACGCCGCCCGGAA |
| | 20 | TGTCAGACCC | CATCAAGCGGGAAGGGCTTTCGCAAATCGTCGTCGCGGGGC |
| | (ISDge10 | AAC | ACGCGGAGACGTTAAACGCTCGGGGAGAGGGTGTCAGACC |
| | TnpB) | | TGCGATAGCGGGCAGCCCTCGAtgaAGCGAGAATCCAACGGC |
| | | | TTTAGCCGTTGGAGTGTCAAGGGCTGTTGTCAGACCCAACtttt |
| | | | atttt |
| seq. 17/19 | DNMT1 | | gGTGGAGCGGTTCACGACCGCGACCTCAACGCCGCCCGGAA |
| | 18 | | CATCAAGCGGGAAGGGCTTTCGCAAATCGTCGTCGCGGGGC |
| | (ISDge10 | | ACGCGGAGACGTTAAACGCTCGGGGAGAGGGTGTCAGACC |
| | TnpB) | | TGCGATAGCGGGCAGCCCTCGAtgaAGCGAGAATCCAACGGC |
| | | | TTTAGCCGTTGGAGTGTCAAGGGCTGTTGTCAGACCCAttttattt |
| | | | t |
| seq. 17/20 | DNMT1 | | gGTGGAGCGGTTCACGACCGCGACCTCAACGCCGCCCGGAA |
| | 16 | | CATCAAGCGGGAAGGGCTTTCGCAAATCGTCGTCGCGGGGC |
| | (ISDge10 | | ACGCGGAGACGTTAAACGCTCGGGGAGAGGGTGTCAGACC |
| | TnpB) | | TGCGATAGCGGGCAGCCCTCGAtgaAGCGAGAATCCAACGGC |
| | | | TTTAGCCGTTGGAGTGTCAAGGGCTGTTGTCAGACCttttatttt |
| seq. 17/21 | DNMT1 | | gGTGGAGCGGTTCACGACCGCGACCTCAACGCCGCCCGGAA |
| | 14 | | CATCAAGCGGGAAGGGCTTTCGCAAATCGTCGTCGCGGGGC |
| | (ISDge10 | | ACGCGGAGACGTTAAACGCTCGGGGAGAGGGTGTCAGACC |
| | TnpB) | | TGCGATAGCGGGCAGCCCTCGAtgaAGCGAGAATCCAACGGC |
| | | | TTTAGCCGTTGGAGTGTCAAGGGCTGTTGTCAGAttttatttt |
| seq. 17/22 | DNMT1 | | gGTGGAGCGGTTCACGACCGCGACCTCAACGCCGCCCGGAA |
| | 12 | | CATCAAGCGGGAAGGGCTTTCGCAAATCGTCGTCGCGGGGC |
| | (ISDge10 | | ACGCGGAGACGTTAAACGCTCGGGGAGAGGGTGTCAGACC |
| | TnpB) | | TGCGATAGCGGGCAGCCCTCGAtgaAGCGAGAATCCAACGGC |
| | | | TTTAGCCGTTGGAGTGTCAAGGGCTGTTGTCAttttatttt |
| seq. 17/23 | DNMT1 | | gGTGGAGCGGTTCACGACCGCGACCTCAACGCCGCCCGGAA |
| | 10 | | CATCAAGCGGGAAGGGCTTTCGCAAATCGTCGTCGCGGGGC |
| | (ISDge10 | | ACGCGGAGACGTTAAACGCTCGGGGAGAGGGTGTCAGACC |
| | TnpB) | | TGCGATAGCGGGCAGCCCTCGAtgaAGCGAGAATCCAACGGC |
| | | | TTTAGCCGTTGGAGTGTCAAGGGCTGTTGTttttatttt |
| seq. 17/24 | DNMT1 | | gGTGGAGCGGTTCACGACCGCGACCTCAACGCCGCCCGGAA |
| | 8 | | CATCAAGCGGGAAGGGCTTTCGCAAATCGTCGTCGCGGGGC |
| | (ISDge10 | | ACGCGGAGACGTTAAACGCTCGGGGAGAGGGTGTCAGACC |
| | TnpB) | | TGCGATAGCGGGCAGCCCTCGAtgaAGCGAGAATCCAACGGC |
| | | | TTTAGCCGTTGGAGTGTCAAGGGCTGTTttttatttt |

### Example 1. Design of Expression Vector for System Optimization for ISDge10TnpB-Based Human-Derived Cell Line and Verification of Operating Efficiency

### Example 1-1. Design of ISDge10 TnpB and Verification of its Operating Efficiency

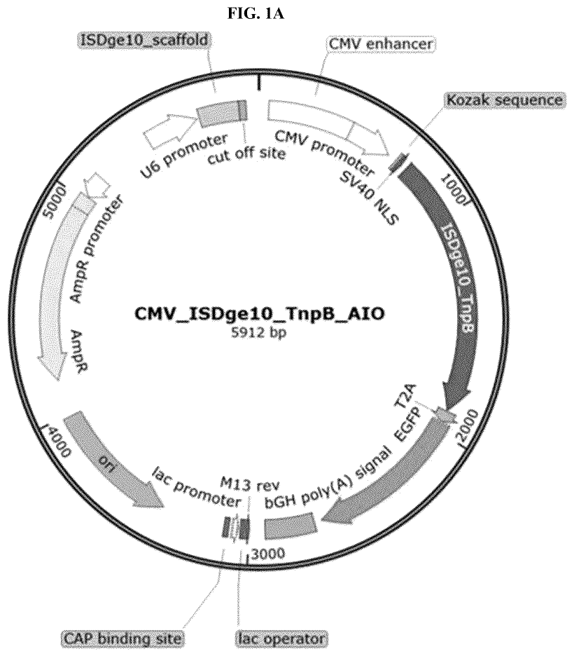

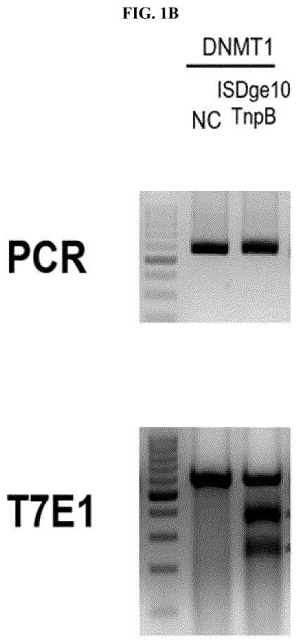

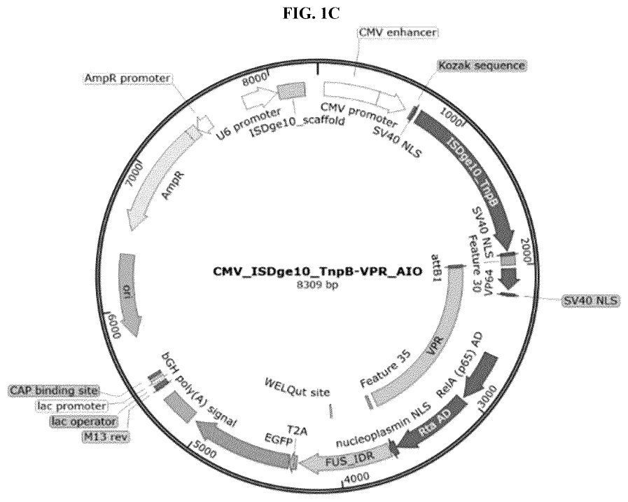

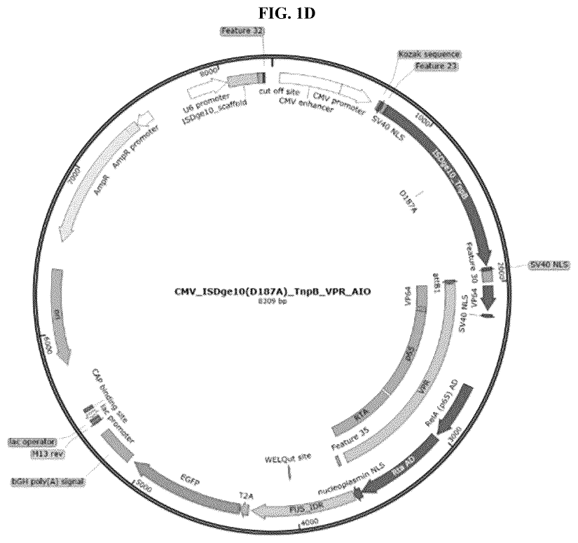

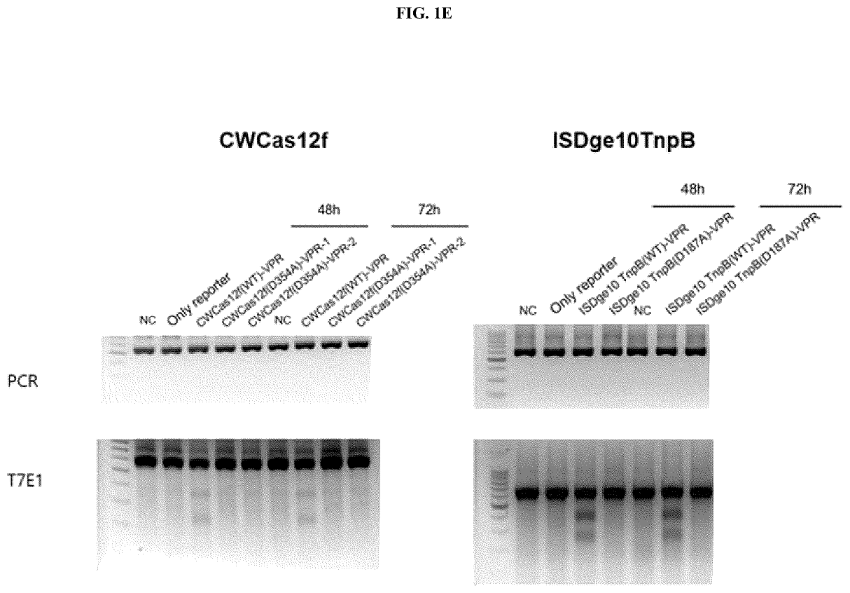

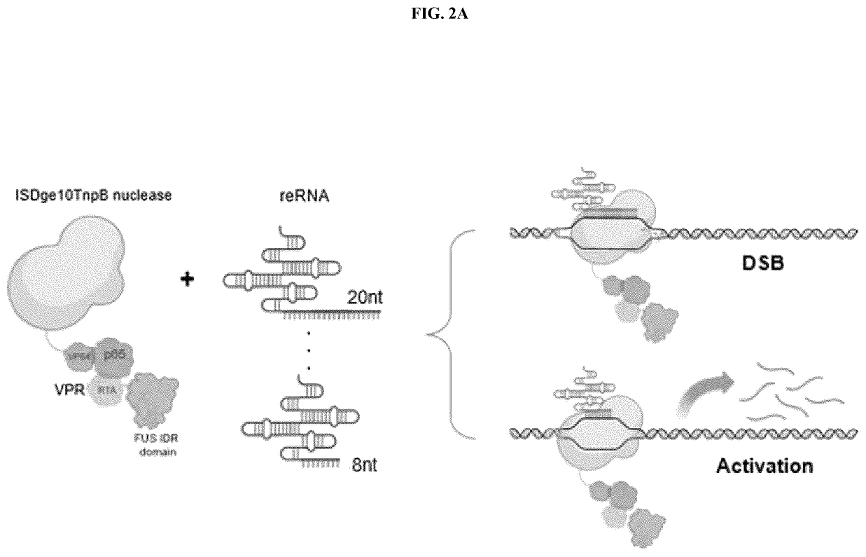

Before designing an activation system, an expression vector was designed to optimize the operating efficiency of ISDge10 TnpB, which is an ultra-miniaturized endonuclease for a human-derived cell line. Here, the expression vector was designed and constructed to simultaneously deliver and express a cytomegalovirus (CMV) promoter-based protein and a U6 promoter-based reRNA (FIG. 1A).

The ISDge10 TnpB expression vector constructed as shown in FIG. 1A was delivered to a 293 FT cell line to confirm operating efficiency. The transformed cells were analyzed by the T7E1 assay, which confirmed the efficient induction of indels in the DNMT1 gene (FIG. 1B).

### Example 1-2. Design of ISDge10 TnpB-VPR and ISDge10 TnpB (D187A)-VPR, and Verification of their Operating Efficiency

Vectors that express ISDge10 TnpB-based ultra-miniaturized gene activation systems ISDge10 TnpB-VPR and ISDge10 TnpB (D187A)-VPR were constructed by introducing a CRISPR activation system (CRISPRa) to the ISDge10 TnpB expression system designed and constructed in Example 1-1.

First, a vector that expresses the ISDge10 TnpB-based ultra-miniaturized gene activation system, ISDge10 TnpB-VPR, was constructed. Here, the system was designed to be simultaneously delivered and expressed together with reRNA imparting target specificity (FIG. 1C). Through the sequence comparison with Cas12-series proteins, it was confirmed that the active site amino acid sequence of each protein was conserved.

Meanwhile, it was confirmed that cleavage activity efficiency disappeared when amino acid 187, aspartic acid (D), was substituted with alanine (A), so a vector that expresses an ultra-miniaturized CRISPR activation system (ISDge10TnpB (D187A)-VPR) in which the aspartic acid (D) 187 of ISDge10TnpB was substituted with alanine (A) (D187A) was designed and constructed (FIG. 1D).

Each expression vector was designed to simultaneously deliver and express a CMV promoter-based ultra-miniaturized CRISPR activation system and a U6 promoter-based guide RNA, and designed in the form that enables GFP co-expression for intracellular expression control. Additionally, the expression vector was designed to connect an RNA-binding protein, the FUS IDR domain, inducing transcription promotion and screen ISDge10TnpB-VPR protein with improved activity.

The constructed ISDge10 TnpB-VPR and ISDge10TnpB (D187A)-VPR expression vectors were delivered to a human-derived cell line (HEK293FT) and subjected to T7E1 assay. Here, as a comparative group, a CWCas12f-VPR system was used. Here, CWCas12f-VPR, ring-shaped CWCas12f-VPR, and cleavage activity-removed CWCas12f (D354A)-VPR were designated as comparative groups. D354A is an amino acid responsible for the activity of conventional CWCas12f, and it is known that it does not cleave DNA even when binding to DNA due to substitution with alanine, and sequence comparison analysis confirmed that D354A is a conserved part.

As a result, it was confirmed through cell experiments that indels were induced in the target when the transcriptional activation domains were linked to the ISDge10TnpB and CWCas12f proteins, indicating that the transcriptional activation domains did not affect the activity of the protein. Based on these results, it was confirmed that the conditions of this experiment could be used as a transcriptional activation system, and it was confirmed that indels were not induced in the case of proteins with substituted amino acids (FIG. 1E).

### Example 2. Construction and Selection of ISDge10 TnpB-Based Ultra-Miniaturized Gene Activation System (ISDge10 TnpB-VPR)

A vector for increasing operating efficiency was constructed based on the ultra-miniaturized CRISPR activation system (ISDge10TnpB-VPR) constructed in Example 1-2. Specifically, based on ISDge10TnpB-VPR, various versions of vectors were constructed by adjusting the length of a spacer hybridized with DNA in reRNA imparting target specificity to ISDge10TnpB through reRNA engineering, thereby developing an optimized version of the ISDge10TnpB-VPR system with better operating efficiency (FIG. 2A).

A fluorescent reporter that was selected to confirm the optimized version of the ISDge10TnpB-VPR system was constructed to perform verification. Specifically, the fluorescent reporter system is based on a small CMV promoter and expresses an RFP fluorescent protein. The fluorescent reporter system was designed to have the TAM sequence TTAT, which can be recognized by ISDge10TnpB, and the sequence of the target gene DNMT1 that is present in front of the promoter (FIG. 2B).

TABLE 3

| No. | Name | sequence(5′→3′/N→S) |
|---|---|---|
| seq. | SV40NLS | CCAAAGAAGAAGCGGAAGGTC |
| 25 | | |
| seq. | SV40NLS_AA | PKKKRKV |
| 26 | | |
| seq. | ISDge10 | ATGACCACCCACCGCAAGGTGTACAGATACCGCATCGAGCCCACCCCCGTGCAGGAGAGCAAG |
| 27 | TnpB | CTGTACATGCTGGCCGGCAGCCGGAGATTCGTGTTCAACTGGGCCCTGGCCCGGAGAAGAGAG |
| | | CACTACGCCGAAACCGGCAAGACCCTGGGATACAACGCCCAGGCAGGAGAGCTGACCGCCCTG |
| | | AAAAACCAAGAAGAAACCAGCTGGCTGAAAGAGTCCGACTCACAACTGCTGCAGCAGGCACTG |
| | | AAAGACGTGGAAAGAGCCTTCGTGAACTTCTTCGAAAAACGGGCCAGATTCCCCAGATTCAAAA |
| | | GCAAGAAAACCGACACCCCCAGATTCAGAATCCCCCAAAGAGTGAGAATAGAGGGCAGCAGAG |
| | | TGTACGTGCCCAAGGTGGGGTGGGTGAAGCTGAGAAAGAGTCAGGAGATAGAAGGAAAAACCA |
| | | AAAGCGCCACCTTCAAGAGGGAGGCAGATGGCCATTGGTACGTGCTGCTGGTGAGTGAGTTCGA |
| | | GATGCCTGATGTGCCTCTGCCCCCAGTGCCCGAGAGCGAGGTGGTGGGAATCGACCTGGGACTG |
| | | AAGGACTTCTACGTCCTGTCTGACGGGGGCAGGAAGGAGGCCCCCAGATTCGCTAGAAAGGGC |
| | | CAGCGAAAACTGAGAAGAGCTGCAAGAAGACATAGCAAGTGCACCCGGGGAAGCAACAGAAA |
| | | GGCCAAGGCAAAGAGGAAGCTGGCAAGAGTGCACAGACAGATCGCAAACCAGAGAAAAGACT |
| | | TCGTGCACAAGGCCACCAGCGGCCTCGTGCAGCAGTACCAAGGCTTCTGCATCGAAAACCTGAG |
| | | CATCAAGGGCATGGCCAAAACCAAACTGTCAAAAAGCGTGCTGGACGCAGCCCTGGGGGAATT |
| | | TAGAAGACAGCTGGCCTATAAAGCCCAGTGGCATAGAAAGTGGCTGGCAGTGATAGACAGATG |
| | | GTTCCCCAGCAGCAAGCTGTGCGGAGAGTGCGGCAGCATTAACGCCGATCTGACCCTGAGCGA |
| | | CAGAGAATGGACCTGCGAGTGTGGCGCAGTGCACGACAGAGATCTGAACGCCGCTCGCAACAT |
| | | CAAGAGAGAGGGCCTGAGTCAGATTGTGGTGGCCGGACATGCCGAAACCCTGAACGCCAGAGG |
| | | GGAGGGCGTGAGACCCGCCATTGCCGGAAGCCCTAGA |
| seq. | ISDge10 | MTTHRKVYRYRIEPTPVQESKLYMLAGSRRFVFNWALARRREHYAETGKTLGYNAQAGELTALKNQE |
| 28 | TnpB_AA | ETSWLKESDSQLLQQALKDVERAFVNFFEKRARFPRFKSKKTDTPRFRIPQRVRIEGSRVVVPKVGWVK |
| | | LRKSQEIEGKTKSATFKREADGHWYVLLVSEFEMPDVPLPPVPESEVVGIDLGLKDFYVLSDGGRKEAP |
| | | RFARKGQRKLRRAARRHSKCTRGSNRKAKAKRKLARVHRQIANQRKDPVHKATSGLVQQYQGFCIE |
| | | NLGIKGMAKTKLSKSVLDAALGEFRRQLAYKAQWHRKWLAVIDRWFPSSKLCGECGSINADLTLSDR |
| | | EWTCECGAVHDRDLNAARNIKREGLSQIVVAGHAETLNARGEGVRPAIAGSPR |
| seq. | ISDge10 | ATGACCACCCACCGCAAGGTGTACAGATACCGCATCGAGCCCACCCCCGTGCAGGAGAGCAAG |
| 29 | TnpB | CTGTACATGCTGGCCGGCAGCCGGAGATTCGTGTTCAACTGGGCCCTGGCCCGGAGAAGAGAG |
| | (D187A) | CACTACGCCGAAACCGGCAAGACCCTGGGATACAACGCCCAGGCAGGAGAGCTGACCGCCCTG |
| | | AAAAACCAAGAAGAAACCAGCTGGCTGAAAGAGTCCGACTCACAACTGCTGCAGCAGGCACTG |
| | | AAAGACGTGGAAAGAGCCTTCGTGAACTTCTTCGAAAAACGGGCCAGATTCCCCAGATTCAAAA |
| | | GCAAGAAAACCGACACCCCCAGATTCAGAATCCCCCAAAGAGTGAGAATAGAGGGCAGCAGAG |
| | | TGTACGTGCCCAAGGTGGGGTGGGTGAAGCTGAGAAAGAGTCAGGAGATAGAAGGAAAAACCA |
| | | AAAGCGCCACCTTCAAGAGGGAGGCAGATGGCCATTGGTACGTGCTGCTGGTGAGTGAGTTCGA |
| | | GATGCCTGATGTGCCTCTGCCCCCAGTGCCCGAGAGCGAGGTGGTGGGAATCGcCCTGGGACTG |
| | | CAGCGAAAACTGAGAAGAGCTGCAAGAAGACATAGCAAGTGCACCCGGGGAAGCAACAGAAA |
| | | GGCCAAGGCAAAGAGGAAGCTGGCAAGAGTGCACAGACAGATCGCAAACCAGAGAAAAGACT |
| | | TCGTGCACAAGGCCACCAGCGGCCTCGTGCAGCAGTACCAAGGCTTCTGCATCGAAAACCTGAG |
| | | CATCAAGGGCATGGCCAAAACCAAACTGTCAAAAAGCGTGCTGGACGCAGCCCTGGGGGAATT |
| | | TAGAAGACAGCTGGCCTATAAAGCCCAGTGGCATAGAAAGTGGCTGGCAGTGATAGACAGATG |
| | | GTTCCCCAGCAGCAAGCTGTGCGGAGAGTGCGGCAGCATTAACGCCGATCTGACCCTGAGCGA |
| | | CAGAGAATGGACCTGCGAGTGTGGCGCAGTGCACGACAGAGATCTGAACGCCGCTCGCAACAT |
| | | CAAGAGAGAGGGCCTGAGTCAGATTGTGGTGGCCGGACATGCCGAAACCCTGAACGCCAGAGG |
| | | GGAGGGCGTGAGACCCGCCATTGCCGGAAGCCCTAGA |
| seq. | ISDge10 | MTTHRKVYRYRIEPTPVQESKLYMLAGSRRFVFNWALARRREHYAETGKTLGYNAQAGELTALKNQE |
| 30 | TnpB | ETSWLKESDSQLLQQALKDVERAFVNFFEKRARFPRFKSKKTDTPRFRIPQRVRIEGSRVVVPKVGWVK |
| | (D187A)_AA | LRKSQEIEGKTKSATFKREADGHWYVLLVSEFEMPDVPLPPVPESEVVGIALGLKDFYVLSDGGRKEAP |
| | | RFARKGQRKLRRAARRHSKCTRGSNRKAKAKRKLARVHRQIANQRKDPVHKATSGLVQQYQGFCIE |
| | | NLGIKGMAKTKLSKSVLDAALGEFRRQLAYKAQWHRKWLAVIDRWFPSSKLCGECGSINADLTLSDR |
| | | EWTCECGAVHDRDLNAARNIKREGLSQIVVAGHAETLNARGEGVRPAIAGSPR |
| seq. | linker | AGCAGGGCTGACCCCAAGAAGAAGAGGAAGGTGTCGCCAGGGATCCGTCGACTTGACGCGTTG |
| 31 | (including | ATATCAACAAGTTTGTACAAAAAAGCAGGCTACAAA |
| | SV40NLS) | |
| seq. | linker | SRADPKKKRKVSPGIRRLDALISTSLYKKAGYK |
| 32 | (including | |
| | SV40NLS)_AA | |
| seq. | VPR | GAGGCCAGCGGTTCCGGACGGGCTGACGCATTGGACGATTTTGATCTGGATATGCTGGGAAGTG |
| 33 | | ACGCCCTCGATGATTTTGACCTTGACATGCTTGGTTCGGATGCCCTTGATGACTTTGACCTCGACA |
| | | TGCTCGGCAGTGACGCCCTTGATGATTTCGACCTGGACATGCTGATTAACTCTAGAAGTTCCGGA |
| | | TCTCCGAAAAAGAAACGCAAAGTTGGTAGCCAGTACCTGCCCGACACCGACGACCGGCACCGG |
| | | ATCGAGGAAAAGGGGAAGGGGACCTACGAGACATTCAAGAGCATCATGAAGAAGTCCCCCTTC |
| | | AGCGGCCCCACCGACCCTAGACCTCCACCTAGAAGAATCGCCGTGCCCAGCAGATCCAGCGCC |
| | | AGCGTGCCAAAACCTGCCCCCCAGCCTTACCCCTTCACCAGCAGCCTGAGCACCATCAACTACG |
| | | ACGAGTTCCCTACCATGGTGTTCCCCAGCGGCCAGATCTCTCAGGCCTCTGCTCTGGCTCCAGCC |
| | | CCTCCTCAGGTGCTGCCTCAGGCTCCTGCTCCTGCACCAGCTCCAGCCATGGTGTCTGCACTGGC |
| | | TCAGGCACCAGCACCCGTGCCTGTGCTGGCTCCTGGACCTCCACAGGCTGTGGCTCCACCAGCC |
| | | CCTAAACCTACACAGGCCGGCGAGGGCACACTGTCTGAAGCTCTGCTGCAGCTGCAGTTCGACG |
| | | ACGAGGATCTGGGAGCCCTGCTGGGAAACAGCACCGATCCTGCCGTGTTCACCGACCTGGCCAG |
| | | CGTGGACAACAGCGAGTTCCAGCAGCTGCTGAACCAGGGCATCCCTGTGGCCCCTCACACCACC |
| | | GAGCCCATGCTGATGGAATACCCCGAGGCCATCACCCGGCTCGTGACAGGCGCTCAGAGGCCT |
| | | CCTGATCCAGCTCCTGCCCCTCTGGGAGCACCAGGCCTGCCTAATGGACTGCTGTCTGGCGACG |
| | | AGGACTTCAGCTCTATCGCCGATATGGATTTCTCAGCCTTGCTGGGCTCTGGCAGCGGCAGCCGG |
| | | GATTCCAGGGAAGGGATGTTTTTGCCGAAGCCTGAGGCCGGCTCCGCTATTAGTGACGTGTTTGA |
| | | GGGCCGCGAGGTGTGCCAGCCAAAACGAATCCGGCCATTTCATCCTCCAGGAAGTCCATGGGCC |
| | | AACCGCCCACTCCCCGCCAGCCTCGCACCAACACCAACCGGTCCAGTACATGAGCCAGTCGGGT |
| | | CACTGACCCCGGCACCAGTCCCTCAGCCACTGGATCCAGCGCCCGCAGTGACTCCCGAGGCCA |
| | | GTCACCTGTTGGAGGATCCCGATGAAGAGACGAGCCAGGCTGTCAAAGCCCTTCGGGAGATGG |
| | | CCGATACTGTGATTCCCCAGAAGGAAGAGGCTGCAATCTGTGGCCAAATGGACCTTTCCCATCC |
| | | GCCCCCAAGGGGCCATCTGGATGAGCTGACAACCACACTTGAGTCCATGACCGAGGATCTGAAC |
| | | CTGGACTCACCCCTGACCCCGGAATTGAACGAGATTCTGGATACCTTCCTGAACGACGAGTGCCT |
| | | CTTGCATGCCATGCATATCAGCACAGGACTGTCCATCTTCGACACATCTCTGTTT |
| seq. | VPR_AA | EASGGGRADALDDFDLDMLGGDALDDFDLDMLGSDALDDFDLDMLGSDALDDFDLDMLINSRSSG |
| 34 | | SPKKKRKVGSQYLPDTDDRHRIEEKRKRTYETFKSIMKKSPFSGPTDPRPPPRRIAVPSRSSASVPKPAP |
| | | QPYPFTSSLSTINYDEFPTMVFPSGQISQASALAPAPPQVLPQAPAPAPAPAMVSALAQAPAPVPVLA |
| | | PGPPQAVAPPAPKPTQAGEGTLSEALLQLQFDDEDLGALLGNSTDPAVFTDLASVDNSEFQQLLNQG |
| | | IPVAPHTTEPMLMEYPEAITRLVTGAQRPPDPAPAPLGAPGLPNGLLSGDEDFSSIADMDFSALLGSGS |
| | | GSRDSREGMFLPKPEAGSAISDVFEGREVCQPKRIRPFHPPGSPWANRPLPASLAPTPTGPVHEPVGS |
| | | LTPAPVPQPLDPAPAVTPEASHLLEDPDEETSQAVKALREMADTVIPQKEEAAICGQMDLSHPPPRGH |
| | | LDELTTTLESMTEDLNLDSPLTPELNEILDTFLNDECLLHAMHISTGLSIFDTSLF |
| seq. | NucleoplasminNLS | AAAAGGCCGGCGGCCACGAAAAAGGCCGGCCAGGCAAAAAAGAAAAAG |
| 35 | | |
| seq. | NucleoplasminNLS_ | KRPAATKKAGQAKKKK |
| 36 | AA | |
| seq. | FUS IDR | ATGGCCTCAAACGATTATACCCAACAAGCAACCCAAAGCTATGGGGCCTACCCCACCCAGCCCG |
| 37 | | GGCAGGGCTATTCCCAGCAGAGCAGTCAGCCCTACGGACAGCAGAGTTACAGTGGTTATAGCCA |
| | | GTCCACGGACACTTCAGGCTATGGCCAGAGCAGCTATTCTTCTTATGGCCAGAGCCAGAACACA |
| | | GGCTATGGAACTCAGTCAACTCCCCAGGGATATGGCTCGACTGGCGGCTATGGCAGTAGCCAGA |
| | | GCTCCCAATCGTCTTACGGGCAGCAGTCCTCCTACCCTGGCTATGGCCAGCAGCCAGCTCCCAG |
| | | CAGCACCTCGGGAAGTTACGGTAGCAGTTCTCAGAGCAGCAGCTATGGGCAGCCCCAGAGTGG |
| | | GAGCTACAGCCAGCAGCCTAGCTATGGTGGACAGCAGCAAAGCTATGGACAGCAGCAAAGCTA |
| | | TAATCCCCCTCAGGGCTATGGACAGCAGAACCAGTACAACAGCAGCAGTGGTGGTGGAGGTGG |
| | | AGGTGGAGGTGGAGGTAACTATGGCCAAGATCAATCCTCCATGAGTAGTGGTGGTGGCAGTGGT |
| | | GGCGGTTATGGCAATCAAGACCAGAGTGGTGGAGGTGGCAGCGGTGGCTATGGACAGCAGGAC |
| | | CGTGGA |
| seq. | FUS IDR_AA | MASNDYTQQATQSYGAYPTQPGQGYSQQSSQPYGQQSYSGYSQSTDTSGYGQSSYSSYGQSQNT |
| 38 | | GYGTQSTPQGYGSTGGYGSSQSSQSSYGQQSSYPGYGQQPAPSSTSGSYGSSSQSSSYGQPQSGSY |
| | | SQQPSYGGQQQSYGQQQSYNPPQGYGQQNQYNSSSGGGGGGGGGGNYGQDGSSMSSGGGSG |
| | | GGYGNQDQSGGGGSGGYGQQDRG |
| seq. | DNMT1 | GTGGAGCGGTTCACGACCGCGACCTCAACGCCGCCCGGAACATCAAGCGGGAAGGGCTTTCGC |
| 39 | 20 | AAATCGTCGTCGCGGGGCACGCGGAGACGTTAAACGCTCGGGGAGAGGGTGTCAGACCTGCGA |
| | (ISDge10 | TAGCGGGCAGCCCTCGAtgaAGCGAGAATCCAACGGCTTTAGCCGTTGGAGTGTCAA |
| | TnpB | |
| | without | |
| | space seq.) | |
| seq. | T2A | GAGGGCAGAGGAAGTCTGCTAACATGCGGTGACGTCGAGGAGAATCCTGGCCCA |
| 40 | | |
| seq. | EGFP | GTGAGCAAGGGCGAGGAGCTGTTCACCGGGGTGGTGCCCATCCTGGTCGAGCTGGACGGCGAC |
| 41 | | GTAAACGGCCACAAGTTCAGCGTGTCCGGCGAGGGCGAGGGCGATGCCACCTACGGCAAGCTG |
| | | ACCCTGAAGTTCATCTGCACCACCGGCAAGCTGCCCGTGCCCTGGCCCACCCTCGTGACCACCC |
| | | TGACCTACGGCGTGCAGTGCTTCAGCCGCTACCCCGACCACATGAAGCAGCACGACTTCTTCAA |
| | | GTCCGCCATGCCCGAAGGCTACGTCCAGGAGCGCACCATCTTCTTCAAGGACGACGGCAACTAC |
| | | AAGACCCGCGCCGAGGTGAAGTTCGAGGGCGACACCCTGGTGAACCGCATCGAGCTGAAGGGC |
| | | ATCGACTTCAAGGAGGACGGCAACATCCTGGGGCACAAGCTGGAGTACAACTACAACAGCCAC |
| | | AACGTCTATATCATGGCCGACAAGCAGAAGAACGGCATCAAGGTGAACTTCAAGATCCGCCACA |
| | | ACATCGAGGACGGCAGCGTGCAGCTCGCCGACCACTACCAGCAGAACACCCCCATCGGCGACG |
| | | GCCCCGTGCTGCTGCCCGACAACCACTACCTGAGCACCCAGTCCGCCCTGAGCAAAGACCCCAA |
| | | CGAGAAGCGCGATCACATGGTCCTGCTGGAGTTCGTGACCGCCGCCGGGATCACTCTCGGCATG |
| | | GACGAGCTGTACAAG |
| seq. | U6 | GAGGGCCTATTTCCCATGATTCCTTCATATTTGCATATACGATACAAGGCTGTTAGAGAGATAAT |
| 42 | | GGAATTAATTTGACTGTAAACACAAAGATATTAGTACAAAATACGTGACGTAGAAAGTAATAATT |
| | | TCTTGGGTAGTTTGCAGTTTTAAAATTATGTTTTAAAATGGACTATCATATGCTTACCGTAACTTGA |
| | | AAGTATTTCGATTTCTTGGCTTTATATATCTTGTGGAAAGGAC |
| seq. | ISDge10TnpB- | ATGACCACCCACCGCAAGGTGTACAGATACCGCATCGAGCCCACCCCCGTGCAGGAGAGCAAG |
| 43 | VPR | CTGTACATGCTGGCCGGCAGCCGGAGATTCGTGTTCAACTGGGCCCTGGCCCGGAGAAGAGAG |
| | | CACTACGCCGAAACCGGCAAGACCCTGGGATACAACGCCCAGGCAGGAGAGCTGACCGCCCTG |
| | | AAAAACCAAGAAGAAACCAGCTGGCTGAAAGAGTCCGACTCACAACTGCTCCAGCAGGCACTG |
| | | AAAGACGTGGAAAGAGCCTTCGTGAACTTCTTCGAAAAACGGGCCAGATTCCCCAGATTCAAAA |
| | | GCAAGAAAACCGACACCCCCAGATTCAGAATCCCCCAAAGAGTGAGAATAGAGGGCAGCAGAG |
| | | TGTACGTGCCCAAGGTGGGGTGGGTGAAGCTGAGAAAGAGTCAGGAGATAGAAGGAAAAACCA |
| | | AAAGCGCCACCTTCAAGAGGGAGGCAGATGGCCATTGGTACGTGCTGCTGGTGAGTGAGTTCGA |
| | | GATGCCTGATGTGCCTCTGCCCCCAGTGCCCGAGAGCGAGGTGGTGGGAATCGACCTGGGACTG |
| | | AAGGACTTCTACGTCCTGTCTGACGGGGGCAGGAAGGAGGCCCCCAGATTCGCTAGAAAGGGC |
| | | CAGCGAAAACTGAGAAGAGCTGCAAGAAGACATAGCAAGTGCACCCGGGGAAGCAACAGAAA |
| | | GGCCAAGGCAAAGAGGAAGCTGGCAAGAGTGCACAGACAGATCGCAAACCAGAGAAAAGACT |
| | | TCGTGCACAAGGCCACCAGCGGCCTCGTGCAGCAGTACCAAGGCTTCTGCATCGAAAACCTGAG |
| | | CATCAAGGGCATGGCCAAAACCAAACTGTCAAAAAGCGTGCTGGACGCAGCCCTGGGGGAATT |
| | | TAGAAGACAGCTGGCCTATAAAGCCCAGTGGCATAGAAAGTGGCTGGCAGTGATAGACAGATG |
| | | GTTCCCCAGCAGCAAGCTGTGCGGAGAGTGCGGCAGCATTAACGCCGATCTGACCCTGAGCGA |
| | | CAGAGAATGGACCTGCGAGTGTGGCGCAGTGCACGACAGAGATCTGAACGCCGCTCGCAACAT |
| | | CAAGAGAGAGGGCCTGAGTCAGATTGTGGTGGCCGGACATGCCGAAACCCTGAACGCCAGAGG |
| | | GGAGGGCGTGAGACCCGCCATTGCCGGAAGCCCTAGAAGCAGGGCTGACCCCAAGAAGAAGA |
| | | GGAAGGTGTCGCCAGGGATCCGTCGACTTGACGCGTTGATATCAACAAGTTTGTACAAAAAAGC |
| | | AGGCTACAAAGAGGCCAGCGGTTCCGGACGGGCTGACGCATTGGACGATTTTGATCTGGATATG |
| | | CTGGGAAGTGACGCCCTCGATGATTTTGACCTTGACATGCTTGGTTCGGATGCCCTTGATGACTTT |
| | | GACCTCGACATGCTCGGCAGTGACGCCCTTGATGATTTCGACCTGGACATGCTGATTAACTCTAG |
| | | AAGTTCCGGATCTCCGAAAAAGAAACGCAAAGTTGGTAGCCAGTACCTGCCCGACACCGACGAC |
| | | CGGCACCGGATCGAGGAAAAGCGGAAGCGGACCTACGAGACATTCAAGAGCATCATGAAGAA |
| | | GTCCCCCTTCAGCGGCCCCACCGACCCTAGACCTCCACCTAGAAGAATCGCCGTGCCCAGCAGA |
| | | TCCAGCGCCAGCGTGCCAAAACCTGCCCCCCAGCCTTACCCCTTCACCAGCAGCCTGAGCACCA |
| | | TCAACTACGACGAGTTCCCTACCATGGTGTTCCCCAGCGGCCAGATCTCTCAGGCCTCTGCTCTG |
| | | GCTCCAGCCCCTCCTCAGGTGCTGCCTCAGGCTCCTGCTCCTGCACCAGCTCCAGCCATGGTGTC |
| | | TGCACTGGCTCAGGCACCAGCACCCGTGCCTGTGCTGGCTCCTGGACCTCCACAGGCTGTGGCT |
| | | CCACCAGCCCCTAAACCTACACAGGCCGGCGAGGGCACACTGTCTGAAGCTCTGCTGCAGCTGC |
| | | AGTTCGACGACGAGGATCTGGGAGCCCTGCTGGGAAACAGCACCGATCCTGCCGTGTTCACCGA |
| | | CCTGGCCAGCGTGGACAACAGCGAGTTCCAGCAGCTGCTGAACCAGGGCATCCCTGTGGCCCCT |
| | | CACACCACCGAGCCCATGCTGATGGAATACCCCGAGGCCATCACCCGGCTCGTGACAGGCGCTC |
| | | AGAGGCCTCCTGATCCAGCTCCTGCCCCTCTGGGAGCACCAGGCCTGCCTAATGGACTGCTGTCT |
| | | GGCGACGAGGACTTCAGCTCTATCGCCGATATGGATTTCTCAGCCTTGCTGGGCTCTGGCAGCGG |
| | | CAGCCGGGATTCCAGGGAAGGGATGTTTTTGCCGAAGCCTGAGGCCGGCTCCGCTATTAGTGAC |
| | | GTGTTTGAGGGCCGCGAGGTGTGCCAGCCAAAACGAATCCGGCCATTTCATCCTCCAGGAAGTC |
| | | CATGGGCCAACCGCCCACTCCCCGCCAGCCTCGCACCAACACCAACCGGTCCAGTACATGAGCC |
| | | AGTCGGGTCACTGACCCCGGCACCAGTCCCTCAGCCACTGGATCCAGCGCCCGCAGTGACTCCC |
| | | GAGGCCAGTCACCTGTTGGAGGATCCCGATGAAGAGACGAGCCAGGCTGTCAAAGCCCTTCGG |
| | | GAGATGGCCGATACTGTGATTCCCCAGAAGGAAGAGGCTGCAATCTGTGGCCAAATGGACCTTT |
| | | CCCATCCGCCCCCAAGGGGCCATCTGGATGAGCTGACAACCACACTTGAGTCCATGACCGAGGA |
| | | TCTGAACCTGGACTCACCCCTGACCCCGGAATTGAACGAGATTCTGGATACCTTCCTGAACGACG |
| | | AGTGCCTCTTGCATGCCATGCATATCAGCACAGGACTGTCCATCTTCGACACATCTCTGTTT |
| seq. | ISDge10TnpB- | MTTHRKVYRYRIEPTPVQESKLYMLAGSRRFVFNWALARRREHYAETGKTLGYNAQAGELTALKNQE |
| 44 | VPR_AA | ETSWLKESDSQLLQQALKDVERAFVNFFEKRARFPRFKSKKTDTPRFRIPQRVRIEGSRVyVPKVGWVK |
| | | LRKSQEIEGKTKSATFKREADGHWYVLLVSEFEMPDVPLPPVPESEVVGIDLGLKDFYVLSDGGRKEAP |
| | | RFARKGQRKLRRAARRHSKCTRGSNRKAKAKRKLARVHRQIANQRKDPVHKATSGLVQQYQGFCIE |
| | | NLGIKGMAKTKLSKSVLDAALGEFRRQLAYKAQWHRKWLAVIDRWFPSSKLCGECGSINADLTLSDR |
| | | EWTCECGAVHDRDLNAARNIKREGLSQIVVAGHAETLNARGEGVRPAIAGSPRSRADPKKKRKVSPG |
| | | IRRLDALISTSLYKKAGYKEASGSGRADALDDFDLDMLGSDALDDFDLDMLGSDALDDFDLDMLGSD |
| | | ALDDFDLDMLINSRSSGSPKKKRKVGSQYLPDTDDRHRIEEKRKRTYETFKSIMKKSPFSGPTDPRPPP |
| | | RRIAVPSRSSASVPKPAPQPYPFTSSLSTINYDEFPTMVFPSGQISQASALAPAPPQVLPQAPAPAPAP |
| | | AMVSALAQAPAPVPVLAPGPPQAVAPPAPKPTQAGEGTLSEALLQLQFDDEDLGALLGNSTDPAVFT |
| | | DLASVDNSEFQQLLNQGIPVAPHTTEPMLMEYPEAITRLVTGAQRPPDPAPAPLGAPGLPNGLLSGD |
| | | EDFSSIADMDFSALLGSGSGSRDSREGMFLPKPEAGSAISDVFEGREVCQPKRIRPFHPPGSPWANRP |
| | | LPASLAPTPTGPVHEPVGSLTPAPVPQPLDPAPAVTPEASHLLEDPDEETSQAVKALREMADTVIPQKE |
| | | EAAICGQMDLSHPPPRGHLDELTTTLESMTEDLNLDSPLTPELNEILDTFLNDECLLHAMHISTGLSIFD |
| | | TSLF |
| seq. | ISDge10TnpB | ATGACCACCCACCGCAAGGTGTCAGATACCGCATCGAGCCCACCCCCGTGCAGGAGAGCAAG |
| 45 | (D187A)-VPR | CTGTACATGCTGGCCGGCAGCCGGAGATTCGTGTTCAACTGGGCCCTGGCCCGGAGAAGAGAG |
| | | CACTACGCCGAAACCGGCAAGACCCTGGGATACAACGCCCAGGCAGGAGAGCTGACCGCCCTG |
| | | AAAAACCAAGAAGAAACCAGCTGGCTGAAAGAGTCCGACTCACAACTGCTGCAGCAGGCACTG |
| | | AAAGACGTGGAAAGAGCCTTCGTGAACTTCTTCGAAAAACGGGCCAGATTCCCCAGATTCAAAA |
| | | GCAAGAAAACCGACACCCCCAGATTCAGAATCCCCCAAAGAGTGAGAATAGAGGGCAGCAGAG |
| | | TGTACGTGCCCAAGGTGGGGTGGGTGAAGCTGAGAAAGAGTCAGGAGATAGAAGGAAAAACCA |
| | | AAAGCGCCACCTTCAAGAGGGAGGCAGATGGCCATTGGTACGTGCTGCTGGTGAGTGAGTTCGA |
| | | GATGCCTGATGTGCCTCTGCCCCCAGTGCCCGAGAGCGAGGTGGTGGGAATCGcCCTGGGACTG |
| | | AAGGACTTCTACGTCCTGTCTGACGGGGGCAGGAAGGAGGCCCCCAGATTCGCTAGAAAGGGC |
| | | CAGCGAAAACTGAGAAGAGCTGCAAGAAGACATAGCAAGTGCACCCGGGGAAGCAACAGAAA |
| | | GGCCAAGGCAAAGAGGAAGCTGGCAAGAGTGCACAGACAGATCGCAAACCAGAGAAAAGACT |
| | | TCGTGCACAAGGCCACCAGCGGCCTCGTGCAGCAGTACCAAGGCTTCTGCATCGAAAACCTGAG |
| | | CATCAAGGGCATGGCCAAAACCAAACTGTCAAAAAGCGTGCTGGACGCAGCCCTGGGGGAATT |
| | | TAGAAGACAGCTGGCCTATAAAGCCCAGTGGCATAGAAAGTGGCTGGCAGTGATAGACAGATG |
| | | GTTCCCCAGCAGCAAGCTGTGCGGAGAGTGCGGCAGCATTAACGCCGATCTGACCCTGAGCGA |
| | | CAGAGAATGGACCTGCGAGTGTGGCGCAGTGCACGACAGAGATCTGAACGCCGCTCGCAACAT |
| | | CAAGAGAGAGGGCCTGAGTCAGATTGTGGTGGCCGGACATGCCGAAACCCTGAACGCCAGAGG |
| | | GGAGGGCGTGAGACCCGCCATTGCCGGAAGCCCTAGAAGCAGGGCTGACCCCAAGAAGAAGA |
| | | GGAAGGTGTCGCCAGGGATCCGTCGACTTGACGCGTTGATATCAACAAGTTTGTACAAAAAAGC |
| | | AGGCTACAAAGAGGCCAGCGGTTCCGGACGGGCTGACGCATTGGACGATTTTGATCTGGATATG |
| | | CTGGGAAGTGACGCCCTCGATGATTTTGACCTTGACATGCTTGGTTCGGATGCCCTTGATGACTTT |
| | | GACCTCGACATGCTCGGCAGTGACGCCCTTGATGATTTCGACCTGGACATGCTGATTAACTCTAG |
| | | AAGTTCCGGATCTCCGAAAAAGAAACGCAAAGTTGGTAGCCAGTACCTGCCCGACACCGACGAC |
| | | CGGCACCGGATCGAGGAAAAGCGGAAGCGGACCTACGAGACATTCAAGAGCATCATGAAGAA |
| | | GTCCCCCTTCAGCGGCCCCACCGACCCTAGACCTCCACCTAGAAGAATCGCCGTGCCCAGCAGA |
| | | TCCAGCGCCAGCGTGCCAAAACCTGCCCCCCAGCCTTACCCCTTCACCAGCAGCCTGAGCACCA |
| | | TCAACTACGACGAGTTCCCTACCATGGTGTTCCCCAGCGGCCAGATCTCTCAGGCCTCTGCTCTG |
| | | GCTCCAGCCCCTCCTCAGGTGCTGCCTCAGGCTCCTGCTCCTGCACCAGCTCCAGCCATGGTGTC |
| | | TGCACTGGCTCAGGCACCAGCACCCGTGCCTGTGCTGGCTCCTGGACCTCCACAGGCTGTGGCT |
| | | CCACCAGCCCCTAAACCTACACAGGCCGGCGAGGGCACACTGTCTGAAGCTCTGCTGCAGCTGC |
| | | AGTTCGACGACGAGGATCTGGGAGCCCTGCTGGGAAACAGCACCGATCCTGCCGTGTTCACCGA |
| | | CCTGGCCAGCGTGGACAACAGCGAGTTCCAGCAGCTGCTGAACCAGGGCATCCCTGTGGCCCCT |
| | | CACACCACCGAGCCCATGCTGATGGAATACCCCGAGGCCATCACCCGGCTCGTGACAGGCGCTC |
| | | AGAGGCCTCCTGATCCAGCTCCTGCCCCTCTGGGAGCACCAGGCCTGCCTAATGGACTGCTGTCT |
| | | GGCGACGAGGACTTCAGCTCTATCGCCGATATGGATTTCTCAGCCTTGCTGGGCTCTGGCAGCGG |
| | | CAGCCGGGATTCCAGGGAAGGGATGTTTTTGCCGAAGCCTGAGGCCGGCTCCGCTATTAGTGAC |
| | | GTGTTTGAGGGCCGCGAGGTGTGCCAGCCAAAACGAATCCGGCCATTTCATCCTCCAGGAAGTC |
| | | CATGGGCCAACCGCCCACTCCCCGCCAGCCTCGCACCAACACCAACCGGTCCAGTACATGAGCC |
| | | AGTCGGGTCACTGACCCCGGCACCAGTCCCTCAGCCACTGGATCCAGCGCCCGCAGTGACTCCC |
| | | GAGGCCAGTCACCTGTTGGAGGATCCGATGAAGAGACGAGCCAGGCTGTCAAAGCCCTTCGG |
| | | GAGATGGCCGATACTGTGATTCCCCAGAAGGAAGAGGCTGCAATCTGTGGCCAAATGGACCTTT |
| | | CCCATCCGCCCCCAAGGGGCCATCTGGATGAGCTGACAACCACACTTGAGTCCATGACCGAGGA |
| | | TCTGAACCTGGACTCACCCCTGACCCCGGAATTGAACGAGATTCTGGATACCTTCCTGAACGACG |
| | | AGTGCCTCTTGCATGCCATGCATATCAGCACAGGACTGTCCATCTTCGACACATCTCTGTTTAAAA |
| | | GGCCGGCGGCCACGAAAAAGGCCGGCCAGGCAAAAAAGAAAAAGATGGCCTCAAACGATTAT |
| | | ACCCAACAAGCAACCCAAAGCTATGGGGCCTACCCCACCCAGCCCGGGCAGGGCTATTCCCAG |
| | | CAGAGCAGTCAGCCCTACGGACAGCAGAGTTACAGTGGTTATAGCCAGTCCACGGACACTTCAG |
| | | GCTATGGCCAGAGCAGCTATTCTTCTTATGGCCAGAGCCAGAACACAGGCTATGGAACTCAGTC |
| | | AACTCCCCAGGGATATGGCTCGACTGGCGGCTATGGCAGTAGCCAGAGCTCCCAATCGTCTTAC |
| | | GGGCAGCAGTCCTCCTACCCTGGCTATGGCCAGCAGCCAGCTCCCAGCAGCACCTCGGGAAGTT |
| | | ACGGTAGCAGTTCTCAGAGCAGCAGCTATGGGCAGCCCCAGAGTGGGAGCTACAGCCAGCAGC |
| | | CTAGCTATGGTGGACAGCAGCAAAGCTATGGACAGCAGCAAGCTATAATCCCCCTCAGGGCTA |
| | | TGGACAGCAGAACCAGTACAACAGCAGCAGTGGTGGTGGAGGTGGAGGTGGAGGTGGAGGTA |
| | | ACTATGGCCAAGATCAATCCTCCATGAGTAGTGGTGGTGGCAGTGGTGGCGGTTATGGCAATCA |
| | | AGACCAGAGTGGTGGAGGTGGCAGCGGTGGCTATGGACAGCAGGACCGTGGA |
| seq. | ISDge10TnpB | MTTHRKVYRYRIEPTPVQESKLYMLAGSRRFVFNWALARRREHYAETGKTLGYNAQAGELTALKNQE |
| 46 | (D187A)-VPR_ | ETSWLKESDSQLLQQALKDVERAFVNFFEKRARFPRFKSKKTDTPRFRIPQRVRIEGSRVyVPKVGWVK |
| | AA | LRKSQEIEGKTKSATFKREADGHWYVLLVSEFEMPDVPLPPVPESEVVGIALGLKDFYVLSDGGRKEAP |
| | | RFARKGQRKLRRAARRHSKCTRGSNRKAKAKRKLARVHRQIANQRKDPVHKATSGLVQQYQGFCIE |
| | | NLGIKGMAKTKLSKSVLDAALGEFRRQLAYKAQWHRKWLAVIDRWFPSSKLCGECGSINADLTLSDR |
| | | EWTCECGAVHDRDLNAARNIKREGLSQIVVAGHAETLNARGEGVRPAIAGSPRSRADPKKKRKVSPG |
| | | IRRLDALISTSLYKKAGYKEASGSGRADALDDFDLDMLGSDALDDFDLDMLGSDALDDFDLDMLGSD |
| | | ALDDFDLDMLINSRSSGSPKKKRKVGSQYLPDTDDRHRIEEKRKRTYETFKSIMKKSPFSGPTDPRPPP |
| | | RRIAVPSRSSASVPKPAPQPYPFTSSLSTINYDEFPTMVFPSGQISQASALAPAPPQVLPQAPAPAPAP |
| | | AMVSALAQAPAPVPVLAPGPPQAVAPPAPKPTQAGEGTLSEALLQLQFDDEDLGALLGNSTDPAVFT |
| | | DLASVDNSEFQQLLNQGIPVAPHTTEPMLMEYPEAITRLVTGAQRPPDPAPAPLGAPGLPNGLLSGD |
| | | EDFSSIADMDFSALLGSGSGSRDSREGMFLPKPEAGSAISDVFEGREVCQPKRIRPFHPPGSPWANRP |
| | | LPASLAPTPTGPVHEPVGSLTPAPVPQPLDPAPAVTPEASHLLEDPDEETSQAVKALREMADTVIPQKE |
| | | EAAICGQMDLSHPPPRGHLDELTTTLESMTEDLNLDSPLTPELNEILDTFLNDECLLHAMHISTGLSIFD |
| | | TSLFKRPAATKKAGQAKKKKMASNDYTQQATQSYGAYPTQPGQGYSQQSSQPYGQQSYSGYSQST |
| | | DTSGYGQSSYSSYGQSQNTGYGTQSTPQGYGSTGGYGSSQSSQSSYGQQSSYPGYGQQPAPSSTS |
| | | GSYGSSSQSSSYGQPQSGSYSQQPSYGGQQQSYGQQQSYNPPQGYGQQNQYNSSSGGGGGGG |
| | | GGGNYGQDQSSMSSGGGSGGGYGNQDQSGGGGSGGYGQQDRG |
| seq. | ISDge10TnpB- | ATGACCACCCACCGCAAGGTGTACAGATACCGCATCGAGCCCACCCCCGTGCAGGAGAGCAAG |
| 47 | VPR- | CTGTACATGCTGGCCGGCAGCCGGAGATTCGTGTTCAACTGGGCCCTGGCCCGGAGAAGAGAG |
| | Nucleoplasmin | CACTACGCCGAAACCGGCAAGACCCTGGGATACAACGCCCAGGCAGGAGAGCTGACCGCCCTG |
| | NLS-FUS IDR | AAAAACCAAGAAGAAACCAGCTGGCTGAAAGAGTCCGACTCACAACTGCTGCAGCAGGCACTG |
| | | AAAGACGTGGAAAGAGCCTTCGTGAACTTCTTCGAAAAACGGGCCAGATTCCCCAGATTCAAAA |
| | | GCAAGAAAACCGACACCCCCAGATTCAGAATCCCCCAAAGAGTGAGAATAGAGGGCAGCAGAG |
| | | TGTACGTGCCCAAGGTGGGGTGGGTGAAGCTGAGAAAGAGTCAGGAGATAGAAGGAAAAACCA |
| | | AAAGCGCCACCTTCAAGAGGGAGGCAGATGGCCATTGGTACGTGCTGCTGGTGAGTGAGTTCGA |
| | | GATGCCTGATGTGCCTCTGCCCCCAGTGCCCGAGAGCGAGGTGGTGGGAATCGACCTGGGACTG |
| | | AAGGACTTCTACGTCCTGTCTGACGGGGGCAGGAAGGAGGCCCCCAGATTCGCTAGAAAGGGC |
| | | CAGCGAAAACTGAGAAGAGCTGCAAGAAGACATAGCAAGTGCACCCGGGGAAGCAACAGAAA |
| | | GGCCAAGGCAAAGAGGAAGCTGGCAAGAGTGCACAGACAGATCGCAAACCAGAGAAAAGACT |
| | | TCGTGCACAAGGCCACCAGCGGCCTCGTGCAGCAGTACCAAGGCTTCTGCATCGAAAACCTGAG |
| | | CATCAAGGGCATGGCCAAAACCAAACTGTCAAAAAGCGTGCTGGACGCAGCCCTGGGGGAATT |
| | | TAGAAGACAGCTGGCCTATAAAGCCCAGTGGCATAGAAAGTGGCTGGCAGTGATAGACAGATG |
| | | GTTCCCCAGCAGCAAGCTGTGCGGAGAGTGCGGCAGCATTAACGCCGATCTGACCCTGAGCGA |
| | | CAGAGAATGGACCTGCGAGTGTGGCGCAGTGCACGACAGAGATCTGAACGCCGCTCGCAACAT |
| | | CAAGAGAGAGGGCCTGAGTCAGATTGTGGTGGCCGGACATGCCGAAACCCTGAACGCCAGAGG |
| | | GGAGGGCGTGAGACCCGCCATTGCCGGAAGCCCTAGAAGCAGGGCTGACCCCAAGAAGAAGA |
| | | GGAAGGTGTCGCCAGGGATCCGTCGACTTGACGCGTTGATATCAACAAGTTTGTACAAAAAAGC |
| | | AGGCTACAAAGAGGCCAGCGGTTCCGGACGGGCTGACGCATTGGACGATTTTGATCTGGATATG |
| | | CTGGGAAGTGACGCCCTCGATGATTTTGACCTTGACATGCTTGGTTCGGATGCCCTTGATGACTTT |
| | | GACCTCGACATGCTCGGCAGTGACGCCCTTGATGATTTCGACCTGGACATGCTGATTAACTCTAG |
| | | AAGTTCCGGATCTCCGAAAAAGAAACGCAAAGTTGGTAGCCAGTACCTGCCCGACACCGACGAC |
| | | CGGCACCGGATCGAGGAAAAGCGGAAGCGGACCTACGAGACATTCAAGAGCATCATGAAGAA |
| | | GTCCCCCTTCAGCGGCCCCACCGACCCTAGACCTCCACCTAGAAGAATCGCCGTGCCCAGCAGA |
| | | TCCAGCGCCAGCGTGCCAAAACCTGCCCCCCAGCCTTACCCCTTCACCAGCAGCCTGAGCACCA |
| | | TCAACTACGACGAGTTCCCTACCATGGTGTTCCCCAGCGGCCAGATCTCTCAGGCCTCTGCTCTG |
| | | GCTCCAGCCCCTCCTCAGGTGCTGCCTCAGGCTCCTGCTCCTGCACCAGCTCCAGCCATGGTGTC |
| | | TGCACTGGCTCAGGCACCAGCACCCGTGCCTGTGCTGGCTCCTGGACCTCCACAGGCTGTGGCT |
| | | CCACCAGCCCCTAAACCTACACAGGCCGGCGAGGGCACACTGTCTGAAGCTCTGCTGCAGCTGC |
| | | AGTTCGACGACGAGGATCTGGGAGCCCTGCTGGGAAACAGCACCGATCCTGCCGTGTTCACCGA |
| | | CCTGGCCAGCGTGGACAACAGCGAGTTCCAGCAGCTGCTGAACCAGGGCATCCCTGTGGCCCCT |
| | | CACACCACCGAGCCCATGCTGATGGAATACCCCGAGGCCATCACCCGGCTCGTGACAGGCGCTC |
| | | AGAGGCCTCCTGATCCAGCTCCTGCCCCTCTGGGAGCACCAGGCCTGCCTAATGGACTGCTGTCT |
| | | GGCGACGAGGACTTCAGCTCTATCGCCGATATGGATTTCTCAGCCTTGCTGGGCTCTGGCAGCGG |
| | | CAGCCGGGATTCCAGGGAAGGGATGTTTTTGCCGAAGCCTGAGGCCGGCTCCGCTATTAGTGAC |
| | | GTGTTTGAGGGCCGCGAGGTGTGCCAGCCAAAACGAATCCGGCCATTTCATCCTCCAGGAAGTC |
| | | CATGGGCCAACCGCCCACTCCCCGCCAGCCTCGCACCAACACCAACCGGTCCAGTACATGAGCC |
| | | AGTCGGGTCACTGACCCCGGCACCAGTCCCTCAGCCACTGGATCCAGCGCCCGCAGTGACTCCC |
| | | GAGGCCAGTCACCTGTTGGAGGATCCCGATGAAGAGACGAGCCAGGCTGTCAAAGCCCTTCGG |
| | | GAGATGGCCGATACTGTGATTCCCCAGAAGGAAGAGGCTGCAATCTGTGGCCAAATGGACCTTT |
| | | CCCATCCGCCCCCAAGGGGCCATCTGGATGAGCTGACAACCACACTTGAGTCCATGACCGAGGA |
| | | TCTGAACCTGGACTCACCCCTGACCCCGGAATTGAACGAGATTCTGGATACCTTCCTGAACGACG |
| | | AGTGCCTCTTGCATGCCATGCATATCAGCACAGGACTGTCCATCTTCGACACATCTCTGTTTAAAA |
| | | GGCCGGCGGCCACGAAAAAGGCCGGCCAGGCAAAAAAGAAAAAGATGGCCTCAAACGATTAT |
| | | ACCCAACAAGCAACCCAAAGCTATGGGGCCTACCCCACCCAGCCCGGGCAGGGCTATTCCCAG |
| | | CAGAGCAGTCAGCCCTACGGACAGCAGAGTTACAGTGGTTATAGCCAGTCCACGGACACTTCAG |
| | | GCTATGGCCAGAGCAGCTATTCTTCTTATGGCCAGAGCCAGAACACAGGCTATGGAACTCAGTC |
| | | AACTCCCCAGGGATATGGCTCGACTGGCGGCTATGGCAGTAGCCAGAGCTCCCAATCGTCTTAC |
| | | GGGCAGCAGTCCTCCTACCCTGGCTATGGCCAGCAGCCAGCTCCCAGCAGCACCTCGGGAAGTT |
| | | ACGGTAGCAGTTCTCAGAGCAGCAGCTATGGGCAGCCCCAGAGTGGGAGCTACAGCCAGCAGC |
| | | CTAGCTATGGTGGACAGCAGCAAAGCTATGGACAGCAGCAAAGCTATAATCCCCCTCAGGGCTA |
| | | TGGACAGCAGAACCAGTACAACAGCAGCAGTGGTGGTGGAGGTGGAGGTGGAGGTGGAGGTA |
| | | ACTATGGCCAAGATCAATCCTCCATGAGTAGTGGTGGTGGCAGTGGTGGCGGTTATGGCAATCA |
| | | AGACCAGAGTGGTGGAGGTGGCAGCGGTGGCTATGGACAGCAGGACCGTGGA |
| seq. | ISDge10TnpB- | MTTHRKVYRYRIEPTPVQESKLYMLAGSRRFVFNWALARRREHYAETGKTLGYNAQAGELTALKNQE |
| 48 | VPR- | ETSWLKESDSQLLQQALKDVERAFVNFFEKRARFPRFKSKKTDTPRFRIPQRVRIEGSRVyVPKVGWVK |
| | Nucleoplasmin | LRKSQEIEGKTKSATFKREADGHWYVLLVSEFEMPDVPLPPVPESEVVGIDLGLKDFYVLSDGGRKEAP |
| | NLS-FUS | RFARKGQRKLRRAARRHSKCTRGSNRKAKAKRKLARVHRQIANQRKDPVHKATSGLVQQYQGFCIE |
| | IDR_AA | NLGIKGMAKTKLSKSVLDAALGEFRRQLAYKAQWHRKWLAVIDRWFPSSKLCGECGSINADLTLSDR |
| | | EWTCECGAVHDRDLNAARNIKREGLSQIVVAGHAETLNARGEGVRPAIAGSPRSRADPKKKRKVSPG |
| | | IRRLDALISTSLYKKAGYKEASGSGRADALDDFDLDMLGSDALDDFDLDMLGSDALDDFDLDMLGSD |
| | | ALDDFDLDMLINSRSSGSPKKKRKVGSQYLPDTDDRHRIEEKRKRTYETFKSIMKKSPFSGPTDPRPPP |
| | | RRIAVPSRSSASVPKPAPQPYPFTSSLSTINYDEFPTMVFPSGQISQASALAPAPPQVLPQAPAPAPAP |
| | | AMVSALAQAPAPVPVLAPGPPQAVAPPAPKPTQAGEGTLSEALLQLQFDDEDLGALLGNSTDPAVFT |
| | | DLASVDNSEFQQLLNQGIPVAPHTTEPMLMEYPEAITRLVTGAQRPPDPAPAPLGAPGLPNGLLSGD |
| | | EDFSSIADMDFSALLGSGSGSRDSREGMFLPKPEAGSAISDVFEGREVCQPKRIRPFHPPGSPWANRP |
| | | LPASLAPTPTGPVHEPVGSLTPAPVPQPLDPAPAVTPEASHLLEDPDEETSQAVKALREMADTVIPQKE |
| | | EAAICGQMDLSHPPPRGHLDELTTTLESMTEDLNLDSPLTPELNEILDTFLNDECLLHAMHISTGLSIFD |
| | | TSLFKRPAATKKAGQAKKKKMASNDYTQQATQSYGAYPTQPGQGYSQQSSQPYGQQSYSGYSQST |
| | | DTSGYGQSSYSSYGQSQNTGYGTQSTPQGYGSTGGYGSSQSSQSSYGQQSSYPGYGQQPAPSSTS |
| | | GSYGSSSQSSSYGQPQSGSYSQQPSYGGQQQSYGQQQSYNPPQGYGQQNQYNSSSGGGGGGG |
| | | GGGNYGQDQSSMSSGGGSGGGYGNQDQSGGGGSGGYGQQDRG |
| seq. | ISDge10TnpB | ATGACCACCCACCGCAAGGTGTACAGATACCGCATCGAGCCCACCCCCGTGCAGGAGAGCAAG |
| 49 | (D187A)-VPR- | CTGTACATGCTGGCCGGCAGCCGGAGATTCGTGTTCAACTGGGCCCTGGCCCGGAGAAGAGAG |
| | Nucleoplasmin | CACTACGCCGAAACCGGCAAGACCCTGGGATACAACGCCCAGGCAGGAGAGCTGACCGCCCTG |
| | NLS-FUS | AAAAACCAAGAAGAAACCAGCTGGCTGAAAGAGTCCGACTCACAACTGCTGCAGCAGGCACTG |
| | IDR | AAAGACGTGGAAAGAGCCTTCGTGAACTTCTTCGAAAAACGGGCCAGATTCCCCAGATTCAAAA |
| | | GCAAGAAAACCGACACCCCCAGATTCAGAATCCCCCAAAGAGTGAGAATAGAGGGCAGCAGAG |
| | | TGTACGTGCCCAAGGTGGGGTGGGTGAAGCTGAGAAAGAGTCAGGAGATAGAAGGAAAAACCA |
| | | AAAGCGCCACCTTCAAGAGGGAGGCAGATGGCCATTGGTACGTGCTGCTGGTGAGTGAGTTCGA |
| | | GATGCCTGATGTGCCTCTGCCCCCAGTGCCCGAGAGCGAGGTGGTGGGAATCGCCCTGGGACTG |
| | | AAGGACTTCTACGTCCTGTCTGACGGGGGCAGGAAGGAGGCCCCCAGATTCGCTAGAAAGGGC |
| | | CAGCGAAAACTGAGAAGAGCTGCAAGAAGACATAGCAAGTGCACCCGGGGAAGCAACAGAAA |
| | | GGCCAAGGCAAAGAGGAAGCTGGCAAGAGTGCACAGACAGATCGCAACCAGAGAAAAGACT |
| | | TCGTGCACAAGGCCACCAGCGGCCTCGTGCAGCAGTACCAAGGCTTCTGCATCGAAAACCTGAG |
| | | CATCAAGGGCATGGCCAAAACCAAACTGTCAAAAAGCGTGCTGGACGCAGCCCTGGGGGAATT |
| | | TAGAAGACAGCTGGCCTATAAAGCCCAGTGGCATAGAAAGTGGCTGGCAGTGATAGACAGATG |
| | | GTTCCCCAGCAGCAAGCTGTGCGGAGAGTGCGGCAGCATTAACGCCGATCTGACCCTGAGCGA |
| | | CAGAGAATGGACCTGCGAGTGTGGCGCAGTGCACGACAGAGATCTGAACGCCGCTCGCAACAT |
| | | CAAGAGAGAGGGCCTGAGTCAGATTGTGGTGGCCGGACATGCCGAAACCCTGAACGCCAGAGG |
| | | GGAGGGCGTGAGACCCGCCATTGCCGGAAGCCCTAGAAGCAGGGCTGACCCCAAGAAGAAGA |
| | | GGAAGGTGTCGCCAGGGATCCGTCGACTTGACGCGTTGATATCAACAAGTTTGTACAAAAAAGC |
| | | AGGCTACAAAGAGGCCAGCGGTTCCGGACGGGCTGACGCATTGGACGATTTTGATCTGGATATG |
| | | CTGGGAAGTGACGCCCTCGATGATTTTGACCTTGACATGCTTGGTTCGGATGCCCTTGATGACTTT |
| | | GACCTCGACATGCTCGGCAGTGACGCCCTTGATGATTTCGACCTGGACATGCTGATTAACTCTAG |
| | | AAGTTCCGGATCTCCGAAAAAGAAACGCAAAGTTGGTAGCCAGTACCTGCCCGACACCGACGAC |
| | | CGGCACCGGATCGAGGAAAAGCGGAAGCGGACCTACGAGACATTCAAGAGCATCATGAAGAA |
| | | GTCCCCCTTCAGCGGCCCCACCGACCCTAGACCTCCACCTAGAAGAATCGCCGTGCCCAGCAGA |
| | | TCCAGCGCCAGCGTGCCAAAACCTGCCCCCCAGCCTTACCCCTTCACCAGCAGCCTGAGCACCA |
| | | TCAACTACGACGAGTTCCCTACCATGGTGTTCCCCAGCGGCCAGATCTCTCAGGCCTCTGCTCTG |
| | | GCTCCAGCCCCTCCTCAGGTGCTGCCTCAGGCTCCTGCTCCTGCACCAGCTCCAGCCATGGTGTC |
| | | TGCACTGGCTCAGGCACCAGCACCCGTGCCTGTGCTGGCTCCTGGACCTCCACAGGCTGTGGCT |
| | | CCACCAGCCCCTAAACCTACACAGGCCGGCGAGGGCACACTGTCTGAAGCTCTGCTGCAGCTGC |
| | | AGTTCGACGACGAGGATCTGGGAGCCCTGCTGGGAAACAGCACCGATCCTGCCGTGTTCACCGA |
| | | CCTGGCCAGCGTGGACAACAGCGAGTTCCAGCAGCTGCTGAACCAGGGCATCCCTGTGGCCCCT |
| | | CACACCACCGAGCCCATGCTGATGGAATACCCCGAGGCCATCACCCGGCTCGTGACAGGCGCTC |
| | | AGAGGCCTCCTGATCCAGCTCCTGCCCCTCTGGGAGCACCAGGCCTGCCTAATGGACTGCTGTCT |
| | | GGCGACGAGGACTTCAGCTCTATCGCCGATATGGATTTCTCAGCCTTGCTGGGCTCTGGCAGCGG |
| | | CAGCCGGGATTCCAGGGAAGGGATGTTTTTGCCGAAGCCTGAGGCCGGCTCCGCTATTAGTGAC |
| | | GTGTTTGAGGGCCGCGAGGTGTGCCAGCCAAAACGAATCCGGCCATTTCATCCTCCAGGAAGTC |
| | | CATGGGCCAACCGCCCACTCCCCGCCAGCCTCGCACCAACACCAACCGGTCCAGTACATGAGCC |
| | | AGTCGGGTCACTGACCCCGGCACCAGTCCCTCAGCCACTGGATCCAGCGCCCGCAGTGACTCCC |
| | | GAGCCCAGTCACCTGTTGGAGGATCCCGATGAAGAGACGAGCCAGGCTGTCAAAGCCCTTCGG |
| | | GAGATGGCCGATACTGTGATTCCCCAGAAGGAAGAGGCTGCAATCTGTGGCCAAATGGACCTTT |
| | | CCCATCCGCCCCCAAGGGGCCATCTGGATGAGCTGACAACCACACTTGAGTCCATGACCGAGGA |
| | | TCTGAACCTGGACTCACCCCTGACCCCGGAATTGAACGAGATTCTGGATACCTTCCTGAACGACG |
| | | AGTGCCTCTTGCATGCCATGCATATCAGCACAGGACTGTCCATCTTCGACACATCTCTGTTTAAAA |
| | | GGCCGGCGGCCACGAAAAAGGCCGGCCAGGCAAAAAAGAAAAAGATGGCCTCAAACGATTAT |
| | | ACCCAACAAGCAACCCAAAGCTATGGGGCCTACCCCACCCAGCCCGGGCAGGGCTATTCCCAG |
| | | CAGAGCAGTCAGCCCTACGGACAGCAGAGTTACAGTGGTTATAGCCAGTCCACGGACACTTCAG |
| | | GCTATGGCCAGAGCAGCTATTCTTCTTATGGCCAGAGCCAGAACACAGGCTATGGAACTCAGTC |
| | | AACTCCCCAGGGATATGGCTCGACTGGCGGCTATGGCAGTAGCCAGAGCTCCCAATCGTCTTAC |
| | | GGGCAGCAGTCCTCCTACCCTGGCTATGGCCAGCAGCCAGCTCCCAGCAGCACCTCGGGAAGTT |
| | | ACGGTAGCAGTTCTCAGAGCAGCAGCTATGGGCAGCCCCAGAGTGGGAGCTACAGCCAGCAGC |
| | | CTAGCTATGGTGGACAGCAGCAAAGCTATGGACAGCAGCAAAGCTATAATCCCCCTCAGGGCTA |
| | | TGGACAGCAGAACCAGTACAACAGCAGCAGTGGTGGTGGAGGTGGAGGTGGAGGTGGAGGTA |
| | | ACTATGGCCAAGATCAATCCTCCATGAGTAGTGGTGGTGGCAGTGGTGGCGGTTATGGCAATCA |
| | | AGACCAGAGTGGTGGAGGTGGCAGCGGTGGCTATGGACAGCAGGACCGTGGA |
| seq. | ISDge10TnpB | MTTHRKVYRYRIEPTPVQESKLYMLAGSRRFVFNWALARRREHYAETGKTLGYNAQAGELTALKNQE |
| 50 | (D187A)-VPR- | ETSWLKESDSQLLQQALKDVERAFVNFFEKRARFPRFKSKKTDTPRFRIPQRVRIEGSRVyVPKVGWVK |
| | Nucleoplasmin | LRKSQEIEGKTKSATFKREADGHWYVLLVSEFEMPDVPLPPVPESEVVGIALGLKDFYVLSDGGRKEAP |
| | NLS-FUS | RFARKGQRKLRRAARRHSKCTRGSNRKAKAKRKLARVHRQIANQRKDPVHKATSGLVQQYQGFCIE |
| | IDR_AA | NLGIKGMAKTKLSKSVLDAALGEFRRQLAYKAQWHRKWLAVIDRWFPSSKLCGECGSINADLTLSDR |
| | | EWTCECGAVHDRDLNAARNIKREGLSQIVVAGHAETLNARGEGVRPAIAGSPRSRADPKKKRKVSPG |
| | | IRRLDALISTSLYKKAGYKEASGSGRADALDDFDLDMLGSDALDDFDLDMLGSDALDDFDLDMLGSD |
| | | ALDDFDLDMLINSRSSGSPKKKRKVGSQYLPDTDDRHRIEEKRKRTYETFKSIMKKSPFSGPTDPRPPP |
| | | RRIAVPSRSSASVPKPAPQPYPFTSSLSTINYDEFPTMVFPSGQISQASALAPAPPQVLPQAPAPAPAP |
| | | AMVSALAQAPAPVPVLAPGPPQAVAPPAPKPTQAGEGTLSEALLQLQFDDEDLGALLGNSTDPAVFT |
| | | DLASVDNSEFQQLLNQGIPVAPHTTEPMLMEYPEAITRLVTGAQRPPDPAPAPLGAPGLPNGLLSGD |
| | | EDFSSIADMDFSALLGSGSGSRDSREGMFLPKPEAGSAISDVFEGREVCQPKRIRPFHPPGSPWANRP |
| | | LPASLAPTPTGPVHEPVGSLTPAPVPQPLDPAPAVTPEASHLLEDPDEETSQAVKALREMADTVIPQKE |
| | | EAAICGQMDLSHPPPRGHLDELTTTLESMTEDLNLDSPLTPELNEILDTFLNDECLLHAMHISTGLSIFD |
| | | TSLFKRPAATKKAGQAKKKKMASNDYTQQATQSYGAYPTQPGQGYSQQSSQPYGQQSYSGYSQST |
| | | DTSGYGQSSYSSYGQSQNTGYGTQSTPQGYGSTGGYGSSQSSQSSYGQQSSYPGYGQQPAPSSTS |
| | | GSYGSSSQSSSYGQPQSGSYSQQPSYGGQQQSYGQQQSYNPPQGYGQQNQYNSSSGGGGGGG |
| | | GGGNYGQDQSSMSSGGGSGGGYGNQDQSGGGGSGGYGQQDRG |
| seq. | SV40NLS- | CCAAAGAAGAAGCGGAAGGTCGGTATCCACGGAGTCCCAGCAGCCATGACCACCCACCGCAAG |
| 51 | ISDge10TnpB- | GTGTACAGATACCGCATCGAGCCCACCCCCGTGCAGGAGAGCAAGCTGTACATGCTGGCCGGC |
| | linker-VPR- | AGCCGGAGATTCGTGTTCAACTGGGCCCTGGCCCGGAGAAGAGAGCACTACGCCGAAACCGGC |
| | Nucleoplasmin | AAGACCCTGGGATACAACGCCCAGGCAGGAGAGCTGACCGCCCTGAAAAACCAAGAAGAAACC |
| | NLS-FUS IDR | AGCTGGCTGAAAGAGTCCGACTCACAACTGCTGCAGCAGGCACTGAAAGACGTGGAAAGAGCC |
| | | TTCGTGAACTTCTTCGAAAAACGGGCCAGATTCCCCAGATTCAAAAGCAAGAAAACCGACACCC |
| | | CCAGATTCAGAATCCCCCAAAGAGTGAGAATAGAGGGCAGCAGAGTGTACGTGCCCAAGGTGG |
| | | GGTGGGTGAAGCTGAGAAAGAGTCAGGAGATAGAAGGAAAAACCAAAAGCGCCACCTTCAAGA |
| | | GGGAGGCAGATGGCCATTGGTACGTGCTGCTGGTGAGTGAGTTCGAGATGCCTGATGTGCCTCT |
| | | GCCCCCAGTGCCCGAGAGCGAGGTGGTGGGAATCGACCTGGGACTGAAGGACTTCTACGTCCT |
| | | GTCTGACGGGGGCAGGAAGGAGGCCCCCAGATTCGCTAGAAAGGGCCAGCGAAAACTGAGAA |
| | | GAGCTGCAAGAAGACATAGCAAGTGCACCCGGGGAAGCAACAGAAAGGCCAAGGCAAAGAGG |
| | | AAGCTGGCAAGAGTGCACAGACAGATCGCAAACCAGAGAAAAGACTTCGTGCACAAGGCCACC |
| | | AGCGGCCTCGTGCAGCAGTACCAAGGCTTCTGCATCGAAAACCTGAGCATCAAGGGCATGGCCA |
| | | AAACCAAACTGTCAAAAAGCGTGCTGGACGCAGCCCTGGGGGAATTTAGAAGACAGCTGGCCT |
| | | ATAAAGCCCAGTGGCATAGAAAGTGGCTGGCAGTGATAGACAGATGGTTCCCCAGCAGCAAGCT |
| | | GTGCGGAGAGTGCGGCAGCATTAACGCCGATCTGACCCTGAGCGACAGAGAATGGACCTGCGA |
| | | GTGTGGCGCAGTGCACGACAGAGATCTGAACGCCGCTCGCAACATCAAGAGAGAGGGCCTGAG |
| | | TCAGATTGTGGTGGCCGGACATGCCGAAACCCTGAACGCCAGAGGGGAGGGCGTGAGACCCGC |
| | | CATTGCCGGAAGCCCTAGAAGCAGGGCTGACCCCAAGAAGAAGAGGAAGGTGTCGCCAGGGAT |
| | | CCGTCGACTTGACGCGTTGATATCAACAAGTTTGTACAAAAAAGCAGGCTACAAAGAGGCCAGC |
| | | GGTTCCGGACGGGCTGACGCATTGGACGATTTTGATCTGGATATGCTGGGAAGTGACGCCCTCG |
| | | ATGATTTTGACCTTGACATGCTTGGTTCGGATGCCCTTGATGACTTTGACCTCGACATGCTCGGCA |
| | | GTGACGCCCTTGATGATTTCGACCTGGACATGCTGATTAACTCTAGAAGTTCCGGATCTCCGAAA |
| | | AAGAAACGCAAAGTTGGTAGCCAGTACCTGCCCGACACCGACGACCGGCACCGGATCGAGGAA |
| | | AAGCGGAAGCGGACCTACGAGACATTCAAGAGCATCATGAAGAAGTCCCCCTTCAGCGGCCCC |
| | | ACCGACCCTAGACCTCCACCTAGAAGAATCGCCGTGCCCAGCAGATCCAGCGCCAGCGTGCCA |
| | | AAACCTGCCCCCCAGCCTTACCCCTTCACCAGCAGCCTGAGCACCATCAACTACGACGAGTTCCC |
| | | TACCATGGTGTTCCCCAGCGGCCAGATCTCTCAGGCCTCTGCTCTGGCTCCAGCCCCTCCTCAGG |
| | | TGCTGCCTCAGGCTCCTGCTCCTGCACCAGCTCCAGCCATGGTGTCTGCACTGGCTCAGGCACCA |
| | | GCACCCGTGCCTGTGCTGGCTCCTGGACCTCCACAGGCTGTGGCTCCACCAGCCCCTAAACCTA |
| | | CACAGGCCGGCGAGGGCACACTGTCTGAAGCTCTGCTGCAGCTGCAGTTCGACGACGAGGATCT |
| | | GGGAGCCCTGCTGGGAAACAGCACCGATCCTGCCGTGTTCACCGACCTGGCCAGCGTGGACAA |
| | | CAGCGAGTTCCAGCAGCTGCTGAACCAGGGCATCCCTGTGGCCCCTCACACCACCGAGCCCATG |
| | | CTGATGGAATACCCCGAGGCCATCACCCGGCTCGTGACAGGCGCTCAGAGGCCTCCTGATCCAG |
| | | CTCCTGCCCCTCTGGGAGCACCAGGCCTGCCTAATGGACTGCTGTCTGGCGACGAGGACTTCAG |
| | | CTCTATCGCCGATATGGATTTCTCAGCCTTGCTGGGCTCTGGCAGCGGCAGCCGGGATTCCAGGG |
| | | AAGGGATGTTTTTGCCGAAGCCTGAGGCCGGCTCCGCTATTAGTGACGTGTTTGAGGGCCGCGA |
| | | GGTGTGCCAGCCAAAACGAATCCGGCCATTTCATCCTCCAGGAAGTCCATGGGCCAACCGCCCA |
| | | CTCCCCGCCAGCCTCGCACCAACACCAACCGGTCCAGTACATGAGCCAGTCGGGTCACTGACCC |
| | | CGGCACCAGTCCCTCAGCCACTGGATCCAGGGCCCGCAGTGACTCCCGAGGCCAGTCACCTGTT |
| | | GGAGGATCCCGATGAAGAGACGAGCCAGGCTGTCAAAGCCCTTCGGGAGATGGCCGATACTGT |
| | | GATTCCCCAGAAGGAAGAGGCTGCAATCTGTGGCCAAATGGACCTTTCCCATCCGCCCCCAAGG |
| | | GGCCATCTGGATGAGCTGACAACCACACTTGAGTCCATGACCGAGGATCTGAACCTGGACTCAC |
| | | CCCTGACCCCGGAATTGAACGAGATTCTGGATACCTTCCTGAACGACGAGTGCCTCTTGCATGCC |
| | | ATGCATATCAGCACAGGACTGTCCATCTTCGACACATCTCTGTTTAAAAGGCCGGCGGCCACGAA |
| | | AAAGGCCGGCCAGGCAAAAAAGAAAAAGATGGCCTCAAACGATTATACCCAACAAGCAACCCA |
| | | AAGCTATGGGGCCTACCCCACCCAGCCCGGGCAGGGCTATTCCCAGCAGAGCAGTCAGCCCTA |
| | | CGGACAGCAGAGTTACAGTGGTTATAGCCAGTCCACGGACACTTCAGGCTATGGCCAGAGCAGC |
| | | TATTCTTCTTATGGCCAGAGCCAGAACACAGGCTATGGAACTCAGTCAACTCCCCAGGGATATGG |
| | | CTCGACTGGCGGCTATGGCAGTAGCCAGAGCTCCCAATCGTCTTACGGGCAGCAGTCCTCCTAC |
| | | CCTGGCTATGGCCAGCAGCCAGCTCCCAGCAGCACCTCGGGAAGTTACGGTAGCAGTTCTCAGA |
| | | GCAGCAGCTATGGGCAGCCCCAGAGTGGGAGCTACAGCCAGCAGCCTAGCTATGGTGGACAGC |
| | | AGCAAAGCTATGGACAGCAGCAAAGCTATAATCCCCCTCAGGGCTATGGACAGCAGAACCAGT |
| | | ACAACAGCAGCAGTGGTGGTGGAGGTGGAGGTGGAGGTGGAGGTAACTATGGCCAAGATCAAT |
| | | CCTCCATGAGTAGTGGTGGTGGCAGTGGTGGCGGTTATGGCAATCAAGACCAGAGTGGTGGAG |
| | | GTGGCAGCGGTGGCTATGGACAGCAGGACCGTGGA |
| seq. | vector_ | TTTAGCGCGTGCGCCAATTCTGCAGACAAATGGCTCTAGAGGTACCGACATTGATTATTGACTAG |
| 52 | ISDge10TnpB- | TTATTAATAGTAATCAATTACGGGGTCATTAGTTCATAGCCCATATATGGAGTTCCGCGTTACATA |
| | VPR-reRNA) | ACTTACGGTAAATGGCCCGCCTGGCTGACCGCCCAACGACCCCCGCCCATTGACGTCAATAATG |
| | (20 nt + WT | ACGTATGTTCCCATAGTAACGCCAATAGGGACTTTCCATTGACGTCAATGGGTGGAGTATTTACG |
| | TnpB) | GTAAACTGCCCACTTGGCAGTACATCAAGTGTATCATATGCCAAGTACGCCCCCTATTGACGTCA |
| | | ATGACGGTAAATGGCCCGCCTGGCATTATGCCCAGTACATGACCTTATGGGACTTTCCTACTTGG |
| | | CAGTACATCTACGTATTAGTCATCGCTATTACCATGGTGATGCGGTTTTGGCAGTACATCAATGGG |
| | | CGTGGATAGCGGTTTGACTCACGGGGATTTCCAAGTCTCCACCCCATTGACGTCAATGGGAGTTT |
| | | GTTTTGGCACCAAAATCAACGGGACTTTCCAAAATGTCGTAACAACTCCGCCCCATTGACGCAAA |
| | | TGGGCGGTAGGCGTGTACGGTGGGAGGTCTATATAAGCAGAGCTGGTTTAGTGAACCGTCAGAT |
| | | CCGCTAGAGATCCGCGGCCGCGCCACCATGGCCCCAAAGAAGAAGCGGAAGGTCGGTATCCAC |
| | | GGAGTCCCAGCAGCCATGACCACCCACCGCAAGGTGTACAGATACCGCATCGAGCCCACCCCC |
| | | GTGCAGGAGAGCAAGCTGTACATGCTGGCCGGCAGCCGGAGATTCGTGTTCAACTGGGCCCTG |
| | | GCCCGGAGAAGAGAGCACTACGCCGAAACCGGCAAGACCCTGGGATACAACGCCCAGGCAGG |
| | | AGAGCTGACCGCCCTGAAAAACCAAGAAGAAACCAGCTGGCTGAAAGAGTCCGACTCACAACT |
| | | GCTGCAGCAGGCACTGAAAGACGTGGAAAGAGCCTTCGTGAACTTCTTCGAAAAACGGGCCAG |
| | | ATTCCCCAGATTCAAAAGCAAGAAAACCGACACCCCCAGATTCAGAATCCCCCAAAGAGTGAGA |
| | | ATAGAGGGCAGCAGAGTGTACGTGCCCAAGGTGGGGTGGGTGAAGCTGAGAAAGAGTCAGGA |
| | | GATAGAAGGAAAAACCAAAAGCGCCACCTTCAAGAGGGAGGCAGATGGCCATTGGTACGTGCT |
| | | GCTGGTGAGTGAGTTCGAGATGCCTGATGTGCCTCTGCCCCCAGTGCCCGAGAGCGAGGTGGTG |
| | | GGAATCGACCTGGGACTGAAGGACTTCTACGTCCTGTCTGACGGGGGCAGGAAGGAGGCCCCC |
| | | AGATTCGCTAGAAAGGGCCAGCGAAAACTGAGAAGAGCTGCAAGAAGACATAGCAAGTGCACC |
| | | CGGGGAAGCAACAGAAAGGCCAAGGCAAAGAGGAAGCTGGCAAGAGTGCACAGACAGATCGC |
| | | AAACCAGAGAAAAGACTTCGTGCACAAGGCCACCAGCGGCCTCGTGCAGCAGTACCAAGGCTT |
| | | CTGCATCGAAAACCTGAGCATCAAGGGCATGGCCAAAACCAAACTGTCAAAAAGCGTGCTGGA |
| | | CGCAGCCCTGGGGGAATTTAGAAGACAGCTGGCCTATAAAGCCCAGTGGCATAGAAAGTGGCT |
| | | GGCAGTGATAGACAGATGGTTCCCCAGCAGCAAGCTGTGCGGAGAGTGCGGCAGCATTAACGC |
| | | CGATCTGACCCTGAGCGACAGAGAATGGACCTGCGAGTGTGGCGCAGTGCACGACAGAGATCT |
| | | GAACGCCGCTCGCAACATCAAGAGAGAGGGCCTGAGTCAGATTGTGGTGGCCGGACATGCCGA |
| | | AACCCTGAACGCCAGAGGGGAGGGCGTGAGACCCGCCATTGCCGGAAGCCCTAGAAGCAGGG |
| | | CTGACCCCAAGAAGAAGAGGAAGGTGTCGCCAGGGATCCGTCGACTTGACGCGTTGATATCAAC |
| | | AAGTTTGTACAAAAAAGCAGGCTACAAAGAGGCCAGCGGTTCCGGACGGGCTGACGCATTGGA |
| | | CGATTTTGATCTGGATATGCTGGGAAGTGACGCCCTCGATGATTTTGACCTTGACATGCTTGGTTC |
| | | GGATGCCCTTGATGACTTTGACCTCGACATGCTCGGCAGTGACGCCCTTGATGATTTCGACCTGG |
| | | ACATGCTGATTAACTCTAGAAGTTCCGGATCTCCGAAAAAGAAACGCAAAGTTGGTAGCCAGTA |
| | | CCTGCCCGACACCGACGACCGGCACCGGATCGAGGAAAAGCGGAAGCGGACCTACGAGACATT |
| | | CAAGAGCATCATGAAGAAGTCCCCCTTCAGCGGCCCCACCGACCCTAGACCTCCACCTAGAAGA |
| | | ATCGCCGTGCCCAGCAGATCCAGCGCCAGCGTGCCAAAACCTGCCCCCCAGCCTTACCCCTTCA |
| | | CCAGCAGCCTGAGCACCATCAACTACGACGAGTTCCCTACCATGGTGTTCCCCAGCGGCCAGAT |
| | | CTCTCAGGCCTCTGCTCTGGCTCCAGCCCCTCCTCAGGTGCTGCCTCAGGCTCCTGCTCCTGCAC |
| | | CAGCTCCAGCCATGGTGTCTGCACTGGCTCAGGCACCAGCACCCGTGCCTGTGCTGGCTCCTGG |
| | | ACCTCCACAGGCTGTGGCTCCACCAGCCCCTAAACCTACACAGGCCGGCGAGGGCACACTGTCT |
| | | GAAGCTCTGCTGCAGCTGCAGTTCGACGACGAGGATCTGGGAGCCCTGCTGGGAAACAGCACC |
| | | GATCCTGCCGTGTTCACCGACCTGGCCAGCGTGGACAACAGCGAGTTCCAGCAGCTGCTGAACC |
| | | AGGGCATCCCTGTGGCCCCTCACACCACCGAGCCCATGCTGATGGAATACCCCGAGGCCATCAC |
| | | CCGGCTCGTGACAGGCGCTCAGAGGCCTCCTGATCCAGCTCCTGCCCCTCTGGGAGCACCAGGC |
| | | CTGCCTAATGGACTGCTGTCTGGCGACGAGGACTTCAGCTCTATCGCCGATATGGATTTCTCAGC |
| | | CTTGCTGGGCTCTGGCAGCGGCAGCCGGGATTCCAGGGAAGGGATGTTTTTGCCGAAGCCTGAG |
| | | GCCGGCTCCGCTATTAGTGACGTGTTTGAGGGCCGCGAGGTGTGCCAGCCAAAACGAATCCGGC |
| | | CATTTCATCCTCCAGGAAGTCCATGGGCCAACCGCCCACTCCCCGCCAGCCTCGCACCAACACC |
| | | AACCGGTCCAGTACATGAGCCAGTCGGGTCACTGACCCCGGCACCAGTCCCTCAGCCACTGGAT |
| | | CCAGCGCCCGCAGTGACTCCCGAGGCCAGTCACCTGTTGGAGGATCCCGATGAAGAGACGAGC |
| | | CAGGCTGTCAAAGCCCTTCGGGAGATGGCCGATACTGTGATTCCCCAGAAGGAAGAGGCTGCAA |
| | | TCTGTGGCCAAATGGACCTTTCCCATCCGCCCCCAAGGGGCCATCTGGATGAGCTGACAACCAC |
| | | ACTTGAGTCCATGACCGAGGATCTGAACCTGGACTCACCCCTGACCCCGGAATTGAACGAGATT |
| | | CTGGATACCTTCCTGAACGACGAGTGCCTCTTGCATGCCATGCATATCAGCACAGGACTGTCCAT |
| | | CTTCGACACATCTCTGTTTAAAAGGCCGGCGGCCACGAAAAAGGCCGGCCAGGCAAAAAAGAA |
| | | AAAGATGGCCTCAAACGATTATACCCAACAAGCAACCCAAAGCTATGGGGCCTACCCCACCCAG |
| | | CCCGGGCAGGGCTATTCCCAGCAGAGCAGTCAGCCCTACGGACAGCAGAGTTACAGTGGTTATA |
| | | GCCAGTCCACGGACACTTCAGGCTATGGCCAGAGCAGCTATTCTTCTTATGGCCAGAGCCAGAA |
| | | CACAGGCTATGGAACTCAGTCAACTCCCCAGGGATATGGCTCGACTGGCGGCTATGGCAGTAGC |
| | | CAGAGCTCCCAATCGTCTTACGGGCAGCAGTCCTCCTACCCTGGCTATGGCCAGCAGCCAGCTC |
| | | CCAGCAGCACCTCGGGAAGTTACGGTAGCAGTTCTCAGAGCAGCAGCTATGGGCAGCCCCAGA |
| | | GTGGGAGCTACAGCCAGCAGCCTAGCTATGGTGGACAGCAGCAAAGCTATGGACAGCAGCAAA |
| | | GCTATAATCCCCCTCAGGGCTATGGACAGCAGAACCAGTACAACAGCAGCAGTGGTGGTGGAG |
| | | GTGGAGGTGGAGGTGGAGGTAACTATGGCCAAGATCAATCCTCCATGAGTAGTGGTGGTGGCA |
| | | GTGGTGGCGGTTATGGCAATCAAGACCAGAGTGGTGGAGGTGGCAGCGGTGGCTATGGACAGC |
| | | AGGACCGTGGAGAATTCGGCAGTGGAGAGGGCAGAGGAAGTCTGCTAACATGCGGTGACGTCG |
| | | AGGAGAATCCTGGCCCAGTGAGCAAGGGCGAGGAGCTGTTCACCGGGGTGGTGCCCATCCTGG |
| | | TCGAGCTGGACGGCGACGTAAACGGCCACAAGTTCAGCGTGTCCGGCGAGGGCGAGGGCGATG |
| | | CCACCTACGGCAAGCTGACCCTGAAGTTCATCTGCACCACCGGCAAGCTGCCCGTGCCCTGGCC |
| | | CACCCTCGTGACCACCCTGACCTACGGCGTGCAGTGCTTCAGCCGCTACCCCGACCACATGAAG |
| | | CAGCACGACTTCTTCAAGTCCGCCATGCCCGAAGGCTACGTCCAGGAGCGCACCATCTTCTTCAA |
| | | GGACGACGGCAACTACAAGACCCGCGCCGAGGTGAAGTTCGAGGGCGACACCCTGGTGAACCG |
| | | CATCGAGCTGAAGGGCATCGACTTCAAGGAGGACGGCAACATCCTGGGGCACAAGCTGGAGTA |
| | | CAACTACAACAGCCACAACGTCTATATCATGGCCGACAAGCAGAAGAACGGCATCAAGGTGAA |
| | | CTTCAAGATCCGCCACAACATCGAGGACGGCAGCGTGCAGCTCGCCGACCACTACCAGCAGAA |
| | | CACCCCCATCGGCGACGGCCCCGTGCTGCTGCCCGACAACCACTACCTGAGCACCCAGTCCGCC |
| | | CTGAGCAAAGACCCCAACGAGAAGCGCGATCACATGGTCCTGCTGGAGTTCGTGACCGCCGCC |
| | | GGGATCACTCTCGGCATGGACGAGCTGTACAAGGAATTCTAACTAGAGCTCGCTGATCAGCCTC |
| | | GACTGTGCCTTCTAGTTGCCAGCCATCTGTTGTTTGCCCCTCCCCCGTGCCTTCCTTGACCCTGGA |
| | | AGGTGCCACTCCCACTGTCCTTTCCTAATAAAATGAGGAAATTGCATCGCATTGTCTGAGTAGGT |
| | | GTCATTCTATTCTGGGGGGTGGGGTGGGGCAGGACAGCAAGGGGGAGGATTGGGAAGAGAATA |
| | | GCAGGCATGCTGGGGATGCGGTGGGCTCTATGGCTTCTGAGGCGGAAAGAACCAGCTGGGGCT |
| | | CGATACCGTCGACCTCTAGCTAGAGCTTGGCGTAATCATGGTCATAGCTGTTTCCTGTGTGAAATT |
| | | GTTATCCGCTCACAATTCCACACAACATACGAGCCGGAAGCATAAAGTGTAAAGCCTAGGGTGC |
| | | CTAATGAGTGAGCTAACTCACATTAATTGCGTTGCGCTCACTGCCCGCTTTCCAGTCGGGAAACC |
| | | TGTCGTGCCAGCTGCATTAATGAATCGGCCAACGCGCGGGGAGAGGCGGTTTGCGTATTGGGCG |
| | | CTCTTCCGCTTCCTCGCTCACTGACTCGCTGCGCTCGGTCGTTCGGCTGCGGCGAGCGGTATCAG |
| | | CTCACTCAAAGGCGGTAATACGGTTATCCACAGAATCAGGGGATAACGCAGGAAAGAACATGTG |
| | | AGCAAAAGGCCAGCAAAAGGCCAGGAACCGTAAAAAGGCCGCGTTGCTGGCGTTTTTCCATAG |
| | | GCTCCGCCCCCCTGACGAGCATCACAAAAATCGACGCTCAAGTCAGAGGTGGCGAAACCCGAC |
| | | AGGACTATAAAGATACCAGGCGTTTCCCCCTGGAAGCTCCCTCGTGCGCTCTCCTGTTCCGACCC |
| | | TGCCGCTTACCGGATACCTGTCCGCCTTTCTCCCTTCGGGAAGCGTGGCGCTTTCTCATAGCTCAC |
| | | GCTGTAGGTATCTCAGTTCGGTGTAGGTCGTTCGCTCCAAGCTGGGCTGTGTGCACGAACCCCCC |
| | | GTTCAGCCCGACCGCTGCGCCTTATCCGGTAACTATCGTCTTGAGTCCAACCCGGTAAGACACGA |
| | | CTTATCGCCACTGGCAGCAGCCACTGGTAACAGGATTAGCAGAGCGAGGTATGTAGGCGGTGCT |
| | | ACAGAGTTCTTGAAGTGGTGGCCTAACTACGGCTACACTAGAAGAACAGTATTTGGTATCTGCGC |
| | | TCTGCTGAAGCCAGTTACCTTCGGAAAAAGAGTTGGTAGCTCTTGATCCGGCAAACAAACCACC |
| | | GCTGGTAGCGGTGGTTTTTTTGTTTGCAAGCAGCAGATTACGCGCAGAAAAAAAGGATCTCAAGA |
| | | AGATCCTTTGATCTTTTCTACGGGGTCTGACGCTCAGTGGAACGAAAACTCACGTTAAGGGATTTT |
| | | GGTCATGAGATTATCAAAAAGGATCTTCACCTAGATCCTTTTAAATTAAAAATGAAGTTTTAAATC |
| | | AATCTAAAGTATATATGAGTAAACTTGGTCTGACAGTTACCAATGCTTAATCAGTGAGGCACCTAT |
| | | CTCAGCGATCTGTCTATTTCGTTCATCCATAGTTGCCTGACTCCCCGTCGTGTAGATAACTACGAT |
| | | ACGGGAGGGCTTACCATCTGGCCCCAGTGCTGCAATGATACCGCGAGACCCACGCTCACCGGCT |
| | | CCAGATTTATCAGCAATAAACCAGCCAGCCGGAAGGGCCGAGCGCAGAAGTGGTCCTGCAACTT |
| | | TATCCGCCTCCATCCAGTCTATTAATTGTTGCCGGGAAGCTAGAGTAAGTAGTTCGCCAGTTAATA |
| | | GTTTGCGCAACGTTGTTGCCATTGCTACAGGCATCGTGGTGTCACGCTCGTCGTTTGGTATGGCTT |
| | | CATTCAGCTCCGGTTCCCAACGATCAAGGCGAGTTACATGATCCCCCATGTTGTGCAAAAAAGCG |
| | | GTTAGCTCCTTCGGTCCTCCGATCGTTGTCAGAAGTAAGTTGGCCGCAGTGTTATCACTCATGGTT |
| | | ATGGCAGCACTGCATAATTCTCTTACTGTCATGCCATCCGTAAGATGCTTTTCTGTGACTGGTGAG |
| | | TACTCAACCAAGTCATTCTGAGAATAGTGTATGCGGCGACCGAGTTGCTCTTGCCCGGCGTCAAT |
| | | ACGGGATAATACCGCGCCACATAGCAGAACTTTAAAAGTGCTCATCATTGGAAAACGTTCTTCGG |
| | | GGCGAAAACTCTCAAGGATCTTACCGCTGTTGAGATCCAGTTCGATGTAACCCACTCGTGCACCC |
| | | AACTGATCTTCAGCATCTTTTACTTTCACCAGCGTTTCTGGGTGAGCAAAAACAGGAAGGCAAAA |
| | | TGCCGCAAAAAAGGGAATAAGGGCGACACGGAAATGTTGAATACTCATACTCTTCCTTTTTCAAT |
| | | ATTATTGAAGCATTTATCAGGGTTATTGTCTCATGAGCGGATACATATTTGAATGTATTTAGAAAA |
| | | ATAAACAAATAGGGGTTCCGCGCACATTTCCCCGAAAAGTGCCACCTGACGTCGACGGATCGGG |
| | | AGATCGATCTCCCGATCCCCTAGGGTCGACTCTCAGTACAATCTGCTCTGATGCCGCATAGTTAA |
| | | GCCAGTATCTGCTCCCTGCTTGTGTGTTGGAGGTCGCTGAGTAGTGCGCGAGCAAAATTTAAGCT |
| | | ACAACAAGGCAAGGCTTGACCGACAATTGCATGAAGAATCTGCTTAGGGTTAGGCGTTTTGCGCT |
| | | GCTTCGCGATGTACGGGCCAGATATACGCGTGAGGGCCTATTTCCCATGATTCCTTCATATTTGCA |
| | | TATACGATACAAGGCTGTTAGAGAGATAATTGGAATTAATTTGACTGTAAACACAAAGATATTAG |
| | | TACAAAATACGTGACGTAGAAAGTAATAATTTCTTGGGTAGTTTGCAGTTTTAAAATTATGTTTTAA |
| | | AATGGACTATCATATGCTTACCGTAACTTGAAAGTATTTCGATTTCTTGGCTTTATATATCTTGTGG |
| | | AAAGGACGAAACACCGGTGGAGCGGTTCACGACCGCGACCTCAACGCCGCCCGGAACATCAAG |
| | | CGGGAAGGGCTTTCGCAAATCGTCGTCGCGGGGCACGCGGAGACGTTAAACGCTCGGGGAGAG |
| | | GGTGTCAGACCTGCGATAGCGGGCAGCCCTCGATGAAGCGAGAATCCAACGGCTTTAGCCGTTG |
| | | GAGTGTCAAGGGCTGTTGTCAGACCCAACTTTTATTTTTTGTTTTAGAGCTAGAAATAGTAAGTTA |
| | | AAATAAGGCTAGTCCGTT |
| seq. | vector_ | TTTAGCGCGTGCGCCAATTCTGCAGACAAATGGCTCTAGAGGTACCGACATTGATTATTGACTAG |
| 53 | ISDge10TnpB- | TTATTAATAGTAATCAATTACGGGGTCATTAGTTCATAGCCCATATATGGAGTTCCGCGTTACATA |
| | VPR-reRNA) | ACTTACGGTAAATGGCCCGCCTGGCTGACCGCCCAACGACCCCCGCCCATTGACGTCAATAATG |
| | (18 nt-WT | ACGTATGTTCCCATAGTAACGCCAATAGGGACTTTCCATTGACGTCAATGGGTGGAGTATTTACG |
| | TnpB) | GTAAACTGCCCACTTGGCAGTACATCAAGTGTATCATATGCCAAGTACGCCCCCTATTGACGTCA |
| | | ATGACGGTAAATGGCCCGCCTGGCATTATGCCCAGTACATGACCTTATGGGACTTTCCTACTTGG |
| | | CAGTACATCTACGTATTAGTCATCGCTATTACCATGGTGATGCGGTTTTGGCAGTACATCAATGGG |
| | | CGTGGATAGCGGTTTGACTCACGGGGATTTCCAAGTCTCCACCCCATTGACGTCAATGGGAGTTT |
| | | GTTTTGGCACCAAAATCAACGGGACTTTCCAAAATGTCGTAACAACTCCGCCCCATTGACGCAAA |
| | | TGGGCGGTAGGCGTGTACGGTGGGAGGTCTATATAAGCAGAGCTGGTTTAGTGAACCGTCAGAT |
| | | CCGCTAGAGATCCGCGGCCGCGCCACCATGGCCCCAAAGAAGAAGCGGAAGGTCGGTATCCAC |
| | | GGAGTCCCAGCAGCCATGACCACCCACCGCAAGGTGTACAGATACCGCATCGAGCCCACCCCC |
| | | GTGCAGGAGAGCAAGCTGTACATGCTGGCCGGCAGCCGGAGATTCGTGTTCAACTGGGCCCTG |
| | | GCCCGGAGAAGAGAGCACTACGCCGAAACCGGCAAGACCCTGGGATACAACGCCCAGGCAGG |
| | | AGAGCTGACCGCCCTGAAAAACCAAGAAGAAACCAGCTGGCTGAAAGAGTCCGACTCACAACT |
| | | GCTGCAGCAGGCACTGAAAGACGTGGAAAGAGCCTTCGTGAACTTCTTCGAAAAACGGGCCAG |
| | | ATTCCCCAGATTCAAAAGCAAGAAAACCGACACCCCCAGATTCAGAATCCCCCAAAGAGTGAGA |
| | | ATAGAGGGCAGCAGAGTGTACGTGCCCAAGGTGGGGTGGGTGAAGCTGAGAAAGAGTCAGGA |
| | | GATAGAAGGAAAAACCAAAAGCGCCACCTTCAAGAGGGAGGCAGATGGCCATTGGTACGTGCT |
| | | GCTGGTGAGTGAGTTCGAGATGCCTGATGTGCCTCTGCCCCCAGTGCCCGAGAGCGAGGTGGTG |
| | | GGAATCGACCTGGGACTGAAGGACTTCTACGTCCTGTCTGACGGGGGCAGGAAGGAGGCCCCC |
| | | AGATTCGCTAGAAAGGGCCAGCGAAAACTGAGAAGAGCTGCAAGAAGACATAGCAAGTGCACC |
| | | CGGGGAAGCAACAGAAAGGCCAAGGCAAAGAGGAAGCTGGCAAGAGTGCACAGACAGATCGC |
| | | AAACCAGAGAAAAGACTTCGTGCACAAGGCCACCAGCGGCCTCGTGCAGCAGTACCAAGGCTT |
| | | CTGCATCGAAAACCTGAGCATCAAGGGCATGGCCAAAACCAAACTGTCAAAAAGCGTGCTGGA |
| | | CGCAGCCCTGGGGGAATTTAGAAGACAGCTGGCCTATAAAGCCCAGTGGCATAGAAAGTGGCT |
| | | GGCAGTGATAGACAGATGGTTCCCCAGCAGCAAGCTGTGCGGAGAGTGCGGCAGCATTAACGC |
| | | CGATCTGACCCTGAGCGACAGAGAATGGACCTGCGAGTGTGGCGCAGTGCACGACAGAGATCT |
| | | GAACGCCGCTCGCAACATCAAGAGAGAGGGCCTGAGTCAGATTGTGGTGGCCGGACATGCCGA |
| | | AACCCTGAACGCCAGAGGGGAGGGCGTGAGACCCGCCATTGCCGGAAGCCCTAGAAGCAGGG |
| | | CTGACCCCAAGAAGAAGAGGAAGGTGTCGCCAGGGATCCGTCGACTTGACGCGTTGATATCAAC |
| | | AAGTTTGTACAAAAAAGCAGGCTACAAAGAGGCCAGCGGTTCCGGACGGGCTGACGCATTGGA |
| | | CGATTTTGATCTGGATATGCTGGGAAGTGACGCCCTCGATGATTTTGACCTTGACATGCTTGGTTC |
| | | GGATGCCCTTGATGACTTTGACCTCGACATGCTCGGCAGTGACGCCCTTGATGATTTCGACCTGG |
| | | ACATGCTGATTAACTCTAGAAGTTCCGGATCTCCGAAAAAGAAACGCAAAGTTGGTAGCCAGTA |
| | | CCTGCCCGACACCGACGACCGGCACCGGATCGAGGAAAAGCGGAAGCGGACCTACGAGACATT |
| | | CAAGAGCATCATGAAGAAGTCCCCCTTCAGCGGCCCCACCGACCCTAGACCTCCACCTAGAAGA |
| | | ATCGCCGTGCCCAGCAGATCCAGCGCCAGCGTGCCAAAACCTGCCCCCCAGCCTTACCCCTTCA |
| | | CCAGCAGCCTGAGCACCATCAACTACGACGAGTTCCCTACCATGGTGTTCCCCAGCGGCCAGAT |
| | | CTCTCAGGCCTCTGCTCTGGCTCCAGCCCCTCCTCAGGTGCTGCCTCAGGCTCCTGCTCCTGCAC |
| | | CAGCTCCAGCCATGGTGTCTGCACTGGCTCAGGCACCAGCACCCGTGCCTGTGCTGGCTCCTGG |
| | | ACCTCCACAGGCTGTGGCTCCACCAGCCCCTAAACCTACACAGGCCGGCGAGGGCACACTGTCT |
| | | GAAGCTCTGCTGCAGCTGCAGTTCGACGACGAGGATCTGGGAGCCCTGCTGGGAAACAGCACC |
| | | GATCCTGCCGTGTTCACCGACCTGGCCAGCGTGGACAACAGCGAGTTCCAGCAGCTGCTGAACC |
| | | AGGGCATCCCTGTGGCCCCTCACACCACCGAGCCCATCCTGATGGAATACCCCGAGGCCATCAC |
| | | CCGGCTCGTGACAGGCGCTCAGAGGCCTCCTGATCCAGCTCCTGCCCCTCTGGGAGCACCAGGC |
| | | CTGCCTAATGGACTGCTGTCTGGCGACGAGGACTTCAGCTCTATCGCCGATATGGATTTCTCAGC |
| | | CTTGCTGGGCTCTGGCAGCGGCAGCCGGGATTCCAGGGAAGGGATGTTTTTGCCGAAGCCTGAG |
| | | GCCGGCTCCGCTATTAGTGACGTGTTTGAGGGCCGCGAGGTGTGCCAGCCAAAACGAATCCGGC |
| | | CATTTCATCCTCCAGGAAGTCCATGGGCCAACCGCCCACTCCCCGCCAGCCTCGCACCAACACC |
| | | AACCGGTCCAGTACATGAGCCAGTCGGGTCACTGACCCCGGCACCAGTCCCTCAGCCACTGGAT |
| | | CCAGCGCCCGCAGTGACTCCCGAGGCCAGTCACCTGTTGGAGGATCCCGATGAAGAGACGAGC |
| | | CAGGCTGTCAAAGCCCTTCGGGAGATGGCCGATACTGTGATTCCCCAGAAGGAAGAGGCTGCAA |
| | | TCTGTGGCCAAATGGACCTTTCCCATCCGCCCCCAAGGGGCCATCTGGATGAGCTGACAACCAC |
| | | ACTTGAGTCCATGACCGAGGATCTGAACCTGGACTCACCCCTGACCCCGGAATTGAACGAGATT |
| | | CTGGATACCTTCCTGAACGACGAGTGCCTCTTGCATGCCATGCATATCAGCACAGGACTGTCCAT |
| | | CTTCGACACATCTCTGTTTAAAAGGCCGGCGGCCACGAAAAAGGCCGGCCAGGCAAAAAAGAA |
| | | AAAGATGGCCTCAAACGATTATACCCAACAAGCAACCCAAAGCTATGGGGCCTACCCCACCCAG |
| | | CCCGGGCAGGGCTATTCCCAGCAGAGCAGTCAGCCCTACGGACAGCAGAGTTACAGTGGTTATA |
| | | GCCAGTCCACGGACACTTCAGGCTATGGCCAGAGCAGCTATTCTTCTTATGGCCAGAGCCAGAA |
| | | CACAGGCTATGGAACTCAGTCAACTCCCCAGGGATATGGCTCGACTGGCGGCTATGGCAGTAGC |
| | | CAGAGCTCCCAATCGTCTTACGGGCAGCAGTCCTCCTACCCTGGCTATGGCCAGCAGCCAGCTC |
| | | CCAGCAGCACCTCGGGAAGTTACGGTAGCAGTTCTCAGAGCAGCAGCTATGGGCAGCCCCAGA |
| | | GTGGGAGCTACAGCCAGCAGCCTAGCTATGGTGGACAGCAGCAAAGCTATGGACAGCAGCAAA |
| | | GCTATAATCCCCCTCAGGGCTATGGACAGCAGAACCAGTACAACAGCAGCAGTGGTGGTGGAG |
| | | GTGGAGGTGGAGGTGGAGGTAACTATGGCCAAGATCAATCCTCCATGAGTAGTGGTGGTGGCA |
| | | GTGGTGGCGGTTATGGCAATCAAGACCAGAGTGGTGGAGGTGGCAGCGGTGGCTATGGACAGC |
| | | AGGACCGTGGAGAATTCGGCAGTGGAGAGGGCAGAGGAAGTCTGCTAACATGCGGTGACGTCG |
| | | AGGAGAATCCTGGCCCAGTGAGCAAGGGCGAGGAGCTGTTCACCGGGGTGGTGCCATCCTGG |
| | | TCGAGCTGGACGGCGACGTAAACGGCCACAAGTTCAGCGTGTCCGGCGAGGGCGAGGGCGATG |
| | | CCACCTACGGCAAGCTGACCCTGAAGTTCATCTGCACCACCGGCAAGCTGCCCGTGCCCTGGCC |
| | | CACCCTCGTGACCACCCTGACCTACGGCGTGCAGTGCTTCAGCCGCTACCCCGACCACATGAAG |
| | | CAGCACGACTTCTTCAAGTCCGCCATGCCCGAAGGCTACGTCCAGGAGCGCACCATCTTCTTCAA |
| | | GGACGACGGCAACTACAAGACCCGCGCCGAGGTGAAGTTCGAGGGCGACACCCTGGTGAACCG |
| | | CATCGAGCTGAAGGGCATCGACTTCAAGGAGGACGGCAACATCCTGGGGCACAAGCTGGAGTA |
| | | CAACTACAACAGCCACAACGTCTATATCATGGCCGACAAGCAGAAGAACGGCATCAAGGTGAA |
| | | CTTCAAGATCCGCCACAACATCGAGGACGGCAGCGTGCAGCTCGCCGACCACTACCAGCAGAA |
| | | CACCCCCATCGGCGACGGCCCCGTGCTGCTGCCCGACAACCACTACCTGAGCACCCAGTCCGCC |
| | | CTGAGCAAAGACCCCAACGAGAAGCGCGATCACATGGTCCTGCTGGAGTTCGTGACCGCCGCC |
| | | GGGATCACTCTCGGCATGGACGAGCTGTACAAGGAATTCTAACTAGAGCTCGCTGATCAGCCTC |
| | | GACTGTGCCTTCTAGTTGCCAGCCATCTGTTGTTTGCCCCTCCCCCGTGCCTTCCTTGACCCTGGA |
| | | AGGTGCCACTCCCACTGTCCTTTCCTAATAAAATGAGGAAATTGCATCGCATTGTCTGAGTAGGT |
| | | GTCATTCTATTCTGGGGGGTGGGGTGGGGCAGGACAGCAAGGGGGAGGATTGGGAAGAGAATA |
| | | GCAGGCATGCTGGGGATGCGGTGGGCTCTATGGCTTCTGAGGCGGAAAGAACCAGCTGGGGCT |
| | | CGATACCGTCGACCTCTAGCTAGAGCTTGGCGTAATCATGGTCATAGCTGTTTCCTGTGTGAAATT |
| | | GTTATCCGCTCACAATTCCACACAACATACGAGCCGGAAGCATAAAGTGTAAAGCCTAGGGTGC |
| | | CTAATGAGTGAGCTAACTCACATTAATTGCGTTGCGCTCACTGCCCGCTTTCCAGTCGGGAAACC |
| | | TGTCGTGCCAGCTGCATTAATGAATCGGCCAACGCGCGGGGAGAGGCGGTTTGCGTATTGGGCG |
| | | CTCTTCCGCTTCCTCGCTCACTGACTCGCTGCGCTCGGTCGTTCGGCTGCGGCGAGCGGTATCAG |
| | | CTCACTCAAAGGCGGTAATACGGTTATCCACAGAATCAGGGGATAACGCAGGAAAGAACATGTG |
| | | AGCAAAAGGCCAGCAAAAGGCCAGGAACCGTAAAAAGGCCGCGTTGCTGGCGTTTTTCCATAG |
| | | GCTCCGCCCCCCTGACGAGCATCACAAAAATCGACGCTCAAGTCAGAGGTGGCGAAACCCGAC |
| | | AGGACTATAAAGATACCAGGCGTTTCCCCCTGGAAGCTCCCTCGTGCGCTCTCCTGTTCCGACCC |
| | | TGCCGCTTACCGGATACCTGTCCGCCTTTCTCCCTTCGGGAAGCGTGGCGCTTTCTCATAGCTCAC |
| | | GCTGTAGGTATCTCAGTTCGGTGTAGGTCGTTCGCTCCAAGCTGGGCTGTGTGCACGAACCCCCC |
| | | GTTCAGCCCGACCGCTGCGCCTTATCCGGTAACTATCGTCTTGAGTCCAACCCGGTAAGACACGA |
| | | CTTATCGCCACTGGCAGCAGCCACTGGTAACAGGATTAGCAGAGCGAGGTATGTAGGCGGTGCT |
| | | ACAGAGTTCTTGAAGTGGTGGCCTAACTACGGCTACACTAGAAGAACAGTATTTGGTATCTGCGC |
| | | TCTGCTGAAGCCAGTTACCTTCGGAAAAAGAGTTGGTAGCTCTTGATCCGGCAAACAAACCACC |
| | | GCTGGTAGCGGTGGTTTTTTTGTTTGCAAGCAGCAGATTACGCGCAGAAAAAAAGGATCTCAAGA |
| | | AGATCCTTTGATCTTTTCTACGGGGTCTGACGCTCAGTGGAACGAAAACTCACGTTAAGGGATTTT |
| | | GGTCATGAGATTATCAAAAAGGATCTTCACCTAGATCCTTTTAAATTAAAAATGAAGTTTTAAATC |
| | | AATCTAAAGTATATATGAGTAAACTTGGTCTGACAGTTACCAATGCTTAATCAGTGAGGCACCTAT |
| | | CTCAGCGATCTGTCTATTTCGTTCATCCATAGTTGCCTGACTCCCCGTCGTGTAGATAACTACGAT |
| | | ACGGGAGGGCTTACCATCTGGCCCCAGTGCTGCAATGATACCGCGAGACCCACGCTCACCGGCT |
| | | CCAGATTTATCAGCAATAAACCAGCCAGCCGGAAGGGCCGAGCGCAGAAGTGGTCCTGCAACTT |
| | | TATCCGCCTCCATCCAGTCTATTAATTGTTGCCGGGAAGCTAGAGTAAGTAGTTCGCCAGTTAATA |
| | | GTTTGCGCAACGTTGTTGCCATTGCTACAGGCATCGTGGTGTCACGCTCGTCGTTTGGTATGGCTT |
| | | CATTCAGCTCCGGTTCCCAACGATCAAGGCGAGTTACATGATCCCCCATGTTGTGCAAAAAAGCG |
| | | GTTAGCTCCTTCGGTCCTCCGATCGTTGTCAGAAGTAAGTTGGCCGCAGTGTTATCACTCATGGTT |
| | | ATGGCAGCACTGCATAATTCTCTTACTGTCATGCCATCCGTAAGATGCTTTTCTGTGACTGGTGAG |
| | | TACTCAACCAAGTCATTCTGAGAATAGTGTATGCGGCGACCGAGTTGCTCTTGCCCGGCGTCAAT |
| | | ACGGGATAATACCGCGCCACATAGCAGAACTTTAAAAGTGCTCATCATTGGAAAACGTTCTTCGG |
| | | GGCGAAAACTCTCAAGGATCTTACCGCTGTTGAGATCCAGTTCGATGTAACCCACTCGTGCACCC |
| | | AACTGATCTTCAGCATCTTTTACTTTCACCAGCGTTTCTGGGTGAGCAAAAACAGGAAGGCAAAA |
| | | TGCCGCAAAAAAGGGAATAAGGGCGACACGGAAATGTTGAATACTCATACTCTTCCTTTTTCAAT |
| | | ATTATTGAAGCATTTATCAGGGTTATTGTCTCATGAGCGGATACATATTTGAATGTATTTAGAAAA |
| | | ATAAACAAATAGGGGTTCCGCGCACATTTCCCCGAAAAGTGCCACCTGACGTCGACGGATCGGG |
| | | AGATCGATCTCCCGATCCCCTAGGGTCGACTCTCAGTACAATCTGCTCTGATGCCGCATAGTTAA |
| | | GCCAGTATCTGCTCCCTGCTTGTGTGTTGGAGGTCGCTGAGTAGTGCGCGAGCAAAATTTAAGCT |
| | | ACAACAAGGCAAGGCTTGACCGACAATTGCATGAAGAATCTGCTTAGGGTTAGGCGTTTTGCGCT |
| | | GCTTCGCGATGTACGGGCCAGATATACGCGTGAGGGCCTATTTCCCATGATTCCTTCATATTTGCA |
| | | TATACGATACAAGGCTGTTAGAGAGATAATTGGAATTAATTTGACTGTAAACACAAAGATATTAG |
| | | TACAAAATACGTGACGTAGAAAGTAATAATTTCTTGGGTAGTTTGCAGTTTTAAAATTATGTTTTAA |
| | | AATGGACTATCATATGCTTACCGTAACTTGAAAGTATTTCGATTTCTTGGCTTTATATATCTTGTGG |
| | | AAAGGACGAAACACCGGTGGAGCGGTTCACGACCGCGACCTCAACGCCGCCCGGAACATCAAG |
| | | CGGGAAGGGCTTTCGCAAATCGTCGTCGCGGGGCACGCGGAGACGTTAAACGCTCGGGGAGAG |
| | | GGTGTCAGACCTGCGATAGCGGGCAGCCCTCGATGAAGCGAGAATCCAACGGCTTTAGCCGTTG |
| | | GAGTGTCAAGGGCTGTTGTCAGACCCATTTTATTTTTTGTTTTAGAGCTAGAAATAGTAAGTTAAA |
| | | ATAAGGCTAGTCCGTT |
| seq. | vector_ | TTTAGCGCGTGCGCCAATTCTGCAGACAAATGGCTCTAGAGGTACCGACATTGATTATTGACTAG |
| 54 | ISDge10TnpB- | TTATTAATAGTAATCAATTACGGGGTCATTAGTTCATAGCCCATATATGGAGTTCCGCGTTACATA |
| | VPR-reRNA) | ACTTACGGTAAATGGCCCGCCTGGCTGACCGCCCAACGACCCCCGCCCATTGACGTCAATAATG |
| | (16 nt + WT | ACGTATGTTCCCATAGTAACGCCAATAGGGACTTTCCATTGACGTCAATGGGTGGAGTATTTACG |
| | TnpB) | GTAAACTGCCCACTTGGCAGTACATCAAGTGTATCATATGCCAAGTACGCCCCCTATTGACGTCA |
| | | ATGACGGTAAATGGCCCGCCTGGCATTATGCCCAGTACATGACCTTATGGGACTTTCCTACTTGG |
| | | CAGTACATCTACGTATTAGTCATCGCTATTACCATGGTGATGCGGTTTTGGCAGTACATCAATGGG |
| | | CGTGGATAGCGGTTTGACTCACGGGGATTTCCAAGTCTCCACCCCATTGACGTCAATGGGAGTTT |
| | | GTTTTGGCACCAAAATCAACGGGACTTTCCAAAATGTCGTAACAACTCCGCCCCATTGACGCAAA |
| | | TGGGCGGTAGGCGTGTACGGTGGGAGGTCTATATAAGCAGAGCTGGTTTAGTGAACCGTCAGAT |
| | | CCGCTAGAGATCCGCGGCCGCGCCACCATGGCCCCAAAGAAGAAGCGGAAGGTCGGTATCCAC |
| | | GGAGTCCCAGCAGCCATGACCACCCACCGCAAGGTGTACAGATACCGCATCGAGCCCACCCCC |
| | | GTGCAGGAGAGCAAGCTGTACATGCTGGCCGGCAGCCGGAGATTCGTGTTCAACTGGGCCCTG |
| | | GCCCGGAGAAGAGAGCACTACGCCGAAACCGGCAAGACCCTGGGATACAACGCCCAGGCAGG |
| | | AGAGCTGACCGCCCTGAAAAACCAAGAAGAAACCAGCTGGCTGAAAGAGTCCGACTCACAACT |
| | | GCTGCAGCAGGCACTGAAAGACGTGGAAAGAGCCTTCGTGAACTTCTTCGAAAAACGGGCCAG |
| | | ATTCCCCAGATTCAAAAGCAAGAAAACCGACACCCCCAGATTCAGAATCCCCCAAAGAGTGAGA |
| | | ATAGAGGGCAGCAGAGTGTACGTGCCCAAGGTGGGGTGGGTGAAGCTGAGAAAGAGTCAGGA |
| | | GATAGAAGGAAAAACCAAAAGCGCCACCTTCAAGAGGGAGGCAGATGGCCATTGGTACGTGCT |
| | | GCTGGTGAGTGAGTTCGAGATGCCTGATGTGCCTCTGCCCCCAGTGCCCGAGAGCGAGGTGGTG |
| | | GGAATCGACCTGGGACTGAAGGACTTCTACGTCCTGTCTGACGGGGGCAGGAAGGAGGCCCCC |
| | | AGATTCGCTAGAAAGGGCCAGCGAAAACTGAGAAGAGCTGCAAGAAGACATAGCAAGTGCACC |
| | | CGGGGAAGCAACAGAAAGGCCAAGGCAAAGAGGAAGCTGGCAAGAGTGCACAGACAGATCGC |
| | | AAACCAGAGAAAAGACTTCGTGCACAAGGCCACCAGCGGCCTCGTGCAGCAGTACCAAGGCTT |
| | | CTGCATCGAAAACCTGAGCATCAAGGGCATGGCCAAAACCAAACTGTCAAAAAGCGTGCTGGA |
| | | CGCAGCCCTGGGGGAATTTAGAAGACAGCTGGCCTATAAAGCCCAGTGGCATAGAAAGTGGCT |
| | | GGCAGTGATAGACAGATGGTTCCCCAGCAGCAAGCTGTGCGGAGAGTGCGGCAGCATTAACGC |
| | | CGATCTGACCCTGAGCGACAGAGAATGGACCTGCGAGTGTGGCGCAGTGCACGACAGAGATCT |
| | | GAACGCCGCTCGCAACATCAAGAGAGAGGGCCTGAGTCAGATTGTGGTGGCCGGACATGCCGA |
| | | AACCCTGAACGCCAGAGGGGAGGGCGTGAGACCCGCCATTGCCGGAAGCCCTAGAAGCAGGG |
| | | CTGACCCCAAGAAGAAGAGGAAGGTGTCGCCAGGGATCCGTCGACTTGACGCGTTGATATCAAC |
| | | AAGTTTGTACAAAAAAGCAGGCTACAAAGAGGCCAGCGGTTCCGGACGGGCTGACGCATTGGA |
| | | CGATTTTGATCTGGATATGCTGGGAAGTGACGCCCTCGATGATTTTGACCTTGACATGCTTGGTTC |
| | | GGATGCCCTTGATGACTTTGACCTCGACATGCTCGGCAGTGACGCCCTTGATGATTTCGACCTGG |
| | | ACATGCTGATTAACTCTAGAAGTTCCGGATCTCCGAAAAAGAAACGCAAAGTTGGTAGCCAGTA |
| | | CCTGCCCGACACCGACGACCGGCACCGGATCGAGGAAAAGCGGAAGCGGACCTACGAGACATT |
| | | CAAGAGCATCATGAAGAAGTCCCCCTTCAGCGGCCCCACCGACCCTAGACCTCCACCTAGAAGA |
| | | ATCGCCGTGCCCAGCAGATCCAGCGCCAGCGTGCCAAAACCTGCCCCCCAGCCTTACCCCTTCA |
| | | CCAGCAGCCTGAGCACCATCAACTACGACGAGTTCCCTACCATGGTGTTCCCCAGCGGCCAGAT |
| | | CTCTCAGGCCTCTGCTCTGGCTCCAGCCCCTCCTCAGGTGCTGCCTCAGGCTCCTGCTCCTGCAC |
| | | CAGCTCCAGCCATGGTGTCTGCACTGGCTCAGGCACCAGCACCCGTGCCTGTGCTGGCTCCTGG |
| | | ACCTCCACAGGCTGTGGCTCCACCAGCCCCTAAACCTACACAGGCCGGCGAGGGCACACTGTCT |
| | | GAAGCTCTGCTGCAGCTGCAGTTCGACGACGAGGATCTGGGAGCCCTGCTGGGAAACAGCACC |
| | | GATCCTGCCGTGTTCACCGACCTGGCCAGCGTGGACAACAGCGAGTTCCAGCAGCTGCTGAACC |
| | | AGGGCATCCCTGTGGCCCCTCACACCACCGAGCCCATGCTGATGGAATACCCCGAGGCCATCAC |
| | | CCGGCTCGTGACAGGCGCTCAGAGGCCTCCTGATCCAGCTCCTGCCCCTCTGGGAGCACCAGGC |
| | | CTGCCTAATGGACTGCTGTCTGGCGACGAGGACTTCAGCTCTATCGCCGATATGGATTTCTCAGC |
| | | CTTGCTGGGCTCTGGCAGCGGCAGCCGGGATTCCAGGGAAGGGATGTTTTTGCCGAAGCCTGAG |
| | | GCCGGCTCCGCTATTAGTGACGTGTTTGAGGGCCGCGAGGTGTGCCAGCCAAAACGAATCCGGC |
| | | CATTTCATCCTCCAGGAAGTCCATGGGCCAACCGCCCACTCCCCGCCAGCCTCGCACCAACACC |
| | | AACCGGTCCAGTACATGAGCCAGTCGGGTCACTGACCCCGGCACCAGTCCCTCAGCCACTGGAT |
| | | CCAGCGCCCGCAGTGACTCCCGAGGCCAGTCACCTGTTGGAGGATCCCGATGAAGAGACGAGC |
| | | CAGGCTGTCAAAGCCCTTCGGGAGATGGCCGATACTGTGATTCCCCAGAAGGAAGAGGCTGCAA |
| | | TCTGTGGCCAAATGGACCTTTCCCATCCGCCCCCAAGGGGCCATCTGGATGAGCTGACAACCAC |
| | | ACTTGAGTCCATGACCGAGGATCTGAACCTGGACTCACCCCTGACCCCGGAATTGAACGAGATT |
| | | CTGGATACCTTCCTGAACGACGAGTGCCTCTTGCATGCCATGCATATCAGCACAGGACTGTCCAT |
| | | CTTCGACACATCTCTGTTTAAAAGGCCGGCGGCCACGAAAAAGGCCGGCCAGGCAAAAAAGAA |
| | | AAAGATGGCCTCAAACGATTATACCCAACAAGCAACCCAAAGCTATGGGGCCTACCCCACCCAG |
| | | CCCGGGCAGGGCTATTCCCAGCAGAGCAGTCAGCCCTACGGACAGCAGAGTTACAGTGGTTATA |
| | | GCCAGTCCACGGACACTTCAGGCTATGGCCAGAGCAGCTATTCTTCTTATGGCCAGAGCCAGAA |
| | | CACAGGCTATGGAACTCAGTCAACTCCCCAGGGATATGGCTCGACTGGCGGCTATGGCAGTAGC |
| | | CAGAGCTCCCAATCGTCTTACGGGCAGCAGTCCTCCTACCCTGGCTATGGCCAGCAGCCAGCTC |
| | | CCAGCAGCACCTCGGGAAGTTACGGTAGCAGTTCTCAGAGCAGCAGCTATGGGCAGCCCCAGA |
| | | GTGGGAGCTACAGCCAGCAGCCTAGCTATGGTGGACAGCAGCAAAGCTATGGACAGCAGCAAA |
| | | GCTATAATCCCCCTCAGGGCTATGGACAGCAGAACCAGTACAACAGCAGCAGTGGTGGTGGAG |
| | | GTGGAGGTGGAGGTGGAGGTAACTATGGCCAAGATCAATCCTCCATGAGTAGTGGTGGTGGCA |
| | | GTGGTGGCGGTTATGGCAATCAAGACCAGAGTGGTGGAGGTGGCAGCGGTGGCTATGGACAGC |
| | | AGGACCGTGGAGAATTCGGCAGTGGAGAGGGCAGAGGAAGTCTGCTAACATGCGGTGACGTCG |
| | | AGGAGAATCCTGGCCCAGTGAGCAAGGGCGAGGAGCTGTTCACCGGGGTGGTGCCCATCCTGG |
| | | TCGAGCTGGACGGCGACGTAAACGGCCACAAGTTCAGCGTGTCCGGCGAGGGCGAGGGCGATG |
| | | CCACCTACGGCAAGCTGACCCTGAAGTTCATCTGCACCACCGGCAAGCTGCCCGTGCCCTGGCC |
| | | CACCCTCGTGACCACCCTGACCTACGGCGTGCAGTGCTTCAGCCGCTACCCCGACCACATGAAG |
| | | CAGCACGACTTCTTCAAGTCCGCCATGCCCGAAGGCTACGTCCAGGAGCGCACCATCTTCTTCAA |
| | | GGACGACGGCAACTACAAGACCCGCGCCGAGGTGAAGTTCGAGGGCGACACCCTGGTGAACCG |
| | | CATCGAGCTGAAGGGCATCGACTTCAAGGAGGACGGCAACATCCTGGGGCACAAGCTGGAGTA |
| | | CAACTACAACAGCCACAACGTCTATATCATGGCCGACAAGCAGAAGAACGGCATCAAGGTGAA |
| | | CTTCAAGATCCGCCACAACATCGAGGACGGCAGCGTGCAGCTCGCCGACCACTACCAGCAGAA |
| | | CACCCCCATCGGCGACGGCCCCGTGCTGCTGCCCGACAACCACTACCTGAGCACCCAGTCCGCC |
| | | CTGAGCAAAGACCCCAACGAGAAGCGCGATCACATGGTCCTGCTGGAGTTCGTGACCGCCGCC |
| | | GGGATCACTCTCGGCATGGACGAGCTGTACAAGGAATTCTAACTAGAGCTCGCTGATCAGCCTC |
| | | GACTGTGCCTTCTAGTTGCCAGCCATCTGTTGTTTGCCCCTCCCCCGTGCCTTCCTTGACCCTGGA |
| | | AGGTGCCACTCCCACTGTCCTTTCCTAATAAAATGAGGAAATTGCATCGCATTGTCTGAGTAGGT |
| | | GTCATTCTATTCTGGGGGGTGGGGTGGGGCAGGACAGCAAGGGGGAGGATTGGGAAGAGAATA |
| | | GCAGGCATGCTGGGGATGCGGTGGGCTCTATGGCTTCTGAGGCGGAAAGAACCAGCTGGGGCT |
| | | CGATACCGTCGACCTCTAGCTAGAGCTTGGCGTAATCATGGTCATAGCTGTTTCCTGTGTGAAATT |
| | | GTTATCCGCTCACAATTCCACACAACATACGAGCCGGAAGCATAAAGTGTAAAGCCTAGGGTGC |
| | | CTAATGAGTGAGCTAACTCACATTAATTGCGTTGCGCTCACTGCCCGCTTTCGAGTCGGGAAACC |
| | | TGTCGTGCCAGCTGCATTAATGAATCGGCCAACGCGCGGGGAGAGGCGGTTTGCGTATTGGGCG |
| | | CTCTTCCGCTTCCTCGCTCACTGACTCGCTGCGCTCGGTCGTTCGGCTGCGGCGAGCGGTATCAG |
| | | CTCACTCAAAGGCGGTAATACGGTTATCCACAGAATCAGGGGATAACGCAGGAAAGAACATGTG |
| | | AGCAAAAGGCCAGCAAAAGGCCAGGAACCGTAAAAAGGCCGCGTTGCTGGCGTTTTTCCATAG |
| | | GCTCCGCCCCCCTGACGAGCATCACAAAAATCGACGCTCAAGTCAGAGGTGGCGAAACCCGAC |
| | | AGGACTATAAAGATACCAGGCGTTTCCCCCTGGAAGCTCCCTCGTGCGCTCTCCTGTTCCGACCC |
| | | TGCCGCTTACCGGATACCTGTCCGCCTTTCTCCCTTCGGGAAGCGTGGCGCTTTCTCATAGCTCAC |
| | | GCTGTAGGTATCTCAGTTCGGTGTAGGTCGTTCGCTCCAAGCTGGGCTGTGTGCACGAACCCCCC |
| | | GTTCAGCCCGACCGCTGCGCCTTATCCGGTAACTATCGTCTTGAGTCCAACCCGGTAAGACACGA |
| | | CTTATCGCCACTGGCAGCAGCCACTGGTAACAGGATTAGCAGAGCGAGGTATGTAGGCGGTGCT |
| | | ACAGAGTTCTTGAAGTGGTGGCCTAACTACGGCTACACTAGAAGAACAGTATTTGGTATCTGCGG |
| | | TCTGCTGAAGCCAGTTACCTTCGGAAAAAGAGTTGGTAGCTCTTGATCCGGCAAACAAACCACC |
| | | GCTGGTAGCGGTGGTTTTTTTGTTTGCAAGCAGCAGATTACGCGCAGAAAAAAAGGATCTCAAGA |
| | | AGATCCTTTGATCTTTTCTACGGGGTCTGACGCTCAGTGGAACGAAAACTCACGTTAAGGGATTTT |
| | | GGTCATGAGATTATCAAAAAGGATCTTCACCTAGATCCTTTTAAATTAAAAATGAAGTTTTAAATC |
| | | AATCTAAAGTATATATGAGTAAACTTGGTCTGACAGTTACCAATGCTTAATCAGTGAGGCACCTAT |
| | | CTCAGCGATCTGTCTATTTCGTTCATCCATAGTTGCCTGACTCCCCGTCGTGTAGATAACTACGAT |
| | | ACGGGAGGGCTTACCATCTGGCCCCAGTGCTGCAATGATACCGCGAGACCCACGCTCACCGGCT |
| | | CCAGATTTATCAGCAATAAACCAGCCAGCCGGAAGGGCCGAGCGCAGAAGTGGTCCTGCAACTT |
| | | TATCCGCCTCCATCCAGTCTATTAATTGTTGCCGGGAAGCTAGAGTAAGTAGTTCGCCAGTTAATA |
| | | GTTTGCGCAACGTTGTTGCCATTGCTACAGGCATCGTGGTGTCACGCTCGTCGTTTGGTATGGCTT |
| | | CATTCAGCTCCGGTTCCCAACGATCAAGGCGAGTTACATGATCCCCCATGTTGTGCAAAAAAGCG |
| | | GTTAGCTCCTTCGGTCCTCCGATCGTTGTCAGAAGTAAGTTGGCCGCAGTGTTATCACTCATGGTT |
| | | ATGGCAGCACTGCATAATTCTCTTACTGTCATGCCATCCGTAAGATGCTTTTCTGTGACTGGTGAG |
| | | TACTCAACCAAGTCATTCTGAGAATAGTGTATGCGGCGACCGAGTTGCTCTTGCCCGGCGTCAAT |
| | | ACGGGATAATACCGCGCCACATAGCAGAACTTTAAAAGTGCTCATCATTGGAAAACGTTCTTCGG |
| | | GGCGAAAACTCTCAAGGATCTTACCGCTGTTGAGATCCAGTTCGATGTAACCCACTCGTGCACCC |
| | | AACTGATCTTCAGCATCTTTTACTTTCACCAGCGTTTCTGGGTGAGCAAAAACAGGAAGGCAAAA |
| | | TGCCGCAAAAAAGGGAATAAGGGCGACACGGAAATGTTGAATACTCATACTCTTCCTTTTTCAAT |
| | | ATTATTGAAGCATTTATCAGGGTTATTGTCTCATGAGCGGATACATATTTGAATGTATTTAGAAAA |
| | | ATAAACAAATAGGGGTTCCGCGCACATTTCCCCGAAAAGTGCCACCTGACGTCGACGGATCGGG |
| | | AGATCGATCTCCCGATCCCCTAGGGTCGACTCTCAGTACAATCTGCTCTGATGCCGCATAGTTAA |
| | | GCCAGTATCTGCTCCCTGCTTGTGTGTTGGAGGTCGCTGAGTAGTGCGCGAGCAAAATTTAAGCT |
| | | ACAACAAGGCAAGGCTTGACCGACAATTGCATGAAGAATCTGCTTAGGGTTAGGCGTTTTGCGCT |
| | | GCTTCGCGATGTACGGGCCAGATATACGCGTGAGGGCCTATTTCCCATGATTCCTTCATATTTGCA |
| | | TATACGATACAAGGCTGTTAGAGAGATAATTGGAATTAATTTGACTGTAAACACAAAGATATTAG |
| | | TACAAAATACGTGACGTAGAAAGTAATAATTTCTTGGGTAGTTTGCAGTTTTAAAATTATGTTTTAA |
| | | AATGGACTATCATATGCTTACCGTAACTTGAAAGTATTTCGATTTCTTGGCTTTATATATCTTGTGG |
| | | AAAGGACGAAACACCGGTGGAGCGGTTCACGACCGCGACCTCAACGCCGCCCGGAACATCAAG |
| | | CGGGAAGGGCTTTCGCAAATCGTCGTCGCGGGGCACGCGGAGACGTTAAACGCTCGGGGAGAG |
| | | GGTGTCAGACCTGCGATAGCGGGCAGCCCTCGATGAAGCGAGAATCCAACGGCTTTAGCCGTTG |
| | | GAGTGTCAAGGGCTGTTGTCAGACCTTTTATTTTTTGTTTTAGAGCTAGAAATAGTAAGTTAAAAT |
| | | AAGGCTAGTCCGTT |
| seq. | vector_ | TTTAGCGCGTGCGCCAATTCTGCAGACAAATGGCTCTAGAGGTACCGACATTGATTATTGACTAG |
| 55 | ISDge10TnpB- | TTATTAATAGTAATCAATTACGGGGTCATTAGTTCATAGCCCATATATGGAGTTCCGCGTTACATA |
| | VPR-reRNA) | ACTTACGGTAAATGGCCCGCCTGGCTGACCGCCCAACGACCCCCGCCCATTGACGTCAATAATG |
| | (14 nt + WT | ACGTATGTTCCCATAGTAACGCCAATAGGGACTTTCCATTGACGTCAATGGGTGGAGTATTTACG |
| | TnpB) | GTAAACTGCCCACTTGGCAGTACATCAAGTGTATCATATGCCAAGTACGCCCCCTATTGACGTCA |
| | | ATGACGGTAAATGGCCCGCCTGGCATTATGCCCAGTACATGACCTTATGGGACTTTCCTACTTGG |
| | | CAGTACATCTACGTATTAGTCATCGCTATTACCATGGTGATGCGGTTTTGGCAGTACATCAATGGG |
| | | CGTGGATAGCGGTTTGACTCACGGGGATTTCCAAGTCTCCACCCCATTGACGTCAATGGGAGTTT |
| | | GTTTTGGCACCAAAATCAACGGGACTTTCCAAAATGTCGTAACAACTCCGCCCCATTGACGCAAA |
| | | TGGGCGGTAGGCGTGTACGGTGGGAGGTCTATATAAGCAGAGCTGGTTTAGTGAACCGTCAGAT |
| | | CCGCTAGAGATCCGCGGCCGCGCCACCATGGCCCCAAAGAAGAAGCGGAAGGTCGGTATCCAC |
| | | GGAGTCCCAGCAGCCATGACCACCCACCGCAAGGTGTACAGATACCGCATCGAGCCCACCCCC |
| | | GTGCAGGAGAGCAAGCTGTACATGCTGGCCGGCAGCCGGAGATTCGTGTTCAACTGGGCCCTG |
| | | GCCCGGAGAAGAGAGCACTACGCCGAAACCGGCAAGACCCTGGGATACAACGCCCAGGCAGG |
| | | AGAGCTGACCGCCCTGAAAAACCAAGAAGAAACCAGCTGGCTGAAAGAGTCCGACTCACAACT |
| | | GCTGCAGCAGGCACTGAAAGACGTGGAAAGAGCCTTCGTGAACTTCTTCGAAAAACGGGCCAG |
| | | ATTCCCCAGATTCAAAAGCAAGAAAACCGACACCCCCAGATTCAGAATCCCCCAAAGAGTGAGA |
| | | ATAGAGGGCAGCAGAGTGTACGTGCCCAAGGTGGGGTGGGTGAAGCTGAGAAAGAGTCAGGA |
| | | GATAGAAGGAAAAACCAAAAGCGCCACCTTCAAGAGGGAGGCAGATGGCCATTGGTACGTGCT |
| | | GCTGGTGAGTGAGTTCGAGATGCCTGATGTGCCTCTGCCCCCAGTGCCCGAGAGCGAGGTGGTG |
| | | GGAATCGACCTGGGACTGAAGGACTTCTACGTCCTGTCTGACGGGGGCAGGAAGGAGGCCCCC |
| | | AGATTCGCTAGAAAGGGCCAGCGAAAACTGAGAAGAGCTGCAAGAAGACATAGCAAGTGCACC |
| | | CGGGGAAGCAACAGAAAGGCCAAGGCAAAGAGGAAGCTGGCAAGAGTGCACAGACAGATCGC |
| | | AAACCAGAGAAAAGACTTCGTGCACAAGGCCACCAGCGGCCTCGTGCAGCAGTACCAAGGCTT |
| | | CTGCATCGAAAACCTGAGCATCAAGGGCATGGCCAAAACCAAACTGTCAAAAAGCGTGCTGGA |
| | | CGCAGCCCTGGGGGAATTTAGAAGACAGCTGGCCTATAAAGCCCAGTGGCATAGAAAGTGGCT |
| | | GGCAGTGATAGACAGATGGTTCCCCAGCAGCAAGCTGTGCGGAGAGTGCGGCAGCATTAACGC |
| | | CGATCTGACCCTGAGCGACAGAGAATGGACCTGCGAGTGTGGCGCAGTGCACGACAGAGATCT |
| | | GAACGCCGCTCGCAACATCAAGAGAGAGGGCCTGAGTCAGATTGTGGTGGCCGGACATGCCGA |
| | | AACCCTGAACGCCAGAGGGGAGGGCGTGAGACCCGCCATTGCCGGAAGCCCTAGAAGCAGGG |
| | | CTGACCCCAAGAAGAAGAGGAAGGTGTCGCCAGGGATCCGTCGACTTGACGCGTTGATATCAAC |
| | | AAGTTTGTACAAAAAAGCAGGCTACAAAGAGGCCAGCGGTTCCGGACGGGCTGACGCATTGGA |
| | | CGATTTTGATCTGGATATGCTGGGAAGTGACGCCCTCGATGATTTTGACCTTGACATGCTTGGTTC |
| | | GGATGCCCTTGATGACTTTGACCTCGACATGCTCGGCAGTGACGCCCTTGATGATTTCGACCTGG |
| | | ACATGCTGATTAACTCTAGAAGTTCCGGATCTCCGAAAAAGAAACGCAAAGTTGGTAGCCAGTA |
| | | CCTGCCCGACACCGACGACCGGCACCGGATCGAGGAAAAGCGGAAGCGGACCTACGAGACATT |
| | | CAAGAGCATCATGAAGAAGTCCCCCTTCAGCGGCCCCACCGACCCTAGACCTCCACCTAGAAGA |
| | | ATCGCCGTGCCCAGCAGATCCAGCGCCAGCGTGCCAAAACCTGCCCCCCAGCCTTACCCCTTCA |
| | | CCAGCAGCCTGAGCACCATCAACTACGACGAGTTCCCTACCATGGTGTTCCCCAGCGGCCAGAT |
| | | CTCTCAGGCCTCTGCTCTGGCTCCAGCCCCTCCTCAGGTGCTGCCTCAGGCTCCTGCTCCTGCAC |
| | | CAGCTCCAGCCATGGTGTCTGCACTGGCTCAGGCACCAGCACCCGTGCCTGTGCTGGCTCCTGG |
| | | ACCTCCACAGGCTGTGGCTCCACCAGCCCCTAAACCTACACAGGCCGGCGAGGGCACACTGTCT |
| | | GAAGCTCTGCTGCAGCTGCAGTTCGACGACGAGGATCTGGGAGCCCTGCTGGGAAACAGCACC |
| | | GATCCTGCCGTGTTCACCGACCTGGCCAGCGTGGACAACAGCGAGTTCCAGCAGCTGCTGAACC |
| | | AGGGCATCCCTGTGGCCCCTCACACCACCGAGCCCATGCTGATGGAATACCCCGAGGCCATCAC |
| | | CCGGCTCGTGACAGGCGCTCAGAGGCCTCCTGATCCAGCTCCTGCCCCTCTGGGAGCACCAGGC |
| | | CTGCCTAATGGACTGCTGTCTGGCGACGAGGACTTCAGCTCTATCGCCGATATGGATTTCTCAGC |
| | | CTTGCTGGGCTCTGGCAGCGGCAGCCGGGATTCCAGGGAAGGGATGTTTTTGCCGAAGCCTGAG |
| | | GCCGGCTCCGCTATTAGTGACGTGTTTGAGGGCCGCGAGGTGTGCCAGCCAAAACGAATCCGGC |
| | | CATTTCATCCTCCAGGAAGTCCATGGGCCAACCGCCCACTCCCCGCCAGCCTCGCACCAACACC |
| | | AACCGGTCCAGTACATGAGCCAGTCGGGTCACTGACCCCGGCACCAGTCCCTCAGCCACTGGAT |
| | | CCAGCGCCCGCAGTGACTCCCGAGGCCAGTCACCTGTTGGAGGATCCCGATGAAGAGACGAGC |
| | | CAGGCTGTCAAAGCCCTTCGGGAGATGGCCGATACTGTGATTCCCCAGAAGGAAGAGGCTGCAA |
| | | TCTGTGGCCAAATGGACCTTTCCCATCCGCCCCCAAGGGGCCATCTGGATGAGCTGACAACCAC |
| | | ACTTGAGTCCATGACCGAGGATCTGAACCTGGACTCACCCCTGACCCCGGAATTGAACGAGATT |
| | | CTGGATACCTTCCTGAACGACGAGTGCCTCTTGCATGCCATGCATATCAGCACAGGACTGTCCAT |
| | | CTTCGACACATCTCTGTTTAAAAGGCCGGCGGCCACGAAAAAGGCCGGCCAGGCAAAAAAGAA |
| | | AAAGATGGCCTCAAACGATTATACCCAACAAGCAACCCAAAGCTATGGGGCCTACCCCACCCAG |
| | | CCCGGGCAGGGCTATTCCCAGCAGAGCAGTCAGCCCTACGGACAGCAGAGTTACAGTGGTTATA |
| | | GCCAGTCCACGGACACTTCAGGCTATGGCCAGAGCAGCTATTCTTCTTATGGCCAGAGCCAGAA |
| | | CACAGGCTATGGAACTCAGTCAACTCCCCAGGGATATGGCTCGACTGGCGGCTATGGCAGTAGC |
| | | CAGAGCTCCCAATCGTCTTACGGGCAGCAGTCCTCCTACCCTGGCTATGGCCAGCAGCCAGCTC |
| | | CCAGCAGCACCTCGGGAAGTTACGGTAGCAGTTCTCAGAGCAGCAGCTATGGGCAGCCCCAGA |
| | | GTGGGAGCTACAGCCAGCAGCCTAGCTATGGTGGACAGCAGCAAAGCTATGGACAGCAGCAAA |
| | | GCTATAATCCCCCTCAGGGCTATGGACAGCAGAACCAGTACAACAGCAGCAGTGGTGGTGGAG |
| | | GTGGAGGTGGAGGTGGAGGTAACTATGGCCAAGATCAATCCTCCATGAGTAGTGGTGGTGGCA |
| | | GTGGTGGCGGTTATGGCAATCAAGACCAGAGTGGTGGAGGTGGCAGCGGTGGCTATGGACAGC |
| | | AGGACCGTGGAGAATTCGGCAGTGGAGAGGGCAGAGGAAGTCTGCTAACATGCGGTGACGTCG |
| | | AGGAGAATCCTGGCCCAGTGAGCAAGGGCGAGGAGCTGTTCACCGGGGTGGTGCCCATCCTGG |
| | | TCGAGCTGGACGGCGACGTAAACGGCCACAAGTTCAGCGTGTCCGGCGAGGGCGAGGGCGATG |
| | | CCACCTACGGCAAGCTGACCCTGAAGTTCATCTGCACCACCGGCAAGCTGCCCGTGCCCTGGCC |
| | | CACCCTCGTGACCACCCTGACCTACGGCGTGCAGTGCTTCAGCCGCTACCCCGACCACATGAAG |
| | | CAGCACGACTTCTTCAAGTCCGCCATGCCCGAAGGCTACGTCCAGGAGCGCACCATCTTCTTCAA |
| | | GGACGACGGCAACTACAAGACCCGCGCCGAGGTGAAGTTCGAGGGCGACACCCTGGTGAACCG |
| | | CATCGAGCTGAAGGGCATCGACTTCAAGGAGGACGGCAACATCCTGGGGCACAAGCTGGAGTA |
| | | CAACTACAACAGCCACAACGTCTATATCATGGCCGACAAGCAGAAGAACGGCATCAAGGTGAA |
| | | CTTCAAGATCCGCCACAACATCGAGGACGGCAGCGTGCAGCTCGCCGACCACTACCAGCAGAA |
| | | CACCCCCATCGGCGACGGCCCCGTGCTGCTGCCCGACAACCACTACCTGAGCACCCAGTCCGCC |
| | | CTGAGCAAAGACCCCAACGAGAAGCGCGATCACATGGTCCTGCTGGAGTTCGTGACCGCCGCC |
| | | GGGATCACTCTCGGCATGGACGAGCTGTACAAGGAATTCTAACTAGAGCTCGCTGATCAGCCTC |
| | | GACTGTGCCTTCTAGTTGCCAGCCATCTGTTGTTTGCCCCTCCCCCGTGCCTTCCTTGACCCTGGA |
| | | AGGTGCCACTCCCACTGTCCTTTCCTAATAAAATGAGGAAATTGCATCGCATTGTCTGAGTAGGT |
| | | GTCATTCTATTCTGGGGGGTGGGGTGGGGCAGGACAGCAAGGGGGAGGATTGGGAAGAGAATA |
| | | GCAGGCATGCTGGGGATGCGGTGGGCTCTATGGCTTCTGAGGCGGAAAGAACCAGCTGGGGCT |
| | | CGATACCGTCGACCTCTAGCTAGAGCTTGGCGTAATCATGGTCATAGCTGTTTCCTGTGTGAAATT |
| | | GTTATCCGCTCACAATTCCACACAACATACGAGCCGGAAGCATAAAGTGTAAAGCCTAGGGTGC |
| | | CTAATGAGTGAGCTAACTCACATTAATTGCGTTGCGCTCACTGCCCGCTTTCCAGTCGGGAAACC |
| | | TGTCGTGCCAGCTGCATTAATGAATCGGCCAACGCGCGGGGAGAGGCGGTTTGCGTATTGGGCG |
| | | CTCTTCCGCTTCCTCGCTCACTGACTCGCTGCGCTCGGTCGTTCGGCTGCGGCGAGCGGTATCAG |
| | | CTCACTCAAAGGCGGTAATACGGTTATCCACAGAATCAGGGGATAACGCAGGAAAGAACATGTG |
| | | ACCAAAAGGCCAGCAAAAGGCCAGGAACCGTAAAAAGGCCGCGTTGCTGGCGTTTTTCCATAG |
| | | GCTCCGCCCCCCTGACGAGCATCACAAAAATCGACGCTCAAGTCAGAGGTGGCGAAACCCGAC |
| | | AGGACTATAAAGATACCAGGCGTTTCCCCCTGGAAGCTCCCTCGTGCGCTCTCCTGTTCCGACCC |
| | | TGCCGCTTACCGGATACCTGTCCGCCTTTCTCCCTTCGGGAAGCGTGGCGCTTTCTCATAGCTCAC |
| | | GCTGTAGGTATCTCAGTTCGGTGTAGGTCGTTCGCTCCAAGCTGGGCTGTGTGCACGAACCCCCC |
| | | GTTCAGCCCGACCGCTGCGCCTTATCCGGTAACTATCGTCTTGAGTCCAACCCGGTAAGACACGA |
| | | CTTATCGCCACTGGCAGCAGCCACTGGTAACAGGATTAGCAGAGCGAGGTATGTAGGCGGTGCT |
| | | ACAGAGTTCTTGAAGTGGTGGCCTAACTACGGCTACACTAGAAGAACAGTATTTGGTATCTGCGC |
| | | TCTGCTGAAGCCAGTTACCTTCGGAAAAAGAGTTGGTAGCTCTTGATCCGGCAAACAAACCACC |
| | | GCTGGTAGCGGTGGTTTTTTTGTTTGCAAGCAGCAGATTACGCGCAGAAAAAAAGGATCTCAAGA |
| | | AGATCCTTTGATCTTTTCTACGGGGTCTGACGCTCAGTGGAACGAAAACTCACGTTAAGGGATTTT |
| | | GGTCATGAGATTATCAAAAAGGATCTTCACCTAGATCCTTTTAAATTAAAAATGAAGTTTTAAATC |
| | | AATCTAAAGTATATATGAGTAAACTTGGTCTGACAGTTACCAATGCTTAATCAGTGAGGCACCTAT |
| | | CTCAGCGATCTGTCTATTTCGTTCATCCATAGTTGCCTGACTCCCCGTCGTGTAGATAACTACGAT |
| | | ACGGGAGGGCTTACCATCTGGCCCCAGTGCTGCAATGATACCGCGAGACCCACGCTCACCGGCT |
| | | CCAGATTTATCAGCAATAAACCAGCCAGCCGGAAGGGCCGAGCGCAGAAGTGGTCCTGCAACTT |
| | | TATCCGCCTCCATGCAGTCTATTAATTGTTGCCGGGAAGCTAGAGTAAGTAGTTCGCCAGTTAATA |
| | | GTTTGCGCAACGTTGTTGCCATTGCTACAGGCATCGTGGTGTCACGCTCGTCGTTTGGTATGGCTT |
| | | CATTCAGCTCCGGTTCCCAACGATCAAGGCGAGTTACATGATCCCCCATGTTGTGCAAAAAAGCG |
| | | GTTAGCTCCTTCGGTCCTCCGATCGTTGTCAGAAGTAAGTTGGCCGCAGTGTTATCACTCATGGTT |
| | | ATGGCAGCACTGCATAATTCTCTTACTGTCATGCCATCCGTAAGATGCTTTTCTGTGACTGGTGAG |
| | | TACTCAACCAAGTCATTCTGAGAATAGTGTATGCGGCGACCGAGTTGCTCTTGCCCGGCGTCAAT |
| | | ACGGGATAATACCGCGCCACATAGCAGAACTTTAAAAGTGCTCATCATTGGAAAACGTTCTTCGG |
| | | GGCGAAAACTCTCAAGGATCTTACCGCTGTTGAGATCCAGTTCGATGTAACCCACTCGTGCACCC |
| | | AACTGATCTTCAGCATCTTTTACTTTCACCAGCGTTTCTGGGTGAGCAAAAACAGGAAGGCAAAA |
| | | TGCCGCAAAAAAGGGAATAAGGGCGACACGGAAATGTTGAATACTCATACTCTTCCTTTTTCAAT |
| | | ATTATTGAAGCATTTATCAGGGTTATTGTCTCATGAGCGGATACATATTTGAATGTATTTAGAAAA |
| | | ATAAACAAATAGGGGTTCCGCGCACATTTCCCCGAAAAGTGCCACCTGACGTCGACGGATCGGG |
| | | AGATCGATCTCCCGATCCCCTAGGGTCGACTCTCAGTACAATCTGCTCTGATGCCGCATAGTTAA |
| | | GCCAGTATCTGCTCCCTGCTTGTGTGTTGGAGGTCGCTGAGTAGTGCGCGAGCAAAATTTAAGCT |
| | | ACAACAAGGCAAGGCTTGACCGACAATTGCATGAAGAATCTGCTTAGGGTTAGGCGTTTTGCGCT |
| | | GCTTCGCGATGTACGGGCCAGATATACGCGTGAGGGCCTATTTCCCATGATTCCTTCATATTTGCA |
| | | TATACGATACAAGGCTGTTAGAGAGATAATTGGAATTAATTTGACTGTAAACACAAAGATATTAG |
| | | TACAAAATACGTGACGTAGAAAGTAATAATTTCTTGGGTAGTTTGCAGTTTTAAAATTATGTTTTAA |
| | | AATGGACTATCATATGCTTACCGTAACTTGAAAGTATTTCGATTTCTTGGCTTTATATATCTTGTGG |
| | | AAAGGACGAAACACCGGTGGAGCGGTTCACGACCGCGACCTCAACGCCGCCCGGAACATCAAG |
| | | CGGGAAGGGCTTTCGCAAATCGTCGTCGCGGGGCACGCGGAGACGTTAAACGCTCGGGGAGAG |
| | | GGTGTCAGACCTGCGATAGCGGGCAGCCCTCGATGAAGCGAGAATCCAACGGCTTTAGCCGTTG |
| | | GAGTGTCAAGGGCTGTTGTCAGATTTTATTTTTTGTTTTAGAGCTAGAAATAGTAAGTTAAAATAA |
| | | GGCTAGTCCGTT |
| seq. | vector_ | TTTAGCGCGTGCGCCAATTCTGCAGACAAATGGCTCTAGAGGTACCGACATTGATTATTGACTAG |
| 56 | ISDge10TnpB- | TTATTAATAGTAATCAATTACGGGGTCATTAGTTCATAGCCCATATATGGAGTTCCGCGTTACATA |
| | VPR-reRNA) | ACTTACGGTAAATGGCCCGCCTGGCTGACCGCCCAACGACCCCCGCCCATTGACGTCAATAATG |
| | (12 nt + WT | ACGTATGTTCCCATAGTAACGCCAATAGGGACTTTCCATTGACGTCAATGGGTGGAGTATTTACG |
| | TnpB) | GTAAACTGCCCACTTGGCAGTACATCAAGTGTATCATATGCCAAGTACGCCCCCTATTGACGTCA |
| | | ATGACGGTAAATGGCGCGCCTGGCATTATGCCCAGTACATGACCTTATGGGACTTTCCTACTTGG |
| | | CAGTACATCTACGTATTAGTCATCGCTATTACCATGGTGATGCGGTTTTGGCAGTACATCAATGGG |
| | | CGTGGATAGCGGTTTGACTCACGGGGATTTCCAAGTCTCCACCCCATTGACGTCAATGGGAGTTT |
| | | GTTTTGGCACCAAAATCAACGGGACTTTCCAAAATGTCGTAACAACTCCGCCCCATTGACGCAAA |
| | | TGGGCGGTAGGCGTGTACGGTGGGAGGTCTATATAAGCAGAGCTGGTTTAGTGAACCGTCAGAT |
| | | CCGCTAGAGATCCGCGGCCGCGCCACCATGGCCCCAAAGAAGAAGCGGAAGGTCGGTATCCAC |
| | | GGAGTCCCAGCAGCCATGACCACCCACCGCAAGGTGTACAGATACCGCATCGAGCCCACCCCC |
| | | GTGCAGGAGAGCAAGCTGTACATGCTGGCCGGCAGCCGGAGATTCGTGTTCAACTGGGCCCTG |
| | | GCCCGGAGAAGAGAGCACTACGCCGAAACCGGCAAGACCCTGGGATACAACGCCCAGGCAGG |
| | | AGAGCTGACCGCCCTGAAAAACCAAGAAGAAACCAGCTGGCTGAAAGAGTCCGACTCACAACT |
| | | GCTGCAGCAGGCACTGAAAGACGTGGAAAGAGCCTTCGTGAACTTCTTCGAAAAACGGGCCAG |
| | | ATTCCCCAGATTCAAAAGCAAGAAAACCGACACCCCCAGATTCAGAATCCCCCAAAGAGTGAGA |
| | | ATAGAGGGCAGCAGAGTGTACGTGCCCAAGGTGGGGTGGGTGAAGCTGAGAAAGAGTCAGGA |
| | | GATAGAAGGAAAAACCAAAAGCGCCACCTTCAAGAGGGAGGCAGATGGCCATTGGTACGTGCT |
| | | GCTGGTGAGTGAGTTCGAGATGCCTGATGTGCCTCTGCCCCCAGTGCCCGAGAGCGAGGTGGTG |
| | | GGAATCGACCTGGGACTGAAGGACTTCTACGTCCTGTCTGACGGGGGCAGGAAGGAGGCCCCC |
| | | AGATTCGCTAGAAAGGGCCAGCGAAAACTGAGAAGAGCTGCAAGAAGACATAGCAAGTGCACC |
| | | CGGGGAAGCAACAGAAACCCCAAGGCAAAGAGGAAGCTGGCAAGAGTGCACAGACAGATCGC |
| | | AAACCAGAGAAAAGACTTCGTGCACAAGGCCACCAGCGGCCTCGTGCAGCAGTACCAAGGCTT |
| | | CTGCATCGAAAACCTGAGCATCAAGGGCATGGCCAAAACCAAACTGTCAAAAAGCGTGCTGGA |
| | | CGCAGCCCTGGGGGAATTTAGAAGACAGCTGGCCTATAAAGCCCAGTGGCATAGAAAGTGGCT |
| | | GGCAGTGATAGACAGATGGTTCCCCAGCAGCAAGCTGTGCGGAGAGTGCGGCAGCATTAACGC |
| | | CGATCTGACCCTGAGCGACAGAGAATGGACCTGCGAGTGTGGCGCAGTGCACGACAGAGATCT |
| | | GAACGCCGCTCGCAACATCAAGAGAGAGGGCCTGAGTCAGATTGTGGTGGCCGGACATGCCGA |
| | | AACCCTGAACGCCAGAGGGGAGGGCGTGAGACCCGCCATTGCCGGAAGCCCTAGAAGCAGGG |
| | | CTGACCCCAAGAAGAAGAGGAAGGTGTCGCCAGGGATCCGTCGACTTGACGCGTTGATATCAAC |
| | | AAGTTTGTACAAAAAAGCAGGCTACAAAGAGGCCAGCGGTTCCGGACGGGCTGACGCATTGGA |
| | | CGATTTTGATCTGGATATGCTGGGAAGTGACGCCCTCGATGATTTTGACCTTGACATGCTTGGTTC |
| | | GGATGCCCTTGATGACTTTGACCTCGACATGCTCGGCAGTGACGCCCTTGATGATTTCGACCTGG |
| | | ACATGCTGATTAACTCTAGAAGTTCCGGATCTCCGAAAAAGAAACGCAAAGTTGGTAGCCAGTA |
| | | CCTGCCCGACACCGACGACCGGCACCGGATCGAGGAAAAGCGGAAGCGGACCTACGAGACATT |
| | | CAAGAGCATCATGAAGAAGTCCCCCTTCAGCGGCCCCACCGACCCTAGACCTCCACCTAGAAGA |
| | | ATCGCCGTGCCCAGCAGATCCAGCGCCAGCGTGCCAAAACCTGCCCCCCAGCCTTACCCCTTCA |
| | | CCAGCAGCCTGAGCACCATCAACTACGACGAGTTCCCTACCATGGTGTTCCCCAGCGGCCAGAT |
| | | CTCTCAGGCCTCTGCTCTGGCTCCAGCCCCTCCTCAGGTGCTGCCTCAGGCTCCTGCTCCTGCAC |
| | | CAGCTCCAGCCATGGTGTCTGCACTGGCTCAGGCACCAGCACCCGTGCCTGTGCTGGCTCCTGG |
| | | ACCTCCACAGGCTGTGGCTCCACCAGCCCCTAAACCTACACAGGCCGGCGAGGGCACACTGTCT |
| | | GAAGCTCTGCTGCAGCTGCAGTTCGACGACGAGGATCTGGGAGCCCTGCTGGGAAACAGCACC |
| | | GATCCTGCCGTGTTCACCGACCTGGCCAGCGTGGACAACAGCGAGTTCCAGCAGCTGCTGAACC |
| | | AGGGCATCCCTGTGGCCCCTCACACCACCGAGCCCATGCTGATGGAATACCCCGAGGCCATCAC |
| | | CCGGCTCGTGACAGGCGCTCAGAGGCCTCCTGATCCAGCTCCTGCCCCTCTGGGAGCACCAGGC |
| | | CTGCCTAATGGACTGCTGTCTGGCGACGAGGACTTCAGCTCTATCGCCGATATGGATTTCTCAGC |
| | | CTTGCTGGGCTCTGGCAGCGGCAGCCGGGATTCCAGGGAAGGGATGTTTTTGCCGAAGCCTGAG |
| | | GCCGGCTCCGCTATTAGTGACGTGTTTGAGGGCCGCGAGGTGTGCCAGCCAAAACGAATCCGGC |
| | | CATTTCATCCTCCAGGAAGTCCATGGGCCAACCGCCCACTCCCCGCCAGCCTCGCACCAACACC |
| | | AACCGGTCCAGTACATGAGCCAGTCGGGTCACTGACCCCGGCACCAGTCCCTCAGCCACTGGAT |
| | | CCAGCGCCCGCAGTGACTCCCGAGGCCAGTCACCTGTTGGAGGATCCCGATGAAGAGACGAGC |
| | | CAGGCTGTCAAAGCCCTTCGGGAGATGGCCGATACTGTGATTCCCCAGAAGGAAGAGGCTGCAA |
| | | TCTGTGGCCAAATGGACCTTTCCCATCCGCCCCCAAGGGGCCATCTGGATGAGCTGACAACCAC |
| | | ACTTGAGTCCATGACCGAGGATCTGAACCTGGACTCACCCCTGACCCCGGAATTGAACGAGATT |
| | | CTGGATACCTTCCTGAACGACGAGTGCCTCTTGCATGCCATGCATATCAGCACAGGACTGTCCAT |
| | | CTTCGACACATCTCTGTTTAAAAGGCCGGCGGCCACGAAAAAGGCCGGCCAGGCAAAAAAGAA |
| | | AAAGATGGCCTCAAACGATTATACCCAACAAGCAACCCAAAGCTATGGGGCCTACCCCACCCAG |
| | | CCCGGGCAGGGCTATTCCCAGCAGAGCAGTCAGCCCTACGGACAGCAGAGTTACAGTGGTTATA |
| | | GCCAGTCCACGGACACTTCAGGCTATGGCCAGAGCAGCTATTCTTCTTATGGCCAGAGCCAGAA |
| | | CACAGGCTATGGAACTCAGTCAACTCCCCAGGGATATGGCTCGACTGGGGGCTATGGCAGTAGC |
| | | CAGAGCTCCCAATCGTCTTACGGGCAGCAGTCCTCCTACCCTGGCTATGGCCAGCAGCCAGCTC |
| | | CCAGCAGCACCTCGGGAAGTTACGGTAGCAGTTCTCAGAGCAGCAGCTATGGGCAGCCCCAGA |
| | | GTGGGAGCTACAGCCAGCAGCCTAGCTATGGTGGACAGCAGCAAAGCTATGGACAGCAGCAAA |
| | | GCTATAATCCCCCTCAGGGCTATGGACAGCAGAACCAGTACAACAGCAGCAGTGGTGGTGGAG |
| | | GTGGAGGTGGAGGTGGAGGTAACTATGGCCAAGATCAATCCTCCATGAGTAGTGGTGGTGGCA |
| | | GTGGTGGCGGTTATGGCAATCAAGACCAGAGTGGTGGAGGTGGCAGCGGTGGCTATGGACAGC |
| | | AGGACCGTGGAGAATTCGGCAGTGGAGAGGGCAGAGGAAGTCTGCTAACATGCGGTGACGTCG |
| | | AGGAGAATCCTGGCCCAGTGAGCAAGGGCGAGGAGCTGTTCACCGGGGTGGTGCCCATCCTGG |
| | | TCGAGCTGGACGGCGACGTAAACGGCCACAAGTTCAGCGTGTCCGGCGAGGGCGAGGGCGATG |
| | | CCACCTACGGCAAGCTGACCCTGAAGTTCATCTGCACCACCGGCAAGCTGCCCGTGCCCTGGCC |
| | | CACCCTCGTGACCACCCTGACCTACGGCGTGCAGTGCTTCAGCCGCTACCCCGACCACATGAAG |
| | | CAGCACGACTTCTTCAAGTCCGCCATGCCCGAAGGCTACGTCCAGGAGCGCACCATCTTCTTCAA |
| | | GGACGACGGCAACTACAAGACCCGCGCCGAGGTGAAGTTCGAGGGCGACACCCTGGTGAACCG |
| | | CATCGAGCTGAAGGGCATCGACTTCAAGGAGGACGGCAACATCCTGGGGCACAAGCTGGAGTA |
| | | CAACTACAACAGCCACAACGTCTATATCATGGCCGACAAGCAGAAGAACGGCATCAAGGTGAA |
| | | CTTCAAGATCCGCCACAACATCGAGGACGGCAGCGTGCAGCTCGCCGACCACTACCAGCAGAA |
| | | CACCCCCATCGGCGACGGCCCCGTGCTGCTGCCCGACAACCACTACCTGAGCACCCAGTCCGCC |
| | | CTGAGCAAAGACCCCAACGAGAAGCGCGATCACATGGTCCTGCTGGAGTTCGTGACCGCCGCC |
| | | GGGATCACTCTCGGCATGGACGAGCTGTACAAGGAATTCTAACTAGAGCTCGCTGATCAGCCTC |
| | | GACTGTGCCTTCTAGTTGCCAGCCATCTGTTGTTTGCCCCTCCCCCGTGCCTTCCTTGACCCTGGA |
| | | AGGTGCCACTCCCACTGTCCTTTCCTAATAAAATGAGGAAATTGCATCGCATTGTCTGAGTAGGT |
| | | GTCATTCTATTCTGGGGGGTGGGGTGGGGCAGGACAGCAAGGGGGAGGATTGGGAAGAGAATA |
| | | GCAGGCATGCTGGGGATGCGGTGGGCTCTATGGCTTCTGAGGCGGAAAGAACCAGCTGGGGCT |
| | | CGATACCGTCGACCTCTAGCTAGAGCTTGGCGTAATCATGGTCATAGCTGTTTCCTGTGTGAAATT |
| | | GTTATCCGCTCACAATTCCACACAACATACGAGCCGGAAGCATAAAGTGTAAAGCCTAGGGTGC |
| | | CTAATGAGTGAGCTAACTCACATTAATTGCGTTGCGCTCACTGCCCGCTTTCCAGTCGGGAAACC |
| | | TGTCGTGCCAGCTGCATTAATGAATCGGCCAACGCGCGGGGAGAGGCGGTTTGCGTATTGGGCG |
| | | CTCTTCCGCTTCCTCGCTCACTGACTCGCTGGGCTCGGTCGTTCGGCTGCGGCGAGCGGTATCAG |
| | | CTCACTCAAAGGCGGTAATACGGTTATCCACAGAATCAGGGGATAACGCAGGAAAGAACATGTG |
| | | AGCAAAAGGCCAGCAAAAGGCCAGGAACCGTAAAAAGGCCGCGTTGCTGGCGTTTTTCCATAG |
| | | GCTCCGCCCCCCTGACGAGCATCACAAAAATCGACGCTCAAGTCAGAGGTGGCGAAACCCGAC |
| | | AGGACTATAAAGATACCAGGCGTTTCCCCCTGGAAGCTCCCTCGTGCGCTCTCCTGTTCCGACCC |
| | | TGCCGCTTACCGGATACCTGTCCGCCTTTCTCCCTTCGGGAAGCGTGGCGCTTTCTCATAGCTCAC |
| | | GCTGTAGGTATCTCAGTTCGGTGTAGGTCGTTCGCTCCAAGCTGGGCTGTGTGCACGAACCCCCC |
| | | GTTCAGCCCGACCGCTGCGCCTTATCCGGTAACTATCGTCTTGAGTCCAACCCGGTAAGACACGA |
| | | CTTATCGCCACTGGCAGCAGCCACTGGTAACAGGATTAGCAGAGCGAGGTATGTAGGCGGTGCT |
| | | ACAGAGTTCTTGAAGTGGTGGCCTAACTACGGCTACACTAGAAGAACAGTATTTGGTATCTGCGC |
| | | TCTGCTGAAGCCAGTTACCTTCGGAAAAAGAGTTGGTAGCTCTTGATCCGGCAAACAAACCACC |
| | | GCTGGTAGCGGTGGTTTTTTTGTTTGCAAGCAGCAGATTACGCGCAGAAAAAAAGGATCTCAAGA |
| | | AGATCCTTTGATCTTTTCTACGGGGTCTGACGCTCAGTGGAACGAAAACTCACGTTAAGGGATTTT |
| | | GGTCATGAGATTATCAAAAAGGATCTTCACCTAGATCCTTTTAAATTAAAAATGAAGTTTTAAATC |
| | | AATCTAAAGTATATATGAGTAAACTTGGTCTGACAGTTACCAATGCTTAATCAGTGAGGCACCTAT |
| | | CTCAGCGATCTGTCTATTTCGTTCATCCATAGTTGCCTGACTCCCCGTCGTGTAGATAACTACGAT |
| | | ACGGGAGGGCTTACCATCTGGCCCCAGTGCTGCAATGATACCGCGAGACCCACGCTCACCGGCT |
| | | CCAGATTTATCAGCAATAAACCAGCCAGCCGGAAGGGCCGAGCGCAGAAGTGGTCCTGCAACTT |
| | | TATCCGCCTCCATCCAGTCTATTAATTGTTGCCGGGAAGCTAGAGTAAGTAGTTCGCCAGTTAATA |
| | | GTTTGCGCAACGTTGTTGCCATTGCTACAGGCATCGTGGTGTCACGCTCGTCGTTTGGTATGGCTT |
| | | CATTCAGCTCCGGTTCCCAACGATCAAGGCGAGTTACATGATCCCCCATGTTGTGCAAAAAAGCG |
| | | GTTAGCTCCTTCGGTCCTCCGATCGTTGTCAGAAGTAAGTTGGCCGCAGTGTTATCACTCATGGT |
| | | ATGGCAGCACTGCATAATTCTCTTACTGTCATGCCATCCGTAAGATGCTTTTCTGTGACTGGTGAG |
| | | TACTCAACCAAGTCATTCTGAGAATAGTGTATGCGGCGACCGAGTTGCTCTTGCCCGGCGTCAAT |
| | | ACGGGATAATACCGCGCCACATAGCAGAACTTTAAAAGTGCTCATCATTGGAAAACGTTCTTCGG |
| | | GGCGAAAACTCTCAAGGATCTTACCGCTGTTGAGATCCAGTTCGATGTAACCCACTCGTGCACCC |
| | | AACTGATCTTCAGCATCTTTTACTTTCACCAGCGTTTCTGGGTGAGCAAAAACAGGAAGGCAAAA |
| | | TGCCGCAAAAAAGGGAATAAGGGCGACACGGAAATGTTGAATACTCATACTCTTCCTTTTTCAAT |
| | | ATTATTGAAGCATTTATCAGGGTTATTGTCTCATGAGCGGATACATATTTGAATGTATTTAGAAAA |
| | | ATAAACAAATAGGGGTTCCGCGCACATTTCCCCGAAAAGTGCCACCTGACGTCGACGGATCGGG |
| | | AGATCGATCTCCCGATCCCCTAGGGTCGACTCTCAGTACAATCTGCTCTGATGCCGCATAGTTAA |
| | | GCCAGTATCTGCTCCCTGCTTGTGTGTTGGAGGTCGCTGAGTAGTGCGCGAGCAAAATTTAAGCT |
| | | ACAACAAGGCAAGGCTTGACCGACAATTGCATGAAGAATCTGCTTAGGGTTAGGCGTTTTGCGCT |
| | | GCTTCGCGATGTACGGGCCAGATATACGCGTGAGGGCCTATTTCCCATGATTCCTTCATATTTGCA |
| | | TATACGATACAAGGCTGTTAGAGAGATAATTGGAATTAATTTGACTGTAAACACAAAGATATTAG |
| | | TACAAAATACGTGACGTAGAAAGTAATAATTTCTTGGGTAGTTTGCAGTTTTAAAATTATGTTTTAA |
| | | AATGGACTATCATATGCTTACCGTAACTTGAAAGTATTTCGATTTCTTGGCTTTATATATCTTGTGG |
| | | AAAGGACGAAACACCGGTGGAGCGGTTCACGACCGCGACCTCAACGCCGCCCGGAACATCAAG |
| | | CGGGAAGGGCTTTCGCAAATCGTCGTCGCGGGGCACGCGGAGACGTTAAACGCTCGGGGAGAG |
| | | GGTGTCAGACCTGCGATAGCGGGCAGCCCTCGATGAAGCGAGAATCCAACGGCTTTAGCCGTTG |
| | | GAGTGTCAAGGGCTGTTGTCATTTTATTTTTTGTTTTAGAGCTAGAAATAGTAAGTTAAAATAAGG |
| | | CTAGTCCGTT |
| seq. | vector_ | TTTAGCGCGTGCGCCAATTCTGCAGACAAATGGCTCTAGAGGTACCGACATTGATTATTGACTAG |
| 57 | ISDge10TnpB- | TTATTAATAGTAATCAATTACGGGGTCATTAGTTCATAGCCCATATATGGAGTTCCGCGTTACATA |
| | VPR-reRNA) | ACTTACGGTAAATGGCCCGCCTGGCTGACCGCCCAACGACCCCCGCCCATTGACGTCAATAATG |
| | (10 nt + WT | ACGTATGTTCCCATAGTAACCCCAATAGGGACTTTCCATTGACGTCAATGGGTGGAGTATTTACG |
| | TnpB) | GTAAACTGCCCACTTGGCAGTACATCAAGTGTATCATATGCCAAGTACGCCCCCTATTGACGTCA |
| | | ATGACGGTAAATGGCCCGCCTGGCATTATGCCCAGTACATGACCTTATGGGACTTTCCTACTTGG |
| | | CAGTACATCTACGTATTAGTCATCGCTATTACCATGGTGATGCGGTTTTGGCAGTACATCAATGGG |
| | | CGTGGATAGCGGTTTGACTCACGGGGATTTCCAAGTCTCCACCCCATTGACGTCAATGGGAGTTT |
| | | GTTTTGGCACCAAAATCAACGGGACTTTCCAAAATGTCGTAACAACTCCGCCCCATTGACGCAAA |
| | | TGGGCGGTAGGCGTGTACGGTGGGAGGTCTATATAAGCAGAGCTGGTTTAGTGAACCGTCAGAT |
| | | CCGCTAGAGATCCGCGGCCGCGCCACCATGGCCCCAAAGAAGAAGCGGAAGGTCGGTATCCAC |
| | | GGAGTCCCAGCAGCCATGACCACCCACCGCAAGGTGTACAGATACCGCATCGAGCCCACCCCC |
| | | GTGCAGGAGAGCAAGCTGTACATGCTGGCCGGCAGCCGGAGATTCGTGTTCAACTGGGCCCTG |
| | | GCCCGGAGAAGAGAGCACTACGCCGAAACCGGCAAGACCCTGGGATACAACGCCCAGGCAGG |
| | | AGAGCTGACCGCCCTGAAAAACCAAGAAGAAACCAGCTGGCTGAAAGAGTCCGACTCACAACT |
| | | GCTGCAGCAGGCACTGAAAGACGTGGAAAGAGCCTTCGTGAACTTCTTCGAAAAACGGGCCAG |
| | | ATTCCCCAGATTCAAAAGCAAGAAAACCGACACCCCCAGATTCAGAATCCCCCAAAGAGTGAGA |
| | | ATAGAGGGCAGCAGAGTGTACGTGCCCAAGGTGGGGTGGGTGAAGCTGAGAAAGAGTCAGGA |
| | | GATAGAAGGAAAAACCAAAAGCGCCACCTTCAAGAGGGAGGCAGATGGCCATTGGTACGTGCT |
| | | GCTGGTGAGTGAGTTCGAGATGCCTGATGTGCCTCTGCCCCCAGTGCCCGAGAGCGAGGTGGTG |
| | | GGAATCGACCTGGGACTGAAGGACTTCTACGTCCTGTCTGACGGGGGCAGGAAGGAGGCCCCC |
| | | AGATTCGCTAGAAAGGGCCAGCGAAAACTGAGAAGAGCTGCAAGAAGACATAGCAAGTGCACC |
| | | CGGGGAAGCAACAGAAAGGCCAAGGCAAAGAGGAAGCTGGCAAGAGTGCACAGACAGATCGC |
| | | AAACCAGAGAAAAGACTTCGTGCACAAGGCCACCAGCGGCCTCGTGCAGCAGTACCAAGGCTT |
| | | CTGCATCGAAAACCTGAGCATCAAGGGCATGGCCAAAACCAAACTGTCAAAAAGCGTGCTGGA |
| | | CGCAGCCCTGGGGGAATTTAGAAGACAGCTGGCCTATAAAGCCCAGTGGCATAGAAAGTGGCT |
| | | GGCAGTGATAGACAGATGGTTCCCCAGCAGCAAGCTGTGCGGAGAGTGCGGCAGCATTAACGC |
| | | CGATCTGACCCTGAGCGACAGAGAATGGACCTGCGAGTGTGGCGCAGTGCACGACAGAGATCT |
| | | GAACGCCGCTCGCAACATCAAGAGAGAGGGCCTGAGTCAGATTGTGGTGGCCGGACATGCCGA |
| | | AACCCTGAACGCCAGAGGGGAGGGCGTGAGACCCGCCATTGCCGGAAGCCCTAGAAGCAGGG |
| | | CTGACCCCAAGAAGAAGAGGAAGGTGTCGCCAGGGATCCGTCGACTTGACGCGTTGATATCAAC |
| | | AAGTTTGTACAAAAAAGCAGGCTACAAAGAGGCCAGCGGTTCCGGACGGGCTGACGCATTGGA |
| | | CGATTTTGATCTGGATATGCTGGGAAGTGACGCCCTCGATGATTTTGACCTTGACATGCTTGGTTC |
| | | GGATGCCCTTGATGACTTTGACCTCGACATGCTCGGCAGTGACGCCCTTGATGATTTCGACCTGG |
| | | ACATGCTGATTAACTCTAGAAGTTCCGGATCTCCGAAAAAGAAACGCAAAGTTGGTAGCCAGTA |
| | | CCTGCCCGACACCGACGACCGGCACCGGATCGAGGAAAAGCGGAAGCGGACCTACGAGACATT |
| | | CAAGAGCATCATGAAGAAGTCCCCCTTCAGCGGCCCCACCGACCCTAGACCTCCACCTAGAAGA |
| | | ATCGCCGTGCCCAGCAGATCCAGCGCCAGCGTGCCAAAACCTGCCCCCCAGCCTTACCCCTTCA |
| | | CCAGCAGCCTGAGCACCATCAACTACGACGAGTTCCCTACCATGGTGTTCCCCAGCGGCCAGAT |
| | | CTCTCAGGCCTCTGCTCTGGCTCCAGCCCCTCCTCAGGTGCTGCCTCAGGCTCCTGCTCCTGCAC |
| | | CAGCTCCAGCCATGGTGTCTGCACTGGCTCAGGCACCAGCACCCGTGCCTGTGCTGGCTCCTGG |
| | | ACCTCCACAGGCTGTGGCTCCACCAGCCCCTAAACCTACACAGGCCGGCGAGGGCACACTGTCT |
| | | GAAGCTCTGCTGCAGCTGCAGTTCGACGACGAGGATCTGGGAGCCCTGCTGGGAAACAGCACC |
| | | GATCCTGCCGTGTTCACCGACCTGGCCAGCGTGGACAACAGCGAGTTCCAGCAGCTGCTGAACC |
| | | AGGGCATCCCTGTGGCCCCTCACACCACCGAGCCCATGCTGATGGAATACCCCGAGGCCATCAC |
| | | CCGGCTCGTGACAGGCGCTCAGAGGCCTCCTGATCCAGCTCCTGCCCCTCTGGGAGCACCAGGC |
| | | CTGCCTAATGGACTGCTGTCTGGCGACGAGGACTTCAGCTCTATCGCCGATATGGATTTCTCAGC |
| | | CTTGCTGGGCTCTGGCAGCGGCAGCCGGGATTCCAGGGAAGGGATGTTTTTGCCGAAGCCTGAG |
| | | GCCGGCTCCGCTATTAGTGACGTGTTTGAGGGCCGCGAGGTGTGCCAGCCAAAACGAATCCGGC |
| | | CATTTCATCCTCCAGGAAGTCCATGGGCCAACCGCCCACTCCCCGCCAGCCTCGCACCAACACC |
| | | AACCGGTCCAGTACATGAGCCAGTCGGGTCACTGACCCCGGCACCAGTCCCTCAGCCACTGGAT |
| | | CCAGCGCCCGCAGTGACTCCCGAGGCCAGTCACCTGTTGGAGGATCCCGATGAAGAGACGAGC |
| | | CAGGCTGTCAAAGCCCTTCGGGAGATGGCCGATACTGTGATTCCCCAGAAGGAAGAGGCTGCAA |
| | | TCTGTGGCCAAATGGACCTTTCCCATCCGCCCCCAAGGGGCCATCTGGATGAGCTGACAACCAC |
| | | ACTTGAGTCCATGACCGAGGATCTGAACCTGGACTCACCCCTGACCCCGGAATTGAACGAGATT |
| | | CTGGATACCTTCCTGAACGACGAGTGCCTCTTGCATGCCATGCATATCAGCACAGGACTGTCCAT |
| | | CTTCGACACATCTCTGTTTAAAAGGCCGGCGGCCACGAAAAAGGCCGGCCAGGCAAAAAAGAA |
| | | AAAGATGGCCTCAAACGATTATACCCAACAAGCAACCCAAAGCTATGGGGCCTACCCCACCCAG |
| | | CCCGGGCAGGGCTATTCCCAGCAGAGCAGTCAGCCCTACGGACAGCAGAGTTACAGTGGTTATA |
| | | GCCAGTCCACGGACACTTCAGGCTATGGCCAGAGCAGCTATTCTTCTTATGGCCAGAGCCAGAA |
| | | CACAGGCTATGGAACTCAGTCAACTCCCCAGGGATATGGCTCGACTGGCGGCTATGGCAGTAGC |
| | | CAGAGCTCCCAATCGTCTTACGGGCAGCAGTCCTCCTACCCTGGCTATGGCCAGCAGCCAGCTC |
| | | CCAGCAGCACCTCGGGAAGTTACGGTAGCAGTTCTCAGAGCAGCAGCTATGGGCAGCCCCAGA |
| | | GTGGGAGCTACAGCCAGCAGCCTAGCTATGGTGGACAGCAGCAAAGCTATGGACAGCAGCAAA |
| | | GCTATAATCCCCCTCAGGGCTATGGACAGCAGAACCAGTACAACAGCAGCAGTGGTGGTGGAG |
| | | GTGGAGGTGGAGGTGGAGGTAACTATGGCCAAGATCAATCCTCCATGAGTAGTGGTGGTGGCA |
| | | GTGGTGGCGGTTATGGCAATCAAGACCAGAGTGGTGGAGGTGGCAGCGGTGGCTATGGACAGC |
| | | AGGACCGTGGAGAATTCGGCAGTGGAGAGGGCAGAGGAAGTCTGCTAACATGCGGTGACGTCG |
| | | AGGAGAATCCTGGCCCAGTGAGCAAGGGCGAGGAGCTGTTCACCGGGGTGGTGCCCATCCTGG |
| | | TCGAGCTGGACGGCGACGTAAACGGCCACAAGTTCAGCGTGTCCGGCGAGGGCGAGGGCGATG |
| | | CCACCTACGGCAAGCTGACCCTGAAGTTCATCTGCACCACCGGCAAGCTGCCCGTGCCCTGGCC |
| | | CACCCTCGTGACCACCCTGACCTACGGCGTGCAGTGCTTCAGCCGCTACCCCGACCACATGAAG |
| | | CAGCACGACTTCTTCAAGTCCGCCATGCCCGAAGGCTACGTCCAGGAGCGCACCATCTTCTTCAA |
| | | GGACGACGGCAACTACAAGACCCGCGCCGAGGTGAAGTTCGAGGGCGACACCCTGGTGAACCG |
| | | CATCGAGCTGAAGGGCATCGACTTCAAGGAGGACGGCAACATCCTGGGGCACAAGCTGGAGTA |
| | | CAACTACAACAGCCACAACGTCTATATCATGGCCGACAAGCAGAAGAACGGCATCAAGGTGAA |
| | | CTTCAAGATCCGCCACAACATCGAGGACGGCAGCGTGCAGCTCGCCGACCACTACCAGCAGAA |
| | | CACCCCCATCGGCGACGGCCCCGTGCTGCTGCCCGACAACCACTACCTGAGCACCCAGTCCGCC |
| | | CTGAGCAAAGACCCCAACGAGAAGCGCGATCACATGGTCCTGCTGGAGTTCGTGACCGCCGCC |
| | | GGGATCACTCTCGGCATGGACGAGCTGTACAAGGAATTCTAACTAGAGCTCGCTGATCAGCCTC |
| | | GACTGTGCCTTCTAGTTGCCAGCCATCTGTTGTTTGCCCCTCCCCCGTGCCTTCCTTGACCCTGGA |
| | | AGGTGCCACTCCCACTGTCCTTTCCTAATAAAATGAGGAAATTGCATCGCATTGTCTGAGTAGGT |
| | | GTCATTCTATTCTGGGGGGTGGGGTGGGGCAGGACAGCAAGGGGGAGGATTGGGAAGAGAATA |
| | | GCAGGCATGCTGGGGATGCGGTGGGCTCTATCCCTTCTGAGGCGGAAAGAACCAGCTGGGGCT |
| | | CGATACCGTCGACCTCTAGCTAGAGCTTGGCGTAATCATGGTCATAGCTGTTTCCTGTGTGAAATT |
| | | GTTATCCGCTCACAATTCCACACAACATACGAGCCGGAAGCATAAAGTGTAAAGCCTAGGGTGC |
| | | CTAATGAGTGAGCTAACTCACATTAATTGCGTTGCGCTCACTGCCCGCTTTCCAGTCGGGAAACC |
| | | TGTCGTGCCAGCTGCATTAATGAATCGGCCAACGCGCGGGGAGAGGCGGTTTGCGTATTGGGCG |
| | | CTCTTCCGCTTCCTCGCTCACTGACTCGCTGCGCTCGGTCGTTCGGCTGCGGCGAGCGGTATCAG |
| | | CTCACTCAAAGGCGGTAATACGGTTATCCACAGAATCAGGGGATAACGCAGGAAAGAACATGTG |
| | | AGCAAAAGGCCAGCAAAAGGCCAGGAACCGTAAAAAGGCCGCGTTGCTGGCGTTTTTCCATAG |
| | | GCTCCGCCCCCCTGACGAGCATCACAAAAATCGACGCTCAAGTCAGAGGTGGCGAAACCCGAC |
| | | AGGACTATAAAGATACCAGGCGTTTCCCCCTGGAAGCTCCCTCGTGCGCTCTCCTGTTCCGACCC |
| | | TGCCGCTTACCGGATACCTGTCCGCCTTTCTCCCTTCGGGAAGCGTGGCGCTTTCTCATAGCTCAC |
| | | GCTGTAGGTATCTCAGTTCGGTGTAGGTCGTTCGCTCCAAGCTGGGCTGTGTGCACGAACCCCCC |
| | | GTTCAGCCCGACCGCTGCGCCTTATCCGGTAACTATCGTCTTGAGTCCAACCCGGTAAGACACGA |
| | | CTTATCGCCACTGGCAGCAGCCACTGGTAACAGGATTAGCAGAGCGAGGTATGTAGGCGGTGCT |
| | | ACAGAGTTCTTGAAGTGGTGGCCTAACTACGGCTACACTAGAAGAACAGTATTTGGTATCTGCGC |
| | | TCTGCTGAAGCCAGTTACCTTCGGAAAAAGAGTTGGTAGCTCTTGATCCGGCAAACAAACCACC |
| | | GCTGGTAGCGGTGGTTTTTTTGTTTGCAAGCAGCAGATTACGCGCAGAAAAAAAGGATCTCAAGA |
| | | AGATCCTTTGATCTTTTCTACGGGGTCTGACGCTCAGTGGAACGAAAACTCACGTTAAGGGATTTT |
| | | GGTCATGAGATTATCAAAAAGGATCTTCACCTAGATCCTTTTAAATTAAAAATGAAGTTTTAAATC |
| | | AATCTAAAGTATATATGAGTAAACTTGGTCTGACAGTTACCAATGCTTAATCAGTGAGGCACCTAT |
| | | CTCAGCGATCTGTCTATTTCGTTCATCCATAGTTGCCTGACTCCCCGTCGTGTAGATAACTACGAT |
| | | ACGGGAGGGCTTACCATCTGGCCCCAGTGCTGCAATGATACCGCGAGACCCACGCTCACCGGCT |
| | | CCAGATTTATCAGCAATAAACCAGCCAGCCGGAAGGGCCGAGCGCAGAAGTGGTCCTGCAACTT |
| | | TATCCGCCTCCATCCAGTCTATTAATTGTTGCCGGGAAGCTAGAGTAAGTAGTTCGCCAGTTAATA |
| | | GTTTGCGCAACGTTGTTGCCATTGCTACAGGCATCGTGGTGTCACGCTCGTCGTTTGGTATGGCTT |
| | | CATTCAGCTCCGGTTCCCAACGATCAAGGCGAGTTACATGATCCCCCATGTTGTGCAAAAAAGCG |
| | | GTTAGCTCCTTCGGTCCTCCGATCGTTGTCAGAAGTAAGTTGGCCGCAGTGTTATCACTCATGGTT |
| | | ATGGCAGCACTGCATAATTCTCTTACTGTCATGCCATCCGTAAGATGCTTTTCTGTGACTGGTGAG |
| | | TACTCAACCAAGTCATTCTGAGAATAGTGTATGCGGCGACCGAGTTGCTCTTGCCCGGCGTCAAT |
| | | ACGGGATAATACCGCGCCACATAGCAGAACTTTAAAAGTGCTCATCATTGGAAAACGTTCTTCGG |
| | | GGCGAAAACTCTCAAGGATCTTACCGCTGTTGAGATCCAGTTCGATGTAACCCACTCGTGCACCC |
| | | AACTGATCTTCAGCATCTTTTACTTTCACCAGCGTTTCTGGGTGAGCAAAAACAGGAAGGCAAAA |
| | | TGCCGCAAAAAAGGGAATAAGGGCGACACGGAAATGTTGAATACTCATACTCTTCCTTTTTCAAT |
| | | ATTATTGAAGCATTTATCAGGGTTATTGTCTCATGAGCGGATACATATTTGAATGTATTTAGAAAA |
| | | ATAAACAAATAGGGGTTCCGCGCACATTTCCCCGAAAAGTGCCACCTGACGTCGACGGATCGGG |
| | | AGATCGATCTCCCGATCCCCTAGGGTCGACTCTCAGTACAATCTGCTCTGATGCCGCATAGTTAA |
| | | GCCAGTATCTGCTCCCTGCTTGTGTGTTGGAGGTCGCTGAGTAGTGCGCGAGCAAAATTTAAGCT |
| | | ACAACAAGGCAAGGCTTGACCGACAATTGCATGAAGAATCTGCTTAGGGTTAGGCGTTTTGCGCT |
| | | GCTTCGCGATGTACGGGCCAGATATACGCGTGAGGGCCTATTTCCCATGATTCCTTCATATTTGCA |
| | | TATACGATACAAGGCTGTTAGAGAGATAATTGGAATTAATTTGACTGTAAACACAAAGATATTAG |
| | | TACAAAATACGTGACGTAGAAAGTAATAATTTCTTGGGTAGTTTGCAGTTTTAAAATTATGTTTTAA |
| | | AATGGACTATCATATGCTTACCGTAACTTGAAAGTATTTCGATTTCTTGGCTTTATATATCTTGTGG |
| | | AAAGGACGAAACACCGGTGGAGCGGTTCACGACCGCGACCTCAACGCCGCCCGGAACATCAAG |
| | | CGGGAAGGGCTTTCGCAAATCGTCGTCGCGGGGCACGCGGAGACGTTAAACGCTCGGGGAGAG |
| | | GGTGTCAGACCTGCGATAGCGGGCAGCCCTCGATGAAGCGAGAATCCAACGGCTTTAGCCGTTG |
| | | GAGTGTCAAGGGCTGTTGTTTTTATTTTTTGTTTTAGAGCTAGAAATAGTAAGTTAAAATAAGGCT |
| | | AGTCCGTT |
| seq. | vector_ | TTTAGCGCGTGCGCCAATTCTGCAGACAAATGGCTCTAGAGGTACCGACATTGATTATTGACTAG |
| 58 | ISDge10TnpB- | TTATTAATAGTAATCAATTACGGGGTCATTAGTTCATAGCCCATATATGGAGTTCCGCGTTACATA |
| | VPR-reRNA) | ACTTACGGTAAATGGCCCGCCTGGCTGACCGCCCAACGACCCCCGCCCATTGACGTCAATAATG |
| | (8 nt + WT | ACGTATGTTCCCATAGTAACGCCAATAGGGACTTTCCATTGACGTCAATGGGTGGAGTATTTACG |
| | TnpB) | GTAAACTGCCCACTTGGCAGTACATCAAGTGTATCATATGCCAAGTACGCCCCCTATTGACGTCA |
| | | ATGACGGTAAATGGCCCGCCTGGCATTATGCCCAGTACATGACCTTATGGGACTTTCCTACTTGG |
| | | CAGTACATCTACGTATTAGTCATCGCTATTACCATGGTGATGCGGTTTTGGCAGTACATCAATGGG |
| | | CGTGGATAGCGGTTTGACTCACGGGGATTTCCAAGTCTCCACCCCATTGACGTCAATGGGAGTTT |
| | | GTTTTGGCACCAAAATCAACGGGACTTTCCAAAATGTCGTAACAACTCCGCCCCATTGACGCAAA |
| | | TGGGCGGTAGGCGTGTACGGTGGGAGGTCTATATAAGCAGAGCTGGTTTAGTGAACCGTCAGAT |
| | | CCGCTAGAGATCCGCGGCCGCGCCACCATGGCCCCAAAGAAGAAGCGGAAGGTCGGTATCCAC |
| | | GGAGTCCCAGCAGCCATGACCACCCACCGCAAGGTGTACAGATACCGCATCGAGCCCACCCCC |
| | | GTGCAGGAGAGCAAGCTGTACATGCTGGCCGGCAGCCGGAGATTCGTGTTCAACTGGGCCCTG |
| | | GCCCGGAGAAGAGAGCACTACGCCGAAACCGGCAAGACCCTGGGATACAACGCCCAGGCAGG |
| | | AGAGCTGACCGCCCTCAAAAACCAAGAAGAAACCAGCTGGCTGAAAGAGTCCGACTCACAACT |
| | | GCTGCAGCAGGCACTGAAAGACGTGGAAAGAGCCTTCGTGAACTTCTTCGAAAAACGGGCCAG |
| | | ATTCCCCAGATTCAAAAGCAAGAAAACCGACACCCCCAGATTCAGAATCCCCCAAAGAGTGAGA |
| | | ATAGAGGGCAGCAGAGTGTACGTGCCCAAGGTGGGGTGGGTGAAGCTGAGAAAGAGTCAGGA |
| | | GATAGAAGGAAAAACCAAAAGCGCCACCTTCAAGAGGGAGGCAGATGGCCATTGGTACGTGCT |
| | | GCTGGTGAGTGAGTTCGAGATGCCTGATGTGCCTCTGCCCCCAGTGCCCGAGAGCGAGGTGGTG |
| | | GGAATCGACCTGGGACTGAAGGACTTCTACGTCCTGTCTGACGGGGGCAGGAAGGAGGCCCCC |
| | | AGATTCGCTAGAAAGGGCCAGCGAAAACTGAGAAGAGCTGCAAGAAGACATAGCAAGTGCACC |
| | | CGGGGAAGCAACAGAAAGGCCAAGGCAAAGAGGAAGCTGGCAAGAGTGCACAGACAGATCGC |
| | | AAACCAGAGAAAAGACTTCGTGCACAAGGCCACCAGCGGCCTCGTGCAGCAGTACCAAGGCTT |
| | | CTGCATCGAAAACCTGAGCATCAAGGGCATGGCCAAAACCAAACTGTCAAAAAGCGTGCTGGA |
| | | CGCAGCCCTGGGGGAATTTAGAAGACAGCTGGCCTATAAAGCCCAGTGGCATAGAAAGTGGCT |
| | | GGCAGTGATAGACAGATGGTTCCCCAGCAGCAAGCTGTGCGGAGAGTGCGGCAGCATTAACCC |
| | | CGATCTGACCCTGAGCGACAGAGAATGGACCTGCGAGTGTGGCGCAGTGCACGACAGAGATCT |
| | | GAACGCCGCTCGCAACATCAAGAGAGAGGGCCTGAGTCAGATTGTGGTGGCCGGACATGCCGA |
| | | AACCCTGAACGCCAGAGGGGAGGGCGTGAGACCCGCCATTGCCGGAAGCCCTAGAAGCAGGG |
| | | CTGACCCCAAGAAGAAGAGGAAGGTGTCGCCAGGGATCCGTCGACTTGACGCGTTGATATCAAC |
| | | AAGTTTGTACAAAAAAGCAGGCTACAAAGAGGCCAGCGGTTCCGGACGGGCTGACGCATTGGA |
| | | CGATTTTGATCTGGATATGCTGGGAAGTGACGCCCTCGATGATTTTGACCTTGACATGCTTGGTTC |
| | | GGATGCCCTTGATGACTTTGACCTCGACATGCTCGGCAGTGACGCCCTTGATGATTTCGACCTGG |
| | | ACATGCTGATTAACTCTAGAAGTTCCGGATCTCCGAAAAAGAAACGCAAAGTTGGTAGCCAGTA |
| | | CCTGCCCGACACCGACGACCGGCACCGGATCGAGGAAAAGCGGAAGCGGACCTACGAGACATT |
| | | CAAGAGCATCATGAAGAAGTCCCCCTTCAGCGGCCCCACCGACCCTAGACCTCCACCTAGAAGA |
| | | ATCGCCGTGCCCAGCAGATCCAGCGCCAGCGTGCCAAAACCTGCCCCCCAGCCTTACCCCTTCA |
| | | CCAGCAGCCTGAGCACCATCAACTACGACGAGTTCCCTACCATGGTGTTCCCCAGCGGCCAGAT |
| | | CTCTCAGGCCTCTGCTCTGGCTCCAGCCGGTCCTCAGGTGCTGCCTCAGGCTCCTGCTCCTGCAC |
| | | CAGCTCCAGCCATGGTGTCTGCACTGGCTCAGGCACCAGCACCCGTGCCTGTGCTGGCTCCTGG |
| | | ACCTCCACAGGCTGTGGCTCCACCAGCCCCTAAACCTACACAGGCCGGCGAGGGCACACTGTCT |
| | | GAAGCTCTGCTGCAGCTGCAGTTCGACGACGAGGATCTGGGAGCCCTGCTGGGAAACAGCACC |
| | | GATCCTGCCGTGTTCACCGACCTGGCCAGCGTGGACAACAGCGAGTTCCAGCAGCTGCTGAAC |
| | | AGGGCATCCCTGTGGCCCCTCACACCACCGAGCCCATGCTGATGGAATACCCCGAGGCCATCAC |
| | | CCGGCTCGTGACAGGCGCTCAGAGGCCTCCTGATCCAGCTCCTGCCCCTCTGGGAGCACCAGGC |
| | | CTGCCTAATGGACTGCTGTCTGGCGACGAGGACTTCAGCTCTATCGCCGATATGGATTTCTCAGC |
| | | CTTGCTGGGCTCTGGCAGCGGCAGCCGGGATTCCAGGGAAGGGATGTTTTTGCCGAAGCCTGAG |
| | | GCCGGCTCCGCTATTAGTGACGTGTTTGAGGGCCGCGAGGTGTGCCAGCCAAAACGAATCCGGC |
| | | CATTTCATCCTCCAGGAAGTCCATGGGCCAACCGCCCACTCCCCGCCAGCCTCGCACCAACACC |
| | | AACCGGTCCAGTACATGAGCCAGTCGGGTCACTGACCCCGGCACCAGTCCCTCAGCCACTGGAT |
| | | CCAGCGCCCGCAGTGACTCCCGAGGCCAGTCACCTGTTGGAGGATCCCGATGAAGAGACGAGC |
| | | CAGGCTGTCAAAGCCCTTCGGGAGATGGCCGATACTGTGATTCCCCAGAAGGAAGAGGCTGCAA |
| | | TCTGTGGCCAAATGGACCTTTCCCATCCGCCCCCAAGGGGCCATCTGGATGAGCTGACAACCAC |
| | | ACTTGAGTCCATGACCGAGGATCTGAACCTGGACTCACCCCTGACCCCGGAATTGAACGAGATT |
| | | CTGGATACCTTCCTGAACGACGAGTGCCTCTTGCATGCCATGCATATCAGCACAGGACTGTCCAT |
| | | CTTCGACACATCTCTGTTTAAAAGGCCGGCGGCCACGAAAAAGGCCGGCCAGGCAAAAAAGAA |
| | | AAAGATGGCCTCAAACGATTATACCCAACAAGCAACCCAAAGCTATGGGGCCTACCCCACCCAG |
| | | CCCGGGCAGGGCTATTCCCAGCAGAGCAGTCAGCCCTACGGACAGCAGAGTTACAGTGGTTATA |
| | | GCCAGTCCACGGACACTTCAGGCTATGGCCAGAGCAGCTATTCTTCTTATGGCCAGAGCCAGAA |
| | | CACAGGCTATGGAACTCAGTCAACTCCCCAGGGATATGGCTCGACTGGCGGCTATGGCAGTAGC |
| | | CAGAGCTCCCAATCGTCTTACGGGCAGCAGTCCTCCTACCCTGGCTATGGCCAGCAGCCAGCTC |
| | | CCAGCAGCACCTCGGGAAGTTACGGTAGCAGTTCTCAGAGCAGCAGCTATGGGCAGCCCCAGA |
| | | GTGGGAGCTACAGCCAGCAGCCTAGCTATGGTGGACAGCAGCAAAGCTATGGACAGCAGCAAA |
| | | GCTATAATCCCCCTCAGGGCTATGGACAGCAGAACCAGTACAACAGCAGCAGTGGTGGTGGAG |
| | | GTGGAGGTGGAGGTGGAGGTAACTATGGCCAAGATCAATCCTCCATGAGTAGTGGTGGTGGCA |
| | | GTGGTGGCGGTTATGGCAATCAAGACCAGAGTGGTGGAGGTGGCAGCGGTGGCTATGGACAGC |
| | | AGGACCGTGGAGAATTCGGCAGTGGAGAGGGCAGAGGAAGTCTGCTAACATGCGGTGACGTCG |
| | | AGGAGAATCCTGGCCCAGTGAGCAAGGGCGAGGAGCTGTTCACCGGGGTGGTGCCCATCCTGG |
| | | TCGAGCTGGACGGCGACGTAAACGGCCACAAGTTCAGCGTGTCCGGCGAGGGCGAGGGCGATG |
| | | CCACCTACGGCAAGCTGACCCTGAAGTTCATCTGCACCACCGGCAAGCTGCCCGTGCCCTGGCC |
| | | CACCCTCGTGACCACCCTGACCTACGGCGTGCAGTGCTTCAGCCGCTACCCCGACCACATGAAG |
| | | CAGCACGACTTCTTCAAGTCCGCCATGCCCGAAGGCTACGTCCAGGAGCGCACCATCTTCTTCAA |
| | | GGACGACGGCAACTACAAGACCCGCGCCGAGGTGAAGTTCGAGGGCGACACCCTGGTGAACCG |
| | | CATCGAGCTGAAGGGCATCGACTTCAAGGAGGACGGCAACATCCTGGGGCACAAGCTGGAGTA |
| | | CAACTACAACAGCCACAACGTCTATATCATGGCCGACAAGCAGAAGAACGGCATCAAGGTGAA |
| | | CTTCAAGATCCGCCACAACATCGAGGACGGCAGCGTGCAGCTCGCCGACCACTACCAGCAGAA |
| | | CACCCCCATCGGCGACGGCCCCGTGCTGCTGCCCGACAACCACTACCTGAGCACCCAGTCCGCC |
| | | CTGAGCAAAGACCCCAACGAGAAGCGCGATCACATGGTCCTGCTGGAGTTCGTGACCGCCGCC |
| | | GGGATCACTCTCGGCATGGACGAGCTGTACAAGGAATTCTAACTAGAGCTCGCTGATCAGCCTC |
| | | GACTGTGCCTTCTAGTTGCCAGCCATCTGTTGTTTGCCCCTCCCCCGTGCCTTCCTTGACCCTGGA |
| | | AGGTGCCACTCCCACTGTCCTTTCCTAATAAAATGAGGAAATTGCATCGCATTGTCTGAGTAGGT |
| | | GTCATTCTATTCTGGGGGGTGGGGTGGGGCAGGACAGCAAGGGGGAGGATTGGGAAGAGAATA |
| | | GCAGGCATGCTGGGGATGCGGTGGGCTCTATGGCTTCTGAGGCGGAAAGAACCAGCTGGGGCT |
| | | CGATACCGTCGACCTCTAGCTAGAGCTTGGCGTAATCATGGTCATAGCTGTTTCCTGTGTGAAATT |
| | | GTTATCCGCTCACAATTCCACACAACATACGAGCCGGAAGCATAAAGTGTAAAGCCTAGGGTGC |
| | | CTAATGAGTGAGCTAACTCACATTAATTGCGTTGCGCTCACTGCCCGCTTTCCAGTCGGGAAACC |
| | | TGTCGTGCCAGCTGCATTAATGAATCGGCCAACGCGCGGGGAGAGGCGGTTTGCGTATTGGGCG |
| | | CTCTTCCGCTTCCTCGCTCACTGACTCGCTGCGCTCGGTCGTTCGGCTGCGGCGAGCGGTATCAG |
| | | CTCACTCAAAGGCGGTAATACGGTTATCCACAGAATCAGGGGATAACGCAGGAAAGAACATGTG |
| | | AGCAAAAGGCCAGCAAAAGGCCAGGAACCGTAAAAAGGCCGCGTTGCTGGCGTTTTTCCATAG |
| | | GCTCCGCCCCCCTGACGAGCATCACAAAAATCGACGCTCAAGTCAGAGGTGGCGAAACCCGAC |
| | | AGGACTATAAAGATACCAGGCGTTTCCCCCTGGAAGCTCCCTCGTGCGCTCTCCTGTTCCGACCC |
| | | TGCCGCTTACCGGATACCTGTCCGCCTTTCTCCCTTCGGGAAGCGTGGCGCTTTCTCATAGCTCAC |
| | | GCTGTAGGTATCTCAGTTCGGTGTAGGTCGTTCGCTCCAAGCTGGGCTGTGTGCACGAACCCCCC |
| | | GTTCAGCCCGACCGCTGCGCCTTATCCGGTAACTATCGTCTTGAGTCCAACCCGGTAAGACACGA |
| | | CTTATCGCCACTGGCAGCAGCCACTGGTAACAGGATTAGCAGAGCGAGGTATGTAGGCGGTGCT |
| | | ACAGAGTTCTTGAAGTGGTGGCCTAACTACGGCTACACTAGAAGAACAGTATTTGGTATCTGCGC |
| | | TCTGCTGAAGCCAGTTACCTTCGGAAAAAGAGTTGGTAGCTCTTGATCCGGCAAACAAACCACC |
| | | GCTGGTAGCGGTGGTTTTTTTGTTTGCAAGCAGCAGATTACGCGCAGAAAAAAAGGATCTCAAGA |
| | | AGATCCTTTGATCTTTTCTACGGGGTCTGACGCTCAGTGGAACGAAAACTCACGTTAAGGGATTTT |
| | | GGTCATGAGATTATCAAAAAGGATCTTCACCTAGATCCTTTTAAATTAAAAATGAAGTTTTAAATC |
| | | AATCTAAAGTATATATGAGTAAACTTGGTCTGACAGTTACCAATGCTTAATCAGTGAGGCACCTAT |
| | | CTCAGCGATCTGTCTATTTCGTTCATCCATAGTTGCCTGACTCCCCGTCGTGTAGATAACTACGAT |
| | | ACGGGAGGGCTTACCATCTGGCCCCAGTGCTGCAATGATACCGCGACACCCACGCTCACCGGCT |
| | | CCAGATTTATCAGCAATAAACCAGCCAGCCGGAAGGGCCGAGCGCAGAAGTGGTCCTGCAACTT |
| | | TATCCGCCTCCATCCAGTCTATTAATTGTTGCCGGGAAGCTAGAGTAAGTAGTTCGCCAGTTAATA |
| | | GTTTGCGCAACGTTGTTGCCATTGCTACAGGCATCGTGGTGTCACGCTCGTCGTTTGGTATGGCTT |
| | | CATTCAGCTCCGGTTCCCAACGATCAAGGCGAGTTACATGATCCCCCATGTTGTGCAAAAAAGCG |
| | | GTTAGCTCCTTCGGTCCTCCGATCGTTGTCAGAAGTAAGTTGGCCGCAGTGTTATCACTCATGGTT |
| | | ATGGCAGCACTGCATAATTCTCTTACTGTCATGCCATCCGTAAGATGCTTTTCTGTGACTGGTGAG |
| | | TACTCAACCAAGTCATTCTGAGAATAGTGTATGCGGCGACCGAGTTGCTCTTGCCCGGCGTCAAT |
| | | ACGGGATAATACCGCGCCACATAGCAGAACTTTAAAAGTGCTCATCATTGGAAAACGTTCTTCGG |
| | | GGCGAAAACTCTCAAGGATCTTACCGCTGTTGAGATCCAGTTCGATGTAACCCACTCGTGCACCC |
| | | AACTGATCTTCAGCATCTTTTACTTTCACCAGCGTTTCTGGGTGAGCAAAAACAGGAAGGCAAAA |
| | | TGCCGCAAAAAAGGGAATAAGGGCGACACGGAAATGTTGAATACTCATACTCTTCCTTTTTCAAT |
| | | ATTATTGAAGCATTTATCAGGGTTATTGTCTCATGAGCGGATACATATTTGAATGTATTTAGAAAA |
| | | ATAAACAAATAGGGGTTCCGCGCACATTTCCCCGAAAAGTGCCACCTGACGTCGACGGATCGGG |
| | | AGATCGATCTCCCGATCCCCTAGGGTCGACTCTCAGTACAATCTGCTCTGATGCCGCATAGTTAA |
| | | GCCAGTATCTGCTCCCTGCTTGTGTGTTGGAGGTCGCTGAGTAGTGCGCGAGCAAAATTTAAGCT |
| | | ACAACAAGGCAAGGCTTGACCGACAATTGCATGAAGAATCTGCTTAGGGTTAGGCGTTTTGCGCT |
| | | GCTTCGCGATGTACGGGCCAGATATACGCGTGAGGGCCTATTTCCCATGATTCCTTCATATTTGCA |
| | | TATACGATACAAGGCTGTTAGAGAGATAATTGGAATTAATTTGACTGTAAACACAAAGATATTAG |
| | | TACAAAATACGTGACGTAGAAAGTAATAATTTCTTGGGTAGTTTGCAGTTTTAAAATTATGTTTTAA |
| | | AATGGACTATCATATGCTTACCGTAACTTGAAAGTATTTCGATTTCTTGGCTTTATATATCTTGTGG |
| | | AAAGGACGAAACACCGGTGGAGCGGTTCACGACCGCGACCTCAACGCCGCCCGGAACATCAAG |
| | | CGGGAAGGGCTTTCGCAAATCGTCGTCGCGGGGCACGCGGAGACGTTAAACGCTCGGGGAGAG |
| | | GGTGTCAGACCTGCGATAGCGGGCAGCCCTCGATGAAGCGAGAATCCAACGGCTTTAGCCGTTG |
| | | GAGTGTCAAGGGCTGTTTTTTATTTTTTGTTTTAGAGCTAGAAATAGTAAGTTAAAATAAGGCTAG |
| | | TCCGTT |
| seq. | vector_ | TTTAGCGCGTGCGCCAATTCTGCAGACAAATGGCTCTAGAGGTACCGACATTGATTATTGACTAG |
| 58 | ISDge10TnpB- | TTATTAATAGTAATCAATTACGGGGTCATTAGTTCATAGCCCATATATGGAGTTCCGCGTTACATA |
| | VPR-reRNA) | ACTTACGGTAAATGGCCCGCCTGGCTGACCGCCCAACGACCCCCGCCCATTGACGTCAATAATG |
| | (20 nt + TnpB | ACGTATGTTCCCATAGTAACGCCAATAGGGACTTTCCATTGACGTCAATGGGTGGAGTATTTACG |
| | D187A)) | GTAAACTGCCCACTTGGCAGTACATCAAGTGTATCATATGCCAAGTACGCCCCCTATTGACGTCA |
| | | ATGACGGTAAATGGCCCGCCTGGCATTATGCCCAGTACATGACCTTATGGGACTTTCCTACTTGG |
| | | CAGTACATCTACGTATTAGTCATCGCTATTACCATGGTGATGCGGTTTTGGCAGTACATCAATGGG |
| | | CGTGGATAGCGGTTTGACTCACGGGGATTTCCAAGTCTCCACCCCATTGACGTCAATGGGAGTTT |
| | | GTTTTGGCACCAAAATCAACGGGACTTTCCAAAATGTCGTAACAACTCCGCCCCATTGACGCAAA |
| | | TGGGCGGTAGGCGTGTACGGTGGGAGGTCTATATAAGCAGAGCTGGTTTAGTGAACCGTCAGAT |
| | | CCGCTAGAGATCCGCGGCCGCGCCACCATGGCCCCAAAGAAGAAGCGGAAGGTCGGTATCCAC |
| | | GGAGTCCCAGCAGCCATGACCACCCACCGCAAGGTGTACAGATACCGCATCGAGCCCACCCCC |
| | | GTGCAGGAGAGCAAGCTGTACATGCTGGCCGGCAGCCGGAGATTCGTGTTCAACTGGGCCCTG |
| | | GCCCGGAGAAGAGAGCACTACGCCGAAACCGGCAAGACCCTGGGATACAACGCCCAGGCAGG |
| | | AGAGCTGACCGCCCTGAAAAACCAAGAAGAAACCAGCTGGCTGAAAGAGTCCGACTCACAACT |
| | | GCTGCAGCAGGCACTGAAAGACGTGGAAAGAGCCTTCGTGAACTTCTTCGAAAAACGGGCCAG |
| | | ATTCCCCAGATTCAAAAGCAAGAAAACCGACACCCCCAGATTCAGAATCCCCCAAAGAGTGAGA |
| | | ATAGAGGGCAGCAGAGTGTACGTGCCCAAGGTGGGGTGGGTGAAGCTGAGAAAGAGTCAGGA |
| | | GATAGAAGGAAAAACCAAAAGCGCCACCTTCAAGAGGGAGGCAGATGGCCATTGGTACGTGCT |
| | | GCTGGTGAGTGAGTTCGAGATGCCTGATGTGCCTCTGCCCCCAGTGCCCGAGAGCGAGGTGGTG |
| | | GGAATCGCCCTGGGACTGAAGGACTTCTACGTCCTGTCTGACGGGGGCAGGAAGGAGGCCCCC |
| | | AGATTCGCTAGAAAGGGCCAGCGAAAACTGAGAAGAGCTGCAAGAAGACATAGCAAGTGCACC |
| | | CGGGGAAGCAACAGAAAGGCCAAGGCAAAGAGGAAGCTGGCAAGAGTGCACAGACAGATCGC |
| | | AAACCAGAGAAAAGACTTCGTGCACAAGGCCACCAGCGGCCTCGTGCAGCAGTACCAAGGCTT |
| | | CTGCATCGAAAACCTGAGCATCAAGGGCATGGCCAAAACCAAACTGTCAAAAAGCGTGCTGGA |
| | | CGCAGCCCTGGGGGAATTTAGAAGACAGCTGGCCTATAAAGCCCAGTGGCATAGAAAGTGGCT |
| | | GGCAGTGATAGACAGATGGTTCCCCAGCAGCAAGCTGTGCGGAGAGTGCGGCAGCATTAACGC |
| | | CGATCTGACCCTGAGCGACAGAGAATGGACCTGCGAGTGTGGCGCAGTGCACGACAGAGATCT |
| | | GAACGCCGCTCGCAACATCAAGAGAGAGGGCCTGAGTCAGATTGTGGTGGCCGGACATGCCGA |
| | | AACCCTGAACGCCAGAGGGGAGGGCGTGAGACCCGCCATTGCCGGAAGCCCTAGAAGCAGGG |
| | | CTGACCCCAAGAAGAAGAGGAAGGTGTCGCCAGGGATCCGTCGACTTGACGCGTTGATATCAAC |
| | | AAGTTTGTACAAAAAAGCAGGCTACAAAGAGGCCAGCGGTTCCGGACGGGCTGACGCATTGGA |
| | | CGATTTTGATCTGGATATGCTGGGAAGTGACGCCCTCGATGATTTTGACCTTGACATGCTTGGTTC |
| | | GGATGCCCTTGATGACTTTGACCTCGACATGCTCGGCAGTGACGCCCTTGATGATTTCGACCTGG |
| | | ACATGCTGATTAACTCTAGAAGTTCCGGATCTCCGAAAAAGAAACGCAAAGTTGGTAGCCAGTA |
| | | CCTGCCCGACACCGACGACCGGCACCGGATCGAGGAAAAGCGGAAGCGGACCTACGAGACATT |
| | | CAAGAGCATCATGAAGAAGTCCCCCTTCAGCGGCCCCACCGACCCTAGACCTCCACCTAGAAGA |
| | | ATCGCCGTGCCCAGCAGATCCAGCGCCAGCGTGCCAAAACCTGCCCCCCAGCCTTACCCCTTCA |
| | | CCAGCAGCCTGAGCACCATCAACTACGACGAGTTCCCTACCATGGTGTTCCCCAGCGGCCAGAT |
| | | CTCTCAGGCCTCTGCTCTGGCTCCAGCCCCTCCTCAGGTGCTGCCTCAGGCTCCTGCTCCTGCAC |
| | | CAGCTCCAGCCATGGTGTCTCCACTGGCTCAGGCACCAGCACCCGTGCCTGTGCTGGCTCCTGG |
| | | ACCTCCACAGGCTGTGGCTCCACCAGCCCCTAAACCTACACAGGCCGGCGAGGGCACACTGTCT |
| | | GAAGCTCTGCTGCAGCTGCAGTTCGACGACGAGGATCTGGGAGCCCTGCTGGGAAACAGCACC |
| | | GATCCTGCCGTGTTCACCGACCTGGCCAGCGTGGACAACAGCGAGTTCCAGCAGCTGCTGAACC |
| | | AGGGCATCCCTGTGGCCCCTCACACCACCGAGCCCATGCTGATGGAATACCCCGAGGCCATCAC |
| | | CCGGCTCGTGACAGGCGCTCAGAGGCCTCCTGATCCAGCTCCTGCCCCTCTGGGAGCACCAGGC |
| | | CTGCCTAATGGACTGCTGTCTGGCGACGAGGACTTCAGCTCTATCGCCGATATGGATTTCTCAGC |
| | | CTTGCTGGGCTCTGGCAGCGGCAGCCGGGATTCCAGGGAAGGGATGTTTTTGCCGAAGCCTGAG |
| | | GCCGGCTCCGCTATTAGTGACGTGTTTGAGGGCCGCGAGGTGTGCCAGCCAAAACGAATCCGGC |
| | | CATTTCATCCTCCAGGAAGTCCATGGGCCAACCGCCCACTCCCCGCCAGCCTCGCACCAACACC |
| | | AACCGGTCCAGTACATGAGCCAGTCGGGTCACTGACCCCGGCACCAGTCCCTCAGCCACTGGAT |
| | | CCAGCGCCCGCAGTGACTCCCGAGGCCAGTCACCTGTTGGAGGATCCCGATGAAGAGACGAGC |
| | | CAGGCTGTCAAAGCCCTTCGGGAGATGGCCGATACTGTGATTCCCCAGAAGGAAGAGGCTGCAA |
| | | TCTGTGGCCAAATGGACCTTTCCCATCCGCCCCCAAGGGGCCATCTGGATGAGCTGACAACCAC |
| | | ACTTGAGTCCATGACCGAGGATCTGAACCTGGACTCACCCCTGACCCCGGAATTGAACGAGATT |
| | | CTGGATACCTTCCTGAACGACGAGTGCCTCTTGCATGCCATGCATATCAGCACAGGACTGTCCAT |
| | | CTTCGACACATCTCTGTTTAAAAGGCCGGCGGCCACGAAAAAGGCCGGCCAGGCAAAAAAGAA |
| | | AAAGATGGCCTCAAACGATTATACCCAACAAGCAACCCAAAGCTATGGGGCCTACCCCACCCAG |
| | | CCCGGGCAGGGCTATTCCCAGCAGAGCAGTCAGCCCTACGGACAGCAGAGTTACAGTGGTTATA |
| | | GCCAGTCCACGGACACTTCAGGCTATGGCCAGAGCAGCTATTCTTCTTATGGCCAGAGCCAGAA |
| | | CACAGGCTATGGAACTCAGTCAACTCCCCAGGGATATGGCTCGACTGGCGGCTATGGCAGTAGC |
| | | CAGAGCTCCCAATCGTCTTACGGGCAGCAGTCCTCCTACCCTGGCTATGGCCAGCAGCCAGCTC |
| | | CCAGCAGCACCTCGGGAAGTTACGGTAGCAGTTCTCAGAGCAGCAGCTATGGGCAGCCCCAGA |
| | | GTGGGAGCTACAGCCAGCAGCCTAGCTATGGTGGACAGCAGCAAAGCTATGGACAGCAGCAAA |
| | | GCTATAATCCCCCTCAGGGCTATGGACAGCAGAACCAGTACAACAGCAGCAGTGGTGGTGGAG |
| | | GTGGAGGTGGAGGTGGAGGTAACTATGGCCAAGATCAATCCTCCATGAGTAGTGGTGGTGGCA |
| | | GTGGTGGCGGTTATGGCAATCAAGACCAGAGTGGTGGAGGTGGCAGCGGTGGCTATGGACAGC |
| | | AGGACCGTGGAGAATTCGGCAGTGGAGAGGGCAGAGGAAGTCTGCTAACATGCGGTGACGTCG |
| | | AGGAGAATCCTGGCCCAGTGAGCAAGGGCGAGGAGCTGTTCACCGGGGTGGTGCCCATCCTGG |
| | | TCGAGCTGGACGGCGACGTAAACGGCCACAAGTTCAGCGTGTCCGGCGAGGGCGAGGGCGATG |
| | | CCACCTACGGCAAGCTGACCCTGAAGTTCATCTGCACCACCGGCAAGCTGCCCGTGCCCTGGCC |
| | | CACCCTCGTGACCACCCTGACCTACGGCGTGCAGTGCTTCAGCCGCTACCCCGACCACATGAAG |
| | | CAGCACGACTTCTTCAAGTCCGCCATGCCCGAAGGCTACGTCCAGGAGCGCACCATCTTCTTCAA |
| | | GGACGACGGCAACTACAAGACCCGCGCCGAGGTGAAGTTCGAGGGCGACACCCTGGTGAACCG |
| | | CATCGAGCTGAAGGGCATCGACTTCAAGGAGGACGGCAACATCCTGGGGCACAAGCTGGAGTA |
| | | CAACTACAACAGCCACAACGTCTATATCATGGCCGACAAGCAGAAGAACGGCATCAAGGTGAA |
| | | CTTCAAGATCCGCCACAACATCGAGGACGGCAGCGTGCAGCTCGCCGACCACTACCAGCAGAA |
| | | CACCCCCATCGGCGACGGCCCCGTGCTGCTGCCCGACAACCACTACCTGAGCACCCAGTCCGCC |
| | | CTGAGCAAAGACCCCAACGAGAAGCGCGATCACATGGTCCTGCTGGAGTTCGTGACCGCCGCC |
| | | GGGATCACTCTCGGCATGGACGAGCTGTACAAGGAATTCTAACTAGAGCTCGCTGATCAGCCTC |
| | | GACTGTGCCTTCTAGTTGCCAGCCATCTGTTGTTTGCCCCTCCCCCGTGCCTTCCTTGACCCTGGA |
| | | AGGTGCCACTCCCACTGTCCTTTCCTAATAAAATGAGGAAATTGCATCGCATTGTCTGAGTAGGT |
| | | GTCATTCTATTCTGGGGGGTGGGGTGGGGCAGGACAGCAAGGGGGAGGATTGGGAAGAGAATA |
| | | GCAGGCATGCTGGGGATGCGGTGGGCTCTATGGCTTCTGAGGCGGAAAGAACCAGCTGGGGCT |
| | | CGATACCGTCGACCTCTAGCTAGAGCTTGGCGTAATCATGGTCATAGCTGTTTCCTGTGTGAAATT |
| | | GTTATCCGCTCACAATTCCACACAACATACGAGCCGGAAGCATAAAGTGTAAAGCCTAGGGTGC |
| | | CTAATGAGTGAGCTAACTCACATTAATTGCGTTGCGCTCACTGCCCGCTTTCCAGTCGGGAAACC |
| | | TGTCGTGCCAGCTGCATTAATGAATCGGCCAACGCGCGGGGAGAGGCGGTTTGCGTATTGGGCG |
| | | CTCTTCCGCTTCCTCGCTCACTGACTCGCTGCGCTCGGTCGTTCGGCTGCGGCGAGCGGTATCAG |
| | | CTCACTCAAAGGCGGTAATACGGTTATCCACAGAATCAGGGGATAACGCAGGAAAGAACATGTG |
| | | AGCAAAAGGCCAGCAAAAGGCCAGGAACCGTAAAAAGGCCGCGTTGCTGGCGTTTTTCCATAG |
| | | GCTCCGCCCCCCTGACGAGCATCACAAAAATCGACGCTCAAGTCAGAGGTGGCGAAACCCGAC |
| | | AGGACTATAAAGATACCAGGCGTTTCCCCCTGGAAGCTCCCTCGTGCGCTCTCCTGTTCCGACCC |
| | | TGCCGCTTACCGGATACCTGTCCGCCTTTCTCCCTTCGGGAAGCGTGGCGCTTTCTCATAGCTCAC |
| | | GCTGTAGGTATCTCAGTTCGGTGTAGGTCGTTCGCTCCAAGCTGGGCTGTGTGCACGAACCCCCC |
| | | GTTCAGCCCGACCGCTGCGCCTTATCCGGTAACTATCGTCTTGAGTCCAACCCGGTAAGACACGA |
| | | CTTATCGCCACTGGCAGCAGCCACTGGTAACAGGATTAGCAGAGCGAGGTATGTAGGCGGTGCT |
| | | ACAGAGTTCTTGAAGTGGTGGCCTAACTACGGCTACACTAGAAGAACAGTATTTGGTATCTGCGC |
| | | TCTGCTGAAGCCAGTTACCTTCGGAAAAAGAGTTGGTAGCTCTTGATCCGGCAAACAAACCACC |
| | | GCTGGTAGCGGTGGTTTTTTTGTTTGCAAGCAGCAGATTACGCGCAGAAAAAAAGGATCTCAAGA |
| | | AGATCCTTTGATCTTTTCTACGGGGTCTGACGCTCAGTGGAACGAAAACTCACGTTAAGGGATTTT |
| | | GGTCATGAGATTATCAAAAAGGATCTTCACCTAGATCCTTTTAAATTAAAAATGAAGTTTTAAATC |
| | | AATCTAAAGTATATATGAGTAAACTTGGTCTGACAGTTACCAATGCTTAATCAGTGAGGCACCTAT |
| | | CTCAGCGATCTGTCTATTTCGTTCATCCATAGTTGCCTGACTCCCCGTCGTGTAGATAACTACGAT |
| | | ACGGGAGGGCTTACCATCTGGCCCCAGTGCTGCAATGATACCGCGAGACCCACGCTCACCGGCT |
| | | CCAGATTTATCAGCAATAAACCAGCCAGCCGGAAGGGCCGAGCGCAGAAGTGGTCCTGCAACTT |
| | | TATCCGCCTCCATCCAGTCTATTAATTGTTGCCGGGAAGCTAGAGTAAGTAGTTCGCCAGTTAATA |
| | | GTTTGCGCAACGTTGTTGCCATTGCTACAGGCATCGTGGTGTCACGCTCGTCGTTTGGTATGGCTT |
| | | CATTCAGCTCCGGTTCCCAACGATCAAGGCGAGTTACATGATCCCCCATGTTGTGCAAAAAAGCG |
| | | GTTAGCTCCTTCGGTCCTCCGATCGTTGTCAGAAGTAAGTTGGCCGCAGTGTTATCACTCATGGTT |
| | | ATGGCAGCACTGCATAATTCTCTTACTGTCATGCCATCCGTAAGATGCTTTTCTGTGACTGGTGAG |
| | | TACTCAACCAAGTCATTCTGAGAATAGTGTATGCGGCGACCGAGTTGCTCTTGCCCGGCGTCAAT |
| | | ACGGGATAATACCGCGCCACATAGCAGAACTTTAAAAGTGCTCATCATTGGAAAACGTTCTTCGG |
| | | GGCGAAAACTCTCAAGGATCTTACCGCTGTTGAGATCCAGTTCGATGTAACCCACTCGTGCACCC |
| | | AACTGATCTTCAGCATCTTTTACTTTCACCAGCGTTTCTGGGTGAGCAAAAACAGGAAGGCAAAA |
| | | TGCCGCAAAAAAGGGAATAAGGGCGACACGGAAATGTTGAATACTCATACTCTTCCTTTTTCAAT |
| | | ATTATTGAAGCATTTATCAGGGTTATTGTCTCATGAGCGGATACATATTTGAATGTATTTAGAAAA |
| | | ATAAACAAATAGGGGTTCCGCGCACATTTCCCCGAAAAGTGCCACCTGACGTCGACGGATCGGG |
| | | AGATCGATCTCCCGATCCCCTAGGGTCGACTCTCAGTACAATCTGCTCTGATGCCGCATAGTTAA |
| | | GCCAGTATCTGCTCCCTGCTTGTGTGTTGGAGGTCGCTGAGTAGTGCGCGAGCAAAATTTAAGCT |
| | | ACAACAAGGCAAGGCTTGACCGACAATTGCATGAAGAATCTGCTTAGGGTTAGGCGTTTTGCGCT |
| | | GCTTCGCGATGTACGGGCCAGATATACGCGTGAGGGCCTATTTCCCATGATTCCTTCATATTTGCA |
| | | TATACGATACAAGGCTGTTAGAGAGATAATTGGAATTAATTTGACTGTAAACACAAAGATATTAG |
| | | TACAAAATACGTGACGTAGAAAGTAATAATTTCTTGGGTAGTTTGCAGTTTTAAAATTATGTTTTAA |
| | | AATGGACTATCATATGCTTACCGTAACTTGAAAGTATTTCGATTTCTTGGCTTTATATATCTTGTGG |
| | | AAAGGACGAAACACCGGTGGAGCGGTTCACGACCGCGACCTCAACGCCGCCCGGAACATCAAG |
| | | CGGGAAGGGCTTTCGCAAATCGTCGTCGCGGGGCACGCGGAGACGTTAAACGCTCGGGGAGAG |
| | | GGTGTCAGACCTGCGATAGCGGGCAGCCCTCGATGAAGCGAGAATCCAACGGCTTTAGCCGTTG |
| | | GAGTGTCAAGGGCTGTTGTCAGACCCAACTTTTATTTTTTGTTTTAGAGCTAGAAATAGTAAGTTA |
| | | AAATAAGGCTAGTCCGTT |

The operating efficiency of each system was analyzed by the change in expression level of RFP fluorescence by inducing transcription activity according to 5 the operation of ISDge10TnpB-VPR. Here, the time for inducing strong expression of the target gene was 48 or 72 hours.

As a result of the gene expression activation of ISDge10TnpB-VPR by reRNA regulation (FIG. 2C and FIG. 2D), compared to the version with a mutation that eliminated DNA cleavage activity, the gene expression activation was further increased when the spacer length of gRNA or reRNA was regulated. In addition, the shorter the length of reRNA, the more it induced target gene expression activation without inducing DNA cleavage.

Specifically, ISDge10TnpB-VPR was shown to exhibit higher expression activation when reRNA with a spacer of 8 to 14 nt, particularly 10 nt, was used, compared to the amino acid-substituted ISDge10TnpB (D187A)-VPR. Furthermore, ISDge10TnpB-VPR exhibited excellent regulation of expression after 72 hours of operation, compared to 48-hour operation.

In addition, as a result of the T7E1 assay (FIGS. 2E and 2F), it was confirmed that no indels occurred in the version with higher expression activation. According to the above results, an engineered reRNA configuration based on ISDge10TnpB has been established that safely activates target gene expression without causing indels in the target DNA through reRNA engineering, thereby constructing an optimized ultra-miniaturized activation system (ISDge10TnpB-VPR).

Adeno-associated viruses (AAVs) enable relatively safe intracellular delivery of a foreign gene due to low immunogenicity and a low possibility of human gene insertion. Since the size of a gene enabling expression regulation by the ultra-miniaturized ISDge10TnpB-based activation system is within the maximum load capacity of the foreign gene into the AAV system, it is possible to load all genes into a single vector (all-in-one). By introducing such a vector into a target human-derived cell line (HEK293FT), it is possible to produce recombinant AAVs (rAAVs) loading the ultra-miniaturized gene activation system (ISDge10TnpB-VPR) with a high activity titer. Therefore, due to its very small size, the optimized gene regulatory system constructed in Example 2 can be loaded even when multiple additional modules are combined, demonstrating high potential for broad application (FIG. 2G).

### Example 3. Engineering of Scaffold Sequence of reRNA

The reRNA recognized by ISDge10TnpB consists of a spacer part hybridized with DNA and a scaffold sequence recognized by ISDge10TnpB. In Example 2, engineering for the spacer part was performed. In Example 3, due to the long scaffold sequence, to develop a more compact gene expression system, not only the length of the spacer part but also that of the scaffold sequence were adjusted. Here, the scaffold sequence of reRNA recognized by TnpB of ISDge10TnpB was used to predict the structure of the system by a structure prediction program, and portions that form a specific structure, such as a loop structure, were named in order from 1 to 8. Each portion was removed to adjust the length of the scaffold sequence, thereby comparing and confirming the efficiency of gene cleavage in cells.

First, reRNAs in which the scaffold sequence was engineered were constructed. Specifically, SEQ ID NOs: 60 to 70 which include 10 spacer sequences and have engineered scaffold sequences, were manufactured by removing a certain range of nucleotides from the 5′ end based on SEQ ID NO: 23 (Table 4). Here, reRNA_DNMT1-D1 (10 nt) refers to reRNA with 10 spacer sequences contained in the scaffold sequence from which portion 1 was removed, and other names may be interpreted by the same rules. In addition, D12 reRNA may mean reRNA with the scaffold sequence from which both portions 1 and 2 were removed, D123 reRNA may mean the scaffold sequence from which all of portions 1 to 3 were removed, and D1238delta reRNA may mean the scaffold sequence from which all of portions 1 to 3 and 8 were removed.

TABLE 4

| seq. | name. | modification |
|---|---|---|
| seq. 60 | reRNA_DNMT1-D1 | Removal of the sequence of |
| | (10nt) | nucleotides 7 to 21 |
| seq. 61 | reRNA_DNMT1-D2 | Removal of the sequence of |
| | (10nt) | nucleotides 22 to 30 |
| seq. 62 | reRNA DNMT1-D3 | Removal of the sequence of |
| | (10nt) | nucleotides 31 to 58 |
| seq. 63 | reRNA_DNMT1-D4 | Removal of the sequence of |
| | (10nt) | nucleotides 69 to 93 |
| seq. 64 | reRNA_DNMT1-D5 | Removal of the sequence of |
| | (10nt) | nucleotides 94 to 108 |
| seq. 65 | reRNA_DNMT1-D6 | Removal of the sequence of |
| | (10nt) | nucleotides 109 to 142 |
| seq. 66 | reRNA_DNMT1-D7 | Removal of the sequence of |
| | (10nt) | nucleotides 156 to 177 |
| seq. 67 | eRNA_DNMT1-D8delta | Removal of the sequence of |
| | (10nt) | nucleotides 59 to 68 |
| seq. 68 | reRNA_DNMT1-D12 | Removal of the entire sequence of |
| | (10nt) | nucleotides 7 to 30 |
| seq. 69 | eRNA_DNMT1-D123 | Removal of the entire sequence of |
| | (10nt) | nucleotides 7 to 58 |
| seq. 70 | reRNA_DNMT1- | Removal of the entire sequence of |
| | D1238delta (10nt) | nucleotides 7 to 68 |

The sequences of the reRNAs. D1 to D8delta, D12. D123, and D1238delta. constructed according to the above changes are shown in Table 5.

TABLE 5

| No. | Name | sequence(5′→3′) |
|---|---|---|
| seq. | reRNA_ | gGTGGAGACCTCAACGCCGCCCGGAACATCAAG |
| 60 | DNMT1- | CGGGAAGGGCTTTCGCAAATCGTCGTCGCGGGG |
| | D1 | CACGCGGAGACGTTAAACGCTCGGGGAGAGGGT |
| | (10 nt) | GTCAGACCTGCGATAGCGGGCAGCCCTCGAtga |
| | | AGCGAGAATCCAACGGCTTTAGCCGTTGGAGTG |
| | | TCAAGGGCTGTTGTttttatttt |
| seq. | reRNA_ | gGTGGAGCGGTTCACGACCGCGCCCGGAACATC |
| 61 | DNMT1- | AAGCGGGAAGGGCTTTCGCAAATCGTCGTCGCG |
| | D2 | GGGCACGCGGAGACGTTAAACGCTCGGGGAGAG |
| | (10 nt) | GGTGTCAGACCTGCGATAGCGGGCAGCCCTCGA |
| | | tgaAGCGAGAATCCAACGGCTTTAGCCGTTGGA |
| | | GTGTCAAGGGCTGTTGTttttatttt |
| seq. | reRNA_ | gGTGGAGCGGTTCACGACCGCGACCTCAACTTT |
| 62 | DNMT1- | CGCAAATCGTCGTCGCGGGGCACGCGGAGACGT |
| | D3 | TAAACGCTCGGGGAGAGGGTGTCAGACCTGCGA |
| | (10 nt) | TAGCGGGCAGCCCTCGAtgaAGCGAGAATCCAA |
| | | CGGCTTTAGCCGTTGGAGTGTCAAGGGCTGTTG |
| | | Tttttatttt |
| seq. | reRNA_ | gGTGGAGCGGTTCACGACCGCGACCTCAACGCC |
| 63 | DNMT1- | GCCCGGAACATCAAGCGGGAAGGGCTTTCGCAA |
| | D4 | ATTTAAACGCTCGGGGAGAGGGTGTCAGACCTG |
| | (10 nt) | CGATAGCGGGCAGCCCTCGAtgaAGCGAGAATC |
| | | CAACGGCTTTAGCCGTTGGAGTGTCAAGGGCTG |
| | | TTGTttttatttt |
| seq. | reRNA_ | gGTGGAGCGGTTCACGACCGCGACCTCAACGCC |
| 64 | DNMT1- | GCCCGGAACATCAAGCGGGAAGGGCTTTCGCAA |
| | D5 | ATCGTCGTCGCGGGGCACGCGGAGACGGAGGGT |
| | (10 nt) | GTCAGACCTGCGATAGCGGGCAGCCCTCGAtga |
| | | AGCGAGAATCCAACGGCTTTAGCCGTTGGAGTG |
| | | TCAAGGGCTGTTGTttttatttt |
| seq. | reRNA_ | gGTGGAGCGGTTCACGACCGCGACCTCAACGCC |
| 65 | DNMT1- | GCCCGGAACATCAAGCGGGAAGGGCTTTCGCAA |
| | D6 | ATCGTCGTCGCGGGGCACGCGGAGACGTTAAAC |
| | (10 nt) | GCTCGGGGAGAtgaAGCGAGAATCCAACGGCTT |
| | | TAGCCGTTGGAGTGTCAAGGGCTGTTGTtttta |
| | | tttt |
| seq. | reRNA_ | gGTGGAGCGGTTCACGACCGCGACCTCAACGCC |
| 66 | DNMT1- | GCCCGGAACATCAAGCGGGAAGGGCTTTCGCAA |
| | D7 | ATCGTCGTCGCGGGGCACGCGGAGACGTTAAAC |
| | (10 nt) | GCTCGGGGAGAGGGTGTCAGACCTGCGATAGCG |
| | | GGCAGCCCTCGAtgaAGCGAGAAGTGTCAAGGG |
| | | CTGTTGTttttatttt |
| seq. | [symbol] RNA_ | gGTGGAGCGGTTCACGACCGCGACCTCAACGCC |
| 67 | DNMT1- | GCCCGGAACATCAAGCGGGAAGGGCCGTCGTCG |
| | D8delta | CGGGGCACGCGGAGACGTTAAACGCTCGGGGAG |
| | (10 nt) | AGGGTGTCAGACCTGCGATAGCGGGCAGCCCTC |
| | | GATGAAGCGAGAATCCAACGGCTTTAGCCGTTG |
| | | GAGTGTCAAGGGCTGTTGTttttatttt |
| seq. | reRNA_ | gGTGGAGCCGCCCGGAACATCAAGCGGGAAGGG |
| 68 | DNMT1- | CTTTCGCAAATCGTCGTCGCGGGGCACGCGGAG |
| | D12 | ACGTTAAACGCTCGGGGAGAGGGTGTCAGACCT |
| | (10 nt) | GGGATAGCGGGCAGCCCTCGAtgaAGCGAGAAT |
| | | CCAACGGCTTTAGCCGTTGGAGTGTCAAGGGCT |
| | | GTTGTttttatttt |
| seq. | [symbol] RNA_ | gGTGGATTTCGCAAATCGTCGTCGCGGGGCACG |
| 69 | DNMT1- | CGGAGACGTTAAACGCTCGGGGAGAGGGTGTCA |
| | D123 | GACCTGCGATAGCGGGCAGCCCTCGAtgaAGCG |
| | (10 nt) | AGAATCCAACGGCTTTAGCCGTTGGAGTGTCAA |
| | | GGGCTGTTGTttttatttt |
| seq. | reRNA_ | gGTGGACGTCGTCGCGGGGCACGCGGAGACGTT |
| 70 | DNMT1- | AAACGCTCGGGGAGAGGGTGTCAGACCTGCGAT |
| | D1238delta | AGCGGGCAGCCCTCGAtgaAGCGAGAATCCAAC |
| | (10 nt) | GGCTTTAGCCGTTGGAGTGTCAAGGGCTGTTGT |
| | | ttttatttt |
| seq. | reRNA_ | gGTGGAGACCTCAACGCCGCCCGGAACATCAAG |
| 71 | DNMT1- | CGGGAAGGGCTTTCGCAAATCGTCGTCGCGGGG |
| | D1 | CACGCGGAGACGTTAAACGCTCGGGGAGAGGGT |
| | (20 nt) | GTCAGACCTGCGATAGCGGGCAGCCCTCGAtga |
| | | AGCGAGAATCCAACGGCTTTAGCCGTTGGAGTG |
| | | TCAAGGGCTGTTGTCAGACCCAACttttatttt |
| seq. | reRNA_ | gGTGGAGCGGTTCACGACCGCGCCGCCCGGAAC |
| 72 | DNMT1- | ATCAAGCGGGAAGGGCTTTCGCAAATCGTCGTC |
| | D2 | GCGGGGCACGCGGAGACGTTAAACGCTCGGGGA |
| | (20 nt) | GAGGGTGTCAGACCTGCGATAGCGGGCAGCCCT |
| | | CGAtgaAGCGAGAATCCAACGGCTTTAGCCGTT |
| | | GGAGTGTCAAGGGCTGTTGTCAGACCCAACttt |
| | | tatttt |
| seq. | reRNA_ | gGTGGAGCGGTTCACGACCGCGACCTCAACTTT |
| 73 | DNMT1- | CGCAAATCGTCGTCGCGGGGCACGCGGAGACGT |
| | D3 | TAAACGCTCGGGGAGAGGGTGTCAGACCTGCGA |
| | (20 nt) | TAGCGGGCAGCCCTCGAtgaAGCGAGAATCCAA |
| | | CGGCTTTAGCCGTTGGAGTGTCAAGGGCTGTTG |
| | | TCAGACCCAACttttatttt |
| seq. | reRNA_ | gGTGGAGCGGTTCACGACCGCGACCTCAACGCC |
| 74 | DNMT1- | GCCCGGAACATCAAGCGGGAAGGGCTTTCGCAA |
| | D4 | ATTTAAACGCTCGGGGAGAGGGTGTCAGACCTG |
| | (20 nt) | CGATAGCGGGCAGCCCTCGAtgaAGCGAGAATC |
| | | CAACGGCTTTAGCCGTTGGAGTGTCAAGGGCTG |
| | | TTGTCAGACCCAACttttatttt |
| seq. | reRNA_ | gGTGGAGCGGTTCACGACCGCGACCTCAACGCC |
| 75 | DNMT1- | GCCCGGAACATCAAGCGGGAAGGGCTTTCGCAA |
| | D5 | ATCGTCGTCGCGGGGCACGCGGAGACGGAGGGT |
| | (20 nt) | GTCAGACCTGCGATAGCGGGCAGCCCTCGAtga |
| | | AGCGAGAATCCAACGGCTTTAGCCGTTGGAGTG |
| | | TCAAGGGCTGTTGTCAGACCCAACttttatttt |
| seq. | reRNA_ | gGTGGAGCGGTTCACGACCGCGACCTCAACGCC |
| 76 | DNMT1- | GCCCGGAACATCAAGCGGGAAGGGCTTTCGCAA |
| | D6 | ATCGTCGTCGCGGGGCACGCGGAGACGTTAAAC |
| | (20 nt) | GCTCGGGGAGAtgaAGCGAGAATCCAACGGCTT |
| | | TAGCCGTTGGAGTGTCAAGGGCTGTTGTCAGAC |
| | | CCAACttttatttt |
| seq. | reRNA_ | gGTGGAGCGGTTCACGACCGCGACCTCAACGCC |
| 77 | DNMT1- | GCCCGGAACATCAAGCGGGAAGGGCTTTCGCAA |
| | D7 | ATCGTCGTCGCGGGGCACGCGGAGACGTTAAAC |
| | (20 nt) | GCTCGGGGAGAGGGTGTCAGACCTGCGATAGCG |
| | | GGCAGCCCTCGAtgaAGCGAGAAGTGTCAAGGG |
| | | CTGTTGTCAGACCCAACttttatttt |
| seq. | DNMT1- | gGTGGAGCGGTTCACGACCGCGACCTCAACGCC |
| 78 | D8delta | GCCCGGAACATCAAGCGGGAAGGGCCGTCGTCG |
| | (20 nt) | CGGGGCACGCGGAGACGTTAAACGCTCGGGGAG |
| | | AGGGTATCAGACCTGCGATAGCGGGCAGCCCTC |
| | | GATGAAGCGAGAATCCAACGGCTTTAGCCGTTG |
| | | GAGTGTCAAGGGCTGTTGTCAGACCCAACtttt |
| | | atttt |
| seq. | reRNA_ | gGTGGAGCCGCCCGGAACATCAAGCGGGAAGGG |
| 79 | DNMT1- | CTTTCGCAAATCGTCGTCGCGGGGCACGCGGAG |
| | D12 | ACGTTAAACGCTCGGGGAGAGGGTGTCAGACCT |
| | (20 nt) | GCGATAGCGGGCAGCCCTCGAtgaAGCGAGAAT |
| | | CCAACGGCTTTAGCCGTTGGAGTGTCAAGGGCT |
| | | GTTGTCAGACCCAACttttatttt |
| seq. | reRNA_ | gGTGGATTTCGCAAATCGTCGTCGCGGGGCACG |
| 80 | DNMT1- | CGGAGACGTTAAACGCTCGGGGAGAGGGTGTCA |
| | D123 | GACCTGCGATAGCGGGCAGCCCTCGAtgaAGCG |
| | (20 nt) | AGAATCCAACGGCTTTAGCCGTTGGAGTGTCAA |
| | | GGGCTGTTGTCAGACCCAACttttatttt |
| seq. | reRNA_ | gGTGGACGTCGTCGCGGGGCACGCGGAGACGTT |
| 81 | DNMT1- | AAACGCTCGGGGAGAGGGTGTCAGACCTGCGAT |
| | D1238delta | AGCGGGCAGCCCTCGAtgaAGCGAGAATCCAAC |
| | (20 nt) | GGCTTTAGCCGTTGGAGTGTCAAGGGCTGTTGT |
| | | CAGACCCAACttttatttt |

[symbol]

indicates data missing or illegible when filed

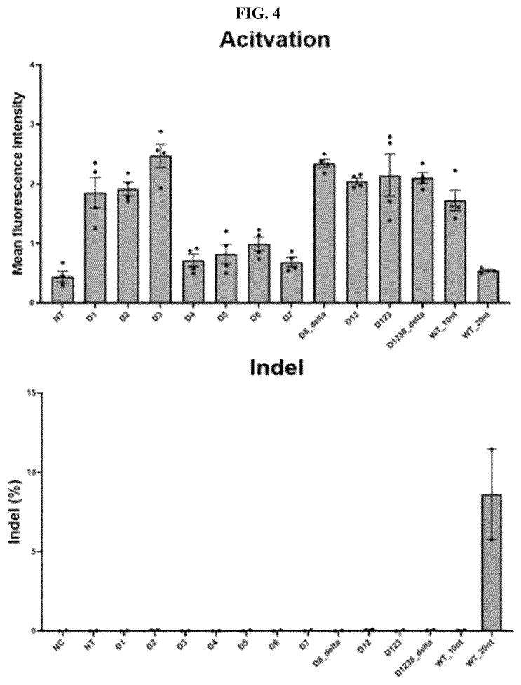

The results of comparing the efficiency of intracellular gene cleavage using the reRNAs in Table 4 were confirmed as shown in FIG. 3.

Specifically, it was confirmed that the cleavage efficiency was maintained in the reRNA versions in which the length of the scaffold sequence was shortened by removing D1, D2, D3, and D8. In addition, by combining these reRNAs, it was confirmed that the cleavage efficiency of reRNAs including the smallest version D1238delta manufactured by removing all of portions 1 to 3 and 8 from the scaffold sequence was not significantly different from that of reRNA with the original entire sequence even through the scaffold sequence of the reRNA was reduced from 183 nt to 121 nt.

Accordingly, through engineering of the scaffold sequence, reRNA that is smaller in size but can perform a gene expression regulation function was identified.

### Example 4. Confirmation of Gene Expression Efficiency Using Engineered Scaffold Sequence

The gene activation efficiency of various versions with an engineered scaffold sequence of the reRNA recognized by ISDge10TnpB constructed in Example 3 was compared using a reporter system. Here, WT_10nt, and WT_20nt were used as controls. WT_10nt or WT_20nt means reRNA including a 10nt or 20nt spacer 10 without applying engineering for each scaffold sequence.

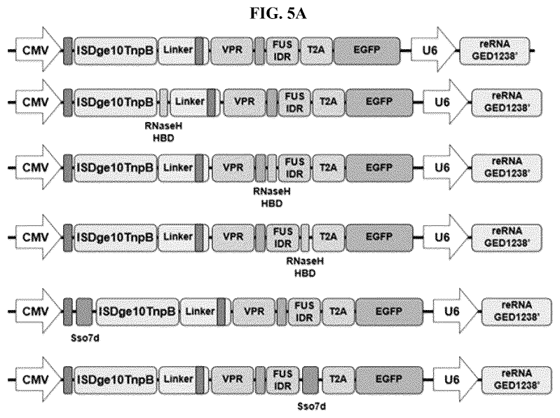

As a result, in all versions using the reRNA with the 10-nt spacer identified to exhibit the most efficient gene expression in Example 2, it was confirmed that the activation of gene expression efficiency was effectively induced without cleavage efficiency (FIG. 4).

Particularly, the gene expression rate of the smallest version D1238delta was found to be similar to WT_10nt. According to the above-described experimental results, the experimental group with the 10-nt spacer sequence and engineered scaffold sequence was demonstrated to induce no gene cleavage, but to have the same or a similar level of gene expression activation compared to that of the original version without an engineered scaffold sequence.

On the other hand, WT_20nt with reRNA containing a full-length spacer and un-engineered scaffold sequence efficiently induced cleavage, resulting in lower gene activation, contrary to the above results.

Consequently, it was confirmed that, when the scaffold sequence was additionally engineered in the reRNA in which the spacer sequence of the present invention was engineered, gene expression activation was only effectively induced without a gene cleavage effect. By engineering both the spacer sequence and the scaffold sequence of reRNA in ISDge10TnpB, ultrasmall reRNA was developed and an ultra-miniaturized gene expression system capable of regulating gene expression was effectively and safely constructed.

### Example 5. Confirmation of Gene Expression Efficiency Using Engineered Scaffold Sequence

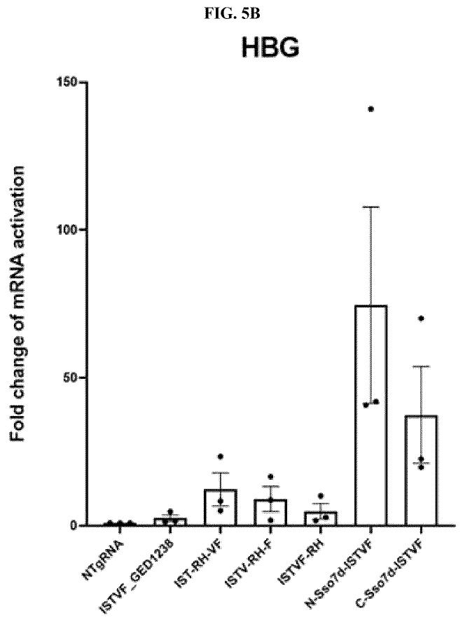

Various domains were connected to increase the efficiency of ISDge10TnpB-based gene activation technology. A hybridization domain (HBD) and a DNA-binding domain Sso7d of RNaseH recognizing a DNA-RNA duplex were used as domains used herein. That is, HBD or Sso7d was connected to various locations of ISDge10TnpB-VPR, thereby confirming whether a change in gene expression level in cells was induced. Here, for all experimental groups, when the length of the spacer sequence was fixed to 10 nt and the scaffold sequence was fixed to D1238, a vector was designed as shown in FIG. 5A to compare the gene expression efficiency by changing only the type or location of a domain. In addition, NTgRNA was used as a comparative group. NTgRNA is non-targeting reRNA that has engineered scaffold and spacer sequences but lacks a genetic information sequence that does not target the target position HBG gene, and is represented by SEQ ID NO: 70. Since only the spacer sequence information does not have a target gene sequence, NTgRNA was set as a control to confirm whether the above-mentioned result is the non-specific TnpB effect.

TABLE 6

| No. | Name | sequence (5'→3') |
|---|---|---|
| seq. | RNaseH | [symbol] |
| 82 | HBD | [symbol] |
| | | [symbol] |
| seq. | RNaseH | [symbol] |
| 83 | HBD_AA | |
| seq. | Sso7d | [symbol] |
| 84 | | [symbol] |
| | | [symbol] |
| seq. | Sso7d_AA | [symbol] |
| 85 | | |
| seq. | ISTVF_GED | [symbol] |
| 86 | 1238 | [symbol] |
| | (SV40NLS- | [symbol] |
| | ISDge10TnpB- | [symbol] |
| | linker- | [symbol] |
| | VPR-NulceoplasminNLS- | [symbol] |
| | FUS | [symbol] |
| | IDR) | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| seq. | ISTVF-GED | [symbol] |
| 87 | 1238 | [symbol] |
| | (SV40NLS- | [symbol] |
| | ISDge10TnpB- | [symbol] |
| | linker- | [symbol] |
| | VPR-NulceoplasminNLS- | [symbol] |
| | FUS | [symbol] |
| | IDR)_AA | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| seq. | IST-RH-VF | [symbol] |
| 88 | (SV40NLS- | [symbol] |
| | ISDge10TnpB- | [symbol] |
| | linker- | [symbol] |
| | RNaseH | [symbol] |
| | HBD-linker | [symbol] |
| | VPR-NulceoplasminNLS- | [symbol] |
| | FUS | [symbol] |
| | IDR) | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| seq. | IST-RH-VF | [symbol] |
| 89 | (SV40NLS- | [symbol] |
| | ISDge10TnpB- | [symbol] |
| | linker- | [symbol] |
| | RNaseH | [symbol] |
| | HBD-linker- | [symbol] |
| | VPR-NulceoplasminNLS- | [symbol] |
| | FUS | [symbol] |
| | IDR)_AA | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| seq. | ISTV-RH-F | [symbol] |
| 90 | (SV40NLS- | [symbol] |
| | ISDge10TnpB- | [symbol] |
| | linker- | [symbol] |
| | VPR-NulceoplasminNLS- | [symbol] |
| | linker-RNbaseH | [symbol] |
| | HBD-linker- | [symbol] |
| | FUS IDR) | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| seq. | ISTV-RH-F | [symbol] |
| 91 | (SV40NLS- | [symbol] |
| | ISDge10TnpB- | [symbol] |
| | linker- | [symbol] |
| | VPR-NulceoplasminNLS- | [symbol] |
| | linker-RNaseH | [symbol] |
| | HBD-linker- | [symbol] |
| | FUS | [symbol] |
| | IDR)_AA | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| seq. | ISTVF-RH | [symbol] |
| 92 | (SV40NLS- | [symbol] |
| | ISDge10Tn-pB- | [symbol] |
| | linker- | [symbol] |
| | VPR-NulceoplasminNLS- | [symbol] |
| | FUS | [symbol] |
| | IDR-linker- | [symbol] |
| | RNaseH | [symbol] |
| | HBD) | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| seq. | ISTVF-RH | [symbol] |
| 93 | (SV40NLS- | [symbol] |
| | ISDge10Tn-pB- | [symbol] |
| | linker- | [symbol] |
| | VPR-NulceoplasminNLS- | [symbol] |
| | FUS | [symbol] |
| | IDR-linker- | [symbol] |
| | RNaseH | [symbol] |
| | HBD)_AA | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| seq. | N-Sso7d-ISTVF | [symbol] |
| 94 | (SV40NLS- | [symbol] |
| | Sso7d-linker- | [symbol] |
| | ISDge10TnpB- | [symbol] |
| | linker- | [symbol] |
| | VPR-NulceplasminNLS- | [symbol] |
| | FUS | [symbol] |
| | IDR) | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| seq. | N-Sco7d-ISTVF | [symbol] |
| 95 | (SV40NLS- | [symbol] |
| | Sso7d-linker- | [symbol] |
| | ISDge10TnpB- | [symbol] |
| | linker- | [symbol] |
| | VPR-NulceplasminNLS- | [symbol] |
| | FUS | [symbol] |
| | IDR)_AA | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| seq. | C-Sso7d-ISTVF | [symbol] |
| 96 | (SV40NLS- | [symbol] |
| | ISDge10TnpB- | [symbol] |
| | linker- | [symbol] |
| | VPR-NulceoplasminNLS- | [symbol] |
| | FUS | [symbol] |
| | IDR-linker- | [symbol] |
| | Sso7d) | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| seq. | C-Sso7d-ISTVF | [symbol] |
| 97 | ISV40NLS- | [symbol] |
| | ISDge10TnpB- | [symbol] |
| | linker- | [symbol] |
| | VPR-NulceoplasminNLS- | [symbol] |
| | FUS | [symbol] |
| | IDR-linker- | [symbol] |
| | Sso7d)_AA | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| seq. | ISTVF_GED | [symbol] |
| 98 | 1238 | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| seq. | IST-RH-VF | [symbol] |
| 99 | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| seq. | ISTV-RH-F | [symbol] |
| 100 | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| seq. | ISTVF-RH | [symbol] |
| 101 | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| seq. | N-Sso7d-ISTVF | [symbol] |
| 102 | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| seq. | C-Sso7d-ISTVF | [symbol] |
| 103 | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |
| | | [symbol] |

[symbol]

indicates data missing or illegible when filed

As a result (FIG. 5B), it was shown that gene activation was more effectively induced when HBD or Sso7d were connected than when no domain was added to ISTVF_GED1238. Particularly, when the Sso7d domain was connected to the N-terminus, it was confirmed that the highest gene expression activation was induced.

Consequently, an ultra-miniaturized gene regulatory system was developed that efficiently and safely regulates gene expression by inducing reRNA engineering of ISDge10TnpB and connecting a specific domain to ISDge10TnpB.

The foregoing description of the present invention is intended for illustrative purposes, and it will be understood by those of ordinary skill in the art to which the invention pertains that various modifications may be made in other specific forms without altering the technical spirit or essential characteristics of the present invention. Therefore, the embodiments described above are to be understood in all respects as illustrative and not restrictive.

What is claimed is:

1. A method of regulating target gene expression, comprising: introducing into a host cell a composition comprising a first component comprising a transposon-associated ribonucleoprotein (TnpB) and a second component comprising a right-end transposon element-derived RNA (reRNA) as active ingredients; or transfecting a host cell with the vector comprising a nucleotide sequence encoding the first component and the second component,
wherein the composition comprises first and second components each selected from the group consisting of the following, in which the first component is any one selected from the group consisting of:
i) TnpB comprising an amino acid sequence represented by SEQ ID NOL 28; and
ii) TnpB engineered to inactivate its DNA cleavage activity; and
the second component is any one selected from the group consisting of:
i) reRNA comprising a nucleotide sequence represented by SEQ ID NO: 18;
ii) engineered reRNA comprising a scaffold sequence and a spacer sequence, in which the reRNA further comprises a spacer sequence based on SEQ ID NO: 39 and is engineered to have a spacer sequence length of 2 to 20 nt; and
iii) reRNA comprising an additionally engineered scaffold sequence and a spacer sequence, in which the reRNA comprises a scaffold sequence that is the same or added based on engineered reRNA comprising SEQ ID NO: 70 and is further engineered to have a scaffold sequence length of 121 to 183 nt.

2. The method of claim 1, wherein the engineered TnpB has a mutation at the 187th amino acid from the N-terminus of the amino acid sequence represented by SEQ ID NO:28.

3. The method of claim 2, wherein the engineered TnpB comprises an amino acid sequence with the D187A mutation from the N-terminus of the amino acid sequence represented by SEQ ID NO:28, and wherein the engineered TnpB is encoded by a nucleotide sequence comprising SEQ ID NO: 29.

4. The method of claim 1, wherein the ii) engineered reRNA comprises any one nucleotide sequence selected from the group consisting of SEQ ID NOs: 19 to 24.

5. The method of claim 1, wherein the iii) reRNA with an additionally engineered scaffold sequence is further engineered so that one or more nucleotide from nucleotides 7 to 68 from the 5′ end of a nucleotide sequence represented by SEQ ID NO: 23 is removed.

6. The method of claim 5, wherein the iii) reRNA with an additionally engineered scaffold sequence comprises any one nucleotide sequence selected from the group consisting of SEQ ID NOs: 60 to 70.

7. The method of claim 1, wherein the ii) engineered reRNA or the iii) reRNA with an additionally engineered scaffold sequence induces inactivation of the DNA cleavage activity of TnpB.

8. The method of claim 1, wherein each of the first and second components is any one selected from the group consisting of the following:
i) engineered TnpB comprising an amino acid sequence represented by SEQ ID NO: 30, and reRNA comprising the nucleotide sequence represented by SEQ ID NO: 18;
ii) TnpB comprising the amino acid sequence represented by SEQ ID NO: 28, and reRNA or engineered reRNA comprising any one nucleotide sequence selected from the group consisting of SEQ ID NOs: 18 to 24; and
iii) TnpB comprising the amino acid sequence represented by SEQ ID NO: 28, and reRNA with an additionally engineered scaffold sequence comprising any one nucleotide sequence selected from the group consisting of SEQ ID NOs: 60 to 70.

9. The method of claim 1, wherein the first component comprises an amino acid sequence represented by SEQ ID NO: 44 or 46.

10. The method of claim 1, wherein the first and second components are included as separate compositions or the same composition, and wherein the first and second components are simultaneously, separately, or sequentially administered.

11. A kit comprising the composition of claim 1 or the vector of claim 1; and instruction.

12. An ultra-miniaturized vector for regulating target gene expression, comprising a nucleotide sequence encoding the first component of claim 1 and the second component of claim 1.

13. The vector of claim 12, which comprises any one selected from the group consisting of the following:
i) a nucleotide sequence encoding any one selected from the group consisting of TnpB comprising an amino acid sequence represented by SEQ ID NO: 28, and the engineered TnpB of claim 1; and
ii) any one nucleotide sequence selected from the group consisting of reRNA comprising a nucleotide sequence represented by SEQ ID NO: 18, the engineered reRNA of claim 1, and the reRNA with an additionally engineered scaffold sequence of claim 1.

14. The vector of claim 13, which comprises any one nucleotide sequence selected from the group consisting of the following:
i) a nucleotide sequence encoding engineered TnpB comprising an amino acid sequence represented by SEQ ID NO: 30, and reRNA comprising the nucleotide sequence represented by SEQ ID NO: 18;
ii) a nucleotide sequence encoding TnpB comprising the amino acid sequence represented by SEQ ID NO: 28, and reRNA or engineered reRNA comprising any one nucleotide sequence selected from the group consisting of SEQ ID NOs: 18 to 24; and
iii) a nucleotide sequence encoding TnpB comprising the amino acid sequence represented by SEQ ID NO: 28, and reRNA with an additionally engineered scaffold sequence comprising any one nucleotide sequence selected from the group consisting of SEQ ID NOs: 60 to 70.

15. The vector of claim 12, comprising:
any one selected from the group consisting of the nucleotide sequences represented by SEQ ID NOs: 52 to 59 and SEQ ID NOs: 99 to 103.

16. The vector of claim 12, wherein the vector is any one selected from the group consisting of an adeno-associated virus vector, a lentivirus vector, a retrovirus vector, an adenovirus vector, and a herpes simplex virus vector.
